(19) United States
(12) Patent Application Publication
(10) Pub. No.: US 2009/0106852 A1
(43) Pub. Date: Apr. 23, 2009

(54) Influenza Therapeutic

(75) Inventors: Jianzhu Chen, Lexington, MA (US); Herman N. Eisen, Waban, MA (US); Qing Ge, Cambridge, MA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 11/952,056

(22) Filed: Dec. 6, 2007

Related U.S. Application Data
(63) Continuation of application No. 10/674,159, filed on Sep. 29, 2003.
(60) Provisional application No. 60/414,457, filed on Sep. 28, 2002.
(60) Provisional application No. 60/446,377, filed on Feb. 10, 2003.

(51) Int. Cl. A01K 67/027; C07H 21/02; A61K 31/7105; A61K 9/12; A61P 31/16; C12N 15/63; C12N 5/10

(52) U.S. Cl. 800/14; 536/24.5; 514/44; 536/23.1; 435/325; 800/13; 435/320.1; 424/45

(57) ABSTRACT

The present invention provides methods and compositions for inhibiting influenza infection and/or replication based on the phenomenon of RNA interference (RNAi) well as systems for identifying effective siRNAs and shRNAs for inhibiting influenza virus and systems for studying influenza virus infective mechanisms. The invention also provides methods and compositions for inhibiting infection, pathogenicity and/or replication of other infectious agents, particularly those that infect cells that are directly accessible from outside the body, e.g., skin cells or mucosal cells. In addition, the invention provides compositions comprising an RNAi-inducing entity, e.g., an siRNA, shRNA, or RNAi-inducing vector targeted to an influenza virus transcript and any of a variety of delivery agents. The invention further includes methods of use of the compositions for treatment of influenza.

### CROSS-REFERENCE TO RELATED APPLICATION

This application claims priority to U.S. Provisional Patent Application 60/414,457, filed Sep. 28, 2002, and U.S. Provisional Patent Application 60/446,377, filed Feb. 10, 2003. The contents of each of these applications is incorporated herein by reference.

### GOVERNMENT SUPPORT

The United States Government has provided grant support utilized in the development of the present invention. In particular, National Institutes of Health grant numbers 5-RO1-AI44477, 5—RO1-AI44478, 5-RO1-CA60686, and 1-RO1-AI50631 have supported development of this invention. The United States Government may have certain rights in the invention.

### BACKGROUND OF THE INVENTION

Influenza is one of the most widely spread infections worldwide. It can be deadly: an estimated 20 to 40 million people died during the 1918 influenza A virus pandemic. In the United States between 20 and 40 thousand people die from influenza A virus infection or its complications each year. During epidemics the number of influenza related hospitalizations may reach over 300,000 in a single winter season.

Several properties contribute to the epidemiological success of influenza virus. First, it is spread easily from person to person by aerosol (droplet infection). Second, small changes in influenza virus antigens are frequent (antigenic drift) so that the virus readily escapes protective immunity induced by a previous exposure to a different variant of the virus. Third, new strains of influenza virus can be easily generated by reassortment or mixing of genetic material between different strains (antigenic shift). In the case of influenza A virus, such mixing can occur between subtypes or strains that affect different species. The 1918 pandemic is thought to have been caused by a hybrid strain of virus derived from reassortment between a swine and a human influenza A virus.

Despite intensive efforts, there is still no effective therapy for influenza virus infection and existing vaccines are limited in value in part because of the properties of antigenic shift and drift described above. For these reasons, global surveillance of influenza A virus has been underway for many years, and the National Institutes of Health designates it as one of the top priority pathogens for biodefense. Although current vaccines based upon inactivated virus are able to prevent illness in approximately 70-80% of healthy individuals under age 65, this percentage is far lower in the elderly or immunocompromised. In addition, the expense and potential side effects associated with vaccine administration make this approach less than optimal. Although the four antiviral drugs currently approved in the United States for treatment and/or prophylaxis of influenza are helpful, their use is limited due to concerns about side effects, compliance, and possible emergence of resistant strains. Therefore, there remains a need for the development of effective therapies for the treatment and prevention of influenza infection.

### SUMMARY OF THE INVENTION

The present invention provides novel therapeutics for the treatment of influenza due to influenza virus types A, B, and C based on the phenomenon of RNA interference (RNAi). In particular, the invention provides short interfering RNA (siRNA) and/or short hairpin RNA (shRNA) molecules targeted to one or more target transcripts involved in virus production, virus replication, virus infection, and/or transcription of viral RNA, etc. In addition, the invention provides vectors whose presence within a cell results in transcription of one or more RNAs that self-hybridize or hybridize to each other to form an shRNA or siRNA that inhibits expression of at least one target transcript involved in virus production, virus infection, virus replication, and/or transcription of viral mRNA, etc.

The invention further provides a variety of compositions containing the siRNAs, shRNAs, and/or vectors of the invention. In certain embodiments of the invention the siRNA comprises two RNA strands having complementary regions so that the strands hybridize to each other to form a duplex structure approximately 19 nucleotides in length, wherein each of the strands optionally comprises a single-stranded overhang. In certain embodiments of the invention the shRNA comprises a single RNA molecule having complementary regions that hybridize to each other to form a hairpin (stem/loop) structure with a duplex portion approximately 19 nucleotides in length and a single-stranded loop. Such RNA molecules are said to self-hybridize. The shRNA may optionally include one or more unpaired portions at the 5′ and/or 3′ portion of the RNA. The invention further provides compositions comprising the inventive siRNAs, shRNAs, and/or vectors, and methods of delivery of such compositions.

Thus in one aspect, the invention provides an siRNA or shRNA targeted to a target transcript, wherein the target transcript is an agent-specific transcript, which transcript is involved in the production of, replication of, pathogenicity of, and/or infection by an infectious agent, and/or involved in transcription of agent-specific RNA. For purposes of description an siRNA or shRNA that inhibits expression of a target transcript involved in the production of, replication of, pathogenicity of, and/or infection by an infectious agent, thereby inhibiting production of, replication of, pathogenicity of, and/or infection by the infectious agent will be said to inhibit the infectious agent. According to certain embodiments of the invention the infectious agent is a virus. According to certain preferred embodiments of the invention the infectious agent is a virus that infects cells of the respiratory passages and/or lungs, e.g., respiratory epithelial cells, such as an influenza virus. According to certain embodiments of the invention the target transcript encodes a protein selected from the group consisting of: a polymerase, a nucleocapsid protein, a neuraminidase, a hemagglutinin, a matrix protein, and a nonstructural protein. According to certain embodiments of the invention the target transcript encodes an influenza virus protein selected from the group consisting of hemagglutinin, neuraminidase, membrane protein 1, membrane protein 2, nonstructural protein 1, nonstructural protein 2, polymerase protein PB1, polymerase protein PB2, polymerase protein PA, polymerase protein NP.

In another aspect, the invention provides a vector comprising a nucleic acid operably linked to expression signals (e.g., a promoter or promoter/enhancer) active in a cell so that, when the construct is introduced into the cell, an siRNA or shRNA is produced inside the host cell that is targeted to an agent-specific transcript, which transcript is involved in production of, replication of, and/or infection by an infectious agent, and/or transcription of agent-specific RNA. In certain embodiments of the invention the infectious agent is a virus, e.g., an influenza virus. In certain preferred embodiments of the invention the siRNA or shRNA inhibits influenza virus. The siRNA or shRNA may be targeted to any of the transcripts mentioned above. In general, the vector may be a DNA plasmid or a viral vector such as a retrovirus (e.g., a lentivirus), adenovirus, adeno-associated virus, etc. whose presence within a cell results in transcription of one or more ribonucleic acids (RNAs) that self-hybridize or hybridize to each other to form a short hairpin RNA (shRNA) or short interfering RNA (siRNA) that inhibits expression of at least one influenza virus transcript in the cell. In certain embodiments of the invention the vector comprises a nucleic acid segment operably linked to a promoter, so that transcription from the promoter (i.e., transcription directed by the promoter) results in synthesis of an RNA comprising complementary regions that hybridize to form an shRNA targeted to the target transcript. In certain embodiments of the invention the lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. The invention further provides compositions comprising the vector.

The invention also provides compositions comprising inventive siRNAs, shRNAs, and/or vectors described herein, wherein the composition further comprises any of a variety of substances (referred to herein as delivery agents) that facilitate delivery and/or uptake of the siRNA, shRNA, or vector. These substances include cationic polymers; peptide molecular transporters including arginine-rich peptides and histidine-rich peptides; cationic and neutral lipids; liposomes; certain non-cationic polymers; carbohydrates; and surfactant materials. The invention also encompasses the use of delivery agents that have been modified in any of a variety of ways, e.g., by addition of a delivery-enhancing moiety to the delivery agent.

In certain embodiments of the invention the delivery agent is modified in any of a number of ways to enhance stability, promote cellular uptake of the composition, promote release of siRNA, shRNA, and/or vectors within the cell, reduce cytotoxicity, or direct the composition to a particular cell type, tissue, or organ. For example, in certain embodiments of the invention the delivery agent is a modified cationic polymer (e.g., a cationic polymer substituted with one or more groups selected to reduce the cationic nature of the polymer and thereby reduce cytotoxicity). In certain embodiments of the invention the delivery agent comprises a delivery-enhancing moiety such as an antibody, antibody fragment, or ligand that specifically binds to a molecule that is present on the surface of a cell such as a respiratory epithelial cell.

The present invention further provides methods of treating or preventing infectious diseases, particularly infectious diseases of the respiratory system, e.g., influenza, by administering any of the inventive compositions to a subject within an appropriate time window prior to exposure to the infectious agent, while exposure is occurring, or following exposure, or at any point during which a subject exhibits symptoms of a disease caused by the infectious agent. The siRNAs or shRNAs may be chemically synthesized, produced using in vitro transcription, synthesized in vitro, produced intracellularly, etc. The compositions may be administered by a variety of routes including intravenous, inhalation, intranasally, as an aerosol, intraperitoneally, intramuscularly, intradermally, orally, etc.

The invention provides additional methods of treating or preventing a disease caused by an infectious agent, e.g., a disease caused by influenza virus, employing gene therapy. According to certain of these methods cells (either infected or noninfected) are engineered or manipulated to synthesize inventive siRNAs or shRNAs. According to certain embodiments of the invention the cells are engineered to contain a vector whose presence within the cell results in synthesis of one or more RNAs that hybridize with each other or self-hybridize within the cell to form one or more siRNAs or shRNAs targeted to an appropriate agent-specific target transcript. The cells may be engineered in vitro or while present within the subject to be treated, e.g., within the respiratory passages of the subject.

In another aspect, the invention provides methods for selecting and designing preferred siRNA or shRNA sequences to inhibit an infectious agent. The invention provides methods of selecting and designing siRNAs and shRNAs to inhibit infectious agents characterized in that multiple different strains or variants of the infectious agent exist, in particular wherein strain variation can occur by genetic reassortment or mixing. These methods find particular use in selecting and designing siRNA and shRNA sequences to combat infectious agents whose genomes consist of multiple different segments, wherein genetic reassortment can occur rapidly and unpredictably by substitution of an entire genomic segment from one subtype to another. These aspects of the invention are therefore particularly suited for infectious agents whose genome consists of multiple independent segments, meaning that the genome consists of physically distinct nucleic acid molecules that are not covalently joined to one another. The invention may also find particular utility for infectious agents that exchange genetic information by transfer of plasmids, e.g., plasmids encoding genes that confer resistance to therapeutic compounds.

The present invention also provides a system for identifying compositions comprising one or more RNAi-inducing entities such as siRNAs and/or shRNAs targeted to an influenza virus transcript, and/or comprising vector(s) whose presence within a cell results in production of one or more RNAs that hybridize with each other or self-hybridize to form an siRNA or shRNA that is targeted to an influenza virus transcript, wherein the compositions are useful for the inhibition of influenza virus.

The present invention further provides a system for the analysis and characterization of the mechanism of influenza replication and/or transcription of influenza virus RNAS, as well as for the characterization and analysis of relevant viral components involved in the viral life cycle.

In another aspect, the invention provides methods for designing siRNAs and/or shRNAs to inhibit an infectious agent in cases where multiple variants of the infectious agent exist. For example, the invention provides a method for designing an siRNA or shRNA molecule having a duplex portion, the method comprising steps of (i) identifying a portion of a target transcript, which portion is highly conserved among a plurality of variants of an infectious agent and comprises at least 15 consecutive nucleotides; and (ii) selecting an siRNA or shRNA, wherein the sense strand of the siRNA or the sense portion of the shRNA comprises the highly conserved sequence.

In another aspect, the invention provides siRNAs and siRNAs and methods for design thereof, wherein the siRNA or shRNA is targeted to a transcript whose inhibition results in inhibition of multiple (or all) other viral transcripts. In particular, the invention provides siRNA and shRNA compositions comprising siRNAs or shRNAs targeted to transcripts encoding viral polymerase (DNA or RNA polymerase) or nucleocapsid proteins.

This application refers to various patents, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001.

### BRIEF DESCRIPTION OF THE DRAWING

FIG. 1A, adapted from Julkunen, I., et al., referenced elsewhere herein, presents a schematic of the influenza virus.

FIG. 1B, adapted from Fields' Virology, referenced elsewhere herein, shows the genome structure of the influenza virus and the transcripts derived from the influenza genome. Thin lines at the 5′ and 3′-termini of the mRNAs represent untranslated regions. Shaded or hatched areas represent coding regions in the 0 or +1 reading frames, respectively. Introns are depicted by V-shaped lines. Small rectangles at the 5′ ends of the mRNAs represent heterogenous cellular RNAs covalently linked to the viral nucleic acids. A₍ₙ₎ symbolizes the polyA tail.

FIG. 2, adapted from Julkunen, I., et al., referenced elsewhere herein, shows the influenza virus replication cycle.

FIG. 3 shows the structure of siRNAs observed in the Drosophila system.

FIG. 4 presents a schematic representation of the steps involved in RNA interference in Drosophila.

FIG. 5 shows a variety of exemplary siRNA and shRNA structures useful in accordance with the present invention.

FIG. 6 presents a representation of an alternative inhibitory pathway, in which the DICER enzyme cleaves a substrate having a base mismatch in the stem to generate an inhibitory product that binds to the 3′ UTR of a target transcript and inhibits translation.

FIG. 7 presents one example of a construct that may be used to direct transcription of both strands of an inventive siRNA.

FIG. 8 depicts one example of a construct that may be used to direct transcript of a single RNA molecule that hybridizes to form an shRNA in accordance with the present invention.

FIG. 9 shows a sequence comparison between six strains of influenza virus A that have a human host of origin. Dark shaded areas were used to design siRNAs that were tested as described in Example 2. The base sequence is the sequence of strain A/Puerto Rico/8/34. Lightly shaded letters indicate nucleotides that differ from the base sequence.

FIG. 10 shows a sequence comparison between two strains of influenza virus that have a human host of origin and five strains of influenza virus A that have an animal host of origin. Darkly shaded areas were used to design siRNAs that were tested as described in Example 2. The base sequence is the sequence of strain A/Puerto Rico/8/34. Lightly shaded letters indicate nucleotides that differ from the base sequence.

FIGS. 11A-11F show the results of experiments indicating that siRNA inhibits influenza virus production in MDCK cells. Six different siRNAs that target various viral transcripts were introduced into MDCK cells by electroporation, and cells were infected with virus 8 hours later. FIG. 11A is a time course showing viral titer in culture supernatants as measured by hemagglutinin assay at various times following infection with viral strain A/PR/8/34 (H1N1) (PR8), at a multiplicity of infection (MOI) of 0.01 in the presence or absence of the various siRNAs or a control siRNA. FIG. 11B is a time course showing viral titer in culture supernatants as measured by hemagglutinin assay at various times following infection with influenza virus strain A/WSN/33 (H1N1) (WSN) at an MOI of 0.01 in the presence or absence of the various siRNAs or a control siRNA. FIG. 11C shows a plaque assay showing viral titer in culture supernatants from virus infected cells that were either mock transfected or transfected with siRNA NP-1496. FIG. 11D shows inhibition of influenza virus production at different doses of siRNA. MDCK cells were transfected with the indicated amount of NP-1496 siRNA followed by infection with PR8 virus at an MOI of 0.01. Virus titer was measured 48 hours after infection. Representative data from one of two experiments are shown. FIG. 11E shows inhibition of influenza virus production by siRNA administered after virus infection. MDCK cells were infected with PR8 virus at an MOI of 0.01 for 2 hrs and then transfected with NP-1496 (2.5 mmol). Virus titer was measured at the indicated times after infection. Representative data from one of two experiments are shown.

FIG. 12 shows a sequence comparison between a portion of the 3′ region of NP sequences among twelve influenza A virus subtypes or isolates that have either a human or animal host of origin. The shaded area was used to design siRNAs that were tested as described in Examples 2 and 3. The base sequence is the sequence of strain A/Puerto Rico/8/34. Shaded letters indicate nucleotides that differ from the base sequence.

FIG. 13 shows positions of various siRNAs relative to influenza virus gene segments, correlated with effectiveness in inhibiting influenza virus.

FIG. 14A is a schematic of a developing chicken embryo indicating the area for injection of siRNA and siRNA/delivery agent compositions.

FIG. 14B shows the ability of various siRNAs to inhibit influenza virus production in developing chicken embryos.

FIG. 15 is a schematic showing the interaction of nucleoprotein with viral RNA molecules.

FIGS. 16A and 16B show schematic diagrams illustrating the differences between influenza virus vRNA, mRNA, and cRNA (template RNA) and the relationships between them. The conserved 12 nucleotides at the 3′ end and 13 nucleotides at the 5′ end of each influenza A virus vRNA segment are indicated in FIG. 16B. The mRNAs contain an m⁷ GpppN^{m} cap structure and, on average, 10 to 13 nucleotides derived from a subset of host cell RNAs. Polyadenylation of the mRNAs occurs at a site in the mRNA corresponding to a location 15 to 22 nucleotides before the 5′ end of the vRNA segment. Arrows indicate the positions of primers specific for each RNA species. (Adapted from ref. (1)).

FIG. 17 shows amounts of viral NP and NS RNA species at various times following infection with virus, in cells that were mock transfected or transfected with siRNA NP-1496 6-8 hours prior to infection.

FIG. 18A shows that inhibition of influenza virus production requires a wild type (wt) antisense strand in the duplex siRNA. MDCK cells were first transfected with siRNAs formed from wt and modified (m) strands and infected 8 hrs later with PR8 virus at MOI of 0.1. Virus titers in the culture supernatants were assayed 24 hrs after infection. Representative data from one of the two experiments are shown. FIG. 18B shows that M-specific siRNA inhibits the accumulation of specific mRNA. MDCK cells were transfected with M-37, infected with PR8 virus at MOI of 0.01, and harvested for RNA isolation 1, 2, and 3 hrs after infection. The levels of M-specific mRNA, cRNA, and vRNA were measured by reverse transcription using RNA-specific primers, followed by real time PCR. The level of each viral RNA species is normalized to the level of γ-actin mRNA (bottom panel) in the same sample. The relative levels of RNAs are shown as mean value±S.D. Representative data from one of the two experiments are shown.

FIGS. 19A-D show that NP-specific siRNA inhibits the accumulation of not only NP- but also M- and NS-specific mRNA, vRNA, and cRNA. MDCK (A-C) and Vero (D) cells were transfected with NP-1496, infected with PR8 virus at MOI of 0.1, and harvested for RNA isolation 1, 2, and 3 hrs after infection. The levels of mRNA, cRNA, and vRNA specific for NP, M, and NS were measured by reverse transcription using RNA-specific primers followed by real time PCR. The level of each viral RNA species is normalized to the level of γ-actin mRNA (not shown) in the same sample. The relative levels of RNAs are shown. Representative data from one of three experiments are shown.

FIGS. 19E-G, right side in each figure, show that PA-specific siRNA inhibits the accumulation of not only PA- but also M- and NS-specific mRNA, vRNA, and cRNA. MDCK cells were transfected with PA-1496, infected with PR8 virus at MOI of 0.1, and harvested for RNA isolation 1, 2, and 3 hrs after infection. The levels of mRNA, cRNA, and vRNA specific for PA, M, and NS were measured by reverse transcription using RNA-specific primers followed by real time PCR. The level of each viral RNA species is normalized to the level of γ-actin mRNA (not shown) in the same sample. The relative levels of RNAs are shown.

FIG. 19H shows that NP-specific siRNA inhibits the accumulation of PB1-(top panel), PB2-(middle panel) and PA- (lower panel) specific mRNA. MDCK cells were transfected with NP-1496, infected with PR8 virus at MOI of 0.1, and harvested for RNA isolation 1, 2, and 3 hrs after infection. The levels of mRNA specific for PB1, PB2, and PA mRNA were measured by reverse transcription using RNA-specific primers followed by real time PCR. The level of each viral RNA species is normalized to the level of γ-actin mRNA (not shown) in the same sample. The relative levels of RNAs are shown.

FIG. 20A shows sequences of siRNA CD8-61 and its hairpin derivative CD8-61F.

FIG. 20B shows inhibition of CD8α expression by CD8-61 and CD8-61F. A CD8⁺ CD4⁺ T cell line was transfected with either CD8-61 or CD8-61F by electroporation. CD8α expression was assayed by flow cytometry 48 hrs later. Unlabeled line, mock transfection.

FIG. 20C shows a schematic diagram of the pSLOOP III vector, in which expression of CD8-61F hairpin RNA is driven by H1 RNA pol III promoter. Terminator, termination signal sequence.

FIG. 20D presents plots showing silencing of CD8α in HeLa cells using pSLOOP III. Untransfected cells did not express CD8α. Cells were transfected with the CD8α expression vector and either a promoterless pSLOOP III-CD8-61F construct, synthetic siRNA, or a pSLOOP III-CD8-61F containing a promoter.

FIG. 21A shows schematic diagrams of NP-1496 and GFP-949 siRNA and their hairpin derivatives/precursors.

FIG. 21B shows tandem arrays of NP-1496H and GFP-949H in two different orders.

FIG. 21C shows pSLOOP III expression vectors. Hairpin precursors of siRNA are cloned in the pSLOOP III vector alone (top), in tandem arrays (middle), or simultaneously with independent promoter and termination sequence (bottom).

FIG. 22A is a plot showing that siRNA inhibits influenza virus production in mice when administered together with the cationic polymer PEI prior to infection with influenza virus. Filled squares (no treatment); Open squares (GFP siRNA); Open circles (30 μg NP siRNA); Filled circles (60 μg NP siRNA). Each symbol represents an individual animal. p values between different groups are shown.

FIG. 22B is a plot showing that siRNA inhibits influenza virus production in mice when administered together with the cationic polymer PLL prior to infection with influenza virus. Filled squares (no treatment); Open squares (GFP siRNA); Filled circles (60 μg NP siRNA). Each symbol represents an individual animal. p values between different groups are shown.

FIG. 22C is a plot showing that siRNA inhibits influenza virus production in mice when administered together with the cationic polymer jetPEI prior to infection with influenza virus significantly more effectively than when administered in PBS. Open squares (no treatment); Open triangles (GFP siRNA in PBS); Filled triangles (NP siRNA in PBS); Open circles (GFP siRNA with jetPEI); Filled circles (NP siRNA with jetPEI). Each symbol represents an individual animal. p values between different groups are shown.

FIG. 23 is a plot showing that siRNAs targeted to influenza virus NP and PA transcripts exhibit an additive effect when administered together prior to infection with influenza virus. Filled squares (no treatment); Open circles (60 μg NP siRNA); Open triangles (60 μg PA siRNA); Filled circles (60 μg NP siRNA+60 μg PA siRNA). Each symbol represents an individual animal. p values between different groups are shown.

FIG. 24 is a plot showing that siRNA inhibits influenza virus production in mice when administered following infection with influenza virus. Filled squares (no treatment); Open squares (60 μg GFP siRNA); Open triangles (60 μg PA siRNA); Open circles (60 μg NP siRNA); Filled circles (60 μg NP+60 μg PA siRNA). Each symbol represents an individual animal. p values between different groups are shown.

FIG. 25A is a schematic diagram of a lentiviral vector expressing a shRNA. Transcription of shRNA is driven by the U6 promoter. EGFP expression is driven by the CMV promoter. SIN-LTR, Ψ, cPPT, and WRE are lentivirus components. The sequence of NP-1496 shRNA is shown.

FIG. 25B presents plots of flow cytometry results demonstrating that Vero cells infected with the lentivirus depicted in FIG. 25B express EGFP in a dose-dependent manner. Lentivirus was produced by co-transfecting DNA vector encoding NP-1496a shRNA and packaging vectors into 293T cells. Culture supernatants (0.25 ml or 1.0 ml) were used to infect Vero cells. The resulting Vero cell lines (Vero-NP-0.25 and Vero-NP-1.0) and control (uninfected) Vero cells were analyzed for GFP expression by flow cytometry. Mean fluorescence intensity of Vero-NP-0.25 (upper portion of figure) and Vero-NP-1.0 (lower portion of figure) cells are shown. The shaded curve represents mean fluorescence intensity of control (uninfected) Vero cells.

FIG. 25C is a plot showing inhibition of influenza virus production in Vero cells that express NP-1496 shRNA. Parental and NP-1496 shRNA expressing Vero cells were infected with PR8 virus at MOI of 0.04, 0.2 and 1. Virus titers in the supernatants were determined by hemagglutination (HA) assay 48 hrs after infection.

FIG. 26 is a plot showing that influenza virus production in mice is inhibited by administration of DNA vectors that express siRNA targeted to influenza virus transcripts. Sixty μg of DNA encoding RSV, NP-1496 (NP) or PB1-2257 (PB1) shRNA were mixed with 40 μl Infasurf and were administered into mice by instillation. For no treatment (NT) group, mice were instilled with 60 μl of 5% glucose. Thirteen hrs later, the mice were infected intranasally with PR8 virus, 12000 pfu per mouse. The virus titers in the lungs were measured 24 hrs after infection by MDCK/hemagglutinin assay. Each data point represents one mouse. p values between groups are indicated.

FIG. 27A shows results of an electrophoretic mobility shift assay for detecting complex formation between siRNA and poly-L-lysine (PLL). SiRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 27B shows results of an electrophoretic mobility shift assay for detecting complex formation between siRNA and poly-L-arginine (PLA). SiRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 28A is a plot showing cytotoxicity of siRNA/PLL complexes. Vero cells in 96-well plates were treated with siRNA (400 pmol)/polymer complexes for 6 hrs. The polymer-containing medium was then replaced with DMEM-10% FCS. The metabolic activity of the cells was measured 24 h later by using the MTT assay. Squares=PLL (MW ˜8K); Circles=PLL (MW ˜42K) Filled squares=25%; Open triangles=50%; Filled triangles=75%; X=95%. The data are shown as the average of triplicates.

FIG. 28B is a plot showing cytotoxicity of siRNA/PLA complexes. Vero cells in 96-well plates were treated with siRNA (400 pmol)/polymer complexes for 6 hrs. The polymer-containing medium was then replaced with DMEM-10% FCS. The metabolic activity of the cells was measured 24 h later by using the MTT assay. The data are shown as the average of triplicates.

FIG. 29A is a plot showing that PLL stimulates cellular uptake of siRNA. Vero cells in 24-well plates were incubated with Lipofectamine+siRNA (400 pmol) or with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. Open circles=no treatment; Filled squares=Lipofectamine; Filled triangles=PLL (MW ˜42K); Open triangles=PLL (MW ˜8K).

FIG. 29B is a plot showing that poly-L-arginine stimulates cellular uptake of siRNA. Vero cells in 24-well plates were incubated with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. 0, 25, 50, 75, and 95% refer to percentage of ε-amino groups on PLL substituted with imidazole acetyl groups. Closed circles=no transfection; Open circles=Lipofectamine; Open and filled squares=0% and 25% (Note that the data points for 0% and 25% are identical); Filled triangles=50%; Open triangles=75%; X=95%.

### ABBREVIATIONS

DNA: deoxyribonucleic acid

RNA: ribonucleic acid

vRNA: virion RNA in the influenza virus genome, negative strand

cRNA: complementary RNA, a direct transcript of vRNA, positive strand

mRNA: messenger RNA transcribed from vRNA or cellular genes, a template for protein synthesis

dsRNA: double-stranded RNA

siRNA: short interfering RNA

shRNA: short hairpin RNA

RNAi: RNA interference

### DEFINITIONS

In general, the term antibody refers to an immunoglobulin, whether natural or wholly or partially synthetically produced. In certain embodiments of the invention the term also encompasses any protein comprising a immunoglobulin binding domain. These proteins may be derived from natural sources, or partly or wholly synthetically produced. The antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. In certain embodiments of the invention the term includes “humanized” antibodies in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. An antibody may be polyclonal or monoclonal, though for purposes of the present invention monoclonal antibodies are generally preferred.

As used herein, the terms approximately or about in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The term hybridize, as used herein, refers to the interaction between two complementary nucleic acid sequences. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.; high stringency hybridization (e.g., 6×SSC at about 45° C., followed by one or more washes in 0.2×SSC, 0.1% SDS at 65° C.; and very high stringency hybridization conditions (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based upon various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

The term influenza virus is used here to refer to any strain of influenza virus that is capable of causing disease in an animal or human subject, or that is an interesting candidate for experimental analysis. Influenza viruses are described in Fields, B., et al., Fields' Virology, 4^{th} ed., Philadelphia: Lippincott Williams and Wilkins; ISBN: 0781718325, 2001. In particular, the term encompasses any strain of influenza A virus that is capable of causing disease in an animal or human subject, or that is an interesting candidate for experimental analysis. A large number of influenza A isolates have been partially or completely sequenced. Appendix A presents merely a partial list of complete sequences for influenza A genome segments that have been deposited in a public database (The Influenza Sequence Database (ISD), see Macken, C., Lu, H., Goodman, J., & Boykin, L., “The value of a database in surveillance and vaccine selection.” in Options for the Control of Influenza IV. A.D.M.E. Osterhaus, N. Cox & A. W. Hampson (Eds.) Amsterdam: Elsevier Science, 2001, 103-106). This database also contains complete sequences for influenza B and C genome segments. The database is available on the World Wide Web at the Web site having URL http://www.flu.lanl.gov/ along with a convenient search engine that allows the user to search by genome segment, by species infected by the virus, and by year of isolation. Influenza sequences are also available on Genbank. Sequences of influenza genes are therefore readily available to, or determinable by, those of ordinary skill in the art.

Isolated, as used herein, means 1) separated from at least some of the components with which it is usually associated in nature; 2) prepared or purified by a process that involves the hand of man; and/or 3) not occurring in nature.

Ligand, as used herein, means a molecule that specifically binds to a second molecule, typically a polypeptide or portion thereof, such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

Operably linked, as used herein, refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term regulatory sequence is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc. Alternately, the regulatory sequence may comprise a promoter and/or enhancer that is active in epithelial cells in the nasal passages, respiratory tract and/or the lungs.

As used herein, the term RNAi-inducing entity encompasses RNA molecules and vectors (other than naturally occurring molecules not modified by the hand of man) whose presence within a cell results in RNAi and leads to reduced expression of a transcript to which the RNAi-inducing entity is targeted. The term specifically includes siRNA, shRNA, and RNAi-inducing vectors.

As used herein, an RNAi-inducing vector is a vector whose presence within a cell results in transcription of one or more RNAs that self-hybridize or hybridize to each other to form an shRNA or siRNA. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector provides a template for intracellular synthesis of the RNA or RNAs or precursors thereof. For purposes of inducing RNAi, presence of a viral genome into a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an siRNA or shRNA that is targeted to the transcript, i.e., if presence of the vector within a cell results in production of one or more siRNAs or shRNAs targeted to the transcript.

A short, interfering RNA (siRNA) comprises an RNA duplex that is approximately 19 basepairs long and optionally further comprises one or two single-stranded overhangs. An siRNA may be formed from two RNA molecules that hybridize together, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

The term short hairpin RNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

As used herein, the term specific binding refers to an interaction between a target polypeptide (or, more generally, a target molecule) and a binding molecule such as an antibody, ligand, agonist, or antagonist. The interaction is typically dependent upon the presence of a particular structural feature of the target polypeptide such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

The term subject, as used herein, refers to an individual susceptible to infection with an infectious agent, e.g., an individual susceptible to infection with a virus such as the influenza virus. The term includes birds and animals, e.g., domesticated birds and animals (such as chickens, mammals, including swine, horse, dogs, cats, etc.), and wild animals, non-human primates, and humans.

An siRNA or shRNA or an siRNA or shRNA sequence is considered to be targeted to a target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. An RNA-inducing vector whose presence within a cell results in production of an siRNA or shRNA that is targeted to a transcript is also considered to be targeted to the target transcript. Since the effect of targeting a transcript is to reduce or inhibit expression of the gene that directs synthesis of the transcript, an siRNA or shRNA targeted to a transcript is also considered to target the gene that directs synthesis of the transcript even though the gene itself (i.e., genomic DNA) is not thought to interact with the siRNA, shRNA, or components of the cellular silencing machinery. Thus as used herein, an siRNA, shRNA, or RNAi-inducing vector that targets a transcript is understood to target the gene that provides a template for synthesis of the transcript.

As used herein, treating includes reversing, alleviating, inhibiting the progress of, preventing, or reducing the likelihood of the disease, disorder, or condition to which such term applies, or one or more symptoms or manifestations of such disease, disorder or condition.

In general, the term vector refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

### DETAILED DESCRIPTION OF CERTAIN PREFERRED EMBODIMENTS OF THE INVENTION

I. Influenza Viral Life Cycle and Characteristics

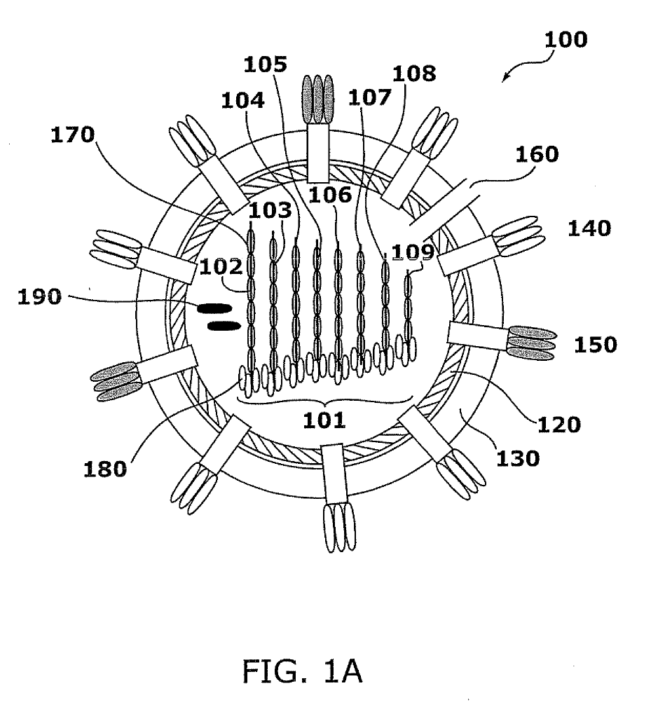

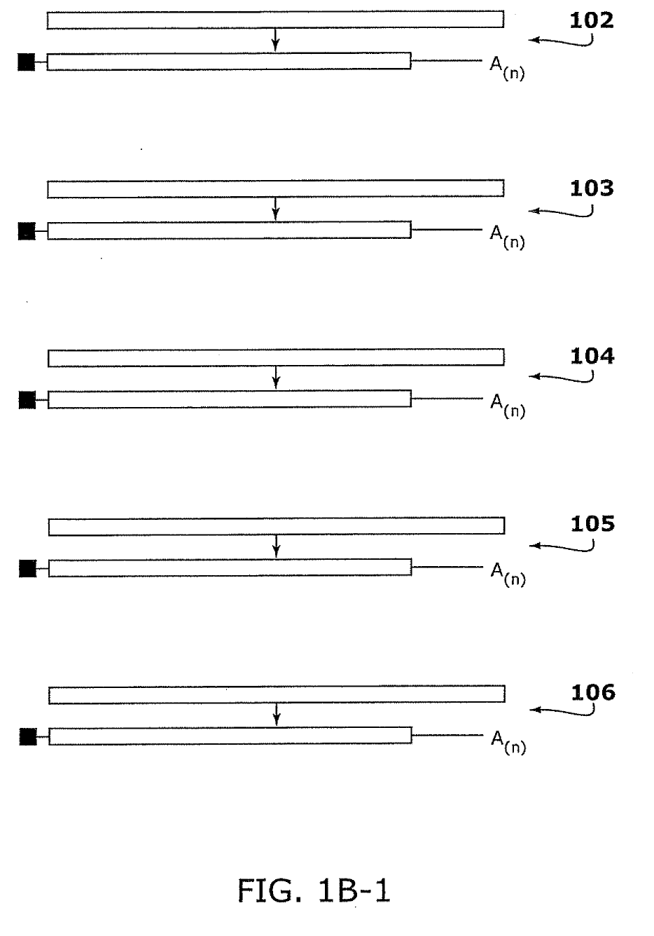

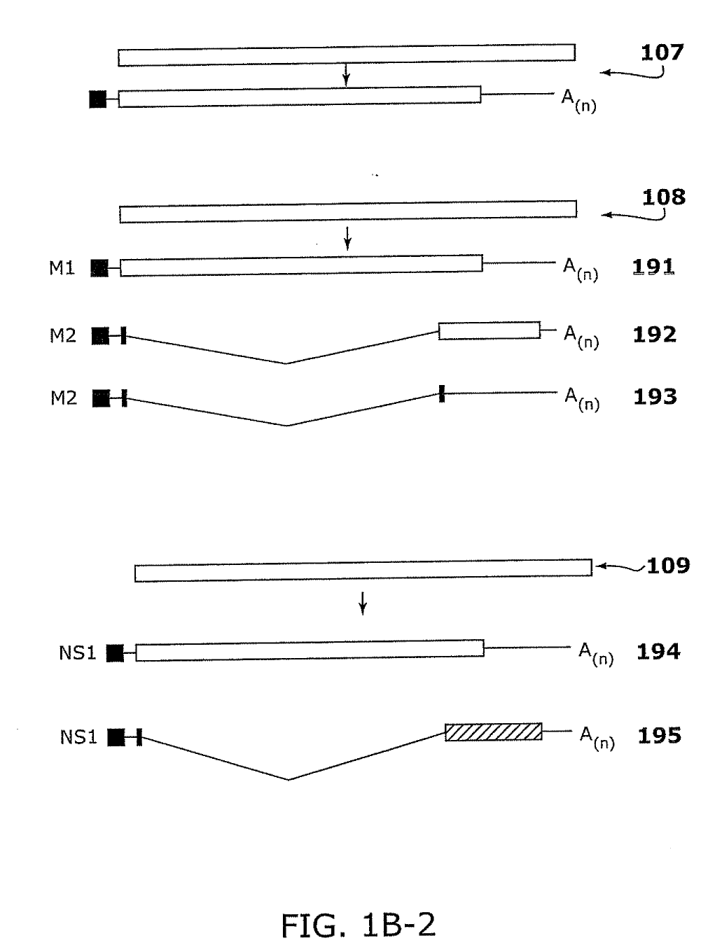

Influenza viruses are enveloped, negative-stranded RNA viruses of the Orthomyxoviridae family. They are classified as influenza types A, B, and C, of which influenza A is the most pathogenic and is believed to be the only type able to undergo reassortment with animal strains. Influenza types A, B, and C can be distinguished by differences in their nucleoprotein and matrix proteins (see FIG. 1). As discussed further below, influenza A subtypes are defined by variation in their hemagglutinin (HA) and neuraminidase (NA) genes and usually distinguished by antibodies that bind to the corresponding proteins.

The influenza A viral genome consists of ten genes distributed in eight RNA segments. The genes encode 10 proteins: the envelope glycoproteins hemagglutinin (HA) and neuraminidase (NA); matrix protein (M1); nucleoprotein (NP); three polymerases (PB1, PB2, and PA) which are components of an RNA-dependent RNA transcriptase also referred to as a polymerase or polymerase complex herein; ion channel protein (M2), and nonstructural proteins (NS1 and NS2). See Julkunen, I., et al., Cytokine and Growth Factor Reviews, 12: 171-180, 2001 for further details regarding the influenza A virus and its molecular pathogenesis. See also Fields, B., et al., Fields' Virology, 4^{th}. ed., Philadelphia: Lippincott Williams and Wilkins; ISBN: 0781718325, 2001. The organization of the influenza B viral genome is extremely similar to that of influenza A whereas the influenza C viral genome contains seven RNA segments and lacks the NA gene.

Influenza A virus classification is based on the hemagglutinin (H1-H15) and neuraminidase (N1-N9) genes. World Health Organization (WHO) nomenclature defines each virus strain by its animal host of origin (specified unless human), geographical origin, strain number, year of isolation, and antigenic description of HA and NA. For example, A/Puerto Rico/8/34 (H1N1) designates strain A, isolate 8, that arose in humans in Puerto Rico in 1934 and has antigenic subtypes 1 of HA and NA. As another example, A/Chicken/Hong Kong/258/97 (H5N₁) designates strain A, isolate 258, that arose in chickens in Hong Kong in 1997 and has antigenic subtype 5 of HA and 1 of NA. Human epidemics have been caused by viruses with HA types H1, H2, and H3 and NA types N1 and N2.

As mentioned above, genetic variation occurs by two primary mechanisms in influenza virus A. Genetic drift occurs via point mutations, which often occur at antigenically significant positions due to selective pressure from host immune responses, and genetic shift (also referred to as reassortment), involving substitution of a whole viral genome segment of one subtype by another. Many different types of animal species including humans, swine, birds, horses, aquatic mammals, and others, may become infected with influenza A viruses. Some influenza A viruses are restricted to a particular species and will not normally infect a different species. However, some influenza A viruses may infect several different animal species, principally birds (particularly migratory water fowl), swine, and humans. This capacity is considered to be responsible for major antigenic shifts in influenza A virus. For example, suppose a swine becomes infected with an influenza A virus from a human and at the same time becomes infected with a different influenza A virus from a duck. When the two different viruses reproduce in the swine cells, the genes of the human strain and duck strain may “mix,” resulting in a new virus with a unique combination of RNA segments. This process is called genetic reassortment. (Note that this type of genetic reassortment is distinct from the exchange of genetic information that occurs between chromosomes during meiosis.)

Like other viruses and certain bacterial species, influenza viruses replicate intracellularly. Influenza A viruses replicate in epithelial cells of the upper respiratory tract. However, monocytes/macrophages and other white blood cells can also be infected. Numerous other cell types with cell surface glycoproteins containing sialic acid are susceptible to infection in vitro since the virus uses these molecules as a receptor.

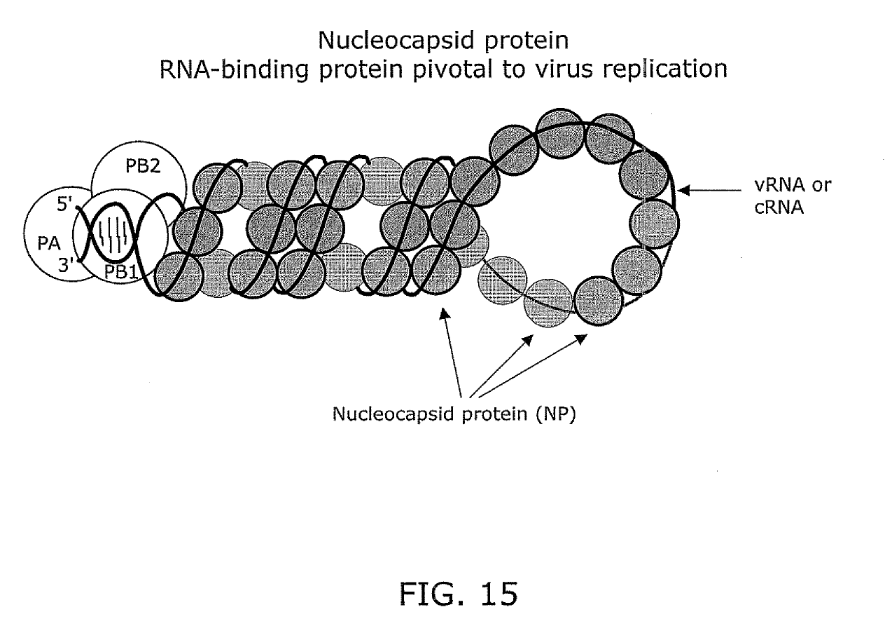

The influenza A infection/replication cycle is depicted schematically in FIG. 1. As shown in FIG. 1A, the influenza A virion 100 comprises genome 101, consisting of eight negative stranded RNA segments: PB2 (102), PB1 (103), PA (104), HA (105), NP (106), NA (107), M (108), and NS (109). There are conventionally numbered from 1 to 8, with PB2=1, PB1=2, PA=3, HA=4, NP=5, NA=6, M=7, and NS=8. The genomic RNA segments are packaged inside a layer of membrane protein M1 120 which is surrounded by a lipid bilayer 130 from which the extracellular domains of the envelope glycoproteins HA 140 and NA 150 and the ion channel M2 160 protrude. RNA segments 102-108 are covered with nucleoprotein MP 170 (depicted schematically in more detail in FIG. 15) and contain the viral polymerase complex 180 consisting of polymerases PB1, PB2, and PA. Nonstructural protein NS2 190 is also found within virions. Nonstructural protein NS1 (not shown) is found within infected cells.

FIG. 1B shows the genome structure of the influenza virus and the transcripts generated from the influenza genome (not drawn to scale). Six of the eight genomic RNA segments (PB1 (102), PB2 (103), PA (104), HA (105), NP (106), and NA (107)) each serve as template for a single, unspliced transcript that encodes the corresponding protein. Three mRNA transcripts have been identified as being derived from influenza virus A segment M (108): a colinear transcript 191 that encodes the M₁ protein, a spliced mRNA 192 that encodes the M₂ protein and contains a 689 nucleotide intron, and another alternatively spliced mRNA 193 that has the potential to encode a 9 amino acid peptide (M3) that has not been detected in virus-infected cells. Two mRNA transcripts are derived from influenza virus A segment NS: an unspliced mRNA 194 that encodes the NS₁ protein and a spliced mRNA 195 that encodes the NS₂ protein and includes a 473 nucleotide intron.

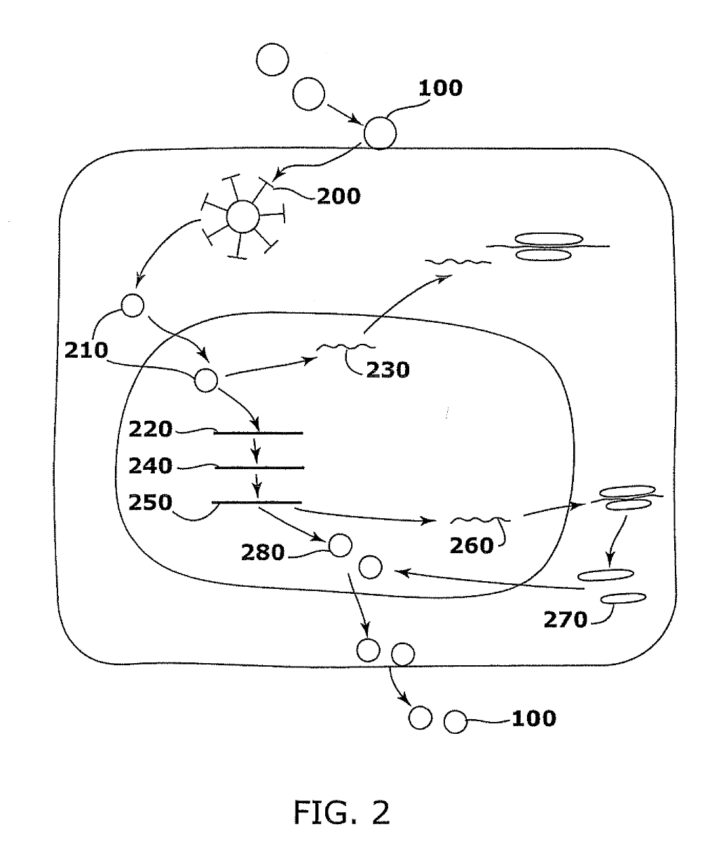

The infective cycle (FIG. 2) begins when the virion 100 attaches via its hemagglutinin to the surface of a susceptible cell through interaction with a sialic acid containing cell surface protein. Attached virus is endocytosed into coated vesicles 200 via clathrin-dependent endocytosis. Low pH in endosomes triggers fusion of viral and endosomal membranes, resulting in liberation of viral ribonucleoprotein (vRNP) compexes (nucleocapsids) 210 into the cytoplasm. Viral nucleocapsids are imported into the cell nucleus, following which primary viral mRNA synthesis is initiated by a viral RNA polymerase complex that consists of the PB1, PB2, and PA polymerases. Primers produced by the endonuclease activity of the PB2 protein on host cell pre-mRNA is used to initiate viral mRNA synthesis using viral RNA (vRNA) 220 as a template. PB1 protein catalyzes the synthesis of virus specific mRNAs 230, which are transported into the cytoplasm and translated.

Newly synthesized polymerases NP, NS₁ and NS₂ are transported into the nucleus and regulate replication and secondary viral mRNA synthesis. Synthesis of complementary RNA (cRNA) 240 from viral RNA (vRNA) is initiated by PB1, PB2, PA, and NP, after which new vRNA molecules 250 are synthesized. The viral polymerase complex uses these vRNAs as templates for synthesis of secondary mRNA 260. Thus transcription of vRNA by the virus-encoded transcriptase produces mRNA that serves as a template for synthesis of viral proteins and also produces complementary RNA (cRNA), which differs from mRNA by lacking the 5′ cap and the 3′ poly A tail, and serves as a template for synthesizing more vRNA for new virion production. Late in infection NS₁ protein regulates splicing of M and NS mRNAs, which results in production of M2 and NS2 mRNAs. Viral mRNAs are transported into the cytoplasm, where viral structural proteins 270 are produced. Proteins PB1, PB2, PA, and NP are transported into the nucleus, the site of assembly of vRNP complexes (nucleocapsids) 280. M1 and NS2 proteins are also transported into the nucleus, where they interact with vRNPs and regulate their nuclear export. Viral vRNA-M1 protein complexes interact with the cytoplasmic portion of HA and NA molecules at the plasma membrane, where budding of mature virions and release of viral particles occur.

Influenza A virus replicates rapidly in cells, resulting in host cell death due to cytolytic effects or apoptosis. Infection causes changes in a wide variety of cellular activities and processes including inhibition of host cell gene expression. The viral polymerase complex binds to and cleaves newly synthesized cellular polymerase II transcripts in the nucleus. NS1 protein blocks cellular pre-mRNA splicing and inhibits nuclear export of host mRNA. Translation of cellular mRNA is greatly inhibited, whereas viral mRNA is efficiently translated. Maintenance of efficient translation of viral mRNAs is achieved in part through viral downregulation of the cellular interferon (IFN) response, a host response which typically acts to inhibit translation in virally infected cells. In particular, viral NS1 protein binds to IFN-induced PKR and inhibits its activity. Thus it is evident that infection with influenza virus results in profound changes in cellular biosynthesis, including changes in the processing and translation of cellular mRNA.

Infected cells respond in a number of ways to limit spread of the virus. Several transcription factor systems are activated, including nuclear factor kappa B (NFκB), activating protein (AP)-1, interferon regulatory factors, signal transducers and activators of transcription (STATs), and nuclear factor-IL-6, among others. Activation of these transcription factor pathways leads to production of chemotactic, proinflammatory, and antiviral cytokines that stimulate migration of inflammatory cells to the site of infection, exert a number of antiviral effects, and play a role in the immune response to viral infection. Type I (IFN-α/β), RANTES, MCP-1, and IL-8 are among the cytokines produced by influenza A virus infected epithelial cells. Influenza A virus infected monocyte/macrophages produce a variety of additional cytokines including MIP-1 α/β, MIP-3 cc, MCP-1, MCP-3, IP-10, IL-1β, IL-6, TNF-α, and IL-18.

Cytolytic death of cells generally occurs approximately 20-40 hours following infection with influenza A virus as a consequence of viral replication, production of viral particles, continued viral protein synthesis and shutdown of host protein synthesis. Changes characteristic of apoptosis, e.g., chromatin condensation, DNA fragmentation, cell shrinkage, and clearance of apoptotic cells by macrophages are also evident.

II. Selection, Design, and Synthesis of siRNAs

The present invention provides compositions containing siRNA(s) and/or shRNA(s) targeted to one or more influenza virus transcripts. As the description of the influenza virus replicative cycle presented above demonstrates, various types of viral RNA transcripts (primary and secondary vRNA, primary and secondary viral mRNA, and viral cRNA) are present within cells infected with influenza virus and play important roles in the viral life cycle. Any of these transcripts are appropriate targets for siRNA mediated inhibition by either a direct or an indirect mechanism in accordance with the present invention. siRNAs and shRNAs that target any viral mRNA transcript will specifically reduce the level of the transcript itself in a direct manner, i.e., by causing degradation of the transcript. In addition, as discussed below, siRNAs and shRNAs that target certain viral transcripts (e.g., NA, PA, PB1) will indirectly cause reduction in the levels of viral transcripts to which they are not specifically targeted. In situations where alternative splicing is possible, as for the mRNA that encodes M₁ and M₂ and the mRNA that encodes NS₁ and NS₂, the unspliced transcript or the spliced transcript may serve as a target transcript.

Potential viral transcripts that may serve as a target for RNAi based therapy according to the present invention include, for example, 1) any influenza virus genomic segment; 2) transcripts that encode any viral proteins including transcripts encoding the proteins PB1, PB2, PA, NP, NS1, NS2, M1, M2, HA, or NA. As will be appreciated, transcripts may be targeted in their vRNA, cRNA, and/or mRNA form(s) by a single siRNA or shRNA, although as discussed further below, the inventors have obtained data suggesting that viral mRNA is the sole or primary target of RNAi.

For any particular gene target that is selected, the design of siRNAs or shRNAs for use in accordance with the present invention will preferably follow certain guidelines. In general, it is desirable to target sequences that are specific to the virus (as compared with the host), and that, preferably, are important or essential for viral function. Although certain viral genes, particularly those encoding HA and NA are characterized by a high mutation rate and are capable of tolerating mutations, certain regions and/or sequences tend to be conserved. According to certain embodiments of the invention such sequences may be particularly appropriate targets. As described further below, such conserved regions can be identified, for example, through review of the literature and/or comparisons of influenza gene sequences, a large number of which are publicly available. Also, in many cases, the agent that is delivered to a cell according to the present invention may undergo one or more processing steps before becoming an active suppressing agent (see below for further discussion); in such cases, those of ordinary skill in the art will appreciate that the relevant agent will preferably be designed to include sequences that may be necessary for its processing.

The inventors have found that a significant proportion of the sequences selected using the design parameters described herein prove to be efficient suppressing sequences when included in an siRNA or shRNA and tested as described below. Approximately 15% of tested siRNAs showed a strong effect and potently inhibited virus production in cells infected with either PR8 or WSN strains of influenza virus; approximately 40% showed a significant effect (i.e., a statistically significant difference (p 0.5) between virus production in the presence versus the absence of siRNA in cells infected with PR8 and/or in cells infected with WSN); approximately 45% showed no or minimal effect. Thus the invention provides siRNAs and shRNAs that inhibit virus production in cells infected with either of at least two different influenza virus subtypes.

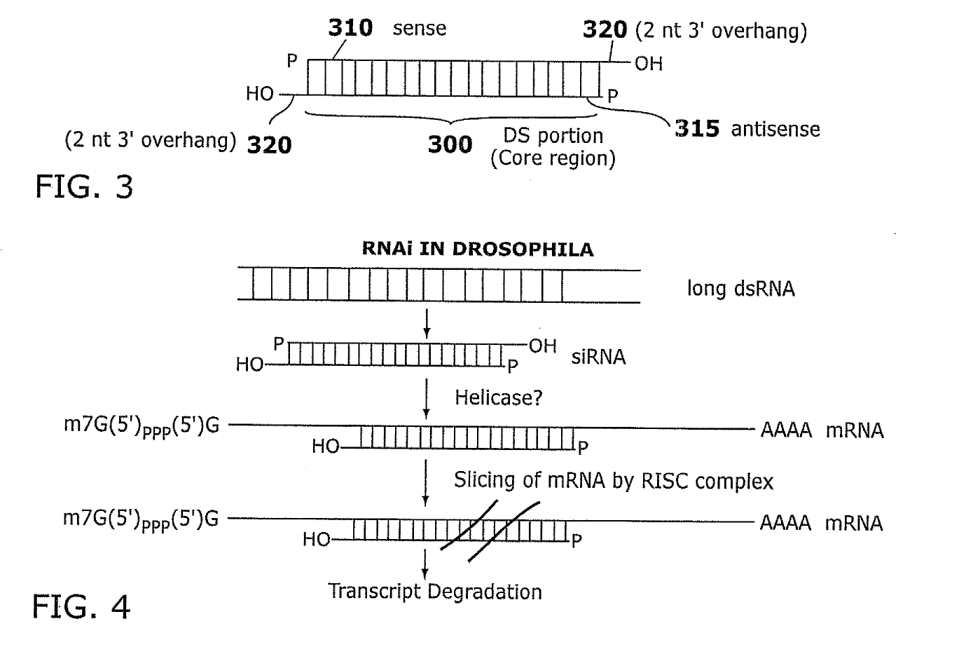

General and specific features of siRNAs and shRNAs in accordance with the invention will now be described. Short interfering RNAs (siRNAs) were first discovered in studies of the phenomenon of RNA interference (RNAi) in Drosophila, as described in WO 01/75164. In particular, it was found that, in Drosophila, long double-stranded RNAs are processed by an RNase III-like enzyme called DICER (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. FIG. 3 shows a schematic diagram of siRNAs found in Drosophila. The structure includes a 19 nucleotide double-stranded (DS) portion 300, comprising a sense strand 310 and an antisense strand 315. Each strand has a 2 nt 3′ overhang 320.

These short dsRNAs (siRNAs) act to silence expression of any gene that includes a region complementary to one of the dsRNA strands, presumably because a helicase activity unwinds the 19 bp duplex in the siRNA, allowing an alternative duplex to form between one strand of the siRNA and the target transcript. This new duplex then guides an endonuclease complex, RISC, to the target RNA, which it cleaves (“slices”) at a single location, producing unprotected RNA ends that are promptly degraded by cellular machinery (FIG. 4). As mentioned below, additional mechanisms of silencing mediated by short RNA species (microRNAs) are also known (see, e.g., Ruvkun, G., Science, 294, 797-799, 2001; Zeng, Y., et al., Molecular Cell, 9, 1-20, 2002). It is noted that the discussion of mechanisms and the figures depicting them are not intended to suggest any limitations on the mechanism of action of the present invention.

Homologs of the DICER enzyme are found in diverse species ranging from C. elegans to humans (Sharp, Genes Dev. 15;485, 2001; Zamore, Nat. Struct. Biol. 8:746, 2001), raising the possibility that an RNAi-like mechanism might be able to silence gene expression in a variety of different cell types including mammalian, or even human, cells. However, long dsRNAs (e.g., dsRNAs having a double-stranded region longer than about 30-50 nucleotides) are known to activate the interferon response in mammalian cells. Thus, rather than achieving the specific gene silencing observed with the Drosophila RNAi mechanism, the presence of long dsRNAs into mammalian cells would be expected to lead to interferon-mediated non-specific suppression of translation, potentially resulting in cell death. Long dsRNAs are therefore not thought to be useful for inhibiting expression of particular genes in mammalian cells.

However, the inventors and others have found that siRNAs, when introduced into mammalian cells, can effectively reduce the expression of target genes, including viral genes. The inventors have shown that siRNAs targeted to a variety of influenza virus RNAs, including RNAs that encode the RNA-dependent RNA transcriptase and nucleoprotein NP, dramatically reduced the level of virus produced in infected mammalian cells (Example 2, 4, 5, 6). The inventors have also shown that siRNAs targeted to influenza virus transcripts can inhibit influenza virus replication in vivo in intact organisms, namely chicken embryos infected with influenza virus (Example 3). In addition, the inventors have demonstrated that siRNAs targeted to influenza virus transcripts can inhibit virus production in mice when administered either before or after viral infection (Examples 12 and 14). Furthermore, the inventors have shown that administration of a DNA vector from which siRNA precursors (shRNAs) can be expressed inhibits influenza virus production in mice. Thus, the present invention demonstrates that treatment with siRNA, shRNA, or with vectors whose presence within a cell leads to expression of siRNA or shRNA are effective strategies for inhibiting influenza virus infection and/or replication.

While not wishing to be bound by any theory, the inventors suggest that this finding is especially significant in view of the profound changes in cellular activities, e.g., metabolic and biosynthetic activities, that take place upon infection with influenza virus as described above. Infection with influenza virus inhibits such fundamental cellular processes as cellular mRNA splicing, transport, and translation and results in inhibition of cellular protein synthesis. Despite these alterations, the finding that siRNA targeted to influenza viral transcripts inhibits viral replication suggests that the cellular mechanisms underlying the RNAi-mediated inhibition of gene expression continue to operate in cells infected with influenza virus at a level sufficient to inhibit influenza gene expression.

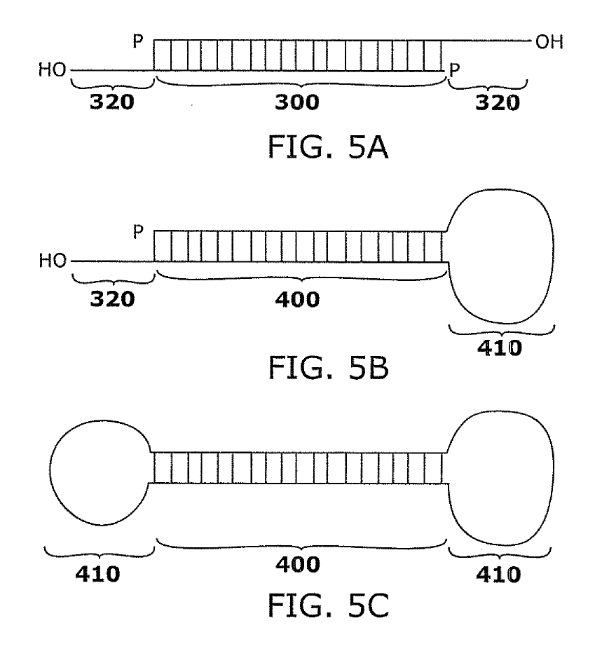

Preferred siRNAs and shRNAs for use in accordance with the present invention include a base-paired region approximately 19 nt long, and may optionally have one or more free or looped ends. For example, FIG. 5 presents various structures that could be utilized as an siRNA or shRNA according to the present invention. FIG. 5A shows the structure found to be active in the Drosophila system described above, and may represent the siRNA species that is active in mammalian cells. The present invention encompasses administration of an siRNA having the structure depicted in FIG. 5A to mammalian cells in order to treat or prevent influenza infection. However, it is not required that the administered agent have this structure. For example, the administered composition may include any structure capable of being processed in vivo to the structure of FIG. 5A, so long as the administered agent does not cause undesired or deleterious events such as induction of the interferon response. (Note that the term in vivo, as used herein with respect to the synthesis, processing, or activity of siRNA or shRNA, generally refers to events that occur within a cell as opposed to in a cell-free system. In general, the cell can be maintained in tissue culture or can be part of an intact organism.) The invention may also comprise administration of agents that are not processed to precisely the structure depicted in FIG. 5A, so long as administration of such agents reduces viral transcript levels sufficiently as discussed herein.

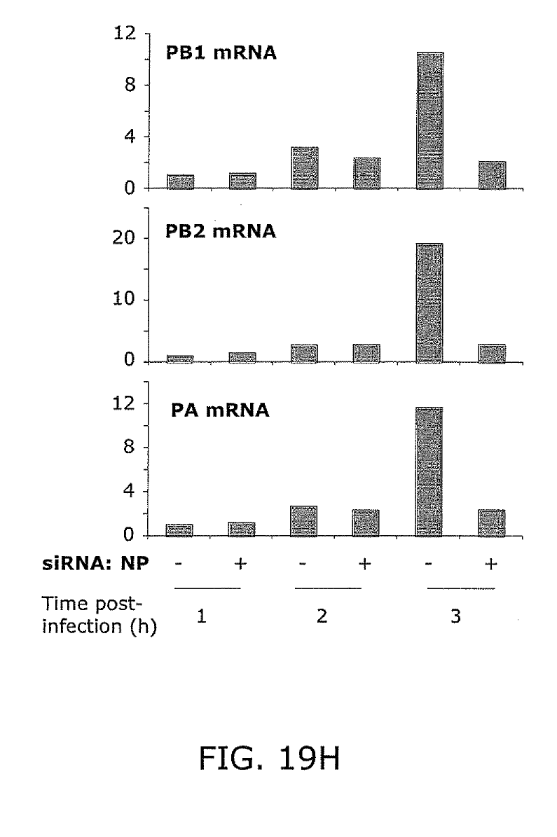

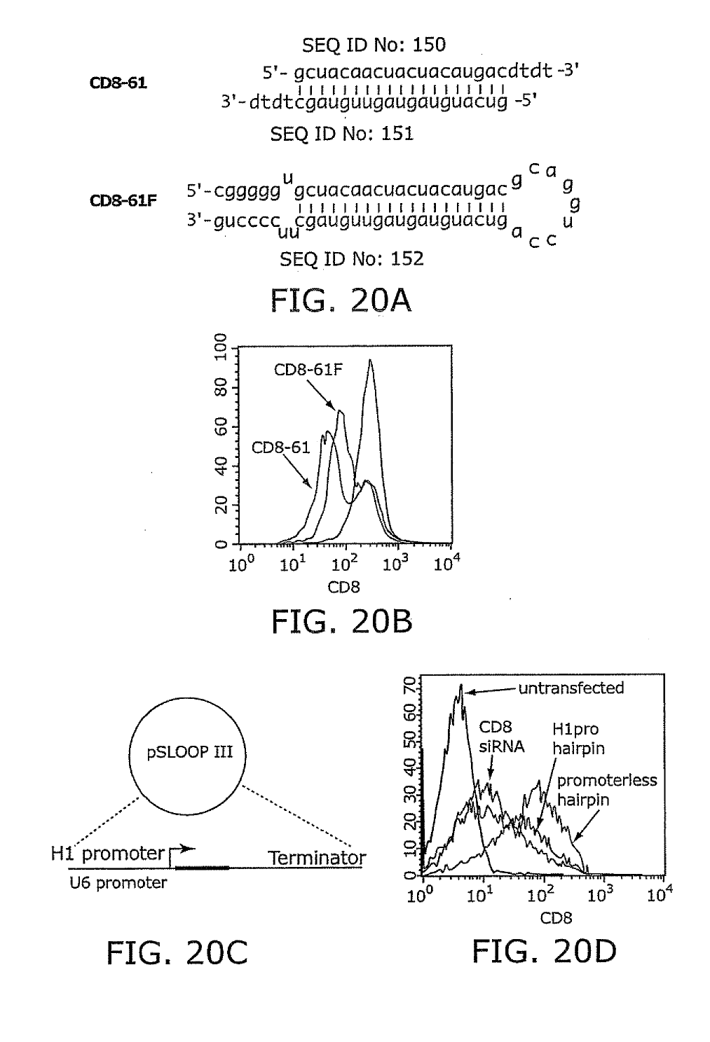

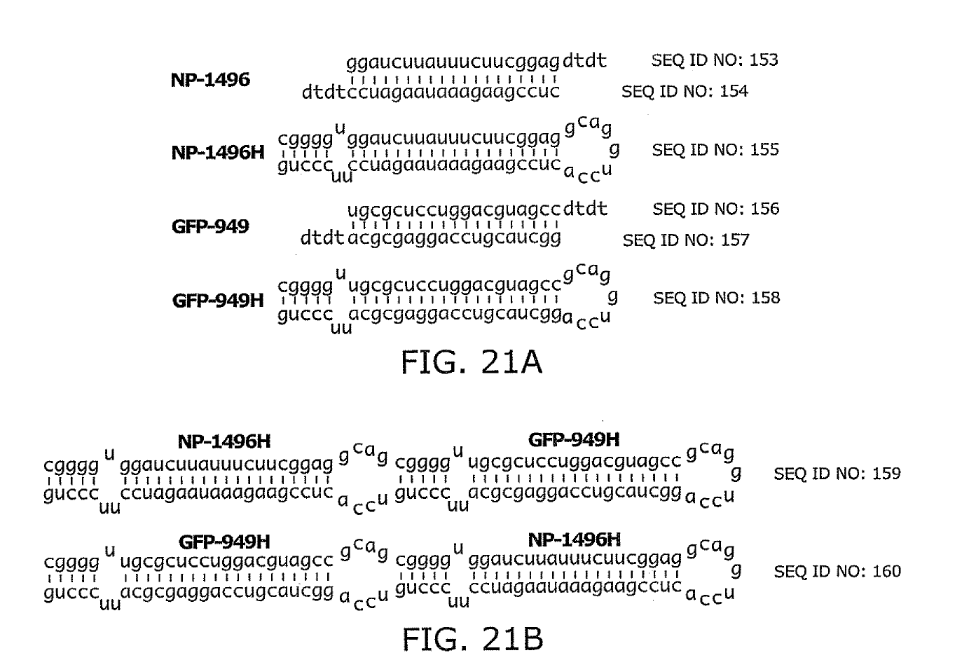

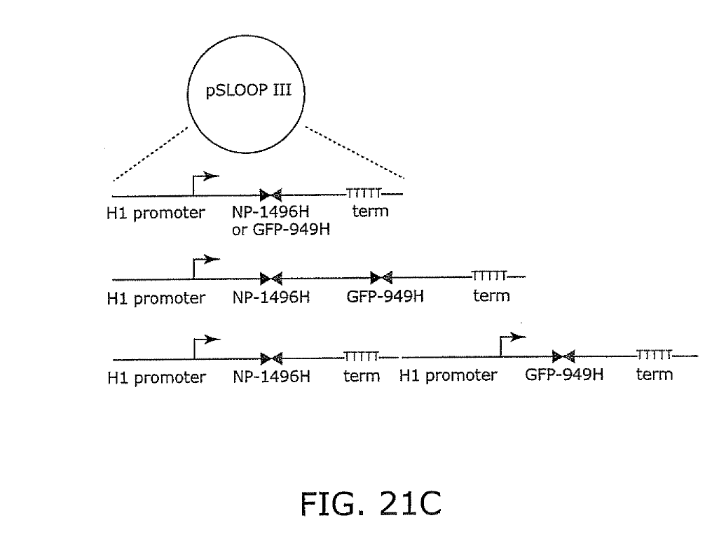

FIGS. 5B and 5C represent additional structures that may be used to mediate RNA interference. These hairpin (stem-loop) structures may function directly as inhibitory RNAs or may be processed intracellularly to yield an siRNA structure such as that depicted in FIG. 5A. FIG. 5B shows an agent comprising an RNA molecule containing two complementary regions that hybridize to one another to form a duplex region represented as stem 400, a loop 410, and an overhang 320. Such molecules will be said to self-hybridize, and a structure of this sort is referred to as an shRNA. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. One of ordinary skill in the art will appreciate that loops of 4 nucleotides or greater are less likely subject to steric constraints than are shorter loops and therefore may be preferred. In some embodiments, the overhang includes a 5′ phosphate and a 3′ hydroxyl. As discussed below, an agent having the structure depicted in FIG. 5B can readily be generated by in vivo or in vitro transcription; in several preferred embodiments, the transcript tail will be included in the overhang, so that often the overhang will comprise a plurality of U residues, e.g., between 1 and 5 U residues. It is noted that synthetic siRNAs that have been studied in mammalian systems often have 2 overhanging U residues. See also FIGS. 20 and 21 for examples of shRNA structures. The loop may be located at either the 5′ or 3′ end of the region that is complementary to the target transcript whose inhibition is desired (i.e., the antisense portion of the shRNA).

FIG. 5C shows an agent comprising an RNA circle that includes complementary elements sufficient to form a stem 400 approximately 19 bp long. Such an agent may show improved stability as compared with various other siRNAs described herein.

In describing siRNAs it will frequently be convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to comprise sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below will frequently refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to the design, selection, and delivery of inventive shRNAs.

It will be appreciated by those of ordinary skill in the art that agents having any of the structures depicted in FIG. 5, or any other effective structure as described herein, may be comprised entirely of natural RNA nucleotides, or may instead include one or more nucleotide analogs. A wide variety of such analogs is known in the art; the most commonly-employed in studies of therapeutic nucleic acids being the phosphorothioate (for some discussion of considerations involved when utilizing phosphorothioates, see, for example, Agarwal, Biochim. Biophys. Acta 1489:53, 1999). In particular, in certain embodiments of the invention it may be desirable to stabilize the siRNA structure, for example by including nucleotide analogs at one or more free strand ends in order to reduce digestion, e.g., by exonucleases. The inclusion of deoxynucleotides, e.g., pyrimidines such as deoxythymidines at one or more free ends may serve this purpose. Alternatively or additionally, it may be desirable to include one or more nucleotide analogs in order to increase or reduce stability of the 19 bp stem, in particular as compared with any hybrid that will be formed by interaction of one strand of the siRNA (or one strand of the stem portion of shRNA) with a target transcript.

According to certain embodiments of the invention various nucleotide modifications are used selectively in either the sense or antisense strand of an siRNA. For example, it may be preferable to utilize unmodified ribonucleotides in the antisense strand while employing modified ribonucleotides and/or modified or unmodified deoxyribonucleotides at some or all positions in the sense strand. See Example 5, describing the use of siRNAs having modifications at the 2′ position of nucleotides in the sense strand in order to determine whether siRNA targets viral mRNA, vRNA, and/or cRNA. According to certain embodiments of the invention only unmodified ribonucleotides are used in the duplex portion of the antisense and/or the sense strand of the siRNA while the overhang(s) of the antisense and/or sense strand may include modified ribonucleotides and/or deoxyribonucleotides. In certain embodiments of the invention one or both siRNA strands comprises one or more O-methylated ribonucleotides.

Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1^{st} edition (2001) and references therein. As will be appreciated by one of ordinary skill in the art, analogs and modifications may be tested using, e.g., the assays described herein or other appropriate assays, in order to select those that effectively reduce expression of viral genes. See references 137-139 for further discussion of modifications that have been found to be useful in the context of siRNA. The invention encompasses use of such modifications.

In certain embodiments of the invention the analog or modification results in an siRNA with increased absorbability (e.g., increased absorbability across a mucus layer, increased oral absorption, etc.), increased stability in the blood stream or within cells, increased ability to cross cell membranes, etc. As will be appreciated by one of ordinary skill in the art, analogs or modifications may result in altered Tm, which may result in increased tolerance of mismatches between the siRNA sequence and the target while still resulting in effective suppression or may result in increased or decreased specificity for desired target transcripts.

It will further be appreciated by those of ordinary skill in the art that effective siRNA agents for use in accordance with the present invention may comprise one or more moieties that is/are not nucleotides or nucleotide analogs.

In general, one strand of inventive siRNAs will preferably include a region (the “inhibitory region”) that is substantially complementary to that found in a portion of the target transcript, so that a precise hybrid can form in vivo between one strand or portion of the siRNA (the antisense strand) and the target transcript. In those embodiments of the invention in which an shRNA structure is employed, this substantially complementary region preferably includes most or all of the stem structure depicted in FIG. 5B. In certain preferred embodiments of the invention, the relevant inhibitor region of the siRNA or shRNA is perfectly complementary with the target transcript; in other embodiments, one or more non-complementary residues are located within the siRNA/template duplex. It may be preferable to avoid mismatches in the central portion of the siRNA/template duplex (see, for example, Elbashir et al., EMBO J. 20:6877, 2001, incorporated herein by reference).

In general, preferred siRNAs hybridize with a target site that includes exonic sequences in the target transcript. Hybridization with intronic sequences is not excluded, but generally appears not to be preferred in mammalian cells. In certain preferred embodiments of the invention, the siRNA hybridizes exclusively with exonic sequences. In some embodiments of the invention, the siRNA hybridizes with a target site that includes only sequences within a single exon; in other embodiments the target site is created by splicing or other modification of a primary transcript. In general, any site that is available for hybridization with an siRNA resulting in slicing and degradation of the transcript may be utilized in accordance with the present invention. Nonetheless, those of ordinary skill in the art will appreciate that, in some instances, it may be desirable to select particular regions of target transcript as siRNA hybridization targets. For example, it may be desirable to avoid sections of target transcript that may be shared with other transcripts whose degradation is not desired. In general, coding regions and regions closer to the 3′ end of the transcript than to the 5′ end are preferred.

siRNAs may be selected according to a variety of approaches. In general, as mentioned above, inventive siRNAs will preferably include a region (the “inhibitory region” or “duplex region”) that is perfectly complementary or substantially complementary to that found in a portion of the target transcript (the “target portion”), so that a hybrid can form in vivo between the antisense strand of the siRNA and the target transcript. This duplex region, also referred to as the “core region” is understood not to include overhangs, although overhangs, if present, may also be complementary to the target transcript. Preferably, this perfectly or substantially complementary region includes most or all of the double-stranded structure depicted in FIGS. 3, 4, and 5. The relevant inhibitor region of the siRNA is preferably perfectly complementary with the target transcript. However, siRNAs including one or more non-complementary residues have also been shown to mediate silencing, though the extent of inhibition may be less than that achievable using siRNAs with duplex portions that are perfectly complementary to the target transcript. In general, mismatches in the 3′ half of the siRNA duplex portion appear to result in less reduction in the inhibitory effect than mismatches in the 5′ half of the siRNA duplex portion.

For purposes of description herein, the length of an siRNA core region will be assumed to be 19 nucleotides, and a 19 nucleotide sequence is referred to as N19. However, the core region may range in length from 15 to 29 nucleotides. In addition, it is assumed that the siRNA N19 inhibitory region will be chosen so that the core region of the antisense strand of the siRNA (i.e., the portion that is complementary to the target transcript) is perfectly complementary to the target transcript, though as mentioned above one or more mismatches may be tolerated. In general it is desirable to avoid mismatches in the duplex region if an siRNA having maximal ability to reduce expression of the target transcript via the classical pathway is desired. However, as described below, it may be desirable to select an siRNA that exhibits less than maximal ability to reduce expression of the target transcript, or it may be desirable to employ an siRNA that acts via the alternative pathway. In such situations it may be desirable to incorporate one or more mismatches in the duplex portion of the siRNA. In general, preferably fewer than four residues or alternatively less than about 15% of residues in the inhibitory region are mismatched with the target.

In some cases the siRNA sequence is selected such that the entire antisense strand (including the 3′ overhang if present) is perfectly complementary to the target transcript. However, it is not necessary that overhang(s) are either complementary or identical to the target transcript. Any desired sequence (e.g., UU) may simply be appended to the 3′ ends of antisense and/or sense 19 bp core regions of an siRNA to generate 3′ overhangs. In general, overhangs containing one or more pyrimidines, usually U, T, or dT, are employed. When synthesizing siRNAs it may be more convenient to use T rather than U, while use of dT rather than T may confer increased stability. As indicated above, the presence of overhangs is optional and, where present, they need not have any relationship to the target sequence itself. It is noted that since shRNAs have only one 3′ end, only a single 3′ overhang is possible prior to processing to form siRNA.

In summary, in general an siRNA may be designed by selecting any core region of appropriate length, e.g., 19 nt, in the target transcript, and selecting an siRNA having an antisense strand whose sequence is substantially or perfectly complementary to the core region and a sense strand whose sequence is complementary to the antisense strand of the siRNA. 3′ overhangs such as those described above may then be added to these sequences to generate an siRNA structure. Thus there is no requirement that the overhang in the antisense strand is complementary to the target transcript or that the overhang in the sense strand corresponds with sequence present in the target transcript. It will be appreciated that, in general, where the target transcript is an mRNA, siRNA sequences may be selected with reference to the corresponding sequence of double-stranded cDNA rather than to the mRNA sequence itself, since according to convention the sense strand of the cDNA is identical to the mRNA except that the cDNA contains T rather than U. (Note that in the context of the influenza virus replication cycle, double-stranded cDNA is not generated, and the cDNA present in the cell is single-stranded and is complementary to viral mRNA.)

Not all siRNAs are equally effective in reducing or inhibiting expression of any particular target gene. (See, e.g., Holen, T., et al., Nucleic Acids Res., 30(8):1757-1766, reporting variability in the efficacy of different siRNAs), and a variety of considerations may be employed to increase the likelihood that a selected siRNA may be effective. For example, it may be preferable to select target portions within exons rather than introns. In general, target portions near the 3′ end of a target transcript may be preferred to target portions near the 5′ end or middle of a target transcript. siRNAs may generally be designed in accordance with principles described in Technical Bulletin # 003-Revision B, “siRNA Oligonucleotides for RNAi Applications”, available from Dharmacon Research, Inc., Lafayette, Colo. 80026, a commercial supplier of RNA reagents. Technical Bulletins #003 (accessible on the World Wide Web at www.dharmacon.com/tech/tech003B.html) and #004 available at www.dharmacon.com/tech/tech004.html from Dharmacon contain a variety of information relevant to siRNA design parameters, synthesis, etc., and are incorporated herein by reference. Additional design considerations that may also be employed are described in Semizarov, D., et al., Proc. Natl. Acad. Sci., Vol. 100, No. 11, pp. 6347-6352.

One aspect of the present invention is the recognition that when multiple strains, subtypes, etc. (referred to collectively as variants), of an infectious agent exist, whose genomes vary in sequence, it will often be desirable to select and/or design siRNAs and shRNAs that target regions that are highly conserved among different variants. In particular, by comparing a sufficient number of sequences and selecting highly conserved regions, it will be possible to target multiple variants with a single siRNA whose duplex portion includes such a highly conserved region. Generally such regions should be of sufficient length to include the entire duplex portion of the siRNA (e.g., 19 nucleotides) and, optionally, one or more 3′ overhangs, though regions shorter than the full length of the duplex can also be used (e.g., 15, 16, 17, or 18 nucleotides). According to certain embodiments of the invention a region is highly conserved among multiple variants if it is identical among the variants. According to certain embodiments of the invention a region (of whatever length is to be included in the duplex portion of the siRNA, e.g., 15, 16, 17, 18, or, preferably, 19 nucleotides) is highly conserved if it differs by at most one nucleotide (i.e., 0 or 1 nucleotide) among the variants. According to certain embodiments of the invention such a region is highly conserved among multiple variants if it differs by at most two nucleotides (i.e., 0, 1, or 2 nucleotides) among the variants. According to certain embodiments of the invention a region is highly conserved among multiple variants if it differs by at most three nucleotides or (i.e., 0, 1, 2, or 3 nucleotides) among the variants. According to certain embodiments of the invention an siRNA includes a duplex portion that targets a region that is highly conserved among at least 5 variants, at least 10 variants, at least 15 variants, at least 20 variants, at least 25 variants, at least 30 variants, at least 40 variants, or at least 50 or more variants.

In order to determine whether a region is highly conserved among a set of multiple variants, the following procedure may be used. One member of the set of sequences is selected as the base sequence, i.e., the sequence to which other sequences are to be compared. Typically the length of the base sequence will be the length desired for the duplex portion of the siRNA, e.g, 15, 16, 17, 18, or, preferably 19 nucleotides. According to different embodiments of the invention the base sequence may be either one of the sequences in the set being compared or may be a consensus sequence derived, e.g., by determining for each position the most frequently found nucleotide at that position among the sequences in the set.

Having selected a base sequence, the sequence of each member of the set of multiple variants is compared with the base sequence. The number of differences between the base sequence and any member of the set of multiple variants over a region of the sequence is used to determine whether the base sequence and that member are highly conserved over the particular region of interest. As noted above, in various embodiments of the invention if the number of sequence differences between two regions is either 0; 0 or 1, 0, 1, or 2; or 0, 1, 2, or 3, the regions are considered highly conserved. At the positions where differences occur, the siRNA sequence may be selected to be identical to the base sequence or to one of the other sequences. Generally the nucleotide present in the base sequence will be selected. However in certain embodiments of the invention, particularly if a nucleotide present at a particular position in a second sequence in the set being compared is found in more of the sequences being compared than the nucleotide in the base sequence, then the siRNA sequence may be selected to be identical to the second sequence. In addition according to certain embodiments of the invention, if the consensus nucleotide (most commonly occurring nucleotide) at the position where the difference occurs is different to that found in the base sequence, the consensus nucleotide may be used. Note that this may result in a sequence that is not identical to any of the sequences being compared (as may the use of a consensus sequence as the base sequence).

Example 1 shows the selection of siRNA sequences based on comparison of a set of sequences from six influenza A strains having a human host of origin and comparison of a set of sequences from seven influenza A strains having different animal hosts of origin (including human). It is to be understood that different methods of selecting highly conserved regions may be used. However, the invention encompasses siRNAs whose duplex portions (and, optionally, any overhangs included in the siRNA) are selected based on highly conserved regions that meet the criteria provided herein, regardless of how the highly conserved regions are selected. It is also to be understood that the invention encompasses siRNAs targeted to portions of influenza virus transcripts that do not meet the criteria for highly conserved regions described herein. Although such siRNAs may be less preferred to those that are targeted to highly conserved regions, they are still effective inhibitors of influenza virus production for those viruses whose transcripts they target.

Table 1A lists 21-nucleotide regions that are highly conserved among a set of influenza virus sequences for each of the viral gene segments. The sequences in Table 1A are listed in 5′ to 3′ direction according to the sequence present in viral mRNA except that T is used instead of U. The numbers indicate the locations of the sequences in the viral genome. For example, PB2-117/137 denotes a sequence extending from position 117 to position 137 in segment PB2. According to certain embodiments of the invention, to design siRNAs based on these sequences, nucleotides 3-21 are selected as the core regions of siRNA sense strand sequences. A two nt 3′ overhang consisting of dTdT is added to each. A sequence complementary to nucleotides 1-21 of each sequence is selected as the corresponding antisense strand. For example, to design an siRNA based on the highly conserved sequence PA-44/64, i.e., AATGCTTCAATCCGATGATTG (SEQ ID NO: 22) a 19 nt core region having the sequence TGCTTCAATCCGATGATTG (SEQ ID NO: 109) is selected. A two nt 3′ overhang consisting of dTdT is added, resulting (after replacement of T by U) in the sequence 5′-UGCUUCAAUCCGAUGAUUGdTdT-3′ (SEQ ID NO: 79). This is the sequence of the siRNA sense strand. The sequence of the antisense siRNA strand sequence (in the 5′ to 3′ direction) is complementary to SEQ ID NO: 22, i.e., CAAUCAUCGGAUUGAAGCAdTdT (SEQ ID NO: 80) where T has been replaced by U except for the 2 nt 3′ overhang, in which T is replaced by dT. Sense and antisense siRNA sequences may be similarly obtained from each sequence listed in Table 1A. Twenty such siRNA sequences are listed in Table 2.

Each sequence listed in Table 1A includes a 19 nt region (nt 3-21) and an initial 2 nt sequence that is not present in the sense strand of the corresponding siRNA but is complementary to the 3′ overhang of the antisense strand of the siRNA. It will be appreciated that the 19 nt region may be used as the sense strand to design a variety of siRNA molecules having different 3′ overhangs in either or both the sense and antisense strands. Nucleotides 3 to 21 in each of the sequences listed in Table 1A correspond to sense sequences for siRNAs, listed from left to right in the 5′ to 3′ direction. The corresponding antisense sequence is complementary to nucleotides 1 to 21 of the listed sequence. Hybridization of sense and antisense strands having these sequences (with addition of a 3′OH overhang to the sense strand sequence and replacement of T with U in both sequences) thus results in an siRNA having a 19 base pair core duplex region, with each strand having a 2 nucleotide 3′ OH overhang. However, in accordance with the description presented above, the sequences presented in Table 1A may be used to design a variety of siRNAs that do not have precisely this structure. For example, the sequence of the overhangs may be varied, and the presence of one or both of the overhangs may not be essential for effective siRNA mediated inhibition of gene expression. In addition, although the preferred length of the duplex portion of an siRNA may be 19 nucleotides, shorter or longer duplex portions may be effective. Thus siRNAs designed in accordance with the highly conserved sequences presented in Table 1A may include only some of those nucleotides in the region between positions 3 and 21 in the sense strand of the siRNA. (Note that when the word “between” is followed by a range of values, the range is taken to include the endpoints).

Table 1B lists additional siRNAs designed based on highly conserved regions of influenza virus. Both sense and antisense strands are shown in a 5′ to 3′ direction. A dTdT 3′ overhang is appended to each strand. Nucleotides 1 to 19 in each of the sense strand sequences listed in Table 1B has an identical sequence to a highly conserved region of an influenza virus transcript. The corresponding antisense sequence is complementary to the sense strand. For purposes of the following description, a “highly conserved region” refers to nucleotides 3-21 in any of the sequences listed in Table 1A or nucleotides 1-19 of any of the sense strands listed in Table 1B. These are the regions that are present in double-stranded form in an inventive siRNA or shRNA. The sequences of these regions are referred to as “highly conserved sequences”.

The invention provides siRNAs having sense strands with sequences that include all or a portion of the highly conserved sequences listed in Tables 1A and 1B. The invention further provides shRNAs having sense portions with sequences that include all or a portion of the highly conserved sequences listed in Tables 1A and 1B. For brevity, the discussion below describes siRNAs. However, it is to be understood that the invention encompasses corresponding shRNAs, wherein the sense portion of the shRNA includes all or a portion of the highly conserved sequences listed in Tables 1A and 1B.

Generally, the sequence of the sense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides of the listed highly conserved sequence. Generally the sequence of the antisense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides that are perfectly complementary to a portion of the sequence of the listed highly conserved sequence. Thus the invention encompasses siRNAs that are “shifted” by 1 or more nucleotides, e.g, up to 9 nucleotides, from the highly conserved sequences in Table 1A or Table 1B with respect to the portion of the target transcript with which they are complementary.

In certain embodiments of the invention the sequence of the sense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides of the highly conserved sequence, with one nucleotide difference from the listed sequence. In certain embodiments of the invention the sequence of the antisense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides that are perfectly complementary to a portion of the highly conserved sequence except that one nucleotide may differ.

In certain embodiments of the invention the sequence of the sense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides of the listed highly conserved sequence, with two nucleotides different from the listed sequence. In certain embodiments of the invention the sequence of the antisense strand of an siRNA designed in accordance with a highly conserved sequence presented in Table 1A or Table 1B will include at least 10 consecutive nucleotides, more preferably at least 12 consecutive nucleotides, more preferably at least 15 consecutive nucleotides, more preferably at least 17 consecutive nucleotides, and yet more preferably 19 consecutive nucleotides that are perfectly complementary to the highly conserved sequence except that two nucleotides may differ.

According to certain embodiments of the invention the siRNA includes a duplex portion that is highly conserved among variants that naturally infect organisms of at least two different species. According to certain embodiments of the invention the siRNA includes a duplex portion that is highly conserved among variants that originate in organisms of at least two different species. According to certain embodiments of the invention the siRNA includes a duplex portion that is highly conserved among variants that originate in organisms of at least three different species, at least four different species, or at least five different species. The species may include human, equine (horse), avian (e.g., duck, chicken), swine and others. In certain preferred embodiments of the invention the species include humans. In the case of many infectious agents, e.g., numerous previously identified influenza A subtypes, the ability of the subtype to infect a host of a particular species is known. In addition, the species of origin of numerous influenza subtypes is known as reflected in the names of the subtypes. One of ordinary skill in the art will be able to determine whether an infectious agent naturally infects any particular host species and/or to determine the species of origin of the agent either by review of the literature or in accordance with methods that have been used for influenza A virus subtypes. It may also be desirable to select variants that were isolated in different years and/or variants that express different NA and HA subtypes. For example, the variants used to select highly conserved sequences for duplex portions of siRNA/shRNA as described in Example 1 included variants isolated from humans as well as a wide variety of different animal source. The variants included viruses isolated in different years and included viruses expressing almost all known HA and NA subtypes.

According to certain embodiments of the invention the infectious agent is an agent whose genome comprises multiple independent nucleic acid segments, e.g., multiple independent RNA segments. Generally the duplex portion includes at least 10 consecutive nucleotides, more preferably 12 consecutive nucleotides, and more preferably at least 15 consecutive nucleotides that are highly conserved among multiple variants. Preferably the duplex portion includes at least 17 consecutive nucleotides that are highly conserved among multiple variants. According to certain embodiments of the invention the duplex portion includes 19 consecutive nucleotides that are highly conserved among multiple variants. In addition to the duplex portion, the siRNA may include a 3′ overhang on one or more strands. An overhang in the sense strand of the siRNA may (but according to certain embodiments of the invention need not) be identical to sequences present in the target transcript 3′ of the target region. An overhang in the antisense strand of the siRNA may (but according to certain embodiments of the invention need not) be complementary to the nucleotides immediately 5′ of the target portion of the target transcript. Overhangs may be 1 nucleotide, 2 nucleotides, or more in length as described elsewhere herein.

One of ordinary skill in the art will appreciate that siRNAs may exhibit a range of melting temperatures (Tm) and dissociation temperatures (Td) in accordance with the foregoing principles. The Tm is defined as the temperature at which 50% of a nucleic acid and its perfect complement are in duplex in solution while the Td, defined as the temperature at a particular salt concentration, and total strand concentration at which 50% of an oligonucleotide and its perfect filter-bound complement are in duplex, relates to situations in which one molecule is immobilized on a filter. Representative examples of acceptable Tms may readily be determined using methods well known in the art, either experimentally or using appropriate empirically or theoretically derived equations, based on the siRNA sequences disclosed in the Examples herein.

One common way to determine the actual Tm is to use a thermostatted cell in a UV spectrophotometer. If temperature is plotted vs. absorbance, an S-shaped curve with two plateaus will be observed. The absorbance reading halfway between the plateaus corresponds to Tm. The simplest equation for Td is the Wallace rule: Td=2(A+T)+4(G+C) Wallace, R. B.; Shaffer, J.; Murphy, R. F.; Bonner, J.; Hirose, T.; Itakura, K., Nucleic Acids Res. 6, 3543 (1979). The nature of the immobilized target strand provides a net decrease in the Tm observed relative to the value when both target and probe are free in solution. The magnitude of the decrease is approximately 7-8° C. Another useful equation for DNA which is valid for sequences longer than 50 nucleotides from pH 5 to 9 within appropriate values for concentration of monovalent cations, is: Tm=81.5+16.6 log M+41(XG+XC)-500/L -0.62 F, where M is the molar concentration of monovalent cations, XG and XC are the mole fractions of G and C in the sequence, L is the length of the shortest strand in the duplex, and F is the molar concentration of formamide (Howley, P. M; Israel, M. F.; Law, M-F.; Martin, M. A., J. Biol. Chem. 254, 4876). Similar equations for RNA are: Tm=79.8+18.5 log M+58.4 (XG+XC)+11.8(XG+XC)2−820/L−0.35 F and for DNA-RNA hybrids: Tm=79.8+18.5 log M+58.4 (XG+XC)+11.8(XG+XC)2−820/L−0.50 F. These equations are derived for immobilized target hybrids. Several studies have derived accurate equations for Tm using thermodynamic basis sets for nearest neighbor interactions. The equation for DNA and RNA is: Tm=(1000ΔH)/A+ΔS+Rln(Ct/4)−273.15+16.6 ln[Na⁺], where ΔH (Kcal/mol) is the sum of the nearest neighbor enthalpy changes for hybrids, A (eu) is a constant containing corrections for helix initiation, ΔS (eu) is the sum of the nearest neighbor entropy changes, R is the Gas Constant (1.987 cal deg⁻¹ mol⁻¹) and Ct is the total molar concentration of strands. If the strand is self complementary, Ct/4 is replaced by Ct. Values for thermodynamic parameters are available in the literature. For DNA see Breslauer, et al., Proc. Natl. Acad. Sci. USA 83, 3746-3750, 1986. For RNA:DNA duplexes see Sugimoto, N., et al, Biochemistry, 34(35): 11211-6, 1995. For RNA see Freier, S. M., et al., Proc. Natl. Acad. Sci. 83, 9373-9377, 1986. Rychlik, W., et al., Nucl. Acids Res. 18(21), 6409-6412, 1990. Various computer programs for calculating Tm are widely available. See, e.g., the Web site having URL www.basic.nwu.edu/biotools/oligocalc.html.

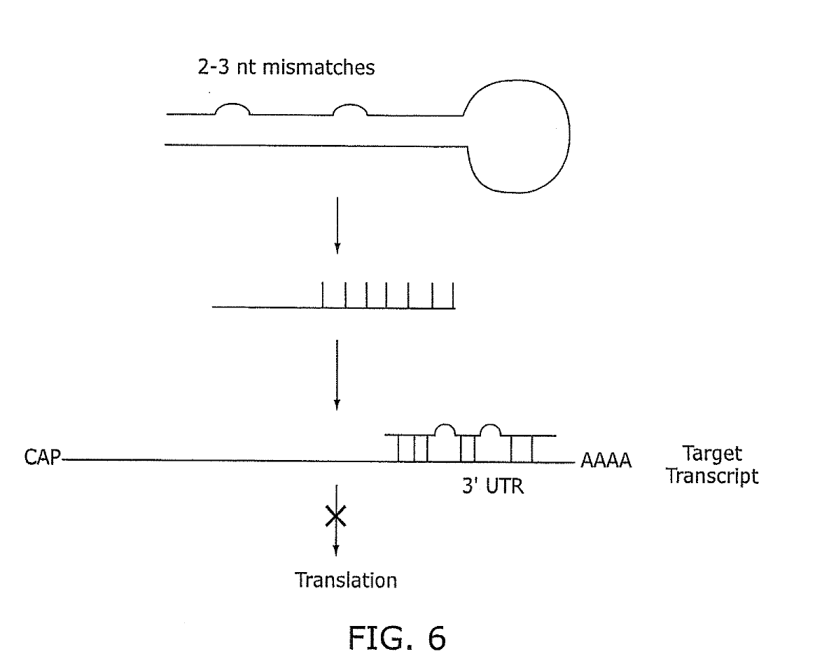

Certain siRNAs hybridize to a target site that includes or consists entirely of 3′ UTR sequences. Such siRNAs may tolerate a larger number of mismatches in the siRNA/template duplex, and particularly may tolerate mismatches within the central region of the duplex. For example, one or both of the strands may include one or more “extra” nucleotides that form a bulge as shown in FIG. 6. Typically the stretches of perfect complementarity are at least 5 nucleotides in length, e.g., 6, 7, or more nucleotides in length, while the regions of mismatch may be, for example, 1, 2, 3, or 4 nucleotides in length. When hybridized with the target transcript such siRNAs frequently include two stretches of perfect complementarity separated by a region of mismatch. A variety of structures are possible. For example, the siRNA may include multiple areas of nonidentity (mismatch). The areas of nonidentity (mismatch) need not be symmetrical, i.e., it is not required that both the target and the siRNA include nonpaired nucleotides.

Some mismatches may be desirable, as siRNA/template duplex formation in the 3′ UTR may inhibit expression of a protein encoded by the template transcript by a mechanism related to but distinct from classic RNA inhibition. In particular, there is evidence to suggest that siRNAs that bind to the 3′ UTR of a template transcript may reduce translation of the transcript rather than decreasing its stability. Specifically, as shown in FIG. 6, the DICER enzyme that generates siRNAs in the Drosophila system discussed above and also in a variety of organisms, is known to also be able to process a small, temporal RNA (stRNA) substrate into an inhibitory agent that, when bound within the 3′ UTR of a target transcript, blocks translation of the transcript (see Grishok, A., et al., Cell 106, 23-24, 2001; Hutvagner, G., et al., Science, 293, 834-838, 2001; Ketting, R., et al., Genes Dev., 15, 2654-2659). For the purposes of the present invention, any partly or fully double-stranded short RNA as described herein, one strand of which binds to a target transcript and reduces its expression (i.e., reduces the level of the transcript and/or reduces synthesis of the polypeptide encoded by the transcript) is considered to be an siRNA, regardless of whether the RNA acts by triggering degradation, by inhibiting translation, or by other means. In certain preferred embodiments of the invention, reducing expression of the transcript involves degradation of the transcript. In addition any precursor structure (e.g., a short hairpin RNA, as described herein) that may be processed in vivo (i.e., within a cell or organism) to generate such an siRNA is useful in the practice of the present invention.

Those of ordinary skill in the art will readily appreciate that inventive RNAi-inducing agents may be prepared according to any available technique including, but not limited to chemical synthesis, enzymatic or chemical cleavage in vivo or in vitro, or template transcription in vivo or in vitro. As noted above, inventive RNA-inducing agents may be delivered as a single RNA molecule including self-complementary portions (i.e., an shRNA that can be processed intracellularly to yield an siRNA), or as two strands hybridized to one another. For instance, two separate 21 nt RNA strands may be generated, each of which contains a 19 nt region complementary to the other, and the individual strands may be hybridized together to generate a structure such as that depicted in FIG. 5A.

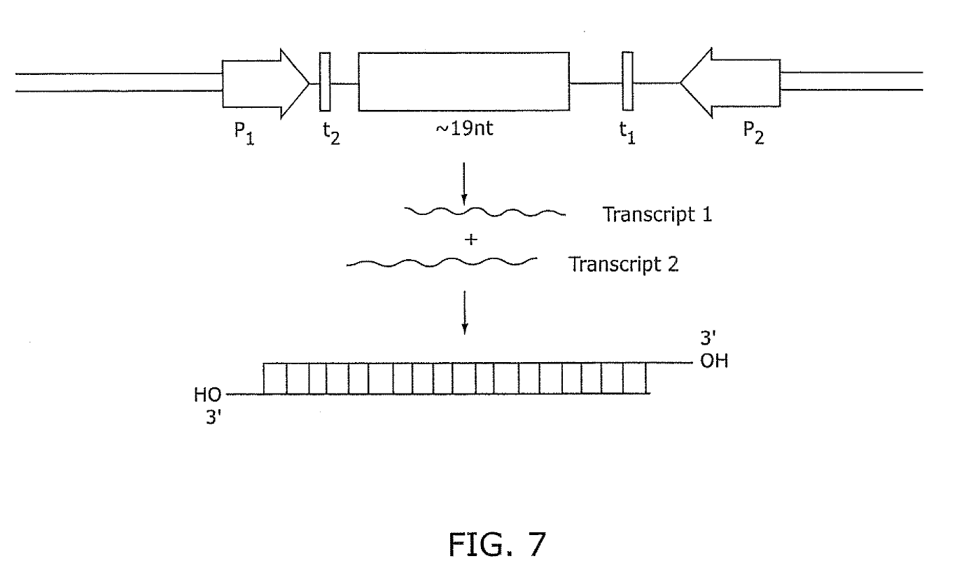

Alternatively, each strand may be generated by transcription from a promoter, either in vitro or in vivo. For instance, a construct may be provided containing two separate transcribable regions, each of which generates a 21 nt transcript containing a 19 nt region complementary with the other. Alternatively, a single construct may be utilized that contains opposing promoters P1 and P2 and terminators t1 and t2 positioned so that two different transcripts, each of which is at least partly complementary to the other, are generated is indicated in FIG. 7.

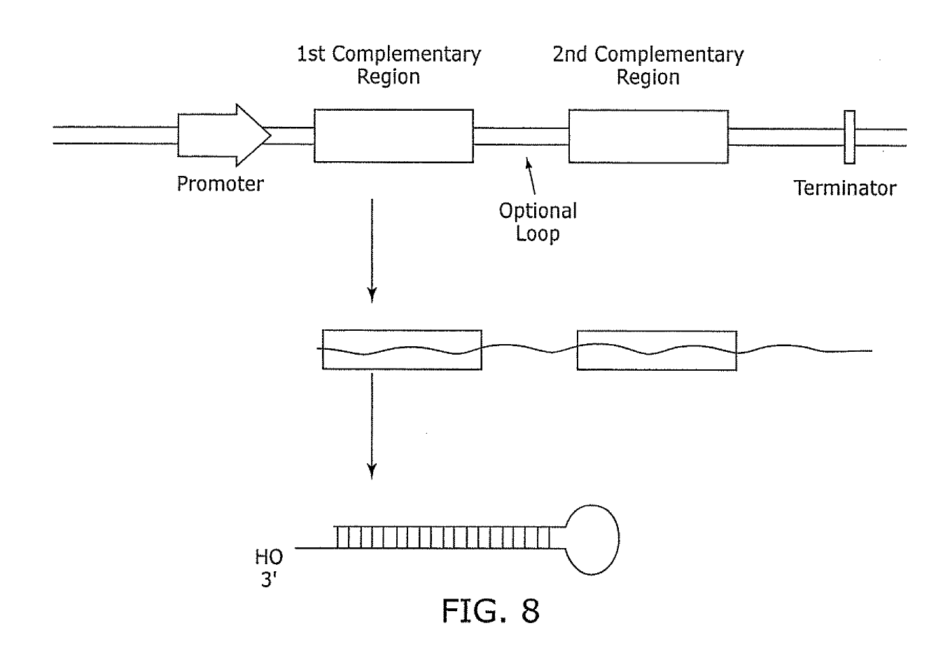

In another embodiment, an inventive RNA-inducing agent is generated as a single transcript, for example by transcription of a single transcription unit encoding self complementary regions. FIG. 8 depicts one such embodiment of the present invention. As indicated, a template is employed that includes first and second complementary regions, and optionally includes a loop region. Such a template may be utilized for in vitro or in vivo transcription, with appropriate selection of promoter (and optionally other regulatory elements, e.g., terminator). The present invention encompasses constructs encoding one or more siRNA strands.

In vitro transcription may be performed using a variety of available systems including the T7, SP6, and T3 promoter/polymerase systems (e.g., those available commercially from Promega, Clontech, New England Biolabs, etc.). As will be appreciated by one of ordinary skill in the art, use of the T7 or T3 promoters typically requires an siRNA sequence having two G residues at the 5′ end while use of the SP6 promoter typically requires an siRNA sequence having a GA sequence at its 5′ end. Vectors including the T7, SP6, or T3 promoter are well known in the art and can readily be modified to direct transcription of siRNAs. When siRNAs are synthesized in vitro they may be allowed to hybridize before transfection or delivery to a subject. It is to be understood that inventive siRNA compositions need not consist entirely of double-stranded (hybridized) molecules. For example, siRNA compositions may include a small proportion of single-stranded RNA. This may occur, for example, as a result of the equilibrium between hybridized and unhybridized molecules, because of unequal ratios of sense and antisense RNA strands, because of transcriptional termination prior to synthesis of both portions of a self-complementary RNA, etc. Generally, preferred compositions comprise at least approximately 80% double-stranded RNA, at least approximately 90% double-stranded RNA, at least approximately 95% double-stranded RNA, or even at least approximately 99-100% double-stranded RNA. However, the siRNA compositions may contain less than 80% hybridized RNA provided that they contain sufficient double-stranded RNA to be effective.

Those of ordinary skill in the art will appreciate that, where inventive siRNA or shRNA agents are to be generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements can readily be selected to allow expression of the relevant transcription units in mammalian cells. In some embodiments of the invention, it may be desirable to utilize a regulatable promoter; in other embodiments, constitutive expression may be desired. It is noted that the term “expression” as used herein in reference to synthesis (transcription) of siRNA or siRNA precursors does not imply translation of the transcribed RNA.

In certain preferred embodiments of the invention, the promoter utilized to direct in vivo expression of one or more siRNA or shRNA transcription units is a promoter for RNA polymerase III (Pol III). Pol III directs synthesis of small transcripts that terminate upon encountering a stretch of 4-5 T residues in the template. Certain Pol III promoters such as the U6 or III promoters do not require cis-acting regulatory elements (other than the first transcribed nucleotide) within the transcribed region and thus are preferred according to certain embodiments of the invention since they readily permit the selection of desired siRNA sequences. In the case of naturally occurring U6 promoters the first transcribed nucleotide is guanosine, while in the case of naturally occurring H1 promoters the first transcribed nucleotide is adenine. (See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Thus in certain embodiments of the invention, e.g., where transcription is driven by a U6 promoter, the 5-nucleotide of preferred siRNA sequences is G. In certain other embodiments of the invention, e.g., where transcription is driven by an H1 promoter, the 5′ nucleotide may be A.

According to certain embodiments of the invention promoters for Pol II may also be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In certain embodiments of the invention tissue-specific, cell-specific, or inducible Pol II promoters may be used, provided the foregoing requirements are met. In addition, in certain embodiments of the invention promoters for Pol I may be used as described, for example, in (McCown 2003).

It will be appreciated that in vivo expression of constructs that provide templates for synthesis of siRNA or shRNA, such as those depicted in FIGS. 7 and 8 can desirably be accomplished by introducing the constructs into a vector, such as, for example, a DNA plasmid or viral vector, and introducing the vector into mammalian cells. Any of a variety of vectors may be selected, though in certain embodiments it may be desirable to select a vector that can deliver the construct(s) to one or more cells that are susceptible to influenza virus infection. The present invention encompasses vectors containing siRNA and/or shRNA transcription units, as well as cells containing such vectors or otherwise engineered to contain transcription units encoding one or more siRNA or shRNA strands. In certain preferred embodiments of the invention, inventive vectors are gene therapy vectors appropriate for the delivery of an siRNA or shRNA expressing construct to mammalian cells (e.g., cells of a domesticated mammal), and most preferably human cells. Such vectors may be administered to a subject before or after exposure to an influenza virus, to provide prophylaxis or treatment for diseases and conditions caused by infection with the virus. The RNAi-inducing vectors of the invention may be delivered in a composition comprising any of a variety of delivery agents as described further below.

The invention therefore provides a variety of viral and nonviral vectors whose presence within a cell results in transcription of one or more RNAs that self-hybridize or hybridize to each other to form an shRNA or siRNA that inhibits expression of at least one influenza virus transcript in the cell. In certain embodiments of the invention two separate, complementary siRNA strands are transcribed using a single vector containing two promoters, each of which directs transcription of a single siRNA strand, i.e., is operably linked to a template for the siRNA so that transcription occurs. The two promoters may be in the same orientation, in which case each is operably linked to a template for one of the siRNA strands. Alternately, the promoters may be in opposite orientation flanking a single template so that transcription from the promoters results in synthesis of two complementary RNA strands.

In other embodiments of the invention a vector containing a promoter that drives transcription of a single RNA molecule comprising two complementary regions (e.g., an shRNA) is employed. In certain embodiments of the invention a vector containing multiple promoters, each of which drives transcription of a single RNA molecule comprising two complementary regions is used. Alternately, multiple different shRNAs may be transcribed, either from a single promoter or from multiple promoters. A variety of configurations are possible. For example, a single promoter may direct synthesis of a single RNA transcript containing multiple self-complementary regions, each of which may hybridize to generate a plurality of stem-loop structures. These structures may be cleaved in vivo, e.g., by DICER, to generate multiple different shRNAs. It will be appreciated that such transcripts preferably contain a termination signal at the 3′ end of the transcript but not between the individual shRNA units. It will also be appreciated that single RNAs from which multiple siRNAs can be generated need not be produced in vivo but may instead be chemically synthesized or produced using in vitro transcription and provided exogenously.

In another embodiment of the invention, the vector includes multiple promoters, each of which directs synthesis of a self-complementary RNA molecule that hybridizes to form an shRNA. The multiple shRNAs may all target the same transcript, or they may target different transcripts. Any combination of viral transcripts may be targeted. Example 11 provides details of the design and testing of shRNAs transcribed from DNA vectors for inhibition of influenza virus infection according to certain embodiments of the invention. See also FIG. 21. In general, according to certain embodiments of the invention the siRNAs and/or shRNAs expressed in the cell comprise a base-paired (duplex) region approximately 19 nucleotides long.

Those of ordinary skill in the art will further appreciate that in vivo expression of siRNAs or shRNAs according to the present invention may allow the production of cells that produce the siRNA or shRNA over long periods of time (e.g., greater than a few days, preferably at least several weeks to months, more preferably at least a year or longer, possibly a lifetime). Such cells may be protected from influenza virus indefinitely.

Preferred viral vectors for use in the compositions to provide intracellular expression of siRNAs and shRNAs include, for example, retroviral vectors and lentiviral vectors. See, e.g., Kobinger, G. P., et al., Nat Biotechnol 19(3):225-30, 2001, describing a vector based on a Filovirus envelope protein-pseudotyped HIV vector, which efficiently transduces intact airway epithelium from the apical surface. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

In certain embodiments of the invention the vector is a lentiviral vector whose presence within a cell results in transcription of one or more RNAs that self-hybridize or hybridize to each other to form an shRNA or siRNA that inhibits expression of at least one transcript in the cell. For purposes of description it will be assumed that the vector is a lentiviral vector such as those described in Rubinson, D., et al, Nature Genetics, Vol. 33, pp. 401-406, 2003. However, it is to be understood that other retroviral or lentiviral vectors may also be used. According to various embodiments of the invention the lentiviral vector may be either a lentiviral transfer plasmid or a lentiviral particle, e.g., a lentivirus capable of infecting cells. In certain embodiments of the invention the lentiviral vector comprises a nucleic acid segment operably linked to a promoter, so that transcription from the promoter (i.e., transcription directed by the promoter) results in synthesis of an RNA comprising complementary regions that hybridize to form an shRNA targeted to the target transcript. According to certain embodiments of the invention the shRNA comprises a base-paired region approximately 19 nucleotides long. According to certain embodiments of the invention the RNA may comprise more than 2 complementary regions, so that self-hybridization results in multiple base-paired regions, separated by loops or single-stranded regions. The base-paired regions may have identical or different sequences and thus may be targeted to the same or different regions of a single transcript or to different transcripts.

In certain embodiments of the invention the lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. According to certain embodiments of the invention the siRNA comprises a base-paired region approximately 19 nucleotides long. In certain embodiments of the invention the lentiviral vector comprises at least two promoters and at least two nucleic acid segments, wherein each promoter is operably linked to a nucleic acid segment, so that transcription from the promoters results in synthesis of two complementary RNAs that hybridize with each other to form an siRNA targeted to the target transcript.

As mentioned above, the lentiviral vectors may be lentiviral transfer plasmids or infectious lentiviral particles (e.g., a lentivirus or pseudotyped lentivirus). See, e.g., U.S. Pat. No. 6,013,516 and references 113-117 for further discussion of lentiviral transfer plasmids, lentiviral particles, and lentiviral expression systems. As is well known in the art, lentiviruses have an RNA genome. Therefore, where the lentiviral vector is a lentiviral particle, e.g., an infectious lentivirus, the viral genome must undergo reverse transcription and second strand synthesis to produce DNA capable of directing RNA transcription. In addition, where reference is made herein to elements such as promoters, regulatory elements, etc., it is to be understood that the sequences of these elements are present in RNA form in the lentiviral particles of the invention and are present in DNA form in the lentiviral transfer plasmids of the invention. Furthermore, where a template for synthesis of an RNA is “provided by” RNA present in a lentiviral particle, it is understood that the RNA must undergo reverse transcription and second strand synthesis to produce DNA that can serve as a template for synthesis of RNA (transcription). Vectors that provide templates for synthesis of siRNA or shRNA are considered to provide the siRNA or shRNA when introduced into cells in which such synthesis occurs.

Inventive siRNAs or shRNAs may be introduced into cells by any available method. For instance, siRNAs, shRNAs, or vectors encoding them can be introduced into cells via conventional transformation or transfection techniques. As used herein, the terms “transformation” and “transfection” are intended to refer to a variety of art-recognized techniques for introducing foreign nucleic acid (e.g., DNA or RNA) into a cell, including calcium phosphate or calcium chloride co-precipitation, DEAE-dextran-mediated transfection, lipofection, injection, or electroporation. As described below, one aspect of the invention includes the use of a variety of delivery agents for introducing siRNAs, shRNAs, and or vectors (either DNA vectors or viral vectors) that provide a template for synthesis of an siRNA or shRNA into cells including, but not limited to, cationic polymers; various peptide molecular transporters including arginine-rich peptides, histidine-rich peptides, and cationic and neutral lipids; various non-cationic polymers; liposomes; carbohydrates; and surfactant materials. The invention also encompasses the use of delivery agents that have been modified in any of a variety of ways, e.g., by addition of a delivery-enhancing moiety to the delivery agent, as described further below.

The present invention encompasses any cell manipulated to contain an inventive siRNA, shRNA, or vector that provides a template for synthesis of an inventive siRNA or shRNA. Preferably, the cell is a mammalian cell, particularly human. Most preferably the cell is a respiratory epithelial cell. Optionally, such cells also contain influenza virus RNA. In some embodiments of the invention, the cells are non-human cells within an organism. For example, the present invention encompasses transgenic animals engineered to contain or express inventive siRNAs or shRNAs. Such animals are useful for studying the function and/or activity of inventive siRNAs and shRNAs, and/or for studying the influenza virus infection/replication system. As used herein, a “transgenic animal” is a non-human animal in which one or more of the cells of the animal includes a transgene. A transgene is exogenous DNA or a rearrangement, e.g., a deletion of endogenous chromosomal DNA, which preferably is integrated into or occurs in the genome of the cells of a transgenic animal. A transgene can direct the expression of an encoded siRNA product in one or more cell types or tissues of the transgenic animal. Preferred transgenic animals are non-human mammals, more preferably rodents such as rats or mice. Other examples of transgenic animals include non-human primates, sheep, dogs, cows, goats, birds such as chickens, amphibians, and the like. According to certain embodiments of the invention the transgenic animal is of a variety used as an animal model (e.g., murine, ferret, or primate) for testing potential influenza therapeutics.

III. Broad Inhibition of Viral RNA Accumulation

One general characteristic of RNAi-mediated inhibition of gene expression is its specificity. In other words, siRNA targeted to a particular transcript sequence typically does not result in degradation of other transcripts. However, as described in Example 6, the inventors have discovered that siRNAs targeted to NP, PA, or PB1 transcripts also result in reduced levels of other viral RNAs, including RNAs having sequences unrelated to the NP or PA sequence. In addition, as shown in Example 5, while it appears likely that the direct target of siRNA is viral mRNA, administration of siRNAs targeted to NP, PA inhibited accumulation of the corresponding vRNA and cRNA in addition to inhibiting accumulation of NP or PA mRNA. As shown in Example 7, these effects are not due to the interferon response or to virus-mediated degradation of viral transcripts. Furthermore, the effect was specific to viral transcripts since there was little or no effect on a variety of cellular transcripts. Potential mechanisms that may mediate this effect are discussed in Example 6. Regardless of the exact mechanism, these findings demonstrate that administration of an siRNA targeted to a second transcript can, under certain conditions, also affect a first transcript or transcripts to which the siRNA is not targeted, including, for example, a first transcript that lacks significant identity or homology to the second transcript. In particular, this may occur where the protein encoded by the second transcript (or, potentially, the transcript itself) is involved in synthesis, processing, or stability of the first transcript.

Thus the invention provides a method of inhibiting a first transcript comprising administering an siRNA targeted to a second transcript, wherein inhibition of the second transcript results in inhibition of the first transcript. In general, the first and second transcripts are non-identical and non-homologous at least over the portion of the second transcript that is targeted. However, in various embodiments of the invention the first and second transcripts may share a region of homology or identity over the portion of the second transcript that is targeted (e.g., a portion corresponding to a 19 nucleotide duplex portion of the siRNA). If the siRNA does not include a region of identity to the first transcript of at least 5 consecutive nucleotides, then the siRNA is not targeted to the first transcript. In general, the siRNA targeted to the second transcript is not targeted to the first transcript. If there is a shared region of homology or identity, such region may, but need not, include part or all of the target sequence. Appropriate second transcripts (target transcripts) include those that encode proteins such as RNA-binding proteins or any other protein that plays a role in stabilizing RNA. In general, the word “inhibition” refers to a reduction in the level or amount of the transcript. However, other mechanisms of inhibition are also included. The method of inhibition may be either direct or indirect.

As discussed further in Example 6, while not wishing to be bound by any theory the inventors suggest that the ability of transcripts targeted to NP to cause reduced levels of accumulation of mRNA, vRNA, and cRNA of the NS, M, NS, PB1, PB2, and PA genes transcripts is probably a result of the importance of NP protein in binding and stabilizing these transcripts, and not because NP-specific siRNA targets RNA degradation non-specifically. In addition, while not wishing to be bound by any theory the inventors suggest that the ability of transcripts targeted to PA to cause reduced levels of accumulation of mRNA, vRNA, and cRNA of the NS, M, NS, PB11, PB2, and PA genes transcripts is probably a result of the importance of PA protein in the synthesis of viral transcripts, and not because PA-specific siRNA targets RNA degradation non-specifically. In the presence of PA-specific siRNA, newly transcribed PA mRNA is degraded, resulting in inhibition of PA protein synthesis. Despite the presence of approximately 30-60 copies of PA protein (RNA transcriptase) per influenza virion (1), without newly synthesized PA protein, further viral transcription and replication are likely inhibited. It is believed that the ability of certain siRNAs to cause a reduction in levels of transcripts to which they are not specifically targeted has not been demonstrated in other systems.

The inventors have recognized that target transcripts that encode proteins that play a role in stabilizing other RNA molecules or in synthesizing RNA may be preferred targets for inhibiting growth, replication, infectivity, etc., of an infectious agent. Thus the invention provides a method of inhibiting the growth, infectivity, or replication of an infectious agent comprising administering an siRNA targeted to a target transcript, wherein inhibition of the target transcript results in inhibition of at least one other transcript, wherein such other transcript is agent-specific. The target transcript may, but need not be, an agent-specific transcript. The at least one other transcript may, but need not, share a region of homology or identity with the target transcript. If there is a shared region of homology or identity, such region may, but need not, include part or all of the target sequence. Appropriate target transcripts include those that encode proteins such as RNA-binding proteins or any other protein that plays a role in stabilizing RNA. Appropriate target transcripts also include those that play a role in RNA synthesis or processing, e.g., polymerases, reverse transcriptases, etc.

The results described herein suggest that, in general, siRNAs targeted to transcripts that encode RNA or DNA binding proteins that normally bind to agent-specific nucleic acids (DNA or RNA) are likely to have broad effects (e.g., effects on other agent-specific transcripts) rather than simply reducing the level of the targeted RNA. Similarly, the results described herein suggest that, in general, siRNAs targeted to the polymerase genes (RNA polymerase, DNA polymerase, or reverse transcriptase) of infectious agents are likely to have broad effects (e.g., effects on other agent-specific transcripts) rather than simply reducing levels of polymerase RNA.

Targeting transcripts that encode proteins that specifically stabilize RNAs of the infectious agent rather than those of the host cell offers the opportunity for selectively reducing the level of agent-specific transcripts while not affecting the level of host cell transcripts. Thus delivery of such siRNAs would not be expected to adversely affect cells of the host organism. This approach is not limited to transcripts that encode proteins that specifically stabilize RNAs of the infectious agent rather than those of the host cell but also applies to transcripts that encode proteins that are specifically involved in any aspect of processing, synthesis, and/or translation of agent-specific transcripts (i.e., transcripts whose template is part of the agent's genome rather than the host cell's genome) rather than host cell transcripts. Such proteins include, but are not limited to, proteins that are involved in synthesizing, splicing, or capping agent-specific transcripts but not host cell transcripts.

IV. Identification and Testing of siRNAs and shRNAs that Inhibit Influenza Virus

As noted above, the present invention provides a system for identifying siRNAs that are useful as inhibitors of influenza virus infection and/or replication. Since, as noted above, shRNAs are processed intracellularly to produce siRNAs having duplex portions with the same sequence as the stem structure of the shRNA, the system is equally useful for identifying shRNAs that are useful as inhibitors of influenza virus infection. For purposes of description this section will refer to siRNAs, but the system also encompasses corresponding shRNAs. Specifically, the present invention demonstrates the successful preparation of siRNAs targeted to viral genes to block or inhibit viral infection and/or replication. The techniques and reagents described herein can readily be applied to design potential new siRNAs, targeted to other genes or gene regions, and tested for their activity in inhibiting influenza virus infection and/or replication as discussed herein. It is expected that influenza viruses will continue to mutate and undergo reassortment and that it may be desirable to continue to develop and test new, differently targeted siRNAs.

In various embodiments of the invention potential influenza virus inhibitors can be tested by introducing candidate siRNA(s) into cells (e.g., by exogenous administration or by introducing a vector or construct that directs endogenous synthesis of siRNA into the cell) prior to, simultaneously with, or after transfection with an influenza genome or portion thereof (e.g., within minutes, hours, or at most a few days) or prior to, simultaneously with, or after infection with influenza virus. Alternately, potential influenza virus inhibitors can be tested by introducing candidate siRNA(s) into cells that are productively infected with influenza virus (i.e., cells that are producing progeny virus). The ability of the candidate siRNA(s) to reduce target transcript levels and/or to inhibit or suppress one or more aspects or features of the viral life cycle such as viral replication, pathogenicity, and/or infectivity is then assessed. For example, production of viral particles and/or production of viral proteins, etc., can be assessed either directly or indirectly using methods well known in the art.

Cells to which inventive siRNA compositions have been delivered (test cells) may be compared with similar or comparable cells that have not received the inventive composition (control cells, e.g., cells that have received either no siRNA or a control siRNA such as an siRNA targeted to a non-viral transcript such as GFP). The susceptibility of the test cells to influenza virus infection can be compared with the susceptibility of control cells to infection. Production of viral protein(s) and/or progeny virus may be compared in the test cells relative to the control cells. Other indicia of viral infectivity, replication, pathogenicity, etc., can be similarly compared. Standard in vitro antiviral assays may utilize inhibition of viral plaques, viral cytopathic effect (CPE), and viral hemagglutinin or other protein, inhibition of viral yield, etc. The CPE can be determined visually and by dye uptake. See, e.g., Sidwell, R. W. and Smee, D. F, “In vitro and in vivo assay systems for study of influenza virus inhibitors” Antiviral Res 2000 October; 48(1):1-16, 2000. Generally, test cells and control cells would be from the same species and of similar or identical cell type. For example, cells from the same cell line could be compared. When the test cell is a primary cell, typically the control cell would also be a primary cell. Typically the same influenza virus strain would be used to compare test cells and control cells.

For example, as described in Example 2, the ability of a candidate siRNA to inhibit influenza virus production may conveniently be determined by (i) delivering the candidate siRNA to cells (either prior to, at the same time as, or after exposure to influenza virus); (ii) assessing the production of viral hemagglutinin using a hemagglutinin assay, and (iii) comparing the amount of hemagglutinin produced in the presence of the siRNA with the amount produced in the absence of the siRNA. (The test need not include a control in which the siRNA is absent but may make use of previous information regarding the amount of hemagglutinin produced in the absence of inhibition.) A reduction in the amount of hemagglutinin strongly suggests a reduction in virus production. This assay may be used to test siRNAs that target any viral transcript and is not limited to siRNAs that target the transcript that encodes the viral hemagglutinin.

The ability of a candidate siRNA to reduce the level of the target transcript may also be assessed by measuring the amount of the target transcript using, for example, Northern blots, nuclease protection assays, reverse transcription (RT)-PCR, real-time RT-PCR, microarray analysis, etc. The ability of a candidate siRNA to inhibit production of a polypeptide encoded by the target transcript (either at the transcriptional or post-transcriptional level) may be measured using a variety of antibody-based approaches including, but not limited to, Western blots, immunoassays, ELISA, flow cytometry, protein microarrays, etc. In general, any method of measuring the amount of either the target transcript or a polypeptide encoded by the target transcript may be used.

In general, certain preferred influenza virus inhibitors reduce the target transcript level at least about 2 fold, preferably at least about 4 fold, more preferably at least about 8 fold, at least about 16 fold, at least about 64 fold or to an even greater degree relative to the level that would be present in the absence of the inhibitor (e.g., in a comparable control cell lacking the inhibitor). In general, certain preferred influenza virus inhibitors inhibit viral replication, so that the level of replication is lower in a cell containing the inhibitor than in a control cell not containing the inhibitor by at least about 2 fold, preferably at least about 4 fold, more preferably at least about 8 fold, at least about 16 fold, at least about 64 fold, at least about 100 fold, at least about 200 fold, or to an even greater degree. In particular, as described in Example 2, the inventors have shown that viral titer, as measured by production of hemagglutinin, was reduced by more than 256 fold in cells infected with influenza virus strain A/PR/8/34 (H1N1) to which a single dose of siRNA (PB1-2257) was administered and by more than 120 fold in cells infected with influenza virus strain A/WSN/33 (H1N1) to which a single dose of siRNA (NP-1496 and others) was administered. When measured by plaque assay at an MOI of 0.001, the fold inhibition was even greater, i.e., at least about 30,000 fold. Even at an MOI of 0.1, NP-1496 inhibited virus production about 200-fold.

Certain preferred influenza virus inhibitors inhibit viral replication so that development of detectable viral titer is prevented for at least 24 hours, at least 36 hours, at least 48 hours, or at least 60 hours following administration of the siRNA and infection of the cells. Certain preferred influenza virus inhibitors prevent (i.e., reduce to undetectable levels) or significantly reduce viral replication for at least 24 hours, at least 36 hours, at least 48 hours, or at least 60 hours following administration of the siRNA. According to various embodiments of the invention a significant reduction in viral replication is a reduction to less than approximately 90% of the level that would occur in the absence of the siRNA, a reduction to less than approximately 75% of the level that would occur in the absence of the siRNA, a reduction to less than approximately 50% of the level that would occur in the absence of the siRNA, a reduction to less than approximately 25% of the level that would occur in the absence of the siRNA, or a reduction to less than approximately 10% of the level that would occur in the absence of the siRNA. Reduction in viral replication may be measured using any suitable method including, but not limited to, measurement of HA titer.

Potential influenza virus inhibitors can also be tested using any of variety of animal models that have been developed. Compositions comprising candidate siRNA(s), constructs or vectors capable of directing synthesis of such siRNAs within a host cell, or cells engineered or manipulated to contain candidate siRNAs may be administered to an animal prior to, simultaneously with, or following infection with an influenza virus. The ability of the composition to prevent viral infection and/or to delay or prevent appearance of influenza-related symptoms and/or lessen their severity relative to influenza-infected animals that have not received the potential influenza inhibitor is assessed. Such models include, but are not limited to, murine, chicken, ferret, and non-human primate models for influenza infection, all of which are known in the art and are used for testing the efficacy of potential influenza therapeutics and vaccines. See, e.g, Sidwell, R. W. and Smee, D. F, referenced above. Such models may involve use of naturally occurring influenza virus strains and/or strains that have been modified or adapted to existence in a particular host (e.g., the WSN or PR8 strains, which are adapted for replication in mice). See Examples 6, 7, 8, 9, and 10 for further discussion of methods for testing siRNA compositions in vitro and in vivo.

V. Composition is for Improved Delivery of siRNA, shRNA, and RNAi-Inducing Vectors

The inventors have recognized that effective RNAi therapy in general, including prevention and therapy of influenza virus infection, will be enhanced by efficient delivery of siRNAs, shRNAs, and/or RNAi-inducing vectors into cells in intact organisms. In the case of influenza virus, such agents must be introduced into cells in the respiratory tract, where influenza infection normally occurs. For use in humans, it may be preferable to employ non-viral methods that facilitate intracellular uptake of siRNA or shRNA. The invention therefore provides compositions comprising any of a variety of non-viral delivery agents for enhanced delivery of siRNA, shRNA, and/or RNAi-inducing vectors to cells in intact organisms, e.g., mammals and birds. As used herein, the concept of “delivery” includes transport of an siRNA, shRNA, or RNAi-inducing vector from its site of entry into the body to the location of the cells in which it is to function, in addition to cellular uptake of the siRNA, shRNA, or vector and any subsequent steps involved in making siRNA or shRNA available to the intracellular RNAi machinery (e.g., release or siRNA or shRNA from endosomes).

The invention therefore encompasses compositions comprising an RNAi-inducing agent such as an siRNA, shRNA, or an RNAi-inducing vector whose presence within a cell results in production of an siRNA or shRNA, wherein the siRNA or shRNA is targeted to an influenza virus transcript, and any of a variety of delivery agents including, but not limited to, cationic polymers, modified cationic polymers, peptide molecular transporters (including arginine or histidine-rich peptides), lipids (including cationic lipids, neutral lipids, and combinations thereof), liposomes, lipopolyplexes, non-cationic polymers, surfactants suitable for introduction into the lung, etc. (It is noted that the “wherein” clause in the foregoing language and elsewhere is intended to refer to siRNAs or shRNAs in the composition in addition to those produced as a result of the presence of a vector within a cell.) Certain of the delivery agents are modified to incorporate a moiety that increases delivery or increases the selective delivery of the siRNA, shRNA, or RNAi-inducing vector to cells in which it is desired to inhibit an influenza virus transcript. In certain embodiments of the invention the delivery agent is biodegradable. Certain of the delivery agents suitable for use in the present invention are described below and in co-pending U.S. patent application entitled “Compositions and Methods for Delivery of Short Interfering RNA and Short Hairpin RNA to Mammals”, filed on even date herewith, which is herein incorporated by reference.

A. Cationic Polymers and Modified Cationic Polymers

Cationic polymer-based systems have been investigated as carriers for DNA transfection (35). The ability of cationic polymers to promote intracellular uptake of DNA is thought to arise partly from their ability to bind to DNA and condense large plasmid DNA molecules into smaller DNA/polymer complexes for more efficient endocytosis. The DNA/cationic polymer complexes also act as bioadhesives because of their electrostatic interaction with negatively charged sialic acid residues of cell surface glycoproteins (36). In addition, some cationic polymers apparently promote disruption of the endosomal membrane and therefore release of DNA into the cytosol (32). The invention therefore provides compositions comprising (i) an RNAi-inducing entity targeted to an influenza virus transcript and (ii) a cationic polymer. The invention further provides methods of inhibiting target gene expression comprising administering a composition comprising an RNA-inducing entity targeted to an influenza virus transcript to a mammalian subject. In particular, the invention provides methods of treating and/or preventing influenza virus infection comprising administering a composition comprising an RNA-inducing entity that targets an influenza virus transcript and a cationic polymer to a mammalian subject. In various embodiments of the invention the RNAi-inducing entity is an siRNA, shRNA, or RNAi-inducing vector.

In general, a cationic polymer is a polymer that is positively charged at approximately physiological pH, e.g., a pH ranging from approximately 7.0 to 7.6, preferably approximately 7.2 to 7.6, more preferably approximately 7.4. Such cationic polymers include, but are not limited to, polylysine (PLL), polyarginine (PLA), polyhistidine, polyethyleneimine (PEI) (37), including linear PEI and low molecular weight PEI as described, for example, in (76), polyvinylpyrrolidone (PVP) (38), and chitosan (39, 40). It will be appreciated that certain of these polymers comprise primary amine groups, imine groups, guanidine groups, and/or imidazole groups. Preferred cationic polymers have relatively low toxicity and high DNA transfection efficiency.

Suitable cationic polymers also include copolymers comprising subunits of any of the foregoing polymers, e.g., lysine-histidine copolymers, etc. The percentage of the various subunits need not be equal in the copolymers but may be selected, e.g., to optimize such properties as ability to form complexes with nucleic acids while minimizing cytotoxicity. Furthermore, the subunits need not alternate in a regular fashion. Appropriate assays to evaluate various polymers with respect to desirable properties are described in the Examples. Preferred cationic polymers also include polymers such as the foregoing, further incorporating any of various modifications. Appropriate modifications are discussed below and include, but are not limited to, modification with acetyl, succinyl, acyl, or imidazole groups (32).

While not wishing to be bound by any theory, it is believed that cationic polymers such as PEI compact or condense DNA into positively charged particles capable of interacting with anionic proteoglycans at the cell surface and entering cells by endocytosis. Such polymers may possess the property of acting as a “proton sponge” that buffers the endosomal pH and protects DNA from degradation. Continuous proton influx also induces endosome osmotic swelling and, rupture, which provides an escape mechanism for DNA particles to the cytoplasm. (See, e.g., references 85-87; U.S. Pat. No. 6,013,240; WO9602655 for further information on PEI and other cationic polymers useful in the practice of the invention) According to certain embodiments of the invention the commercially available PEI reagent known as jetPEI™ (Qbiogene, Carlsbad, Calif.), a linear form of PEI (U.S. Pat. No. 6,013,240) is used.

As described in Example 12, the inventors have shown that compositions comprising PEI, PLL, or PLA and an siRNA that targets an influenza virus RNA significantly inhibit production of influenza virus in mice when administered intravenously either before or after influenza virus infection. The inhibition is dose-dependent and exhibits additive effects when two siRNAs targeted to different influenza virus RNAs were used. Thus siRNA, when combined with a cationic polymer such as PEI, PLL, or PLA, is able to reach the lung, to enter cells, and to effectively inhibit the viral replication cycle. It is believed that these findings represent the first report of efficacy in inhibiting production of infectious virus in a mammal using siRNA (as opposed, for example, to inhibiting production of viral transcripts or intermediates in a viral replicative cycle).

It is noted that other efforts to deliver siRNA intravenously to solid organs and tissues within the body (see, e.g., McCaffrey 2002; McCaffrey 2003; Lewis, D. L., et al.) have employed the technique known as hydrodynamic transfection, which involves rapid delivery of large volumes of fluid into the tail vein of mice and has been shown to result in accumulation of significant amounts of plasmid DNA in solid organs, particularly the liver (Liu 1999; Zhang 1999; Zhang 2000). This technique involves delivery of fluid volumes that are almost equivalent to the total blood volume of the animal, e.g., 1.6 ml for mice with a body weight of 18-20 grams, equivalent to approximately 8-12% of body weight, as opposed to conventional techniques that involve injection of approximately 200 μl of fluid (Liu 1999). In addition, injection using the hydrodynamic transfection approach takes place over a short time interval (e.g., 5 seconds), which is necessary for efficient expression of injected transgenes (Liu 1999).

While the mechanism by which hydrodynamic transfection achieves transfer and high level expression of injected transgenes in the liver is not entirely clear, it is thought to be due to a reflux of DNA solution into the liver via the hepatic vein due to a transient cardiac congestion (Zhang 2000). A comparable approach for therapeutic purposes in humans seems unlikely to be feasible. The inventors, in contrast, have used conventional volumes of fluid (e.g., 200 μl) and have demonstrated effective delivery of siRNA to the lung under conditions that would be expected to lead to minimal expression of injected transgenes even in the liver, the site at which expression is most readily achieved using hydrodynamic transfection.

The invention therefore provides a method of inhibiting expression of a viral transcript, e.g., an influenza virus transcript, in a cell within a mammalian subject comprising the step of introducing a composition comprising an RNAi-inducing entity targeted to the target transcript into the vascular system of the subject using a conventional injection technique, e.g., a technique using conventional pressures and/or conventional volumes of fluid. The RNAi-inducing entity may be an siRNA, shRNA, or RNAi-inducing vector. In certain preferred embodiments of the invention the composition comprises a cationic polymer. In preferred embodiments of the invention the composition is introduced in a fluid volume equivalent to less than 10% of the subject's body weight. In certain embodiments of the invention the fluid volume is equivalent to less than 5%, less than 2%, less than 1%, or less than 0.1% of the subject's body weight. In certain embodiments of the invention the method achieves delivery of effective amounts of siRNA or shRNA in a cell in a body tissue or organ other than the liver. In certain preferred embodiments of the invention the composition is introduced into a vein, e.g., by intravenous injection. However, the composition may also be administered into an artery, delivered using a device such as a catheter, indwelling intravenous line, etc. In certain preferred embodiments of the invention the RNAi-inducing entity inhibits production of the virus.

As described in Example 15, the inventors have also shown that the cationic polymers PLL and PLA are able to form complexes with siRNAs and promote uptake of functional siRNA in cultured cells. Transfection with complexes of PLL and NP-1496 or complexes of PLA and NP-1496 siRNA inhibited production of influenza virus in cells. These results and the results in mice discussed above demonstrate the feasibility of using mixtures of cationic polymers and siRNA for delivery of siRNA to mammalian cells in the body of a subject. The approach described in Example 15 may be employed to test additional polymers, particularly polymers modified by addition of groups e.g., acyl, succinyl, acetyl, or imidazole groups) to reduce cytotoxicity, and to optimize those that are initially effective. In general, certain preferred modifications result in a reduction in the positive charge of the cationic polymer. Certain preferred modifications convert a primary amine into a secondary amine. Methods for modifying cationic polymers to incorporate such additional groups are well known in the art. (See, e.g., reference 32). For example, the ε-amino group of various residues may be substituted, e.g., by conjugation with a desired modifying group after synthesis of the polymer. In general, it is desirable to select a % substitution sufficient to achieve an appropriate reduction in cytotoxicity relative to the unsubstituted polymer while not causing too great a reduction in the ability of the polymer to enhance delivery of the RNAi-inducing entity. Accordingly, in certain embodiments of the invention between 25% and 75% of the residues in the polymer are substituted. In certain embodiments of the invention approximately 50% of the residues in the polymer are substituted. It is noted that similar effects may be achieved by initially forming copolymers of appropriately selected monomeric subunits, i.e., subunits some of which already incorporate the desired modification.

A variety of additional cationic polymers may also be used. Large libraries of novel cationic polymers and oligomers from diacrylate and amine monomers have been developed and tested in DNA transfection. These polymers are referred to herein as poly(β-amino ester) (PAE) polymers. For example, a library of 140 polymers from 7 diacrylate monomers and 20 amine monomers has been described (34) and larger libraries can be produced using similar or identical methodology. Of the 140 members of this library, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, two of the 56 polymers mediated DNA transfection into COS-7 cells. Transfection efficiencies of the novel polymers were 4-8 times higher than PEI and equal or better than Lipofectamine 2000. The invention therefore provides compositions comprising at least one siRNA molecule and a cationic polymer, wherein the cationic polymer is a poly(β-amino ester), and methods of inhibiting target gene expression by administering such compositions. Poly(beta-amino esters) are further described in U.S. published patent application 20020131951, entitled “Biodegradable poly(beta-amino esters) and uses thereof”, filed Sep. 19, 2002, by Langer et al., and Anderson (2003). It is noted that the cationic polymers for use to facilitate delivery of RNAi-inducing entities may be modified so that they incorporate one or more residues other than the major monomeric subunit of which the polymer is comprised. For example, one or more alternate residues may be added to the end of a polymer, or polymers may be joined by a residue other than the major monomer of which the polymer is comprised.

Additional cationic polymers that may also be used to enhance delivery of inventive RNAi-inducing entities include polyamidoamine (PAMAM) dendrimers, poly(2-dimethylamino)ethyl methacrylate (pDMAEMA), and its quaternary amine analog poly(2-triemethylamino)ethyl methacrylate (pTMAEMA), poly [a-(4-aminobutyl)-L-glycolic acid (PAGA), and poly (4-hydroxy-1-proline ester). See Han (2000) for further description of these agents.

B. Peptide Molecular Transporters

Studies have shown that a variety of peptides are able to act as delivery agents for nucleic acids. (As used herein, a polypeptide is considered to be a “peptide” if it shorter than approximately 50 amino acids in length.) For example, transcription factors, including HIV Tat protein (42, 43), VP22 protein of herpes simplex virus (44), and Antennapedia protein of Drosophila (45), can penetrate the plasma membrane from the cell surface. The peptide segments responsible for membrane penetration consist of 11-34 amino acid residues, are highly enriched for arginine, and are often referred to as arginine rich peptides (ARPs) or penetratins. When covalently linked with much larger polypeptides, the ARPs are capable of transporting the fused polypeptide across the plasma membrane (46-48). Similarly, when oligonucleotides were covalently linked to ARPs, they were much more rapidly taken up by cells (49, 50). Recent studies have shown that a polymer of eight arginines is sufficient for this transmembrane transport (51). Like cationic polymers, ARPs are also positively charged and likely capable of binding siRNA, suggesting that it is probably not necessary to covalently link siRNA to ARPs.

The invention therefore provides compositions comprising at least one RNAi-inducing entity, wherein the RNAi-inducing entity is targeted to an influenza virus transcript, and a peptide molecular transporter and methods of inhibiting target gene expression by administering such compositions. The invention provides methods of treating and/or preventing influenza virus infection comprising administering such compositions to a subject at risk of or suffering from influenza. Peptide molecular transporters include, but are not limited to, those described in references 46-51, 120, and 134-136 and variations thereof evident to one of ordinary skill in the art. Arginine-rich peptides include a peptide consisting of arginine residues only.

Generally, preferred peptide molecular transporters are less than approximately 50 amino acids in length. According to certain embodiments of the invention the peptide molecular transporter is a peptide having length between approximately 7 and 34 amino acids. Many of the preferred peptides are arginine-rich. According to certain embodiments of the invention a peptide is arginine-rich if it includes at least 20%, at least 30%, or at least 40%, or at least 50%, or at least 60% or at least 70%, or at least 80%, or at least 90% arginine. According to certain embodiments of the invention the peptide molecular transporter is an arginine-rich peptide that includes between 6 and 20 arginine residues. According to certain embodiments of the invention the arginine-rich peptide consists of between 6 and 20 arginine residues. According to certain embodiments of the invention the siRNA and the peptide molecular transporter are covalently bound, whereas in other embodiments of the invention the siRNA and the peptide molecular transporter are mixed together but are not covalently bound to one another. According to certain embodiments of the invention a histidine-rich peptide is used (88). In accordance with the invention histidine-rich peptides may exhibit lengths and percentage of histidine residues as described for arginine-rich peptides. The invention therefore provides compositions comprising at least one RNAi-inducing entity, wherein the RNAi-inducing entity is targeted to an influenza virus transcript and a histidine-rich peptide and methods of inhibiting target transcript expression by administering such compositions. The invention provides methods of treating and/or preventing influenza virus infection comprising administering such compositions to a subject at risk of or suffering from influenza.

Additional peptides or modified peptides that facilititate the delivery of RNAi-inducing entities to cells in a subject may also be used in the inventive compositions. For example, a family of lysine-rich peptides has been described, generally containing between 8 and approximately 50 lysine residues (McKenzie 2000). While these peptides can enhance uptake of nucleic acids by cells in tissue culture, they are less efficient delivery vehicles for nucleic acids in the body of a subject than longer polypeptides, e.g., PLL comprising more than 50 lysine residues. This may be due in part to insufficient stability of the nucleic acid/peptide complex within the body. Insertion of multiple cysteines at various positions within the peptides results in low molecular weight DNA condensing peptides that spontaneously oxidize after binding plasmid DNA to form interpeptide disulfide bonds. These cross-linked DNA delivery vehicles were more efficient inducers of gene expression when used to deliver plasmids to cells relative to uncrosslinked peptide DNA condensates (McKenzie 2002). In addition, peptides that comprise sulfhydryl residues for formation of disulfide bonds may incorporate polyethylene glycol (PEG), which is believed to reduce nonspecific binding to serum proteins (Park 2002).

Glycopeptides that include moieties such as galactose or mannose residues may also be used to enhance the selective uptake of RNAi-inducing entities in accordance with the present invention, as discussed further below. Such glycopeptides may also include sulfhydryl groups for formation of disulfide bonds (Park 2002). The invention encompasses administration of various agents that enhance exit of nucleic acids from endocytic vesicles. Such agents include chloroquine (Zhang 2003) and bupivacaine (Satishchandran 2000). The exit-enhancing agents may be administered systemically, orally, and/or locally (e.g. at or in close proximity to the desired site of action). They may be delivered together with inventive siRNA, shRNA, or RNAi-inducing vectors or separately.

C. Additional Polymeric Delivery Agents

The invention provides compositions comprising inventive RNAi-inducing entities and any of a variety of polymeric delivery agents, including modified polymers, in addition to those described above. The invention further provides methods of inhibiting expression of an influenza virus transcript in a cell and methods of treating or preventing influenza virus infection by administering the compositions. Suitable delivery agents include various agents that have been shown to enhance delivery of DNA to cells. These include modified versions of cationic polymers such as those mentioned above, e.g., poly(L-histidine)-graft-poly(L-lysine) polymers (Benns 2000), polyhistidine-PEG (Putnam 2003), folate-PEG-graft-polyethyleneimine (Benns 2002), polyethylenimine-dextran sulfate (Tiyaboonchai 2003), etc. The polymers may be branched or linear and may be grafted or ungrafted. According to the invention the polymers form complexes with inventive RNAi-inducing entities, which are then administered to a subject. The complexes may be referred to as nanoparticles or nanocomposites. Any of the polymers may be modified to incorporate PEG or other hydrophilic polymers, which is useful to reduce complement activation and binding of other plasma proteins. Cationic polymers may be multiply modified. For example, a cationic polymer may be modified to incorporate a moiety that reduces the negative charge of the polymer (e.g., imidazole) and may be further modified with a second moiety such as PEG.

In addition, a variety of polymers and polymer matrices distinct from the cationic polymers described above may also be used. Such polymers include a number of non-cationic polymers, i.e., polymers not having positive charge at physiological pH. Such polymers may have certain advantages, e.g., reduced cytotoxicity and, in some cases, FDA approval. A number of suitable polymers have been shown to enhance drug and gene delivery in other contexts. Such polymers include, for example, poly(lactide) (PLA), poly(glycolide) (PLG), and poly(DL-lactide-co-glycolide) (PLGA) (Panyam 2002), which can be formulated into nanoparticles for delivery of inventive RNAi-inducing entities. Copolymers and combinations of the foregoing may also be used. In certain embodiments of the invention a cationic polymer is used to condense the siRNA, shRNA, or vector, and the condensed complex is protected by PLGA or another non-cationic polymer. Other polymers that may be used include noncondensing polymers such as polyvinyl alcohol, or poly(N-ethyl-4-vinylpyridium bromide, which may be complexed with Pluronic 85. Other polymers of use in the invention include combinations between cationic and non-cationic polymers. For example, poly(lactic-co-glycolic acid) (PLGA)-grafted poly(L-lysine) (Jeong 2002) and other combinations including PLA, PLG, or PLGA and any of the cationic polymers or modified cationic polymers such as those discussed above, may be used.

D. Delivery Agents Incorporating Delivery-Enhancing Moieties

The invention encompasses modification of any of the delivery agents to incorporate a moiety that enhances delivery of the agent to cells and/or enhances the selective delivery of the agent to cells in which it is desired to inhibit a target transcript. Any of a variety of moieties may be used including, but not limited to, (i) antibodies or antibody fragments that specifically bind to a molecule expressed by a cell in which inhibition is desired, (e.g., a respiratory epithelial cell); (ii) ligands that specifically bind to a molecule expressed by a cell in which inhibition is desired. Preferably the molecule is expressed on the surface of the cell. Monoclonal antibodies are generally preferred. In the case of respiratory epithelial cells, suitable moieties include antibodies that specifically bind to receptors such as the p2Y2 purinoceptor, bradykinin receptor, urokinase plasminogen activator R, or serpin enzyme complex may be conjugated to various of the delivery agents mentioned above to increase delivery to and selectivity for, respiratory epithelial cells. Similarly, ligands for these various molecules may be conjugated to the delivery agents to increase delivery to and selectivity for respiratory epithelial cells. See, e.g., (Ferrari 2002). In certain preferred embodiments of the invention binding of the antibody or ligand induces internalization of the bound complex. In certain embodiments of the invention the delivery enhancing agent (e.g., antibody, antibody fragment, or ligand), is conjugated to an RNAi-inducing vector (e.g., a DNA vector) to increase delivery or enhance selectivity. Methods for conjugating antibodies or ligands to nucleic acids or to the various delivery agents described herein are well known in the art. See e.g., “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991.

E. Surfactants Suitable for Introduction into the Lung

Natural, endogenous surfactant is a compound composed of phospholipids, neutral lipids, and proteins (Surfactant proteins A, B, C, and D) that forms a layer between the surfaces of alveoli in the lung and the alveolar gas and reduces alveolar collapse by decreasing surface tension within the alveoli (77-84). Surfactant molecules spread within the liquid film which bathes the entire cellular covering of the alveolar walls, where they produce an essentially mono-molecular, all pervasive layer thereon. Surfactant deficiency in premature infants frequently results in respiratory distress syndrome (RDS). Accordingly, a variety of surfactant preparations have been developed for the treatment and/or prevention of this condition. Surfactant can be extracted from animal lung lavage and from human amniotic fluid or produced from synthetic materials (see, e.g., U.S. Pat. Nos. 4,338,301; 4,397,839; 4,312,860; 4,826,821; 5,110,806). Various formulations of surfactant are commercially available, including Infasurf® (manufactured by ONY, Inc., Amherst, N.Y.); Survanta® (Ross Labs, Abbott Park, Ill.), and Exosurf Neonatal® (GlaxoSmithKline, Research Triangle Park, N.C.).

As used herein, the phrase “surfactant suitable for introduction into the lung” includes the particular formulations used in the commercially available surfactant products and the inventive compositions described and claimed in the afore-mentioned patent applications and equivalents thereof. In certain embodiments of the invention the phrase includes preparations comprising 10-20% protein and 80-90% lipid both based on the whole surfactant, which lipid consists of about 10% neutral lipid (e.g., triglyceride, cholesterol) and of about 90% phospholipid both based on the same, while the phosphatidylcholine content based on the total phospholipid is 86%, where both “%” and “part” are on the dried matter basis (see U.S. Pat. Nos. 4,388,301 and 4,397,839).

In certain embodiments of the invention the phrase includes synthetic compositions, which may be entirely or substantially free of protein, e.g., compositions comprising or consisting essentially of dipalmitoyl phosphatidylcholine and fatty alcohols, wherein the dipalmitoyl phosphatidylcholine (DPPC) constitutes the major component of the surfactant composition while the fatty alcohol comprises a minor component thereof, optionally including a non-toxic nonionic surface active agent such as tyloxapol (see U.S. Pat. Nos. 4,312,860; 4,826,821; and 5,110,806). One of ordinary skill in the art will be able to determine, by reference to the tests described in the afore-mentioned patents and literature, whether any particular surfactant composition is suitable for introduction into the lung. While not wishing to be bound by any theory, it is possible that the ability of surfactant to spread and cover the alveoli facilitates and the composition of surfactant itself, facilitate the uptake of siRNA and/or vectors by cells within the lung.

Infasurf is a sterile, non-pyrogenic lung surfactant intended for intratracheal instillation only. It is an extract of natural surfactant from calf lungs which includes phospholipids, neutral lipids, and hydrophobic surfactant-associated proteins B and C. Infasurf is approved by the U.S. Food and Drug Administration for the treatment of respiratory distress syndrome and is thus a safe and tolerated vehicle for administration into the respiratory tract and lung. Survanta is also an extract derived from bovine lung, while Exosurf Neonatal is a protein-free synthetic lung surfactant containing dipalmitoylphosphatidylcholine, cetyl alcohol, and tyloxapol. Both of these surfactant formulations have also been approved by the U.S.F.D.A. for treatment of respiratory distress syndrome.

As described in Example 14, the inventors have shown that DNA vectors that serve as templates for synthesis of shRNAs targeted to influenza RNAs can inhibit influenza virus production when mixed with Infasurf and administered to mice by intranasal instillation. In addition, as described in Example 13, the inventors showed that infection with lentiviruses expressing the same shRNAs inhibits influenza virus production in cells in tissue culture. These results demonstrate that shRNAs targeted to influenza virus RNAs can be delivered to cells and processed into siRNAs that are effective in the treatment and/or prevention of influenza virus infection. The results also demonstrate that surfactant materials such as Infasurf, e.g., materials having a composition and/or properties similar to those of natural lung surfactant, are appropriate vehicles for delivery of shRNAs to the lung. In addition, the results strongly suggest that siRNAs targeted to influenza virus will also effectively inhibit influenza virus production when delivered to the lung and/or respiratory passages. The invention therefore provides a composition comprising (i) at least one RNAi-inducing entity, wherein the RNAi-inducing entity is targeted to an influenza virus transcript and (ii) a surfactant material suitable for introduction into the lung. Inventive compositions comprising surfactant and an RNAi-inducing entity may be introduced into the lung in any of a variety of ways including instillation, by inhalation, by aerosol spray, etc. It is noted that the composition may contain less than 100% surfactant. For example, the composition may contain between approximately 10 and 25% surfactant by weight, between approximately 25 and 50% surfactant by weight, between approximately 50 and 75% surfactant by weight, between approximately 75 and 100% surfactant by weight. The invention provides methods of treating or preventing influenza comprising administering the foregoing compositions to a subject at risk of or suffering from influenza.

F. Additional Agents for Delivery of RNAi-inducing Entities to the Lung

The invention encompasses the use of a variety of additional agents and methods to enhance delivery of inventive RNAi-inducing entities to pulmonary epithelial cells. Methods include CaPO₄ precipitation of vectors prior to delivery or administration together with EGTA to cause calcium chelation. Administration with detergents and thixotrophic solutions may also be used. Perfluorochemical liquids may also be used as delivery vehicles. See (Weiss 2002) for further discussion of these methods and their applicability in gene transfer. In addition, the invention encompasses the use of protein/polyethylenimine complexes incorporating inventive RNAi-inducing entities for delivery to the lung. Such complexes comprise polyethylenimine in combination with albumin (or other soluble proteins). Similar complexes containing plasmids for gene transfer have been shown to result in delivery to lung tissues after intravascular administration (Orson 2002). Protein/PEI complexes comprising an inventive RNAi-inducing entity may also be used to enhance delivery to cells not within the lung.

G. Lipids

As described in Example 3, the inventors have shown that administration of siRNA targeted to an influenza virus transcript by injection into intact chicken embryos in the presence of the lipid agent known as Oligofectamine™ effectively inhibits influenza virus production while administration of the same siRNA in the absence of Oligofectamine did not result in effective inhibition. These results demonstrate the utility of lipid delivery agents for enhancing the efficacy of siRNA in intact organisms. The invention therefore provides a composition comprising (i) at least one RNAi-inducing entity, wherein the RNAi-inducing entity is targeted to an influenza virus transcript and (ii) a lipid. In addition, the invention provides methods for inhibiting influenza virus production and methods for treating influenza infection comprising administering the inventive composition to a subject.

VI. Analysis of Influenza Virus Infection/Replication

As noted above, one use for the RNAi-inducing entities of the present invention is in the analysis and characterization of the influenza virus infection/replication cycle and of the effect of various viral proteins on host cells. siRNAs and shRNAs may be designed that are targeted to any of a variety of viral genes involved in one or more stages of the viral infection and/or replication cycle and/or viral genes that affect host cell functions or activities such as metabolism, biosynthesis, cytokine release, etc. siRNAs, shRNAs, or RNAi-inducing vectors may be introduced into cells prior to, during, or after viral infection, and their effects on various stages of the infection/replication cycle and on cellular activity and function may be assessed as desired.

VII. Therapeutic Applications

As mentioned above, compositions comprising the RNAi-inducing entities of the present invention may be used to inhibit or reduce influenza virus infection or replication. In such applications, an effective amount of an inventive composition is delivered to a cell or organism prior to, simultaneously with, or after exposure to influenza virus. Preferably, the amount of the RNAi-inducing entity is sufficient to reduce or delay one or more symptoms of influenza virus infection. For purposes of description this section will refer to inventive siRNAs, but as will be evident the invention encompasses similar applications for other RNAi-inducing entities targeted to influenza virus transcripts.

Inventive siRNA-containing compositions may comprise a single siRNA species, targeted to a single site in a single target transcript, or may comprise a plurality of different siRNA species, targeted to one or more sites in one or more target transcripts. Example 8 describes a general approach to the systematic identification of siRNAs with superior ability to inhibit influenza virus production either alone or in combination.

In some embodiments of the invention, it will be desirable to utilize compositions containing collections of different siRNA species targeted to different genes. For example, it may be desirable to attack the virus at multiple points in the viral life cycle using a variety of siRNAs directed against different viral transcripts. According to certain embodiments of the invention the siRNA composition contains an siRNA targeted to each viral genome segment.

According to certain embodiments of the invention, inventive siRNA compositions may contain more than one siRNA species targeted to a single viral transcript. To give but one example, it may be desirable to include at least one siRNA targeted to coding regions of a target transcript and at least one siRNA targeted to the 3′ UTR. This strategy may provide extra assurance that products encoded by the relevant transcript will not be generated because at least one siRNA in the composition will target the transcript for degradation while at least one other inhibits the translation of any transcripts that avoid degradation.

As described above, the invention encompasses “therapeutic cocktails”, including, but not limited to, approaches in which multiple siRNA oligonucleotides are administered and approaches in which a single vector directs synthesis of siRNAs that inhibit multiple targets or of RNAs that may be processed to yield a plurality of siRNAs. See Example 11 for further details. According to certain embodiments of the invention the composition includes siRNAs targeted to at least one influenza virus A transcript and at least one influenza virus B transcript. According to certain embodiments of the invention the composition comprises multiple siRNAs having different sequences that target the same portion of a particular segment. According to certain embodiments of the invention the composition comprises multiple siRNAs that inhibit different influenza virus strains or subtypes.

It is significant that the inventors have demonstrated effective siRNA-mediated inhibition of influenza virus replication, as evidenced by greatly reduced production of HA, using whole infectious virus as opposed, for example, to transfected genes, integrated transgenes, integrated viral genomes, infectious molecular clones, etc.

It will be appreciated that influenza viruses undergo both antigenic shift and antigenic drift, as mentioned above. Therefore, the emergence of resistance to therapeutic agents may occur. Thus it may expected that, after an inventive composition has been in use for some time, mutation and/or reassortment may occur so that a variant that is not inhibited by the particular siRNA(s) provided may emerge. The present invention therefore contemplates evolving therapeutic regimes. For example, one or more new siRNAs can be selected in a particular case in response to a particular mutation or reassortment. For instance, it would often be possible to design a new siRNA identical to the original except incorporating whatever mutation had occurred or targeting a newly acquired RNA segment; in other cases, it will be desirable to target a new sequence within the same transcript; in yet other cases, it will be desirable to target a new transcript entirely.

It will often be desirable to combine the administration of inventive siRNAs with one or more other anti-viral agents in order to inhibit, reduce, or prevent one or more symptoms or characteristics of infection. In certain preferred embodiments of the invention, the inventive siRNAs are combined with one or more other antiviral agents such as amantadine or rimantadine (both of which inhibit the ion channel M2 protein involved in viral uncoating), and/or zanamivir, oseltamivir, peramivir (BCX-1812, RWJ-270201) Ro64-0796 (GS 4104) or RWJ-270201 (all of which are NA inhibitors and prevent the proper release of viral particles from the plasma membrane). However, the administration of the inventive siRNA compositions may also be combined with one or more of any of a variety of agents including, for example, influenza vaccines (e.g., conventional vaccines employing influenza viruses or viral antigens as well as DNA vaccines) of which a variety are known. See Palese, P. and Garcia-Sastre, 2002; Cheung and Lieberman, 2002, Lèuscher-Mattli, 2000; and Stiver, 2003, for further information regarding various agents in use or under study for influenza treatment or prevention. In different embodiments of the invention the terms “combined with” or “in combination with” may mean either that the siRNAs are present in the same mixture as the other agent(s) or that the treatment regimen for an individual includes both siRNAs and the other agent(s), not necessarily delivered in the same mixture or at the same time. According to certain embodiments of the invention the antiviral agent is an agent approved by the U.S. Food and Drug Administration such as amantadine, rimantadine, Relenza, or Tamiflu.

The inventive siRNAs offer a complementary strategy to vaccination and may be administered to individuals who have or have not been vaccinated with any of the various vaccines currently available or under development (reviewed in Palese, P. and Garcia-Sastre, A., J. Clin. Invest., 110(1): 9-13, 2002). Current vaccine formulations in the United States contain inactivated virus and must be administered by intramuscular injection. The vaccine is tripartite and contains representative strains from both subtypes of influenza A that are presently circulating (H3N2 and H1N1), in addition to an influenza B type. Each season specific recommendations identify particular strains for use in that season's vaccines. Other vaccine approaches include cold-adapted live influenza virus, which can be administered by nasal spray; genetically engineered live influenza virus vaccines containing deletions or other mutations in the viral genome; replication-defective influenza viruses, and DNA vaccines, in which plasmid DNA encoding one or more of the viral proteins is administered either intramuscularly or topically (see, e.g., Macklin, M. D., et al., J Virol, 72(2):1491-6, 1998; Illum, L., et al., Adv Drug Deliv Rev, 51(1-3):81-96, 2001; Ulmer, J., Vaccine, 20:S74-S76, 2002). It is noted that immunocompromised patients and elderly individuals may gain particular benefit from RNAi-based therapeutics since the efficacy of such therapeutics does not require an effective immune response.

In some embodiments of the invention, it may be desirable to target administration of inventive siRNA compositions to cells infected with influenza virus, or at least to cells susceptible of influenza virus infection (e.g., cells expressing sialic acid-containing receptors). In other embodiments, it will be desirable to have available the greatest breadth of delivery options.

As noted above, inventive therapeutic protocols involve administering an effective amount of an siRNA prior to, simultaneously with, or after exposure to influenza virus. For example, uninfected individuals may be “immunized” with an inventive composition prior to exposure to influenza; at risk individuals (e.g., the elderly, immunocompromised individuals, persons who have recently been in contact with someone who is suspected, likely, or known to be infected with influenza virus, etc.) can be treated substantially contemporaneously with (e.g., within 48 hours, preferably within 24 hours, and more preferably within 12 hours of) a suspected or known exposure. Of course individuals known to be infected may receive inventive treatment at any time.

Gene therapy protocols may involve administering an effective amount of a gene therapy vector capable of directing expression of an inhibitory siRNA to a subject either before, substantially contemporaneously, with, or after influenza virus infection. Another approach that may be used alternatively or in combination with the foregoing is to isolate a population of cells, e.g., stem cells or immune system cells from a subject, optionally expand the cells in tissue culture, and administer a gene therapy vector capable of directing expression of an inhibitory siRNA to the cells in vitro. The cells may then be returned to the subject. Optionally, cells expressing the siRNA (which may thus become resistant to influenza virus infection) can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

As mentioned above, influenza viruses infect a wide variety of species in addition to humans. The present invention includes the use of inventive siRNA compositions for the treatment of nonhuman species, particularly species such as chickens, swine, and horses.

VIII. Pharmaceutical Formulations

Inventive compositions may be formulated for delivery by any available route including, but not limited to parenteral (e.g., intravenous), intradermal, subcutaneous, oral, nasal, bronchial, opthalmic, transdermal (topical), transmucosal, rectal, and vaginal routes. Preferred routes of delivery include parenteral, transmucosal, nasal, bronchial, and oral. Inventive pharmaceutical compositions typically include an siRNA or other agent(s) such as vectors that will result in production of an siRNA after delivery, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive siRNAs, shRNAs, or vectors are preferably delivered in the form of an aerosol spray from a pressured container or dispenser which contains a suitable propellant, e.g., a gas such as carbon dioxide, or a nebulizer. The present invention particularly contemplates delivery of siRNA compositions using a nasal spray. Intranasal administration of DNA vaccines directed against influenza viruses has been shown to induce CD8 T cell responses, indicating that at least some cells in the respiratory tract can take up DNA when delivered by this route. (See, e.g., K. Okuda, A. Ihata, S. Watabe, E. Okada, T. Yamakawa, K. Hamajima, J. Yang, N. Ishii, M. Nakazawa, K. Okuda, K. Ohnari, K. Nakajima, K.-Q. Xin, “Protective immunity against influenza A virus induced by immunization with DNA plasmid containing influenza M gene”, Vaccine 19:3681-3691, 2001). siRNAs are much smaller than plasmid DNA such as that used in the vaccines, suggesting that even greater uptake of siRNA will occur. In addition, according to certain embodiments of the invention delivery agents to facilitate nucleic acid uptake by cells in the airway are included in the pharmaceutical composition. (See, e.g., S.-O. Han, R. I. Mahato, Y. K. Sung, S. W. Kim, “Development of biomaterials for gene therapy”, Molecular Therapy 2:302317, 2000.) According to certain embodiments of the invention the siRNAs compositions are formulated as large porous particles for aerosol administration as described in more detail in Example 10.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In addition to the delivery agents described above, in certain embodiments of the invention, the active compounds (siRNA, shRNA, or vectors) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an siRNA, shRNA, or vector as described herein, can include a single treatment or, in many cases, can include a series of treatments.

Exemplary doses include milligram or microgram amounts of the inventive siRNA per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), doses much smaller than these may be used. It is furthermore understood that appropriate doses of an siRNA depend upon the potency of the siRNA, and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

As mentioned above, the present invention includes the use of inventive siRNA compositions for treatment of nonhuman animals including, but not limited to, horses, swine, and birds. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

As described above, nucleic acid molecules that serve as templates for transcription of siRNA or shRNA can be inserted into vectors which can be used as gene therapy vectors. In general, gene therapy vectors can be delivered to a subject by, for example, intravenous injection, local administration, or by stereotactic injection (see e.g., Chen et al. (1994) Proc. Natl. Acad. Sci. USA 91:3054-3057). In certain embodiments of the invention compositions comprising gene therapy vectors and a delivery agent may be delivered orally or inhalationally and may be encapsulated or otherwise manipulated to protect them from degradation, etc. The pharmaceutical compositions comprising a gene therapy vector can include an acceptable diluent, or can comprise a slow release matrix in which the gene delivery vehicle is imbedded. Alternatively, where the complete gene delivery vector can be produced intact from recombinant cells, e.g., retroviral or lentiviral vectors, the pharmaceutical preparation can include one or more cells which produce the gene delivery system.

Inventive pharmaceutical compositions can be included in a container, pack, or dispenser together with instructions for administration.

### Additional Embodiments

It will be appreciated that many of the teachings provided herein can readily be applied to infections with infectious agents other than influenza virus. The present invention therefore provides methods and compositions for inhibiting infection and/or replication by any infectious agent through administration of an RNAi-inducing entity (e.g., an siRNA, shRNA, or RNAi-inducing vector) that inhibits expression or activity of one or more agent-specific genes involved in the life cycle of the infectious agent. In particular, the present invention provides methods and compositions for inhibiting infection and/or replication by infectious agents that infect cells that are readily accessible from the exterior of the body. Such cells include skin cells and mucosal cells, e.g., cells of the respiratory tract, urogenital tract, and eye.

These conditions include infections due to viral, protozoal, and/or fungal agents. Respiratory tract infections suitable for treatment using inventive siRNA compositions as described herein include, but are not limited to, hantavirus, adenovirus, herpex simplex virus, and coccidiomycosis, and histoplasmosis infection. Urogenital tract and skin infections suitable for treatment using RNAi-inducing compositions include, but are not limited to, papilloma virus (that causes cervical carcinomas among other conditions), and herpes viruses.

In particular, it is noted that RNAi-based therapy may be particularly appropriate for infections for which either (i) no effective vaccine exists; and/or (ii) no other effective medication exists and/or existing therapeutic regimens are lengthy or cumbersome; and/or (iii) the agent undergoes genetic changes that may render older therapies or vaccines ineffective. These agents include many that are candidates for use in biological weapons, and there is therefore great interest in developing effective methods for prophylaxis and therapy. Trypanosomes change surface antigens frequently via a genetic recombination event. The flexibility afforded by the ability to rapidly design siRNAs and shRNAs targeted to the transcripts encoding the new surface antigens suggests that RNAi-based therapies may be appropriate for diseases caused by organisms that can rapidly change surface antigens and thereby elude immune system based approaches.

In each case, the skilled artisan will select one or more agent-specific transcripts necessary or important for effective infection, survival, replication, maturation, etc., of the agent. By agent-specific transcript is meant a transcript having a sequence that differs from the sequence of transcripts normally found in an uninfected host cell over a region sufficiently long to serve as a target for RNAi. In general, such a region is at least 15 nucleotides in length. Note that influenza virus mRNAs, which include sequences derived from host cell mRNAs, are considered agent-specific transcripts. The agent-specific transcript may be present in the genome of the infectious agent or produced subsequently during the infectious process. One or more siRNAs will then be designed according to the criteria presented herein.

The ability of candidate siRNAs to suppress expression of target transcripts and/or the potential efficacy of the siRNA as a therapeutic agent may be tested using appropriate in vitro and/or in vivo (e.g., animal) models to select those siRNA capable of inhibiting expression of the target transcript(s) and/or reducing or preventing infectivity, pathogenicity, replication, etc., of the infectious agent. Appropriate models will vary depending on the infectious agent and can readily be selected by one of ordinary skill in the art. For example, for certain infectious agents and for certain purposes it will be necessary to provide host cells while in other cases the effect of siRNA on the agent may be assessed in the absence of host cells. As described above for influenza infection, siRNAs may be designed that are targeted to any of a variety of agent-specific genes involved in one or more stages of the infection and/or replication cycle. Such siRNAs may be introduced into cells prior to, during, or after infection, and their effects on various stages of the infection/replication cycle may be assessed as desired.

It is significant that the inventors have demonstrated effective RNAi-mediated inhibition of target transcript expression and of entry and replication of an infectious agent using whole infectious virus as opposed, for example, to transfected genes, integrated transgenes, integrated viral genomes, infectious molecular clones, etc. The invention encompasses an RNAi-inducing entity targeted to an agent-specific transcript that is involved in replication, pathogenicity, or infection by an infectious agent. Preferred agent-specific transcripts that may be targeted in accordance with the invention include the agent's genome and/or any other transcript produced during the life cycle of the agent. Preferred targets include transcripts that are specific for the infectious agent and are not found in the host cell. For example, preferred targets may include agent-specific polymerases, sigma factors, transcription factors, etc. Such molecules are well known in the art, and the skilled practitioner will be able to select appropriate targets based on knowledge of the life cycle of the agent. In this regard useful information may be found in, e.g., Fields' Virology, 4^{th} ed., Knipe, D. et al. (eds.) Philadelphia, Lippincott Williams & Wilkins, 2001; Marr, J., et al., Molecular Medical Parasitology; and Georgi's Parasitology for Veterinarians, Bowman, D., et al, W. B. Saunders, 2003.

In some embodiments of the invention a preferred transcript is one that is particularly associated with the virulence of the infectious agent, e.g., an expression product of a virulence gene. Various methods of identifying virulence genes are known in the art, and a number of such genes have been identified. The availability of genomic sequences for large numbers of pathogenic and nonpathogenic viruses, bacteria, etc., facilitates the identification of virulence genes. Similarly, methods for determining and comparing gene and protein expression profiles for pathogenic and non-pathogenic strains and/or for a single strain at different stages in its life cycle agents enable identification of genes whose expression is associated with virulence. See, e.g., Winstanley, “Spot the difference: applications of subtractive hybridisation to the study of bacterial pathogens”, J Med Microbiol 2002 June; 51(6):459-67; Schoolnik, G, “Functional and comparative genomics of pathogenic bacteria”, Curr Opin Microbiol 2002 February; 5(1):20-6. For example, agent genes that encode proteins that are toxic to host cells would be considered virulence genes and may be preferred targets for RNAi. Transcripts associated with agent resistance to conventional therapies are also preferred targets in certain embodiments of the invention. In this regard it is noted that in some embodiments of the invention the target transcript need not be encoded by the agent genome but may instead be encoded by a plasmid or other extrachromosomal element within the agent.

In some embodiments of the invention the virus is a virus other than respiratory syncytial virus. In some embodiments of the invention the virus is a virus other than polio virus.

The RNAi-inducing entities may have any of a variety of structures as described above (e.g., two complementary RNA strands, hairpin, structure, etc.). They may be chemically synthesized, produced by in vitro transcription, or produced within a host cell.

### EXEMPLIFICATION

### Example 1

### Design of siRNAs to Inhibit Influenza A Virus

Genomic sequences from a set of influenza virus strains were compared, and regions of each segment that were most conserved were identified. This group of viruses included viruses derived from bird, swine, horse, and human. To perform the comparison the sequences of individual segments from 12 to 15 strains of influenza A virus from different animal (nonhuman) species isolated in different years and from 12 to 15 strains from humans isolated in different years were aligned. The strains were selected to encompass a wide variety of HA and NA subtypes. Regions that differed either by 0, 1, or 2 nucleotides among the different strains were selected. For example, the following strains were used for selection of siRNAs that target the NP transcript, accession number before each strain name refers to the accession number of the NP sequence and the portions of the sequence that were compared are indicated by nucleotide number.

The order of the entries in the following list is: accession number, strain name, portion of sequence compared, year, subtype. Accession numbers for the other genome segments differ but may be found readily in databases mentioned above. Strains compared were:

| | | | | |
|---|---|---|---|---|
| NC_002019 | A/Puerto Rico/8/34 | 1565 | 1934 | H1N1 |
| M30746 | A/Wilson-Smith/33 | 1565 | 1933 | H1N1 |
| M81583 | A/Leningrad/134/47/57 | 1566 | 1957 | H2N2 |
| AF348180 | A/Hong Kong/1/68 | 1520 | 1968 | H3N2 |
| L07345 | A/Memphis/101/72 | 1565 | 1972 | H3N2 |
| D00051 | A/Udorn/307/72 | 1565 | 1972 | H3N2 |
| L07359 | A/Guangdong/38/77 | 1565 | 1977 | H3N2 |
| M59333 | A/Ohio/201/83 | 1565 | 1983 | H1N1 |
| L07364 | A/Memphis/14/85 | 1565 | 1985 | H3N2 |
| M76610 | A/Wisconsin/3623/88 | 1565 | 1988 | H1N1 |
| U71144 | A/Akita/1/94 | 1497 | 1994 | H3N2 |
| AF084277 | A/Hong Kong/483/97 | 1497 | 1997 | H5N1 |
| AF036359 | A/Hong Kong/156/97 | 1565 | 1997 | H5N1 |
| AF250472 | A/Aquatic bird/Hong Kong/ | 1497 | 1998 | H11N1 |
| | M603/98 | | | |
| ISDN13443 | A/Sydney/274/2000 | 1503 | 2000 | H3N2 |
| M63773 | A/Duck/Manitoba/1/53 | 1565 | 1953 | H10N7 |
| M63775 | A/Duck/Pennsylvania/1/69 | 1565 | 1969 | H6N1 |
| M30750 | A/Equine/London/1416/73 | 1565 | 1973 | H7N7 |
| M63777 | A/Gull/Maryland/5/77 | 1565 | 1977 | H11N9 |
| M30756 | A/gull/Maryland/1815/79 | 1565 | 1979 | H13N6 |
| M63785 | A/Mallard/Astrakhan(Gurjev)/ | 1565 | 1982 | H14N5 |
| | 263/82 | | | |
| M27520 | A/whale/Maine/328/84 | 1565 | 1984 | H13N2 |
| M63768 | A/Swine/Iowa/17672/88 | 1565 | 1988 | H1N1 |
| Z26857 | A/turkey/Germany/3/91 | 1554 | 1991 | H1N1 |
| U49094 | A/Duck/Nanchang/1749/92 | 1407 | 1992 | H11N2 |
| AF156402 | A/Chicken/Hong Kong/G9/97 | 1536 | 1997 | H9N2 |
| AF285888 | A/Swine/Ontario/01911-1/99 | 1532 | 1999 | H4N6 |

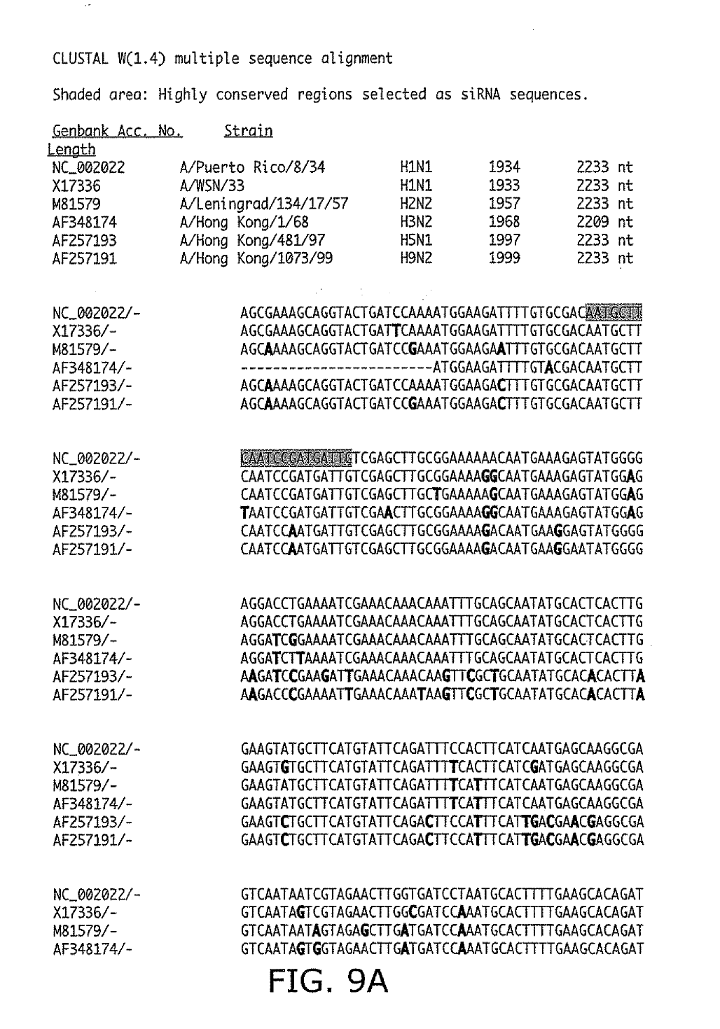

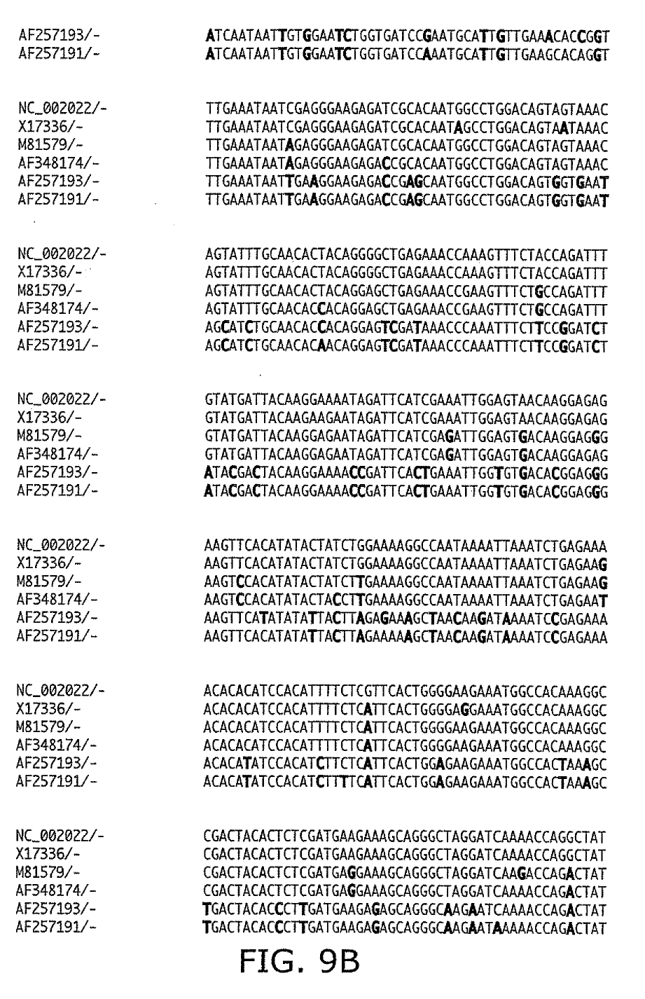

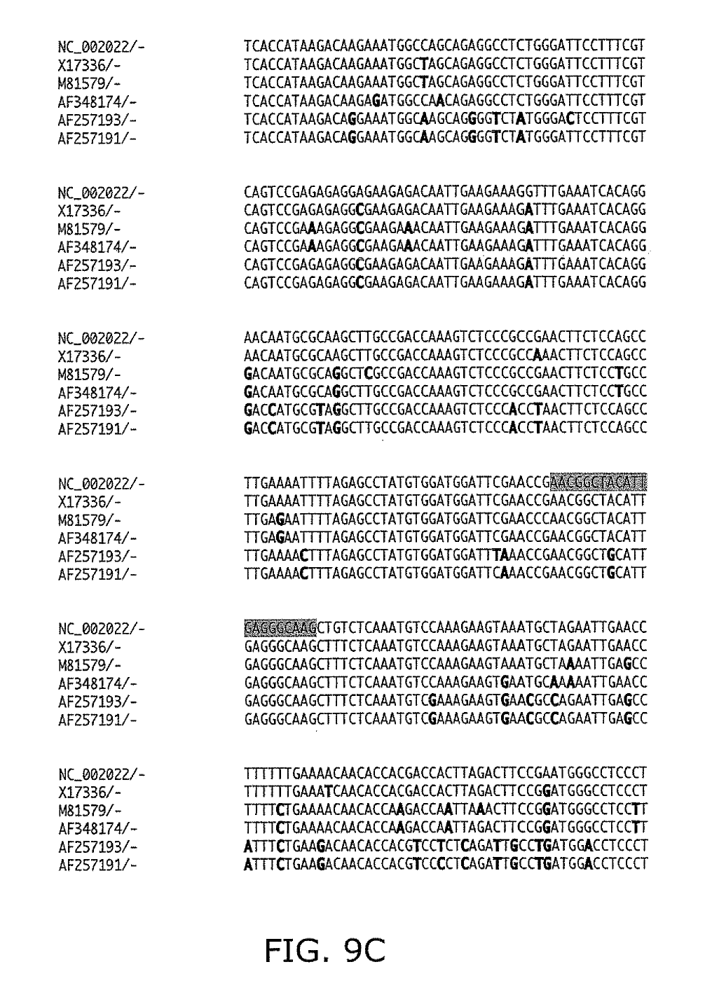

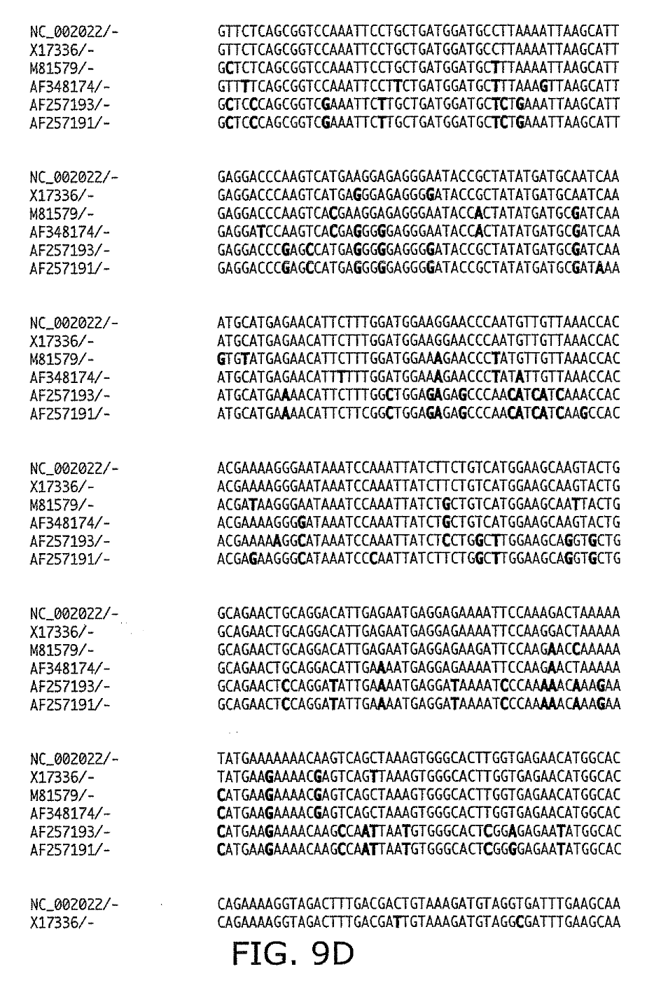

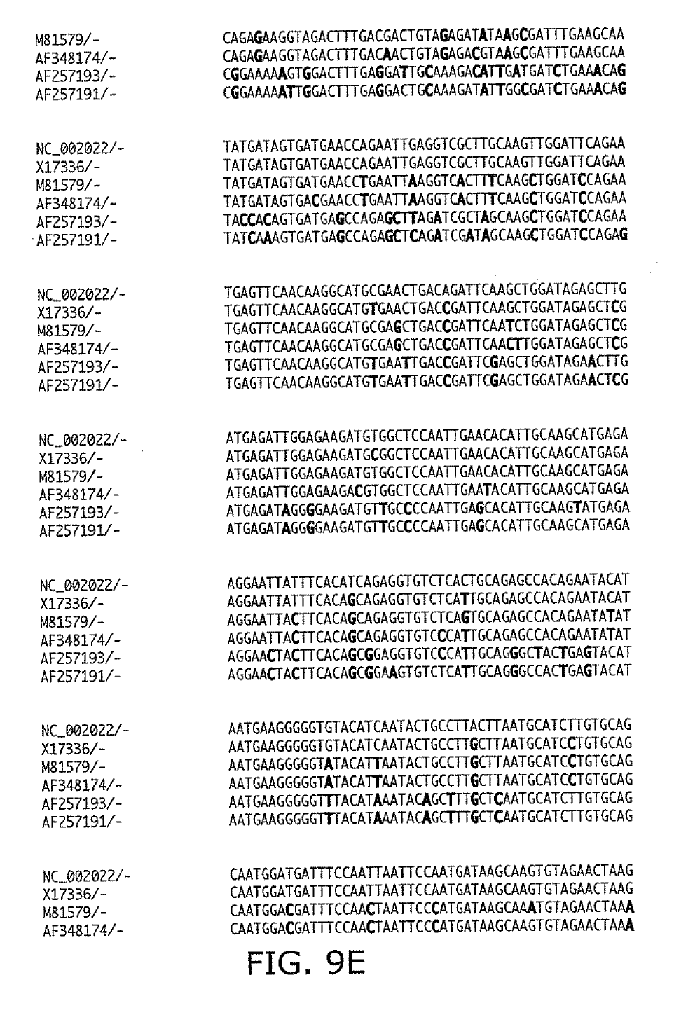

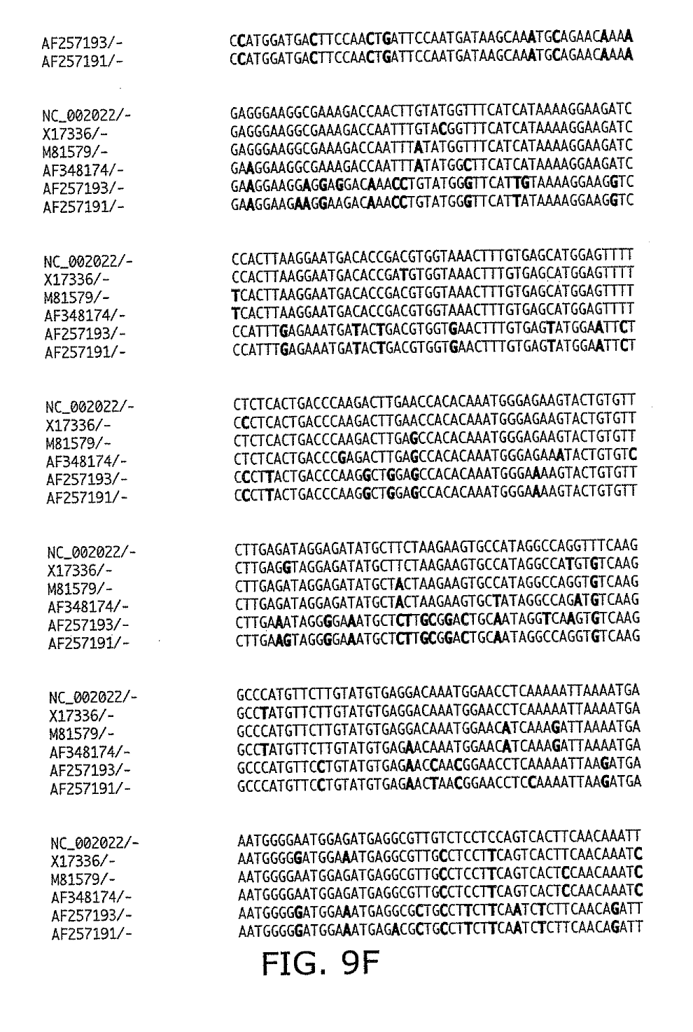

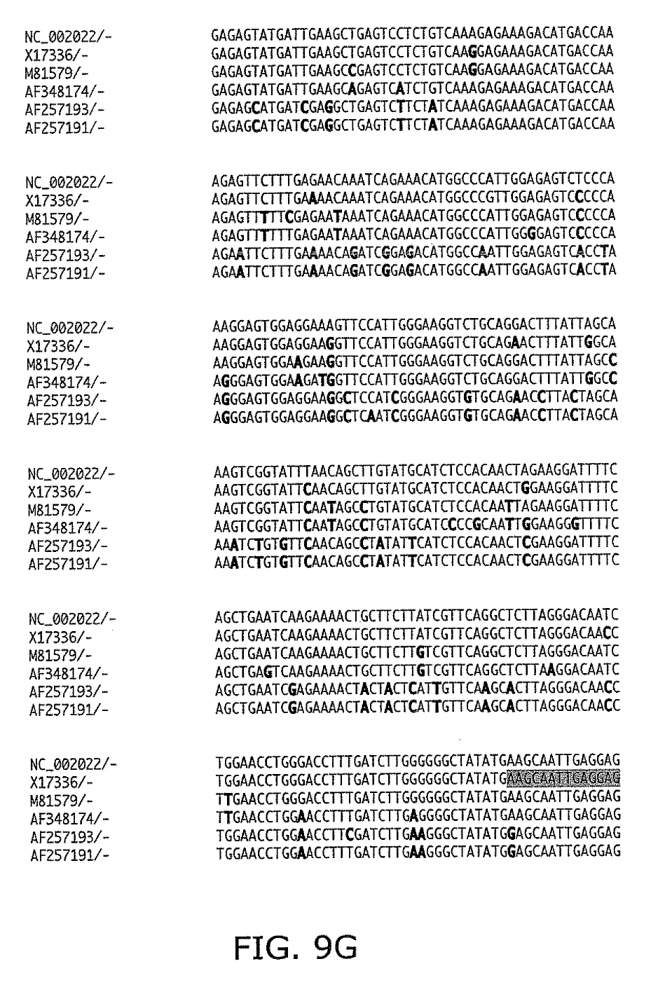

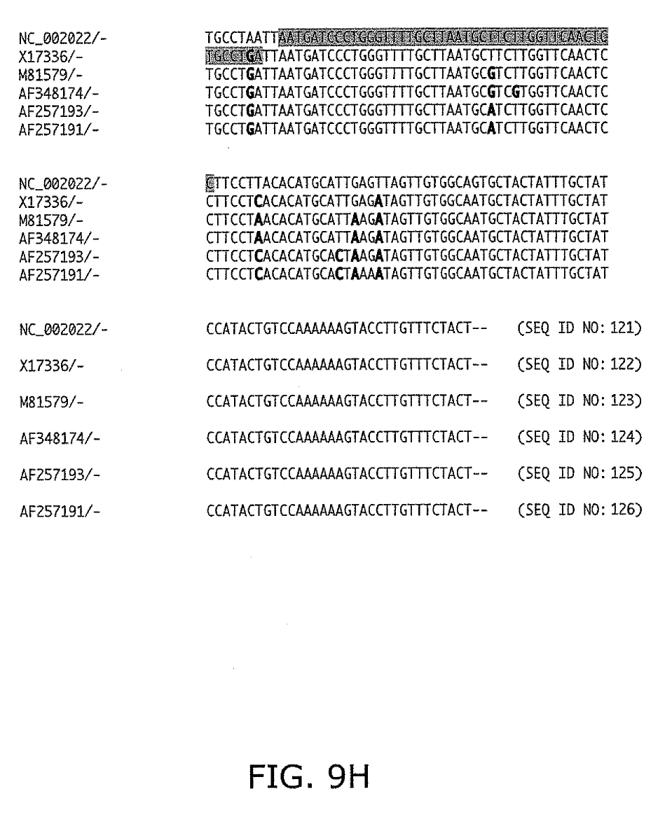

FIG. 9 shows an example of the selection of certain regions of the PA transcript that are highly conserved among six influenza A variants (all of which have a human host of origin), in which regions are considered highly conserved if they differ by either 0, 1, or 2 nucleotides. (Note that the sequences are listed as DNA rather than RNA and therefore contain T rather than U.) The sequence of strain A/Puerto Rico/8/34 (H1N1) was selected as the base sequence, i.e., the sequence with which the other sequences were compared. The other members of the set were A/WSN/33 (H1N1), A/Leningrad/134/17/57 (H2N2), A/Hong Kong/1/68 (H3N2), A/Hong Kong/481/97 (H5N1), and A/Hong Kong/1073/99 (H9N2). The figure presents a multiple sequence alignment produced by the computer program CLUSTAL W (1.4). Nucleotides that differ from the base sequence are shaded.

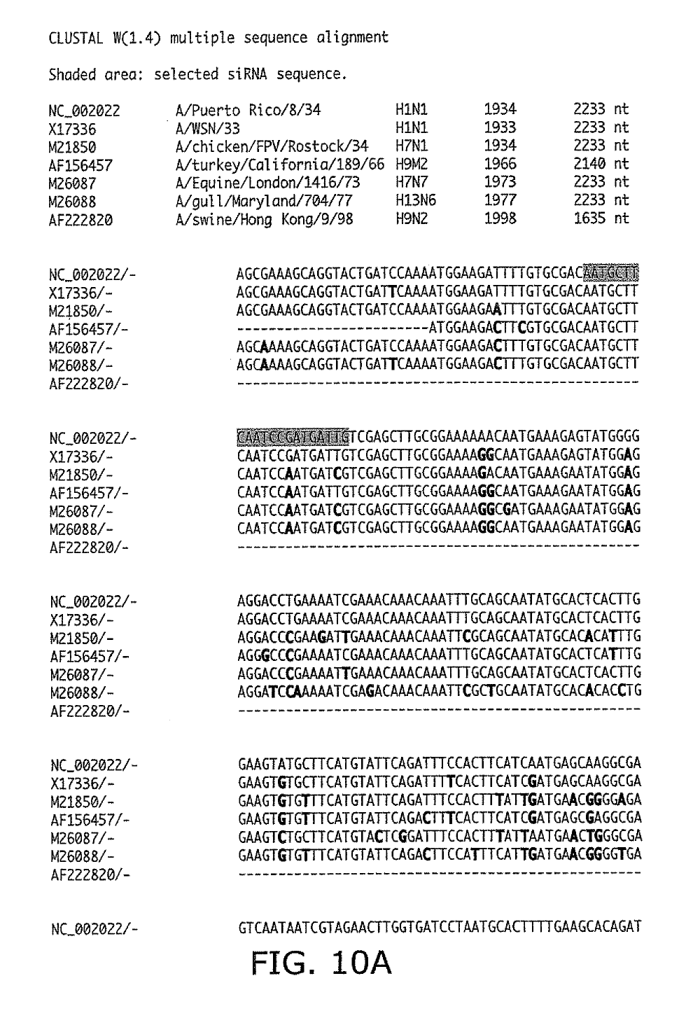

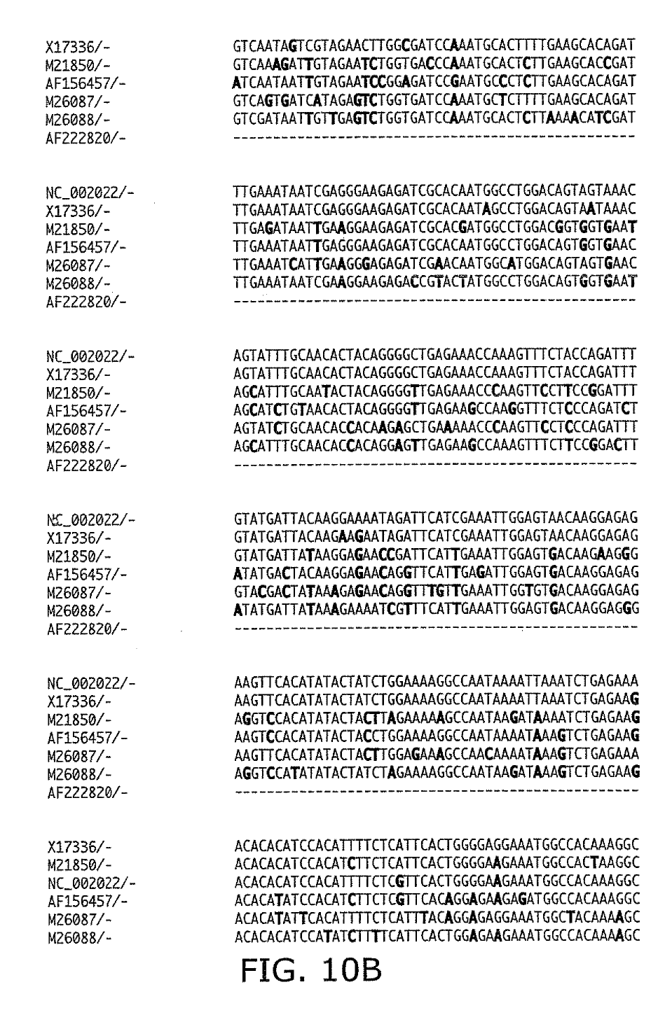

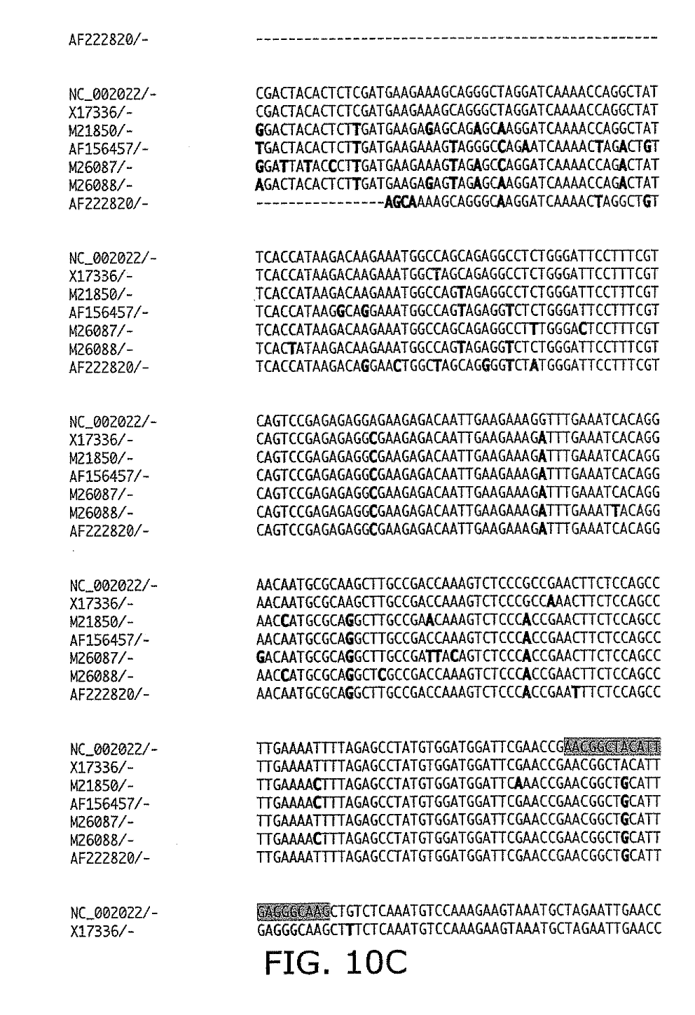

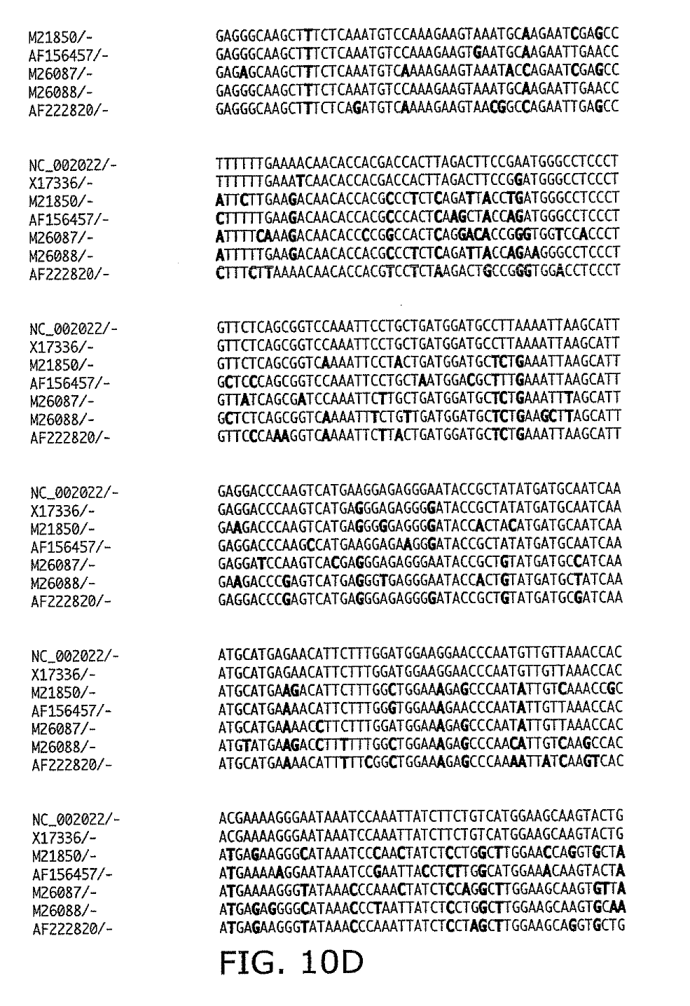

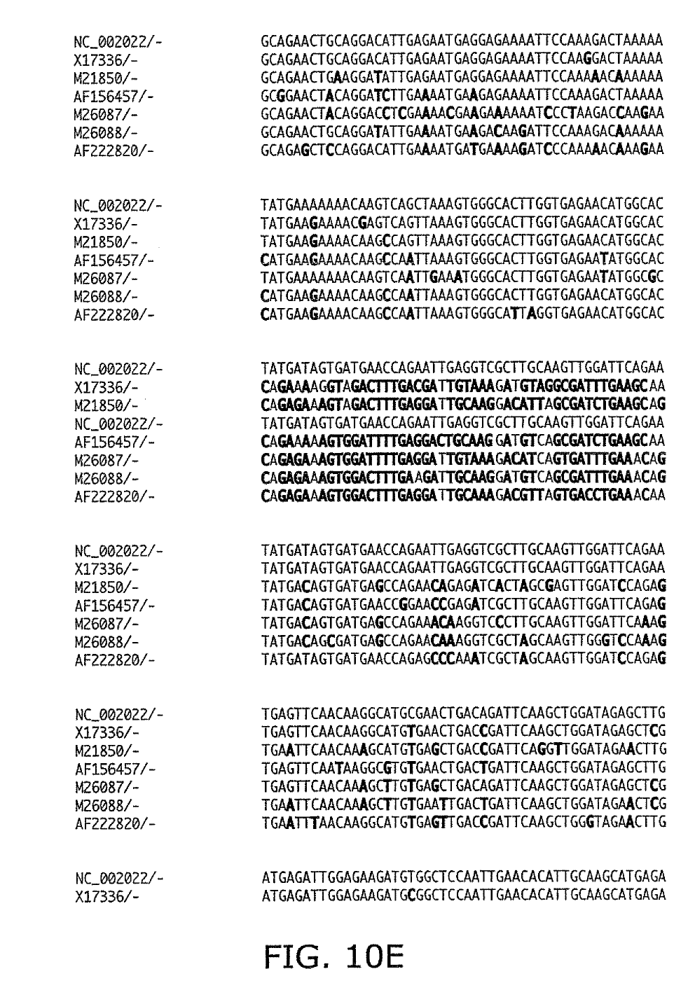

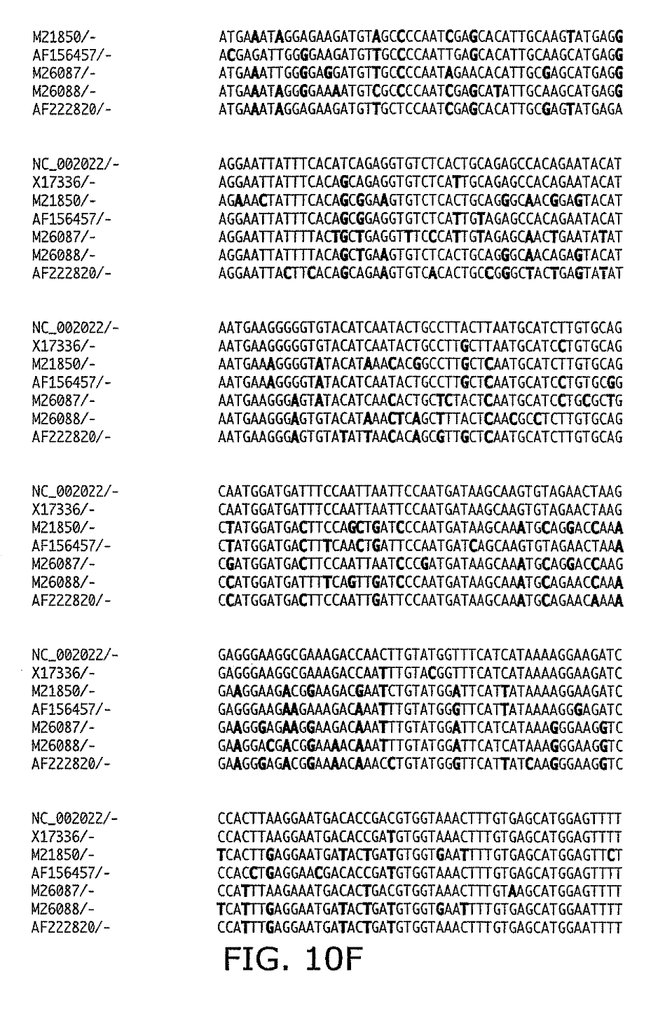

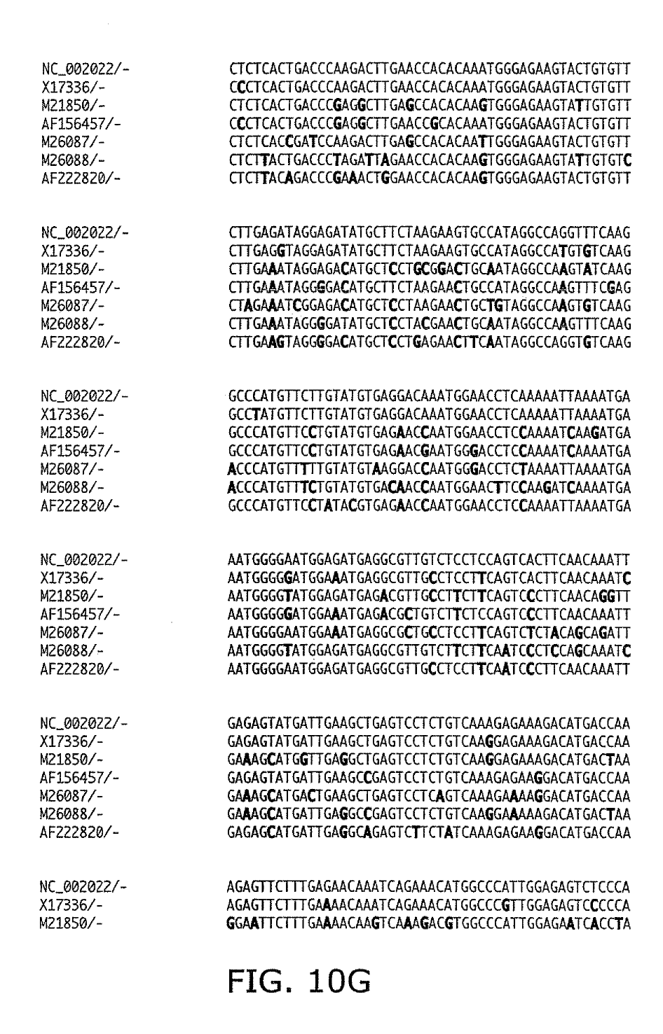

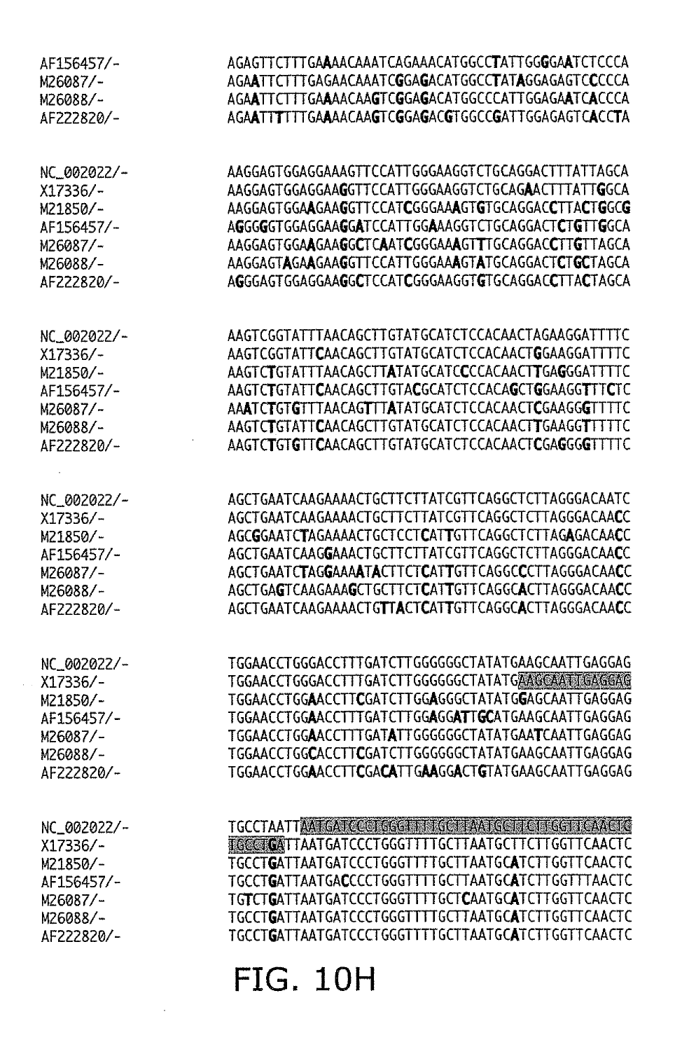

FIG. 10 shows an example of the selection of certain regions of the PA transcript that are highly conserved among five influenza A variants (all of which have different animal hosts of origin) and also among two strains that have a human host of origin, in which regions are considered highly conserved if they differ by either 0, 1, or 2 nucleotides. (Note that the sequences are listed as DNA rather than RNA and therefore contain T rather than U.) The sequence of strain A/Puerto Rico/8/34 (H1N1) was selected as the base sequence, i.e., the sequence with which the other sequences were compared. The other members of the set were A/WSN/33 (H1N1), A/chicken/FPV/Rostock/34 (H7N1), A/turkey/California/189/66 (H9M2), A/Equine/London/1416/73 (H7N7), A/gull/Maryland/704/77 (H13N6), and A/swine/Hong Kong/9/98 (H9N₂). Nucleotides that differ from the base sequence are shaded.

Note that in the sequence comparisons in FIGS. 9 and 10 many different highly conserved regions can be selected since large portions of the sequence meet the criteria for being highly conserved. However, sequences that have AA at the 5′ end provide for a 19 nucleotide core sequence and a 2 nucleotide 3′ UU overhang in the complementary (antisense) siRNA strand. Therefore regions that were highly conserved were scanned to identify 21 nucleotide portions that had AA at their 5′ end so that the complementary nucleotides, which are present in the antisense strand of the siRNA, are UU. For example, each of the shaded sequences has AA at its 5′ end. Note that the UU 3′ overhang in the antisense strand of the resulting siRNA molecule may be replaced by TT or dTdT as shown in Table 2. However, it is not necessary that the 2 nt 3′ overhang of the antisense strand is UU.

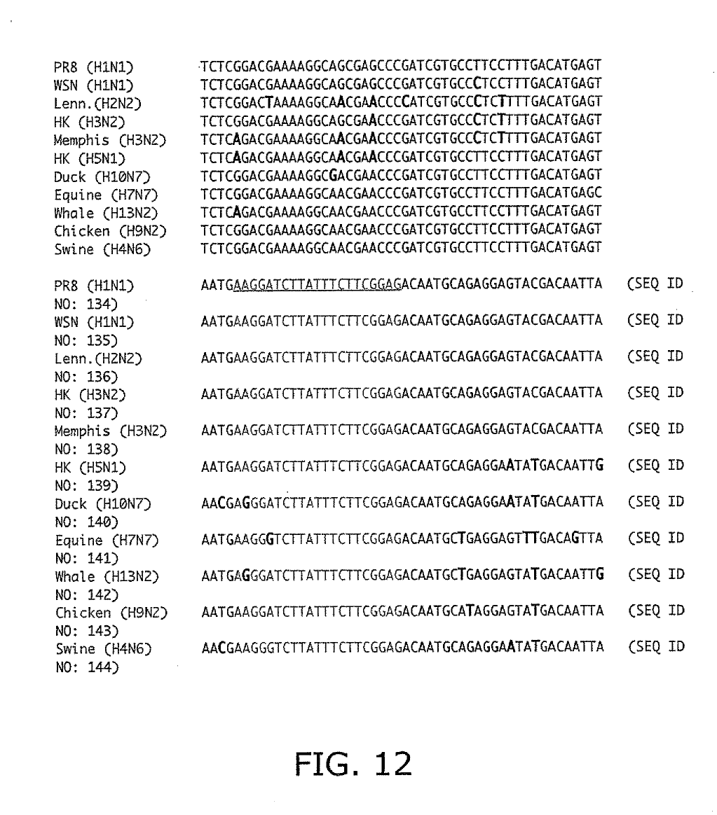

Further illustrating the method, FIG. 12 shows a sequence comparison between a portion of the 3′ region of NP sequences among twelve influenza A virus subtypes or isolates that have either a human or animal host of origin. The underlined sequence and the corresponding portions of the sequences below the underlined sequence were used to design siRNA NP-1496 (see below). These sequences are indicated in FIG. 12. The base sequence is the sequence of strain A/Puerto Rico/8/34. Shaded letters indicate nucleotides that differ from the base sequence.

Table 1 lists 21 nucleotide regions that are highly conserved among the set of influenza virus sequences compared for the PA segment in addition to the seven other viral gene segments. Many of the sequences meet the additional criterion that they have AA at their 5′ end so as to result in a 3′ UU overhang in the complementary strand. For the PA segment, in cases where a one or two nucleotide difference existed, the sequences of the siRNAs were based on the A/PR8/34 (H1N1) strain except for sequence PA-2087/2107 AAGCAATTGAGGAGTGCCTGA (SEQ ID NO: 30), which was based on the A/WSN/33(H1N1) strain. Note that at position 20 five of the six sequences contain a G while the base sequence contains an A. Thus in this case the sequence of the base sequence was not used for siRNA design.

To design siRNAs based on the sequences listed in Table 1A, nucleotides 3-21 were selected as the core regions of siRNA sense strand sequences, and a two nt 3′ overhang consisting of dTdT was added to each resulting sequence. A sequence complementary to nucleotides 1-21 of each sequence was selected as the corresponding antisense strand. For example, to design an siRNA based on the highly conserved sequence PA-44/64, i.e., AATGCTTCAATCCGATGATTG (SEQ ID NO: 22) a 19 nt core region having the sequence TGCTTCAATCCGATGATTG (SEQ ID NO: 109) was selected. A two nt 3′ overhang consisting of dTdT was added, resulting (after replacement of T by U) in the sequence 5′-UGCUUCAAUCCGAUGAUUGdTdT-3′ (SEQ ID NO: 79), which was the sequence of the siRNA sense strand. The sequence of the corresponding antisense siRNA strand sequence is complementary to SEQ ID NO: 22, i.e., CAAUCAUCGGAUUGAAGCAdTdT (SEQ ID NO: 80) where T has been replaced by U except for the 2 nt 3′ overhang, in which T is replaced by dT.

Table 1B lists siRNAs designed based on additional highly conserved regions of influenza virus transcripts. The first 19 nt sequences of the sequences indicated as “sense strand” in Table 1B are sequences of highly conserved regions. The sense strand siRNA sequences are shown with a dTdT overhang at the 3′ end, which does not correspond to influenza virus sequences and is an optional feature of the siRNA. Corresponding antisense strands are also shown, also incorporating a dTdT overhang at the 3′ end as an optional feature. Nomenclature is as in Table 1B. For example, PB2-4/22 sense indicates an siRNA whose sense strand has the sequence of nucleotides 4-22 of the PB2 transcript. PB2-4/22 antisense indicates the complementary antisense strand corresponding to PB2-4/22 sense. For siRNA that target sites in a transcript that span a splice site, the positions within the unspliced transcript are indicated. For example, M-44-52/741-750 indicates that nucleotides corresponding to 44-52 and 741-750 of the genomic sequences are targeted in the spliced mRNA.

Shaded areas in FIGS. 9 and 10 indicate some of the 21 nucleotide regions that meet the criteria for being highly conserved. siRNAs were designed based on these sequences as described above. The actual siRNA sequences that were tested are listed in Table 2.

TABLE 1A

Conserved regions for design of siRNA to interfere
with influenza A virus infection

Segment 1: PB2

| | | |
|---|---|---|
| PB2-117/137 | AATCAAGAAGTACACATCAGG | (SEQ ID NO: 1) |
| PB2-124/144 | AAGTACACATCAGGAAGACAG | (SEQ ID NO: 2) |
| PB2-170/190 | AATGGATGATGGCAATGAAAT | (SEQ ID NO: 3) |
| PB2-195/215 | AATTACAGCAGACAAGAGGAT | (SEQ ID NO: 4) |
| PB2-1614/1634 | AACTTACTCATCGTCAATGAT | (SEQ ID NO: 5) |
| PB2-1942/1962 | AATGTGAGGGGATCAGGAATG | (SEQ ID NO: 6) |
| PB2-2151/2171 | AAGCATCAATGAACTGAGCAA | (SEQ ID NO: 7) |
| PB2-2210/2230 | AAGGAGACGTGGTGTTGGTAA | (SEQ ID NO: 8) |
| PB2-2240/2260 | AACGGGACTCTAGCATACTTA | (SEQ ID NO: 9) |
| PB2-2283/2303 | AAGAATTCGGATGGCCATCAA | (SEQ ID NO: 10) |

Segment 2: PB1

| | | |
|---|---|---|
| PB1-6/26 | AAGCAGGCAAACCATTTGAAT | (SEQ ID NO: 11) |
| PB1-15/35 | AACCATTTGAATGGATGTCAA | (SEQ ID NO: 12) |
| PB1-34/54 | AATCCGACCTTACTTTTCTTA | (SEQ ID NO: 13) |
| PB1-56/76 | AAGTGCCAGCACAAAATGCTA | (SEQ ID NO: 14) |
| PB1-129/149 | AACAGGATACACCATGGATAC | (SEQ ID NO: 15) |
| PB1-1050/1070 | AATGTTCTCAAACAAAATGGC | (SEQ ID NO: 16) |
| PB1-1242/1262 | AATGATGATGGGCATGTTCAA | (SEQ ID NO: 17) |
| PB1-2257/2277 | AAGATCTGTTCCACCATTGAA | (SEQ ID NO: 18) |
| Segment 3: PA | | |
| PA-6/26 | AAGCAGGTACTGATCCAAAAT | (SEQ ID NO: 19) |
| PA-24/44 | AATGGAAGATTTTGTGCGACA | (SEQ ID NO: 20) |
| PA-35/55 | TTGTGCGACAATGCTTCAATC | (SEQ ID NO: 21) |
| pA-44/64 | AATGCTTCAATCCGATGATTG | (SEQ ID NO: 22) |
| PA-52/72 | AATCCGATGATTGTCGAGCTT | (SEQ ID NO: 23) |
| PA-121/141 | AACAAATTTGCAGCAATATGC | (SEQ ID NO: 24) |
| PA-617/637 | AAGAGACAATTGAAGAAAGGT | (SEQ ID NO: 25) |
| PA-711/731 | TAGAGCCTATGTGGATGGATT | (SEQ ID NO: 26) |
| PA-739/759 | AACGGCTACATTGAGGGCAAG | (SEQ ID NO: 27) |
| PA-995/1015 | AACCACACGAAAAGGGAATAA | (SEQ ID NO: 28) |
| PA-2054/2074 | AACCTGGGACCTTTGATCTTG | (SEQ ID NO: 29) |
| PA-2087/2107 | AAGCAATTGAGGAGTGCCTGA | (SEQ ID NO: 30) |
| PA-2110/2130 | AATGATCCCTGGGTTTTGCTT | (SEQ ID NO: 31) |
| PA-2131/2151 | AATGCTTCTTGGTTCAACTCC | (SEQ ID NO: 32) |
| Segment 4: HA | | |
| HA-1119/1139 | TTGGAGCCATTGCCGGTTTTA | (SEQ ID NO: 33) |
| HA-1121/1141 | GGAGCCATTGCCGGTTTTATT | (SEQ ID NO: 34) |
| HA-1571/1591 | AATGGGACTTATGATTATCCC | (SEQ ID NO: 35) |
| Segment 5: NP | | |
| NP-19/39 | AATCACTCACTGAGTGACATC | (SEQ ID NO: 36) |
| NP-42/62 | AATCATGGCGTCCCAAGGCAC | (SEQ ID NO: 37) |
| NP-231/251 | AATAGAGAGAATGGTGCTCTC | (SEQ ID NO: 38) |
| NP-390/410 | AATAAGGCGAATCTGGCGCCA | (SEQ ID NO: 39) |
| NP-393/413 | AAGGCGAATCTGGCGCCAAGC | (SEQ ID NO: 40) |
| NP-708/728 | AATGTGCAACATTCTCAAAGG | (SEQ ID NO: 41) |
| NP-1492/1512 | AATGAAGGATCTTATTTCTTC | (SEQ TD NO: 42) |
| NP-1496/1516 | AAGGATCTTATTTCTTCGGAG | (SEQ ID NO: 43) |
| NP-1519/1539 | AATGCAGAGGAGTACGACAAT | (SEQ ID NO: 44) |
| Segment 6: NA | | |
| NA-20/40 | AATGAATCCAAATCAGAAAAT | (SEQ ID NO: 45) |
| NA704/724 | GAGGACACAAGAGTCTGAATG | (SEQ ID NO: 46) |
| NA-861/881 | GAGGAATGTTCCTGTTACCCT | (SEQ ID NO: 47) |
| NA-901/921 | GTGTGTGCAGAGACAATTGGC | (SEQ ID NO: 48) |
| Segment 7: M | | |
| M-156/176 | AATGGCTAAAGACAAGACCAA | (SEQ ID NO: 49) |
| M-175/195 | AATCCTGTCACCTCTGACTAA | (SEQ ID NO: 50) |
| M-218/238 | ACGCTCACCGTGCCCAGTGAG | (SEQ ID NO: 51) |
| M-244/264 | ACTGCAGCGTAGACGCTTTGT | (SEQ ID NO: 52) |
| M-373/393 | ACTCAGTTATTCTGCTGGTGC | (SEQ ID NO: 53) |
| M-377/397 | AGTTATTCTGCTGGTGCACTT | (SEQ ID NO: 54) |
| M-480/500 | AACAGATTGCTGACTCCCAGC | (SEQ ID NO: 55) |
| M-584/604 | AAGGCTATGGAGCAAATGGCT | (SEQ ID NO: 56) |
| M-598/618 | AATGOCTGGATCGAGTGAGCA | (SEQ ID NO: 57) |
| M-686/706 | ACTCATCCTAGCTCCAGTGCT | (SEQ ID NO: 58) |
| M-731/751 | AATTTGCAGGCCTATCAGAAA | (SEQ ID NO: 59) |
| M-816/836 | ATTGTGGATTCTTGATCGTCT | (SEQ ID NO: 60) |
| M-934/954 | AAGAATATCGAAAGGAACAGC | (SEQ ID NO: 61) |
| M-982/1002 | ATTTTGTCAGCATAGAGCTGG | (SEQ ID NO: 62) |
| Segment 8: NS | | |
| NS-101/121 | AAGAACTAGGTGATGCCCCAT | (SEQ ID NO: 63) |
| NS-104/124 | AACTAGGTGATGCCCCATTCC | (SEQ ID NO: 64) |
| NS-128/148 | ATCGGCTTCGCCGAGATCAGA | (SEQ ID NO: 65) |
| NS-137/157 | GCCGAGATCAGAAATCCCTAA | (SEQ ID NO: 66) |
| NS-562/582 | GGAGTCCTCATCGGAGGACTT | (SEQ ID NO: 67) |
| NS-589/609 | AATGATAACACAGTTCGAGTC | (SEQ ID NO: 68) |

TABLE 1B

Conserved regions for design of siRNA to interfere with influenza A
virus infection

| | | |
|---|---|---|
| Segment 1: PB2 | | |
| PB2-4/22 sense | GAAAGCAGGUCAAUUAUAUdTdT | (SEQ ID NO: 190) |
| PB2-4/22 antisense | AUAUAAUUGACCUGCUUUCdTdT | (SEQ ID NO: 191) |
| PB2-12/30 sense | GUCAAUUAUAUUCAAUAUGdTdT | (SEQ ID NO: 192) |
| PB2-12/30 antisense | CAUAUUGAAUAUAAUUGACdTdT | (SEQ ID NO: 193) |
| PB2-68/86 sense | CUCGCACCCGCGAGAUACUdTdT | (SEQ ID NO: 194) |
| PB2-68/86 antisense | AGUAUCUCGCGGGUGCGAGdTdT | (SEQ ID NO: 195) |
| PB2-115/133 sense | AUAAUCAAGAAGUACACAUdTdT | (SEQ ID NO: 196) |
| PB2-115/133 antisense | AUGUGUACUUCUUGAUUAUdTdT | (SEQ ID NO: 197) |
| PB2-167/185 sense | UGAAAUGGAUGAUGGCAAUdTdT | (SEQ ID NO: 198) |
| PB2-167/185 antisense | AUUGCCAUCAUCCAUUUCAdTdT | (SEQ ID NO: 199) |
| PB2-473/491 sense | CUGGUCAUGCAGAUCUCAGdTdT | (SEQ ID NO: 200) |
| PB2-473/491 antisense | CUGAGAUCUGCAUGACCAGdTdT | (SEQ ID NO: 201) |
| PB2-956/974 sense | UAUGCAAGGCUGCAAUGGGdTdT | (SEQ ID NO: 202) |
| PB2-956/974 antisense | CCCAUUGCAGCCUUGCAUAdTdT | (SEQ ID NO: 203) |
| PB2-1622/1640 sense | CAUCGUCAAUGAUGUGGGAdTdT | (SEQ ID NO: 204) |
| PB2-1622/1640 antisense | UCCCACAUCAUUGACGAUGdTdT | (SEQ ID NO: 205) |
| Segment 2: PB1 | | |
| PB1-1124/1142 sense | AAAUACCUGCAGAAAUGCUdTdT | (SEQ ID NO: 206) |
| PB1-1124/1142 antisense | AGCAUUUCUGCAGGUAUUUdTdT | (SEQ ID NO: 207) |
| PB1-1618/1636 sense | AACAAUAUGAUAAACAAUGdTdT | (SEQ ID NO: 208) |
| PB1-1618/1636 antisense | CAUUGUUUAUCAUAUUGUUdTdT | (SEQ ID NO: 209) |
| Segment 3: PA | | |
| PA-3/21 sense | CGAAAGCAGGUACUGAUCCdTdT | (SEQ ID NO: 210) |
| PA-3/21 antisense | GGAUCAGUACCUGCUUUCGdTdT | (SEQ ID NO: 211) |
| PA-544/562 sense | AGGCUAUUCACCAUAAGACdTdT | (SEQ ID NO: 212) |
| PA-544/562 antisense | GUCUUAUGGUGAAUAGCCUdTdT | (SEQ ID NO: 213) |
| PA-587/605 sense | GGGAUUCCUUUCGUCAGUCdTdT | (SEQ ID NO: 214) |
| PA-587/605 antisense | GACUGACGAAAGGAAUCCCdTdT | (SEQ ID NO: 215) |
| PA-1438/1466 sense | GCAUCUUGUGCAGCAAUGGdTdT | (SEQ ID NO: 216) |
| PA-1438/1466 antisense | CCAUUGCUGCACAAGAUGCdTdT | (SEQ ID NO: 217) |
| PA-2175/2193 sense | GUUGUGGCAGUGCUACUAUdTdT | (SEQ ID NO: 218) |
| PA-2175/2193 antisense | AUAGUAGCACUGCCACAACdTdT | (SEQ ID NO: 219) |
| PA-2188/2206 sense | UACUAUUUGCUAUCCAUACdTdT | (SEQ ID NO: 220) |
| PA-2188/2206 antisense | GUAUGGAUAGCAAAUAGUAdTdT | (SEQ ID NO: 221) |
| Segment 5: NP | | |
| NP-14/32 sense | UAGAUAAUCACUCACUGAGdTdT | (SEQ ID NO: 222) |
| NP-14/32 antisense | CUCAGUGAGUGAUUAUCUAdTdT | (SEQ ID NO: 223) |
| NP-50/68 sense | CGUCCCAAGGCACCAAACGdTdT | (SEQ ID NO: 224) |
| NP-50/68 antisense | CGUUUGGUGCCUUGGGACGdTdT | (SEQ ID NO: 225) |
| NP-1505/1523 sense | AUUUCUUCGGAGACAAUGCdTdT | (SEQ ID NO: 226) |
| NP-1505/1523 antisense | GCAUUGUCUCCGAAGAAAUdTdT | (SEQ ID NO: 227) |
| NP-1521/1539 sense | UGCAGAGGAGUACGACAAUdTdT | (SEQ ID NO: 228) |
| NP-1521/1539 antisense | AUUGUCGUACUCCUCUGCAdTdT | (SEQ ID NO: 229) |
| NP-1488/1506 sense | GAGTAATGAAGGATCTTATdTdT | (SEQ ID NO: 230) |
| NP-1488/1506 antisense | ATAAGATCCTTCATTACTCdTdT | (SEQ ID NO: 231) |
| Segment 7: M | | |
| M-3/21 sense | CGAAAGCAGGUAGAUAUUGdTdT | (SEQ ID NO: 232) |
| M-3/21 antisense | CAAUAUCUACCUGCUUUCGdTdT | (SEQ ID NO: 233) |
| M-13/31 sense | UAGAUAUUGAAAGAUGAGUdTdT | (SEQ ID NO: 234) |
| M-13/31 antisense | ACUCAUCUUUCAAUAUCUAdTdT | (SEQ ID NO: 235) |
| M-150/158 sense | UCAUGGAAUGGCUAAAGACdTdT | (SEQ ID NO: 236) |
| M-150/158 antisense | GUCUUUAGCCAUUCCAUGAdTdT | (SEQ ID NO: 237) |
| M-172/190 sense | ACCAAUCCUGUCACCUCUGdTdT | (SEQ ID NO: 238) |
| M-172/190 antisense | CAGAGGUGACAGGAUUGGUdTdT | (SEQ ID NO: 239) |
| M-211/229 sense | UGUGUUCACGCUCACCGUGdTdT | (SEQ ID NO: 240) |
| M-211/229 antisense | CACGGUGAGCGUGAACACAdTdT | (SEQ ID NO: 241) |
| M-232/250 sense | CAGUGAGCGAGGACUGCAGdTdT | (SEQ ID NO: 242) |
| M-232/250 antisense | CUGCAGUCCUCGCUCACUGdTdT | (SEQ ID NO: 243) |
| M-255/273 sense | GACGCUUUGUCCAAAAUGCdTdT | (SEQ ID NO: 244) |
| M-255/273 antisense | GCAUUUUGGACAAAGCGUCdTdT | (SEQ ID NO: 245) |
| M-645/663 sense | GUCAGGCUAGGCAAAUGGUdTdT | (SEQ ID NO: 246) |
| M-645/663 antisense | ACCAUUUGCCUAGCCUGACdTdT | (SEQ ID NO: 247) |
| M-723/741 sense | UUCUUGAAAAUUUGCAGGCdTdT | (SEQ ID NO: 248) |
| M-723/741 antisense | GCCUGCAAAUUUUCAAGAAdTdT | (SEQ ID NO: 249) |
| M-808/826 sense | UCAUUGGGAUCUUGCACUUdTdT | (SEQ ID NO: 250) |
| M-808/826 antisense | AAGUGCAAGAUCCCAAUGAdTdT | (SEQ ID NO: 251) |
| M-832/850 sense | UGUGGAUUCUUGAUCGUCUdTdT | (SEQ ID NO: 252) |
| M-832/850 antisense | AGACGAUCAAGAAUCCACAdTdT | (SEQ ID NO: 253) |
| M-986/1004 sense | UGUCAGCAUAGAGCUGGAGdTdT | (SEQ ID NO: 254) |
| M-986/1004 antisense | CUCCAGCUCUAUGCUGACAdTdT | (SEQ ID NO: 255) |
| M-44-52/741-750 sense | GTCGAAACGCCTATCAGAAdTdT | (SEQ ID NO: 256) |
| M-44-52/741-750 antisense | UUCUGAUAGGCGUUUCGACdTdT | (SEQ ID NO: 257) |
| Segment 8: NS | | |
| NS-5/23 sense | AAAAGCAGGGUGACAAAGAdTdT | (SEQ ID NO: 258) |
| NS-5/23 antisense | UCUUUGUCACCCUGCUUUUdTdT | (SEQ ID NO: 259) |
| NS-9/27 sense | GCAGGGUGACAAAGACAUAdTdT | (SEQ ID NO: 260) |
| NS-9/27 antisense | UAUGUCUUUGUCACCCUGCdTdT | (SEQ ID NO: 261) |
| NS-543/561 sense | GGAUGUCAAAAAUGCAGUUdTdT | (SEQ ID NO: 262) |
| NS-543/561 antisense | AACUGCAUUUUUGACAUCCdTdT | (SEQ ID NO: 263) |
| NS-623/641 sense | AGAGAUUCGCUUGGAGAAGdTdT | (SEQ ID NO: 264) |
| NS-623/641 antisense | CUUCUCCAAGCGAAUCUCUdTdT | (SEQ ID NO: 265) |
| NS-642/660 sense | CAGUAAUGAGAAUGGGAGAdTdT | (SEQ ID NO: 266) |
| NS-642/660 antisense | UCUCCCAUUCUCAUUACUGdTdT | (SEQ ID NO: 267) |
| NS-831/849 sense | UUGUGGAUUCUUGAUCGUCdTdT | (SEQ ID NO: 268) |
| NS-831/839 antisense | GACGAUCAAGAAUCCACAAdTdT | (SEQ ID NO: 269) |

### Example 2

### siRNAs that Target Viral RNA Polymerase or Nucleoprotein Inhibit Influenza A Virus Production

Materials and Methods

Cell Culture. Madin-Darby canine kidney cells (MDCK), a kind gift from Dr. Peter Palese, Mount Sinai School of Medicine, New York, N.Y., were grown in DMEM medium containing 10% heat-inactivated FCS, 2 mM L-glutamine, 100 units/ml penicillin, and 100 μg/ml streptomycin. Cells were grown at 37° C., 5% CO₂. For electroporation, the cells were kept in serum-free RPMI 1640 medium. Virus infections were done in infection medium (DMEM, 0.3% bovine serum albumin (BSA, Sigma, St. Louis, Mo.), 10 mM Hepes, 100 units/ml penicillin, and 100 μg/ml streptomycin).

Viruses. Influenza viruses A/PR/8/34 (PR8) and A/WSN/33 (WSN), subtypes H1N1, kind gifts from Dr. Peter Palese, Mount Sinai School of Medicine, were grown for 48 h in 10-day-embryonated chicken eggs (Charles River laboratories, MA) at 37° C. Allantoic fluid was harvested 48 h after virus inoculation and stored at −80° C. siRNAs. siRNAs were designed as described above. In addition to conforming to the selection criteria described in Example 1, the siRNAs were generally designed in accordance with principles described in Technical Bulletin # 003-Revision B, “siRNA Oligonucleotides for RNAi Applications”, available from Dharmacon Research, Inc., Lafayette, Colo. 80026, a commercial supplier of RNA reagents. Technical Bulletins #003 (accessible on the World Wide Web at www.dharmacon.com/tech/tech003B.html) and #004 available at www.dharmacon.com/tech/tech004.html from Dharmacon contain a variety of information relevant to siRNA design parameters, synthesis, etc., and are incorporated herein by reference. Sense and antisense sequences that were tested are listed in Table 2.

TABLE 2
siRNA Sequences

| | |
|---|---|
| Name | siRNA sequence (5′-3′) |

| | | |
|---|---|---|
| PB2-2210/2230 (sense) | GGAGACGUGGUGUUGGUAAdTdT | (SEQ ID NO: 69) |
| PB2-2210/2230 (antisense) | UUACCAACACCACGUCUCCdTdT | (SEQ ID NO: 70) |
| PB2-2240/2260 (sense) | CGGGACUCUAGCAUACUUAdTdT | (SEQ ID NO: 71) |
| PB2-2240/2260 (antisense) | UAAGUAUGCUAGAGUCCCGdTdT | (SEQ ID NO: 72) |
| PB1-6/26 (sense) | GCAGGCAAACCAUUUGAAUdTdT | (SEQ ID NO: 73) |
| PB1-6/26 (antisense) | AUUCAAAUGGUUUGCCUGCdTdT | (SEQ ID NO: 74) |
| PB1-129/149 (sense) | CAGGAUACACCAUGGAUACdTdT | (SEQ ID NO: 75) |
| PB1-129/149 (antisense) | GUAUCCAUGGUGUAUCCUGdTdT | (SEQ ID NO: 76) |
| PB1-2257/2277 (sense) | GAUCUGUUCCACCAUUGAAdTdT | (SEQ ID NO: 77) |
| PB1-2257/2277 (antisense) | UUCAAUGGUGGAACAGAUCdTdT | (SEQ ID NO: 78) |
| PA-44/64 (sense) | UGCUUCAAUCCGAUGAUUGdTdT | (SEQ ID NO: 79) |
| PA-44/64 (antisense) | CAAUCAUCGGAUUGAAGCAdTdT | (SEQ ID NO: 80) |
| PA-739/759 (sense) | CGGCUACAUUGAGGGCAAGdTdT | (SEQ ID NO: 81) |
| PA-739/759 (antisense) | CUUGCCCUCAAUGUAGCCGdTdT | (SEQ ID NO: 82) |
| PA-2087/2107 (G) (sense) | GCAAUUGAGGAGUGCCUGAdTdT | (SEQ ID NO: 83) |
| PA-2087/2107 (G) (antisense) | UCAGGCACUCCUCAAUUGCdTdT | (SEQ ID NO: 84) |
| PA-2110/2130 (sense) | UGAUCCCUGGGUUUUGCUUdTdT | (SEQ ID NO: 85) |
| PA-2110/2130 (antisense) | AAGCAAAACCCAGGGAUCAdTdT | (SEQ ID NO: 86) |
| PA-2131/2151 (sense) | UGCUUCUUGGUUCAACUCCdTdT | (SEQ ID NO: 87) |
| PA-2131/2151 (antisense) | GGAGUUGAACCAAGAAGCAdTdT | (SEQ ID NO: 88) |
| NP-231/251 (sense) | UAGAGAGAAUGGUGCUCUCdTdT | (SEQ ID NO: 89) |
| NP-231/251 antisense) | GAGAGCACCAUUCUCUCUAdTdT | (SEQ ID NO: 90) |
| NP-390/410 (sense) | UAAGGCGAAUCUGGCGCCAdTdT | (SEQ ID NO: 91) |
| NP-390/410 (antisense) | UGGCGCCAGAUUCGCCUUAdTdT | (SEQ ID NO: 92) |
| NP-1496/1516 (sense) | GGAUCUUAUUUCUUCGGAGdTdT | (SEQ ID NO: 93) |
| NP-1496/1516 (antisense) | CUCCGAAGAAAUAAGAUCCdTdT | (SEQ ID NO: 94) |
| NP-1496/1516a (sense) | GGAUCUUAUUUCUUCGGAGAdTdT | (SEQ ID NO: 188) |
| NP-1496/1516a (antisense) | UCUCCGAAGAAAUAAGAUCCdTdT | (SEQ ID NO: 189) |
| M-37/57 (sense) | CCGAGGUCGAAACGUACGUdTdT | (SEQ ID NO: 95) |
| M-37/57 (antisense) | ACGUACGUUUCGACCUCGGdTdT | (SEQ ID NO: 96) |
| M-480/500 (sense) | CAGAUUGCUGACUCCCAGCdTdT | (SEQ ID NO: 97) |
| M-480/500 (antisense) | GCUGGGAGUCAGCAAUCUGdTdT | (SEQ ID NO: 98) |
| M-598/618 (sense) | UGGCUGGAUCGAGUGAGCAdTdT | (SEQ ID NO: 99) |
| M-598/618 (antisense) | UGCUCACUCGAUCCAGCCAdTdT | (SEQ ID NO: 100) |
| M-934/954 (sense) | GAAUAUCGAAAGGAACAGCdTdT | (SEQ ID NO: 101) |
| M-934/954 (antisense) | GCUGUUCCUUUCGAUAUUCdTdT | (SEQ ID NO: 102) |
| NS-128/148 (sense) | CGGCUUCGCCGAGAUCAGAdAdT | (SEQ ID NO: 103) |
| NS-128/148 (antisense) | UCUGAUCUCGGCGAAGCCGdAdT | (SEQ ID NO: 104 |
| NS-562/582 (R) (sense) | GUCCUCCGAUGAGGACUCCdTdT | (SEQ ID NO: 105) |
| NS-562/582 (R) (antisense) | GGAGUCCUCAUCGGAGGACdTdT | (SEQ ID NO: 106) |
| NS-589/609 (sense) | UGAUAACACAGUUCGAGUCdTdT | (SEQ ID NO: 107) |
| NS-589/609 (antisense) | GACUCGAACUGUGUUAUCAdTdT | (SEQ ID NO: 108) |

All siRNAs were synthesized by Dharmacon Research (Lafayette, Colo.) using 2′ACE protection chemistry. The siRNA strands were deprotected according to the manufacturer's instructions, mixed in equimolar ratios and annealed by heating to 95° C. and slowly reducing the temperature by 1° C. every 30 s until 35° C. and 1° C. every min until 5° C.

siRNA electroporation. Log-phase cultures of MDCK cells were trypsinized, washed and resuspended in serum-free RPMI 1640 at 2×10⁷ cells per ml. 0.5 ml of cells were placed into a 0.4 cm cuvette and were electroporated using a Gene Pulser apparatus (Bio-Rad) at 400 V, 975 μF with 2.5 nmol siRNAs. Electroporation efficiencies were approximately 30-40% of viable cells. Electroporated cells were divided into 3 wells of a 6-well plate in DMEM medium containing 10% FCS and incubated at 37° C., 5% CO₂.

Viral infection. Six to eight h following electroporation, the serum-containing medium was washed away and 100 μl of PR8 or WSN virus at the appropriate multiplicity of infection was inoculated into the wells, each of which contained approximately 10⁶ cells. Cells were infected with either 1,000 PFU (one virus per 1,000 cells; MOI 0.001) or 10,000 PFU (one virus per 100 cells; MOI=0.01) of virus. After 1 h incubation at room temperature, 2 ml of infection medium with 4 μg/ml of trypsin was added to each well and the cells were incubated at 37° C., 5% CO₂. At indicated times, supernatants were harvested from infected cultures and the titer of virus was determined by hemagglutination of chicken erythrocytes (50 μl, 0.5%, Charles River laboratories, MA).

Measurement of Viral Titer. Supernatants were harvested at 24, 36, 48, and 60 hours after infection. Viral titer was measured using a standard hemagglutinin assay as described in Knipe D M, Howley, P M, Fundamental Virology, 4th edition, p34-35. The hemagglutination assay was done in V-bottomed 96-well plates. Serial 2-fold dilutions of each sample were incubated for 1 h on ice with an equal volume of a 0.5% suspension of chicken erythrocytes (Charles River Laboratories). Wells containing an adherent, homogeneous layer of erythrocytes were scored as positive. For plaque assays, serial 10-fold dilutions of each sample were titered for virus as described in Fundamental Virology, 4^{th} edition, p. 32 (referenced elsewhere herein) and well known in the art.

Results

To investigate the feasibility of using siRNA to suppress influenza virus replication, various influenza virus A RNAs were targeted. Specifically, the MDCK cell line, which is readily infected and widely used to study influenza virus, was utilized. Each siRNA was individually introduced into populations of MDCK cells by electroporation. siRNA targeted to GFP (sense: 5′-GGCUACGUCCAGGAGCGCAUU-3′ (SEQ ID NO: 110); antisense: 5′-UGCGCUCCUGGACGUAGCCUU-3′ (SEQ ID NO: 111)) was used as control. This siRNA is referred to as GFP-949. In subsequent experiments (described in examples below) the UU overhang at the 3′ end of both strands was replaced by dTdT with no effect on results. A mock electroporation was also performed as a control. Eight hours after electroporation cells were infected with either influenza A virus PR8 or WSN at an MOI of either 0.1 or 0.01 and were analyzed for virus production at various time points (24, 36, 48, 60 hours) thereafter using a standard hemagglutination assay. GFP expression was assayed by flow cytometry using standard methods.

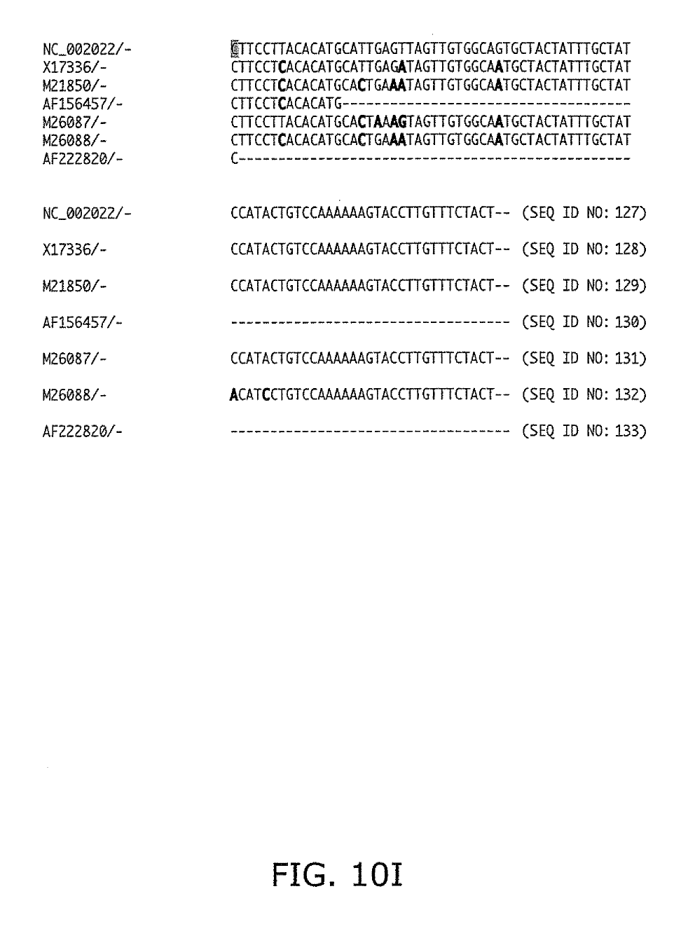

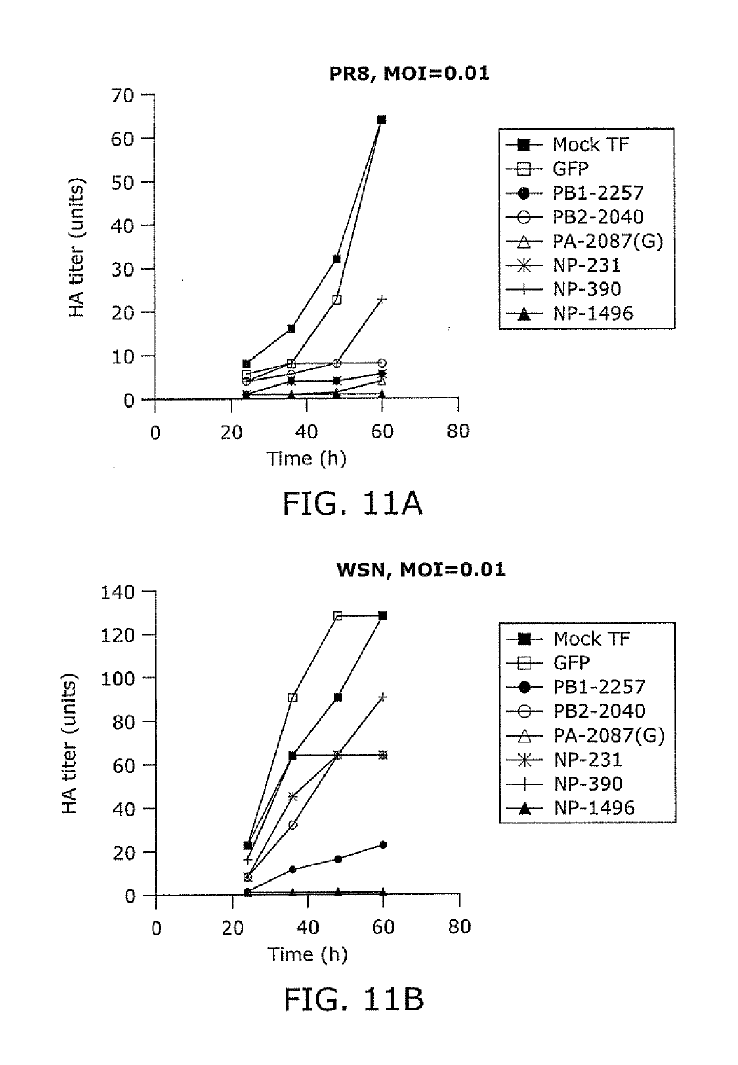

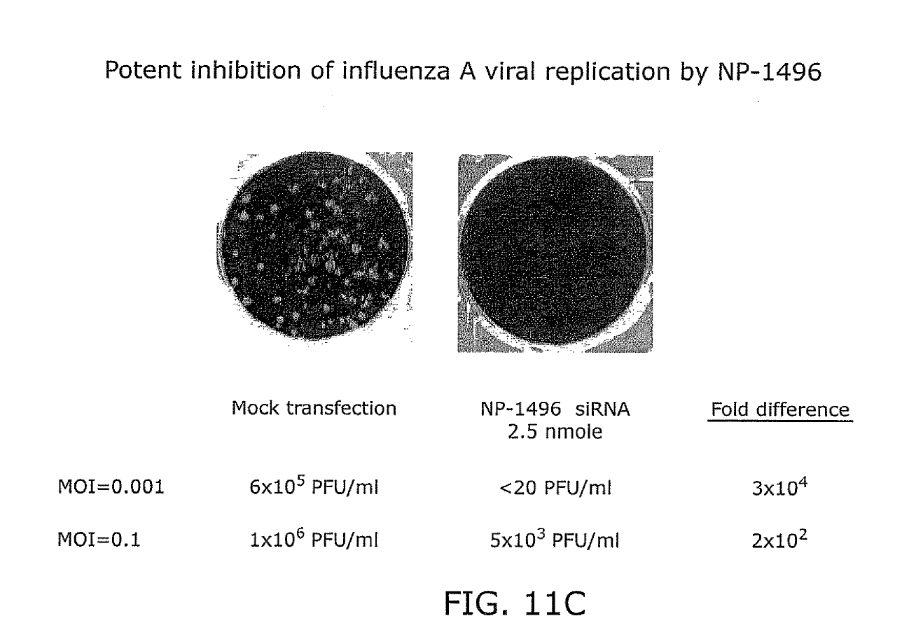

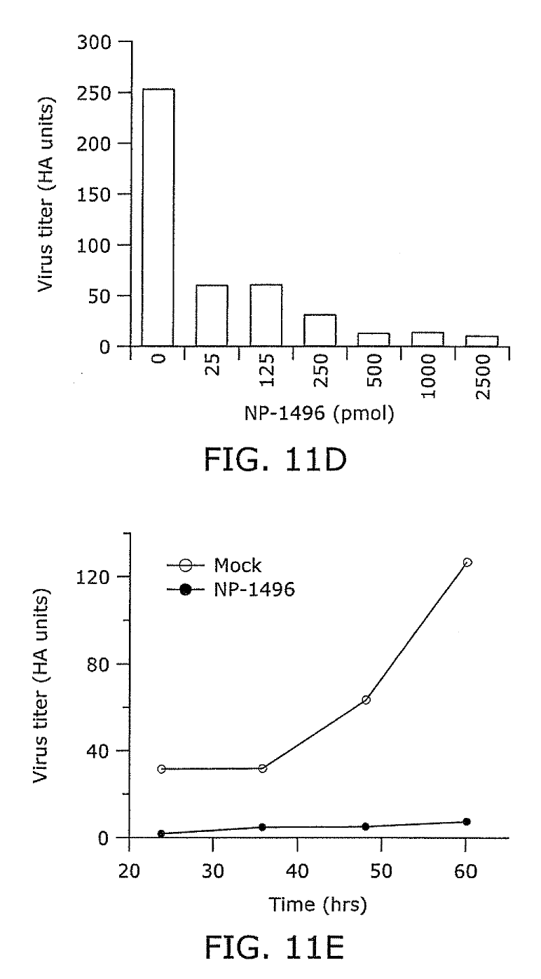

FIGS. 11A and 11B compare results of experiments in which the ability of individual siRNAs to inhibit replication of influenza virus A strain A/Puerto Rico/8/34 (H1N1) (FIG. 11A) or influenza virus A strain A/WSN/33 (H1N1) (FIG. 11B) was determined by measuring HA titer. Thus a high HA titer indicates a lack of inhibition while a low HA titer indicates effective inhibition. MDCK cells were infected at an MOI of 0.01. For these experiments one siRNA that targets the PB11 segment (PB1-2257/2277), one siRNA that targets the PB2 segment (PB2-2240/2260), one siRNA that targets the PA segment (PA-2087/2107 (G)), and three different siRNAs that target the NP genome and transcript (NP-231/251, NP-390/410, and NP-1496/1516) were tested. Note that the legends on FIGS. 11A and 11B list only the 5′ nucleotide of the siRNAs.

Symbols in FIGS. 11A and 11B are as follows: Filled squares represents control cells that did not receive siRNA. Open squares represents cells that received the GFP control siRNA. Filled circles represent cells that received siRNA PB1-2257/2277. Open circles represent cells that received siRNA PB2-2240/2260. Open triangles represent cells that received siRNA PA-2087/2107 (G). The X symbol represents cells that received siRNA NP-231/251. The + symbol represents cells that received siRNA NP-390/410. Closed triangles represent cells that received siRNA NP-1496/1516. Note that in the graphs certain symbols are sometimes superimposed. For example, in FIG. 11B the open and closed triangles are superimposed. Tables 3 and 4, which list the numerical values for each point, may be consulted for clarification.

As shown in FIGS. 11A and 11B (Tables 3 and 4), in the absence of siRNA (mock TF) or the presence of control (GFP) siRNA, the titer of virus increased over time, reaching a peak at approximately 48-60 hours after infection. In contrast, at 60 hours the viral titer was significantly lower in the presence of any of the siRNAs. For example, in strain WSN the HA titer (which reflects the level of virus) was approximately half as great in the presence of siRNAs PB2-2240 or NP-231 than in the controls. In particular, the level of virus was below the detection limit (10,000 PFU/ml) in the presence of siRNA NP-1496 in both strains. This represents a decrease by a factor of more than 60-fold in the PR8 strain and more than 120-fold in the WSN strain. The level of virus was also below the detection limit (10,000 PFU/ml) in the presence of siRNA PA-2087(G) in strain WSN and was extremely low in strain PR8. Suppression of virus production by siRNA was evident even from the earliest time point measured. Effective suppression, including suppression of virus production to undetectable levels (as determined by HA titer) has been observed at time points as great as 72 hours post-infection.

Table 5 summarizes results of siRNA inhibition assays at 60 hours in MDCK cells expressed in terms of fold inhibition. Thus a low value indicates lack of inhibition while a high value indicates effective inhibition. The location of siRNAs within a viral gene is indicated by the number that follows the name of the gene. As elsewhere herein, the number represents the starting nucleotide of the siRNA in the gene. For example, NP-1496 indicates an siRNA specific for NP, the first nucleotide starting at nucleotide 1496 of the NP sequence. Values shown (fold-inhibition) are calculated by dividing hemagglutinin units from mock transfection by hemagglutinin units from transfection with the indicated siRNA; a value of 1 means no inhibition.

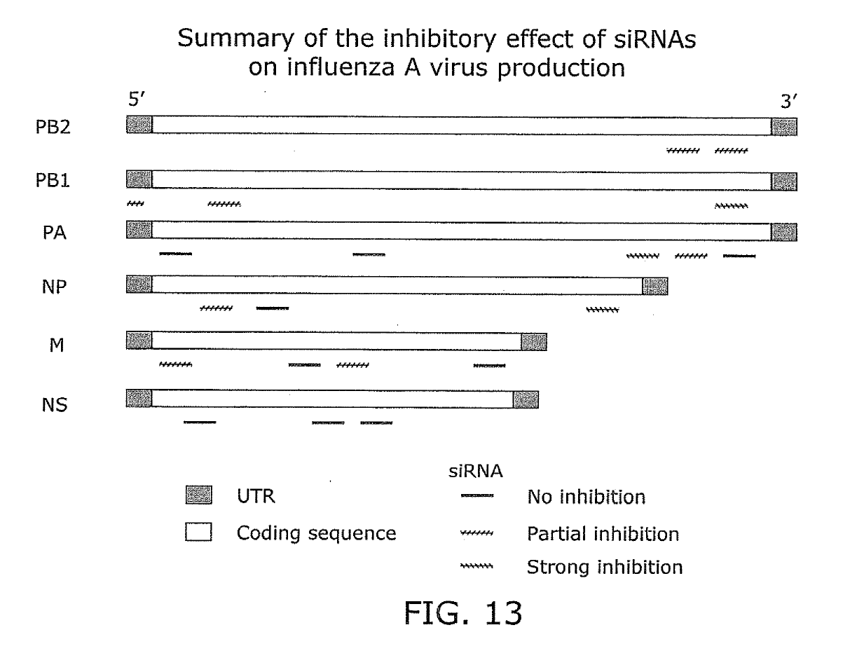

A total of twenty siRNAs, targeted to 6 segments of the influenza virus genome (PB2, PB1, PA, NP, M and NS), have been tested in the MDCK cell line system (Table 5). About 15% of the siRNA (PB1-2257, PA-2087G and NP-1496) tested displayed a strong effect, inhibiting viral production by more than 100 fold in most cases at MOI=0.001 and by 16 to 64 fold at MOI=0.01, regardless of whether PR8 or WSN virus was used. In particular, when siRNA NP-1496 or PA-2087 was used, inhibition was so pronounced that culture supernatants lacked detectable hemagglutinin activity. These potent siRNAs target 3 different viral gene segments: PB1 and PA, which are involved in the RNA transcriptase complex, and NP which is a single-stranded RNA binding nucleoprotein. Consistent with findings in other systems, the sequences targeted by these siRNAs are all positioned relatively close to the 3-prime end of the coding region (FIG. 13).

Approximately 40% of the siRNAs significantly inhibited virus production, but the extent of inhibition varied depending on certain parameters. Approximately 15% of siRNAs potently inhibited virus production regardless of whether PR8 or WSN virus was used. However, in the case of certain siRNAs, the extent of inhibition varied somewhat depending on whether PR8 or WSN was used. Some siRNAs significantly inhibited virus production only at early time points (24 to 36 hours after infection) or only at lower dosage of infection (MOI=0.001), such as PB2-2240, PB1-129, NP-231 and M37. These siRNAs target different viral gene segments, and the corresponding sequences are positioned either close to 3-prime end or 5-prime end of the coding region (FIG. 13 and Table 5).

Approximately 45% of the siRNAs had no discernible effect on the virus titer, indicating that they were not effective in interfering with influenza virus production in MDCK cells. In particular, none of the four siRNAs which target the NS gene segment showed any inhibitory effect.

To estimate virus titers more precisely, plaque assays with culture supernatants were performed (at 60 hrs) from culture supernatants obtained from virus-infected cells that had undergone mock transfection or transfection with NP-1496. Approximately 6×10⁵ pfu/ml was detected in mock supernatant, whereas no plaques were detected in undiluted NP-1496 supernatant (FIG. 1 IC). As the detection limit of the plaque assay is about 20 pfu (plaque forming unit)/ml, the inhibition of virus production by NP-1496 is at least about 30,000 fold. Even at an MOI of 0.1, NP-1496 inhibited virus production about 200-fold.

To determine the potency of siRNA, a graded amount of NP-1496 was transfected into MDCK cells followed by infection with PR8 virus. Virus titers in the culture supernatants were measured by hemagglutinin assay. As the amount of siRNA decreased, virus titer increased in the culture supernatants as shown in FIG. 11D. However, even when as little as 25 pmol of siRNA was used for transfection, approximately 4-fold inhibition of virus production was detected as compared to mock transfection, indicating the potency of NP-1496 siRNA in inhibiting influenza virus production.

For therapy, it is desirable for siRNA to be able to effectively inhibit an existing virus infection. In a typical influenza virus infection, new virions are released beginning at about 4 hours after infection. To determine whether siRNA could reduce or eliminate infection by newly released virus in the face of an existing infection, MDCK cells were infected with PR8 virus for 2 hours and then transfected with NP-1496 siRNA. As shown in FIG. 11E, virus titer increased steadily over time following mock transfection, whereas virus titer increased only slightly in NP-1496 transfected cells. Thus administration of siRNA after virus infection is effective.

Together, these results show that (i) certain siRNAs can potently inhibit influenza virus production; (ii) influenza virus production can be inhibited by siRNAs specific for different viral genes, including those encoding NP, PA, and PB1 proteins; and (iii) siRNA inhibition occurs in cells that were infected previously in addition to cells infected simultaneously with or following administration of siRNAs.

TABLE 3
Inhibition of Virus Strain A/Puerto Rico/8/34 (H1N1) Production by siRNAs
siRNA

| | Mock | GFP | PB1-2257 | PB2-2040 | PA-2087(G) | NP-231 | NP-390 | NP-1496 |
|---|---|---|---|---|---|---|---|---|
| 24 hr | 8 | 8 | 1 | 4 | 1 | 1 | 4 | 1 |
| 36 hr | 16 | 8 | 4 | 8 | 1 | 4 | 8 | 1 |
| 48 hr | 32 | 32 | 4 | 8 | 2 | 4 | 8 | 1 |
| 60 hr | 64 | 64 | 8 | 8 | 4 | 8 | 32 | 1 |

TABLE 4
Inhibition of Virus Strain A/WSN/33 (H1N1) Production by siRNAs
siRNA

| | Mock | GFP | PB1-2257 | PB2-2040 | PA-2087(G) | NP-231 | NP-390 | NP-1496 |
|---|---|---|---|---|---|---|---|---|
| 24 hr | 32 | 32 | 1 | 8 | 1 | 8 | 16 | 1 |
| 36 hr | 64 | 128 | 16 | 32 | 1 | 64 | 64 | 1 |
| 48 hr | 128 | 128 | 16 | 64 | 1 | 64 | 64 | 1 |
| 60 hr | 128 | 128 | 32 | 64 | 1 | 64 | 128 | 1 |

TABLE 5
Effects of 20 siRNAs on influenza virus production in MDCK cells

| | | Infecting virus (MOI) | | | | |
|---|---|---|---|---|---|---|
| | | PR8 | PR8 | PR8 | WSN | WSN |
| | siRNA | (0.001) | (0.01) | (0.1) | (0.001) | (0.01) |
| Exp. 1 | GFP-949 | 2 | 1 | | | |
| | PB2-2210 | 16 | 8 | | | |
| | PB2-2240 | 128 | 16 | | | |
| | PB1-6 | 4 | 4 | | | |
| | PB1-129 | 128 | 16 | | | |
| | PB1-2257 | 256 | 64 | | | |
| Exp. 2 | GFP-949 | 2 | 1 | | | |
| | PA-44 | 2 | 1 | | | |
| | PA-739 | 4 | 2 | | | |
| | PA-2087 | 128 | 16 | | | |
| | PA-2110 | 8 | 4 | | | |
| | PA-2131 | 4 | 2 | | | |
| Exp. 3 | NP-231 | 16 | 4 | | 4 | |
| | NP-390 | 4 | 2 | | 2 | |
| | NP-1496 | 16 | 64 | | 128 | |
| | M-37 | 2 | 2 | | 128 | |
| Exp. 4 | M-37 | | 2 | 1 | 128 | |
| | M-480 | | 2 | 1 | 4 | |
| | M-598 | | 2 | 1 | 128 | |
| | M-934 | | 1 | 1 | 4 | |
| | NS-128 | | 2 | 1 | 2 | |
| | NS-562 | | 1 | 1 | 1 | |
| | NS-589 | | 1 | 1 | 1 | |
| | NP-1496 | | 64 | 16 | 128 | |
| Exp. 5 | GFP-949 | | 1 | | | 1 |
| | PB2-2240 | | 8 | | | 2 |
| | PB1-2257 | | 8 | | | 4 |
| | PA-2087 | | 16 | | | 128 |
| | NP-1496 | | 64 | | | 128 |
| | NP-231 | | 8 | | | 2 |

### Example 3

### siRNAs that Target Viral RNA Polymerase or Nucleoprotein Inhibit Influenza A Virus Production in Chicken Embryos

Materials and Methods

SiRNA-oligofectamine complex formation and chicken embryo inoculation. SiRNAs were prepared as described above. Chicken eggs were maintained under standard conditions. 30 μl of Oligofectamine (product number: 12252011 from Life Technologies, now Invitrogen) was mixed with 30 μl of Opti-MEM I (Gibco) and incubated at RT for 5 min. 2.5 nmol (10 μl) of siRNA was mixed with 30 μl of Opti-MEM I and added into diluted oligofectamine. The siRNA and oligofectamine was incubated at RT for 30 min. 10-day old chicken eggs were inoculated with siRNA-oligofectamine complex together with 100 μl of PR8 virus (5000 pfu/ml). The eggs were incubated at 37° C. for indicated time and allantoic fluid was harvested. Viral titer in allantoic fluid was tested by HA assay as described above.

Results

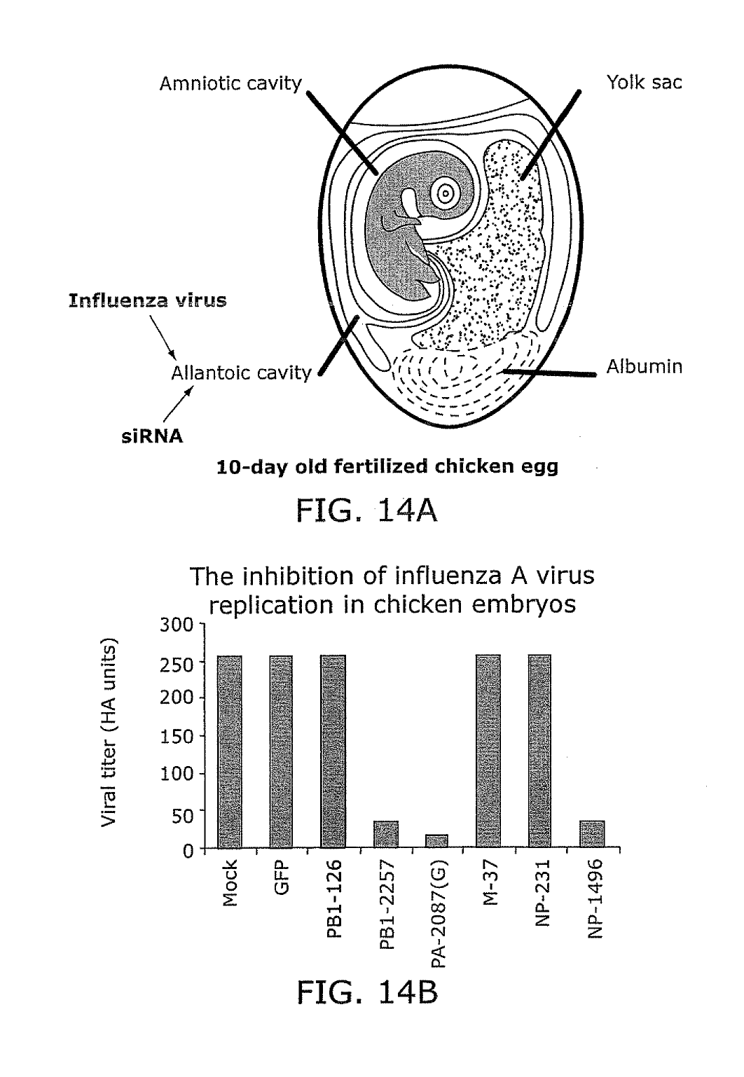

To confirm the results in MDCK cells, the ability of siRNA to inhibit influenza virus production in fertilized chicken eggs was also assayed. Because electroporation cannot be used on eggs, Oligofectamine, a lipid-based agent that has been shown to facilitate intracellular uptake of DNA oligonucleotides as well as siRNAs in vitro was used (25). Briefly, PR8 virus alone (500 pfu) or virus plus siRNA-oligofectamine complex was injected into the allantoic cavity of 10-day old chicken eggs as shown schematically in FIG. 14A. Allantoic fluids were collected 17 hours later for measuring virus titers by hemagglutinin assay. As shown in FIG. 14B, when virus was injected alone (in the presence of Oligofectamine), high virus titers were readily detected. Co-injection of GFP-949 did not significantly affect the virus titer. (No significant reduction in virus titer was observed when Oligofectamine was omitted.)

The injection of siRNAs specific for influenza virus showed results consistent with those observed in MDCK cells: The same siRNAs (NP-1496, PA2087 and PB1-2257) that inhibited influenza virus production in MDCK cells also inhibited virus production in chicken eggs, whereas the siRNAs (NP-231, M-37 and PB1-129) that were less effective in MDCK cells were ineffective in fertilized chicken eggs. Thus, siRNAs are also effective in interfering with influenza virus production in fertilized chicken eggs.

### Example 4

### SiRNA Inhibits Influenza Virus Production at the mRNA Level

Materials and Methods

SiRNA preparation was performed as described above.

RNA extraction, reverse transcription and real time PCR. 1×10⁷ MDCK cells were electroporated with 2.5 nmol of NP-1496 or mock electroporated (no siRNA). Eight hours later, influenza A PR8 virus was inoculated into the cells at MOI=0.1. At times 1, 2, and 3-hour post-infection, the supernatant was removed, and the cells were lysed with Trizol reagent (Gibco). RNA was purified according to the manufacturer's instructions. Reverse transcription (RT) was carried out at 37° C. for 1 hr, using 200 ng of total RNA, specific primers (see below), and Omniscript Reverse transcriptase kit (Qiagen) in a 20-μl reaction mixture according to the manufacturer's instructions. Primers specific for either mRNA, NP vRNA, NP cRNA, NS vRNA, or NS cRNA were as follows:

mRNA, dT₁₈ =

(SEQ ID NO: 112)

5′-TTTTTTTTTTTTTTTTTT-3′
NP vRNA, NP-367:

(SEQ ID NO: 113)

5′-CTCGTCGCTTATGACAAAGAAG-3′.
NP cRNA, NP-1565R:

(SEQ ID NO: 114)

5′-ATATCGTCTCGTATTAGTAGAAACAAGGGTATTTTT-3′.
NS vRNA, NS-527:

(SEQ ID NO: 115)

5′-CAGGACATACTGATGAGGATG-3′.
NS cRNA, NS-890R:

(SEQ ID NO: 116)

5′-ATATCGTCTCGTATTAGTAGAAACAAGGGTGTTTT-3′.

1 μl of RT reaction mixture (i.e., the sample obtained by performing reverse transcription) and sequence-specific primers were used for real-time PCR using SYBR Green PCR master mix (AB Applied Biosystems) including SYBR Green I double-stranded DNA binding dye. PCRs were cycled in an ABI PRISM 7000 sequence detection system (AB applied Biosystem) and analyzed with ABI PRISM 7000 SDS software (AB Applied Biosystems). The PCR reaction was carried out at 50° C., 2 min, 95° C., 10 min, then 95° C., 15 sec and 60° C., 1 min for 50 cycles. Cycle times were analyzed at a reading of 0.2 fluorescence units. All reactions were done in duplicate. Cycle times that varied by more than 1.0 between the duplicates were discarded. The duplicate cycle times were then averaged and the cycle time of β-actin was subtracted from them for a normalized value.

PCR primers were as follows.

| | |
|---|---|
| For NP RNAs: | |
| NP-367: | |
| 5′-CTCGTCGCTTATGACAAAGAAG-3′. | (SEQ ID NO: 117) |
| NP-460R: | |
| 5′-AGATCATCATGTGAGTCAGAC-3′. | (SEQ ID NO: 118) |
| For NS RNAs: | |
| NS-527: | |
| 5′-CAGGACATACTGATGAGGATG-3′. | (SEQ ID NO: 119) |
| NS-617R: | |
| 5′-GTTTCAGAGACTCGAACTGTG-3′. | (SEQ ID NO: 120) |

Results

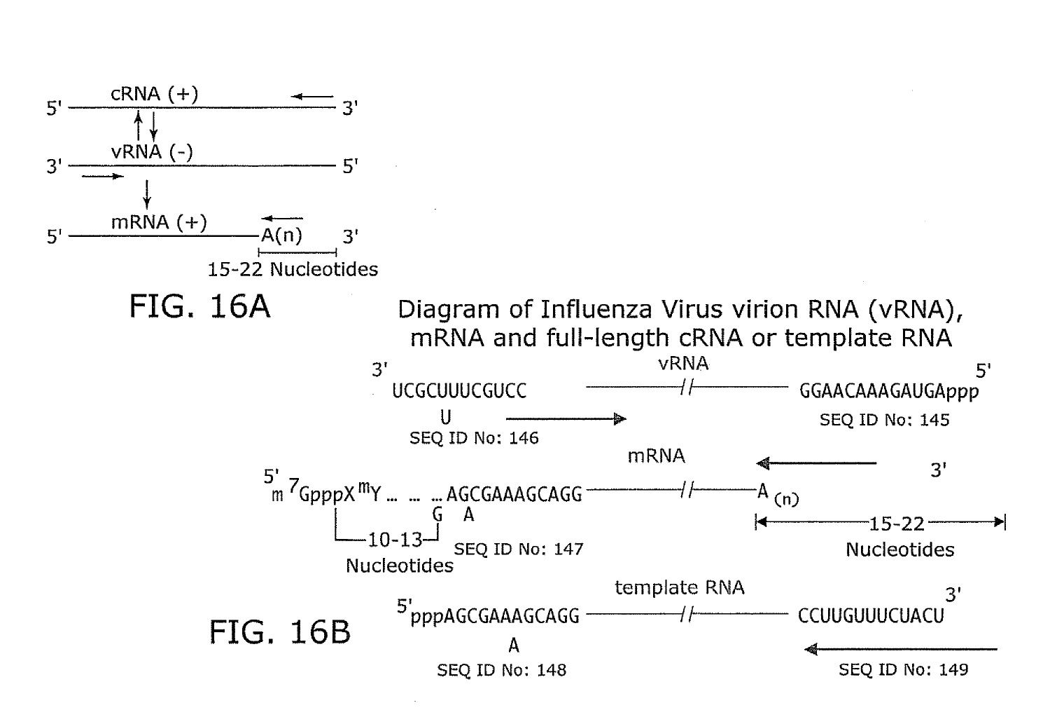

As described above, during replication of influenza virus, vRNA is transcribed to produce cRNA, which serves as a template for more vRNA synthesis, and mRNA, which serves as a template for protein synthesis (1). Although RNAi is known to target the degradation of mRNA in a sequence-specific manner (16-18), there is a possibility that vRNA and cRNA are also targets for siRNA since vRNA of influenza A virus is sensitive to nuclease (1). To investigate the effect of siRNA on the degradation of various RNA species, reverse transcription using sequence-specific primers followed by real time PCR was used to quantify the levels of vRNA, cRNA and mRNA. FIG. 16 shows the relationship between influenza virus vRNA, mRNA, and cRNA. As shown in FIGS. 16A and 16B, cRNA is the exact complement of vRNA, but mRNA contains a cap structure at the 5′ end plus the additional 10 to 13 nucleotides derived from host cell mRNA, and mRNA contains a polyA sequence at the 3′ end, beginning at a site complementary to a site 15-22 nucleotides downstream from the 5′ end of the vRNA segment. Thus compared to vRNA and cRNA, mRNA lacks 15 to 22 nucleotides at the 3′ end. To distinguish among the three viral RNA species, primers specific for vRNA, cRNA and mRNA were used in the first reverse transcription reaction (FIG. 16B). For mRNA, poly dT18 was used as primer. For cRNA, a primer complementary to the 3′ end of the RNA that is missing from mRNA was used. For vRNA, the primer can be almost anywhere along the RNA as long as it is complementary to vRNA and not too close to the 5′ end. The resulting cDNA transcribed from only one of the RNAs was amplified by real time PCR.

Following influenza virus infection, new virions are starting to be packaged and released by about 4 hrs. To determine the effect of siRNA on the first wave of mRNA and cRNA transcription, RNA was isolated early after infection. Briefly, NP-1496 was electroporated into MDCK cells. A mock electroporation (no siRNA) was also performed). Six to eight hours later, cells were infected with PR8 virus at MOI=0.1. The cells were then lysed at 1, 2 and 3 hours post-infection and RNA was isolated. The levels of mRNA, vRNA and cRNA were assayed by reverse transcription using primers for each RNA species, followed by real time PCR.

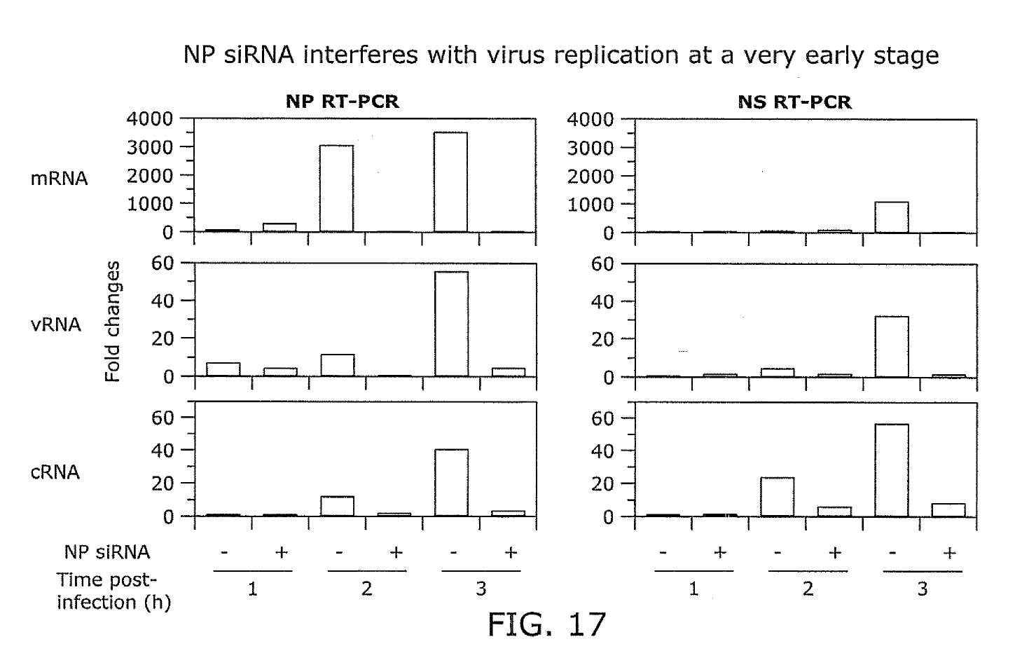

FIG. 17 shows amounts of viral NP and NS RNA species at various times following infection with virus, in cells that were mock transfected or transfected with siRNA NP-1496 approximately 6-8 hours prior to infection. As shown in FIG. 17, 1 hour after infection, there was no significant difference in the amount of NP mRNA between samples with or without NP siRNA transfection. As early as 2 hours post-infection, NP mRNA increased by 38 fold in the mock transfection group, whereas the levels of NP mRNA did not increase (or even slightly decreased) in cells transfected with siRNA. Three hours post-infection, mRNA transcript levels continued to increase in the mock transfection whereas a continuous decrease in the amount of NP mRNA was observed in the cells that received siRNA treatment. NP vRNA and cRNA displayed a similar pattern except that the increase in the amount of vRNA and cRNA in the mock transfection was significant only at 3 hrs post-infection. While not wishing to be bound by any theory, this is probably due to the life cycle of the influenza virus, in which an initial round of mRNA transcription occurs before cRNA and further vRNA synthesis.

These results indicate that, consistent with the results of measuring intact, live virus by hemagglutinin assay or plaque assay, the amounts of all NP RNA species were also significantly reduced by the treatment with NP siRNA. Although it is known that siRNA mainly mediates degradation of mRNA, the data from this experiment does not exclude the possibility of siRNA-mediated degradation of NP cRNA and vRNA although the results described below suggest that reduction in NP protein levels as a result of reduction in NP mRNA results in decreased stability of NP cRNA and/or vRNA.

### Example 5

### Identification of the Target of RNA Interference

Materials and Methods

SiRNA preparation of unmodified siRNAs was performed as described above. Modified RNA oligonucleotides, in which the 2′-hydroxyl group was substituted with a 2′-O-methyl group at every nucleotide residue of either the sense or antisense strand, or both, were also synthesized by Dharmacon. Modified oligonucleotides were deprotected and annealed to the complementary strand as described for unmodified oligonucleotides. siRNA duplexes were analyzed for completion of duplex formation by gel electrophoresis.

Cell culture, transfection with siRNAs, and infection with virus. These were performed essentially as described above. Briefly, for the experiment involving modified NP-1496 siRNA, MDCK cells were first transfected with NP-1496 siRNAs (2.5 nmol) formed from wild type (wt) and modified (m) strands and infected 8 hours later with PR8 virus at a MOI of 0.1. Virus titers in the culture supernatants were assayed 24 hours after infection. For the experiment involving M-37 siRNA, MDCK cells were transfected with M-37 siRNAs (2.5 nmol), infected with PR8 virus at an MOI of 0.01, and harvested for RNA isolation 1, 2, and 3 hours after infection. See Table 2 for M-37 sense and antisense sequences.

RNA extraction, reverse transcription and real time PCR were performed essentially as described above. Primers specific for either mRNA, M-specific vRNA, and M-specific cRNA, used for reverse transcription, were as follows:

mRNA, dT₁₈ =

(SEQ ID NO: 112)

5′-TTTTTTTTTTTTTTTTTT-3′
M vRNA:

(SEQ ID NO: 161)

5′-CGCTCAGACATGAGAACAGAATGG-3′
M cRNA:

(SEQ ID NO: 162)

5′-ATATCGTCTCGTATTAGTAGAAACAAGGTAGTTTTT-3′.

PCR primers for M RNAs were as follows:

| | |
|---|---|
| M forward: | |
| 5′-CGCTCAGACATGAGAACAGAATGG-3′ | (SEQ ID NO: 163) |
| M reverse: | |
| 5′-TAACTAGCCTGACTAGCAACCTC-3′ | (SEQ ID NO: 164) |

Results

To investigate the possibility that siRNA might interfere with vRNA and/or cRNA in addition to mRNA, NP-1496 siRNAs in which either the sense (S or +) or antisense (AS or −) strand was modified were synthesized. The modification, which substitutes a 2′-O-methyl group for the 2′-hydroxyl group in every nucleotide residue, does not affect base-pairing for duplex formation, but the modified RNA strand no longer supports RNA interference. In other words, an siRNA in which the sense strand is modified but the antisense strand is wild type (mS:wtAS) will support degradation of RNAs having a sequence complementary to the antisense strand but not a sequence complementary to the sense strand. Conversely, an siRNA in which the sense strand is wild type but the antisense strand is modified (wtS:mAS) will support degradation of RNAs having a sequence complementary to the sense strand but will not support degradation of RNAs having a sequence complementary to the sense strand. This phenomenon is described in more detail in copending Provisional Patent application Ser. No. 60/446,387 entitled “Reducing RNAi Background”.

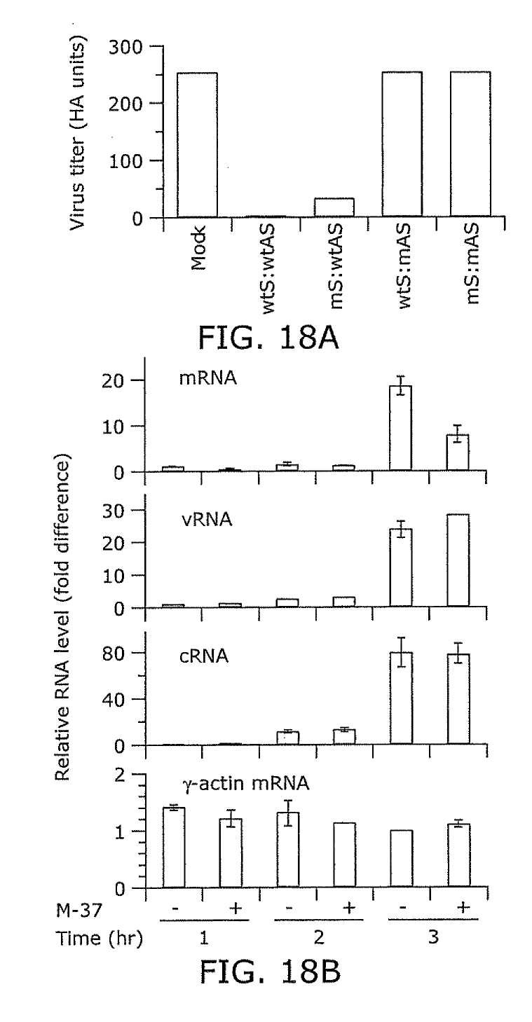

MDCK cells were either mock transfected or transfected with NP-1496 siRNAs in which either the sense strand (mS:wtAS) or the antisense strand (wtS:mAS), was modified while the other strand was wild type. Cells were also transfected with NP-1496 siRNA in which both strands were modified (mS:mAS). Cells were then infected with PR8 virus, and virus titer in supernatants was measured. As shown in FIG. 18A, high virus titers were detected in cultures subjected to mock transfection. As expected, very low virus titers were detected in cultures transfected with wild type siRNA (wtS:wtAS), but high virus titers were detected in cultures transfected with siRNA in which both strands were modified (mS:mAS). Virus titers were high in cultures transfected with siRNA in which the antisense strand was modified (wtAS:mAS), whereas the virus titers were low in cultures transfected with siRNA in which the sense strand only was modified (mS:wtAS). While not wishing to be bound by any theory, the inventors suggest that the requirement for a wild type antisense (−) strand of siRNA duplex to inhibit influenza virus production suggests that the target of RNA interference is either mRNA (+) or cRNA (+) or both.

To further distinguish these possibilities, the effect of siRNA on the accumulation of corresponding mRNA, vRNA, and cRNA was examined. To follow transcription in a cohort of simultaneously infected cells, siRNA-transfected MDCK cells were harvested for RNA isolation 1, 2, and 3 hours after infection (before the release and re-infection of new virions). The viral mRNA, vRNA, and cRNA were first independently converted to cDNA by reverse transcription using specific primers. Then, the level of each cDNA was quantified by real time PCR. As shown in FIG. 18B, when M-specific siRNA M-37 was used, little M-specific mRNA was detected one or two hours after infection. Three hours after infection, M-specific mRNA was readily detected in the absence of M-37. In cells transfected with M-37, the level of M-specific mRNA was reduced by approximately 50%. In contrast, the levels of M-specific vRNA and cRNA were not inhibited by the presence of M-37. While not wishing to be bound by any theory, these results indicate that viral mRNA is probably the target of siRNA-mediated interference.

### Example 6

### Broad Effects of Certain siRNAs on Viral RNA Accumulation

Results

SiRNA preparation was performed as described above.

RNA extraction, reverse transcription and real time PCR were performed as described in Example 3. Primers specific for either mRNA, NP vRNA, NP cRNA, NS vRNA, NS cRNA, M vRNA, or M cRNA were as described in Examples 4 and 5. Primers specific for PB1 vRNA, PB1 cRNA, PB2 vRNA, PB2 cRNA, PA vRNA, or PA cRNA, used for reverse transcription, were as follows:

(SEQ ID NO: 165)

PB1 vRNA: 5′-GTGCAGAAATCAGCCCGAATGGTTC-3′

(SEQ ID NO: 166)

PB1 cRNA: 5′-ATATCGTCTCGTATTAGTAGAAACAAGGCATTT-3′

(SEQ ID NO: 167)

PB2 vRNA: 5′-GCGAAAGGAGAGAAGGCTAATGTG-3′

(SEQ ID NO: 168)

PB2 cRNA: 5′-ATATGGTCTCGTATTAGTAGAAACAAGGTCGTTT-3′

(SEQ ID NO: 169)

PA vRNA: 5′-GCTTCTTATCGTTCAGGCTCTTAGG-3′

(SEQ ID NO: 170)

PA cRNA: 5′-ATATCGTCTCGTATTAGTAGAAACAAGGTACTT-3′

PCR primers for PB1, PB2, and PA RNAs were as follows:

(SEQ ID NO: 171)

PB1 forward: 5′-CGGATTGATGCACGGATTGATTTC-3′

(SEQ ID NO: 172)

PB1 reverse: 5′-GACGTCTGAGCTCTTCAATGGTGGAAC-3′

(SEQ ID NO: 173)

PB2 forward: 5′-GCGAAAGGAGAGAAGGCTAATGTG-3′

(SEQ ID NO: 174)

PB2 reverse: 5′-AATCGCTGTCTGGCTGTCAGTAAG-3′

(SEQ ID NO: 175)

PA forward: 5′-GCTTCTTATCGTTCAGGCTCTTAGG-3′

(SEQ ID NO: 176)

PA reverse: 5′-CCGAGAAGCATTAAGCAAAACCCAG-3′

Results

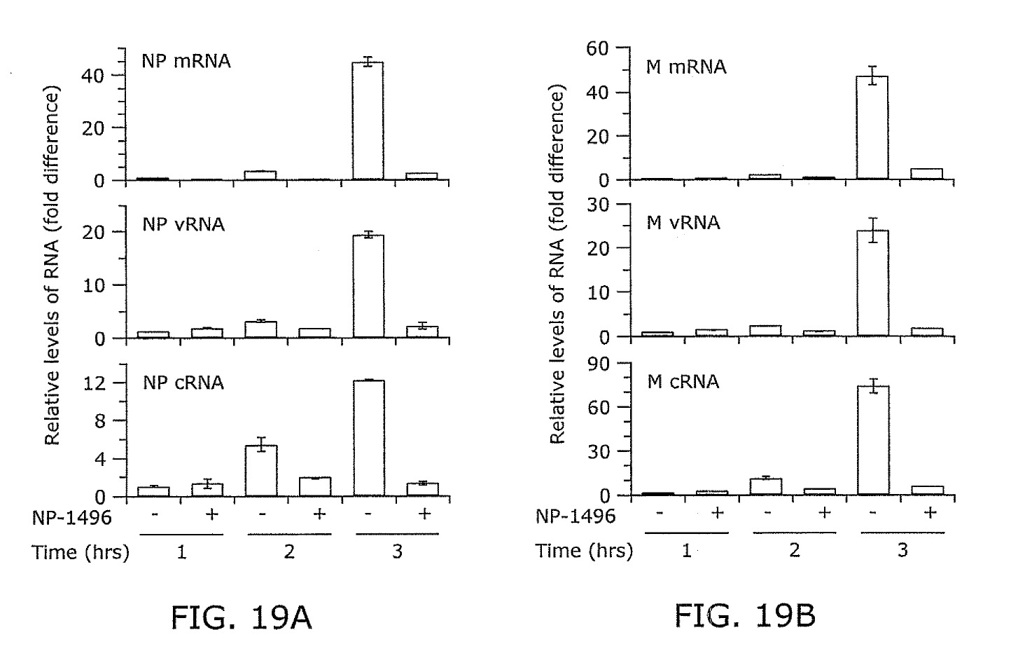

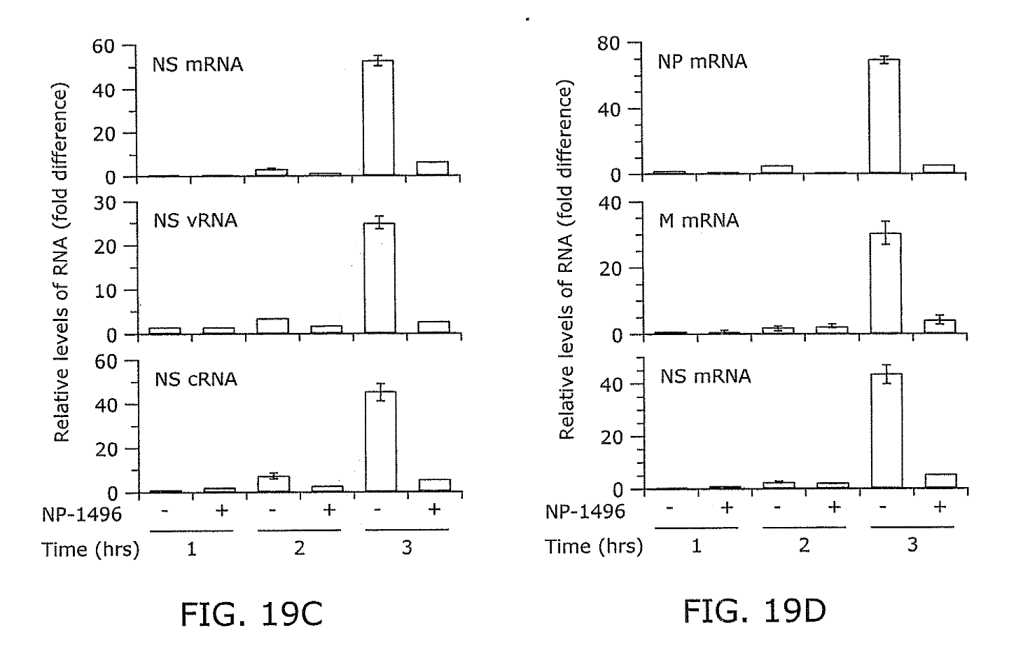

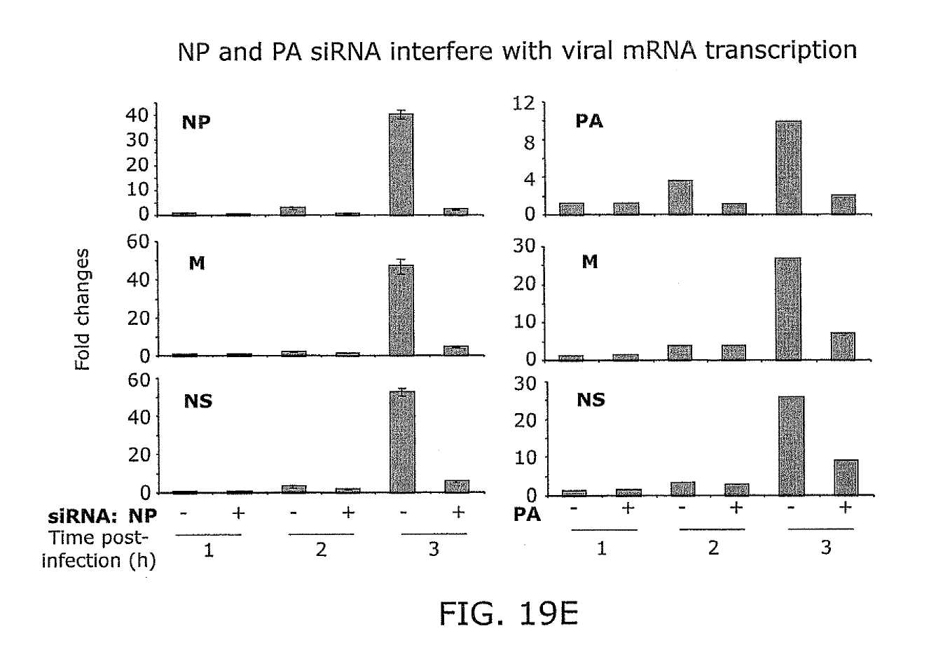

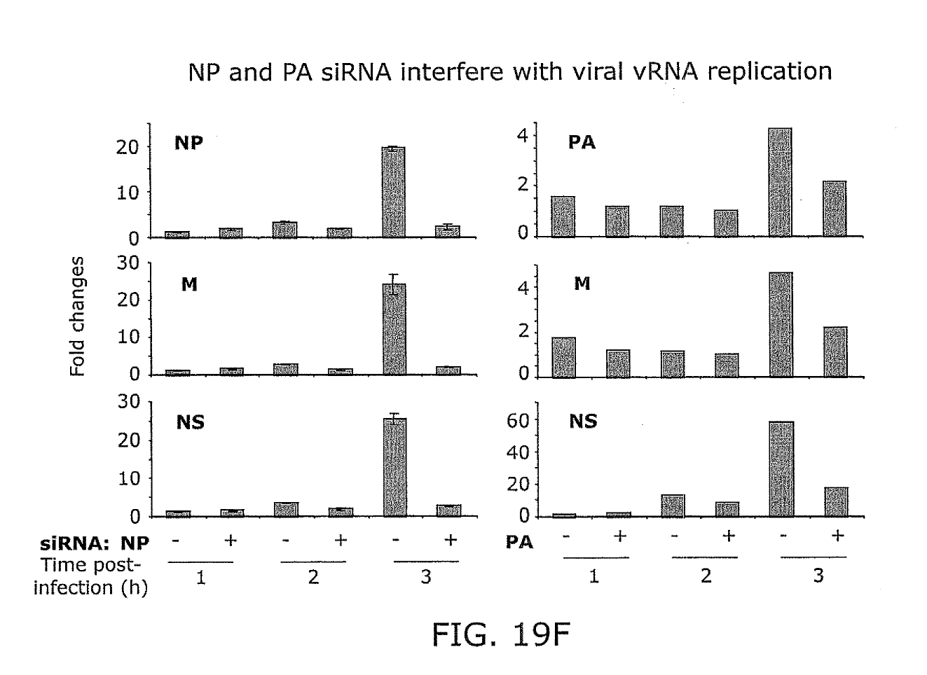

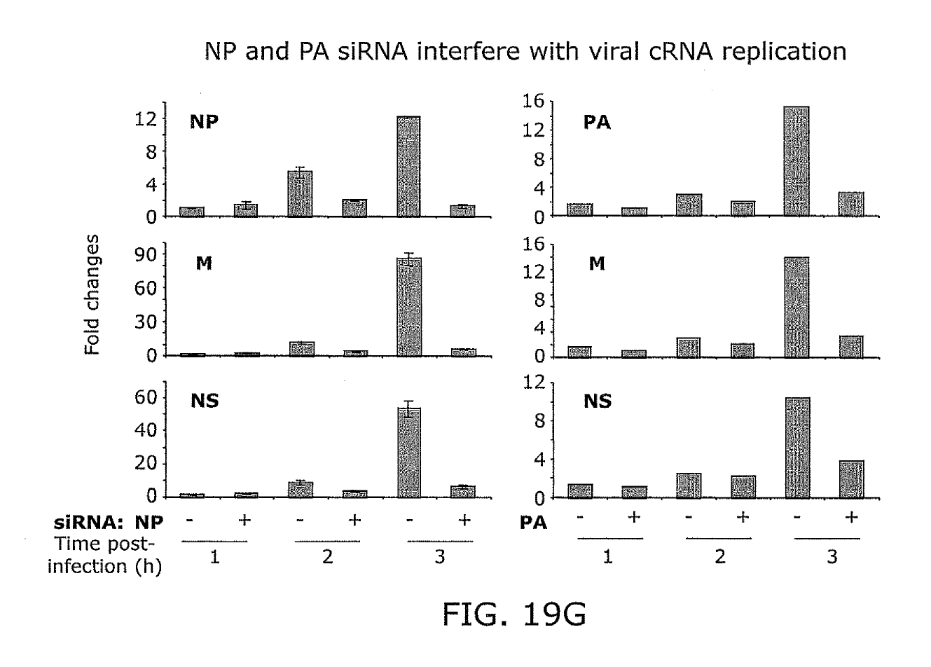

To determine whether NP-1496 targets the degradation of the NP gene segment specifically or whether the levels of viral RNAs other than NP are also affected, primers specific for NS were used for RT and real time PCR to measure the amount of different NS RNA species (mRNA, vRNA, cRNA) as described above (Example 4). As shown in FIG. 19, the changes in NS mRNA, vRNA and cRNA showed the same pattern as that observed for NP RNAs. At 3 hours post-infection, a significant increase in all NS RNA species could be seen in mock transfected cells, whereas no significant changes in NS RNA levels were seen in the cells that received NP-1496 siRNA. This result indicates that the transcription and replication of different viral RNAs are coordinately regulated, at least with respect to NP RNAs. By coordinately regulated is meant that levels of one transcript affect levels of another transcript, either directly or indirectly. No particular mechanism is implied. When NP transcripts are degraded by siRNA treatment the levels of other viral RNAs are also reduced.

To further explore the effect of NP siRNAs on other viral RNAs, accumulation of mRNA, vRNA, and cRNA of all viral genes was measured in cells that had been treated with NP-1496. As shown in FIG. 19A (top panel), NP-specific mRNA was low one or two hours after infection. Three hours after infection, NP mRNA was readily detected in the absence of NP-1496, whereas in the presence of NP-1496, the level of NP mRNA remained at the background level, indicating that siRNA inhibited the accumulation of specific mRNA. As shown in FIG. 19A (middle and bottom panels) levels of NP-specific and NS-specific vRNA and cRNA were greatly inhibited by the presence of NP-1496. These results confirm the results described in Example 4. In addition, in the NP-1496-treated cells, the accumulation of mRNA, vRNA, and cRNA of the M, NS, PB1, PB2, and PA genes was also inhibited (FIGS. 19B, 19C, and 19H). Furthermore, the broad inhibitory effect was also observed for PA-2087. The top, middle, and bottom panels on the left side in FIGS. 19E, 19F, and 19G display the same results as presented in FIGS. 19A, 19B, and 19C, showing the inhibition of viral mRNA transcription and of viral vRNA and cRNA replication by NP-1496 siRNA. The top, middle, and bottom panels on the right side in FIGS. 19E, 19F, and 19G present results of the same experiment performed with PA-2087 siRNA at the same concentration. As shown in FIG. 19E, right upper, middle, and lower panels respectively, at three hours after infection PA, M, and NS mRNA were readily detected in the absence of PA-2087, whereas the presence of PA-2087 inhibited transcription of PA, M, and NS mRNA. As shown in FIG. 19F, right upper, middle, and lower panels respectively, at three hours after infection PA, M, and NS vRNA were readily detected in the absence of PA-2087, whereas the presence of PA-2087 inhibited accumulation of PA, M, and NS vRNA. As shown in FIG. 19G, right upper, middle, and lower panels respectively, at three hours after infection PA, M, and NS cRNA were readily detected in the absence of PA-2087, whereas the presence of PA-2087 inhibited accumulation of PA, M, and NS cRNA. In addition, FIG. 19H shows that NP-specific siRNA inhibits the accumulation of PB1- (top panel), PB2- (middle panel) and PA- (lower panel) specific mRNA.

While not wishing to be bound by any theory, the inventors suggest that the broad effect of NP siRNA is probably a result of the importance of NP in binding and stabilizing vRNA and cRNA, and not because NP-specific siRNA targets RNA degradation non-specifically. The NP gene segment in influenza virus encodes a single-stranded RNA-binding nucleoprotein, which can bind to both vRNA and cRNA (see FIG. 15). During the viral life cycle, NP mRNA is first transcribed and translated. The primary function of the NP protein is to encapsidate the virus genome for the purpose of RNA transcription, replication and packaging. In the absence of NP protein, the full-length synthesis of both vRNA and cRNA is strongly impaired. When NP siRNA induces the degradation of NP RNA, NP protein synthesis is impaired and the resulting lack of sufficient NP protein subsequently affects the replication of other viral gene segments. In this way, NP siRNA could potently inhibit virus production at a very early stage.

The number of NP protein molecules in infected cells has been hypothesized to regulate the levels of mRNA synthesis versus genome RNA (vRNA and cRNA) replication (1). Using a temperature-sensitive mutation in the NP protein, previous studies have shown that cRNA, but not mRNA, synthesis was temperature sensitive both in vitro and in vivo (70, 71). NP protein was shown to be required for elongation and antitermination of the nascent cRNA and vRNA transcripts (71, 72). The results presented above show that NP-specific siRNA inhibited the accumulation of all viral RNAs in infected cells. While not wishing to be bound by any theory, it appears probable that in the presence of NP-specific siRNA, the newly transcribed NP mRNA is degraded, resulting in the inhibition of NP protein synthesis following virus infection. Without newly synthesized NP, further viral transcription and replication, and therefore new virion production is inhibited.

Similarly, in the presence of PA-specific, the newly transcribed PA mRNA is degraded, resulting in the inhibition of PA protein synthesis. Despite the presence of 30-60 copies of RNA transcriptase per influenza virion (1), without newly synthesized RNA transcriptase, further viral transcription and replication are likely inhibited. Similar results were obtained using siRNA specific for PB1. In contrast, the matrix (M) protein is not required until the late phase of virus infection (1). Thus, M-specific siRNA inhibits the accumulation of M-specific mRNA but not vRNA, cRNA, or other viral RNAs. Taken together, these findings demonstrate a critical requirement for newly synthesized nucleoprotein and polymerase proteins in influenza viral RNA transcription and replication. Both mRNA- and virus-specific mechanisms by which NP-, PA-, and PB1-specific siRNAs interfere with mRNA accumulation and other viral RNA transcription suggest that these siRNAs may be especially potent inhibitors of influenza virus infection. In particular, the results described herein suggest that, in general, siRNAs targeted to transcripts that encode RNA or DNA binding proteins that normally bind to agent-specific nucleic acids (DNA or RNA) are likely to have broad effects (e.g., effects on other agent-specific transcripts) rather than simply reducing the level of the targeted RNA. Similarly, the results described herein suggest that, in general, siRNAs targeted to the polymerase genes (RNA polymerase, DNA polymerase, or reverse transcriptase) of infectious agents are likely to have broad effects (e.g., effects on other agent-specific transcripts) rather than simply reducing levels of polymerase RNA.

### Example 7

### Broad Inhibition of Viral RNA Accumulation by Certain siRNAs is not due to the Interferon Response or to Virus-Induced RNA Degradation

Materials and Methods

Measurement of RNA levels. RNA levels were measured using PCR under standard conditions. The following PCR primers were used for measurement of γ-actin RNA.

(SEQ ID NO: 177)

γ-actin forward: 5′-TCTGTCAGGGTTGGAAAGTC-3′

(SEQ ID NO: 178)

γ-actin reverse: 5′-AAATGCAAACCGCTTCCAAC-3′

Culture of Vero cells and measurements of phosphorylated PKR were performed according to standard techniques described in the references cited below.

Results

One possible cause for the broad inhibition of viral RNA accumulation is an interferon response of the infected cells in the presence of siRNA (23, 65, 66). Thus, the above experiments were repeated in Vero cells in which the entire IFN locus, including all α, β, and ω genes, are deleted (67, 68) (Q.G. and J.C. unpublished data). Just as in MDCK cells, the accumulation of NP-, M-, and NS-specific mRNAs were all inhibited by NP-1496 (FIG. 19D). In addition, the effect of siRNA on the levels of transcripts from cellular genes, including β-actin, γ-actin, and GAPDH, was assayed using PCR. No significant difference in the transcript levels was detected in the absence or presence of siRNA (FIG. 18C bottom panel, showing lack of effect of M-37 siRNA on γ-actin mRNA, and data not shown), indicating that the inhibitory effect of siRNA is specific for viral RNAs. These results suggest that the broad inhibition of viral RNA accumulation by certain siRNAs is not a result of a cellular interferon response.

Following influenza virus infection, the presence of dsRNA also activates a cellular pathway that targets RNA for degradation (23). To examine the effect of siRNA on the activation of this pathway, we assayed the levels of phosphorylated protein kinase R (PKR), the most critical component of the pathway (23). Transfection of MDCK cells with NP-1496 in the absence of virus infection did not affect the levels of activated PKR (data not shown). Infection by influenza virus resulted in an increased level of phosphorylated PKR, consistent with previous studies (65, 66, 69). However, the increase was the same in the presence or absence of NP-1496 (data not shown). Thus, the broad inhibition of viral RNA accumulation is not a result of enhanced virus-induced degradation in the presence of siRNA.

### Example 8

### Systematic Identification of siRNAs with Superior Ability to Inhibit Influenza Virus Production Either Alone or in Combination

This example describes a systematic approach to the identification of siRNAs with superior ability to inhibit influenza virus production. Although the example refers to siRNAs, it is to be understood that the same methodology may be employed for the evaluation of shRNAs whose duplex portion is identical to the duplex portion of the siRNAs described below and which contain a loop whose sequence may vary, as described above.

Rationale: For both prophylactic and therapeutic purposes, it is desirable to identify siRNAs that exhibit superior potency for inhibiting influenza virus infection. As described above, 20 siRNAs, 19 of which were based on highly conserved sequences that included AA di-nucleotides at the 5′ end, have been designed and tested. Although the presence of AA di-nucleotides at this position was initially considered important for siRNA function, more recent findings indicate that they are not required because siRNAs based on sequences containing other nucleotides at this position are just as effective (22, 28). Thus, additional siRNAs designed based on sequences not beginning with AA will be designed and tested so as to identify additional siRNAs that effectively inhibit influenza virus production.

The availability of a few potent inhibitory siRNAs will enable their use in combinations. A recent study on siRNA inhibition of poliovirus showed that the use of a single siRNA resulted in the outgrowth of pre-existing variant poliovirus that cannot be targeted by siRNA (24). Because influenza virus is known to mutate at a high rate (4), the use of a single siRNA could possibly promote the outgrowth of resistant viruses and thus potentially render the siRNA ineffective after a period of time. On the other hand, the likelihood that a resistant virus will emerge is reduced by orders of magnitude if two or more different siRNAs are used simultaneously, especially those siRNAs specific for different viral RNAs. Thus, siRNAs will be tested in combinations of two or more so as to find the most effective combinations.

This example describes a systematic approach to achieving the following goals:

1) To design and test additional siRNAs so that the entire conserved region of the influenza virus genome is covered once by non-overlapping siRNAs.

2) To identify the most potent inhibitory siRNAs by screening them with increasingly high multiplicity of infection (MOI).

3) To identify the most potent combinations of effective siRNAs to prevent the emergence of resistant viruses.

Designing and testing additional siRNAs. Additional siRNAs specific for the conserved regions of the viral genome that are not covered by the siRNAs described in Example 1 will be designed. The object is to cover the conserved regions of the viral genome once with non-overlapping siRNAs. Non-overlapping siRNAs are chosen for two reasons. First, simultaneous application of overlapping siRNAs will probably not provide the most effective combinations because some of the target sequences are shared. Mutation in the overlapping region would likely render both siRNAs ineffective. Second, for an extensive screen, the number of overlapping siRNAs may be too large to test within a reasonable period of time. The aim is to obtain at least one potent siRNA for each of PA, PB1, PB2, NP, M, and NS. (By RNA splicing, M and NS genes each encode two proteins. If possible, siRNAs specific for both transcripts from the same gene are designed.) Potent siRNAs specific to NP, PA, and PB1 have already been identified (Table 5) therefore the focus will be on testing more siRNA candidates specific for PB2, M, and NS. If testing non-overlapping siRNAs does not reveal potent siRNAs for these genes overlapping siRNA candidates will be tested. Availability of potent inhibitory siRNA specific for each of the six genes will facilitate the identification of most potent combinations.

To design the additional non-overlapping siRNAs, the same criteria as described in Example 1 and in the detailed description will be used, except that the initial AA di-nucleotides will not be required. Based on these criteria, it is estimated that it may be desirable to test about 40 siRNAs. Single stranded RNA oligonucleotides will be commercially synthesized and annealed to their complementary strands. The resulting siRNA duplexes will be tested for their ability to interfere with influenza virus production (PR8, WSN, or both) in MDCK cells as measured by hemagglutinin assay. Those siRNA that are effective in the cell line will be further evaluated in chicken embryos. SiRNAs that show consistent inhibitory effects with both subtypes of virus and in both cells and embryos are preferred for further investigation.

Comparing the potencies of siRNAs. Once siRNAs that significantly inhibit influenza virus production are identified, their potencies in the same assay will be compared in order to identify the most potent ones. In most of the assays described above using MDCK cells, virus was used at a MOI of either 0.001 or 0.01. It was found that the virus titer in two samples (NP-1496 and PA-2087) was undetectable by hemagglutinin assay and in one sample (NP-1496) undetectable by plaque assay. To distinguish the potencies of these siRNAs, especially those specific for the same gene, the MOI used to infect MDCK cells will be increased to 0.1 or higher. siRNAs will also be tested in chick embryos. Plaque assays will be used to more precisely measure virus titers.

In addition, the potencies of siRNAs will be compared by titrating the amount of siRNA used for transfection. Briefly, different amounts of siRNA (such as 0.025, 0.05, 0.1, and 0.25 nmol) will be electroporated into MDCK cells (1×10⁷). Cells will be infected with PR8 or WSN virus at a fixed MOI (such as 0.01), and culture supernatants will be harvested 60 hrs later to measure virus titers by hemagglutination. Results from these experiments will help to determine not only the relative potencies of each siRNA but also the minimal amount necessary for maximal inhibition. The latter will be useful for determining how much of each siRNA should be used in combinations as described below.

Identifying the most potent combinations of siRNAs. The use of two or more different siRNAs simultaneously may be of considerable use in order to prevent the emergence of variant viruses that can escape interference by a single siRNA. Once potent siRNAs for a number of the eight virus genes are identified, their efficacies in combinations will be examined. Preferably potent siRNAs targeted to at least 2 genes are identified. More preferably potent siRNAs targeted to at least 3, 4, 5, 6, 7, or even all 8 genes are identified. However, it may be desirable to limit the testing initially to less than all 8 genes, e.g., 5 or 6 genes. For these studies, the following considerations are important: i) numbers of different siRNAs used in the same mixture, ii) the minimal amount of each siRNA used in the “cocktail”, and iii) the most efficient ways to identify the most potent combinations.

The mutation rate of influenza virus is estimated to be 1.5×10⁻⁵ per nucleotide per infection cycle (4). If two siRNAs specific for different genes are used simultaneously, the probability of emergence of resistant virus is 2.25×10⁻¹⁰. Considering that siRNAs can sometimes tolerate one nucleotide mismatch (26), especially at the ends (28) and in the 3′ half of the antisense strand, simultaneous use of two siRNAs should be quite effective in preventing the emergence of resistant virus. To be conservative, three siRNAs used in combination should be sufficient. This calculation assumes that each siRNA in a mixture acts independently. Initially, the minimal amount of siRNA that is required for the maximal inhibition of influenza virus production as determined above using that siRNA alone will be used in the combinations. Some studies have shown that the RNAi machinery in mammalian cells and Drosophila may be limiting (27, 29, 30). If this is appears to be the case for RNA interference with influenza virus production, we will test reduced amounts for each siRNA in the combinations, such as half-maximal dose of each siRNA in combination of two, will be tested.

First, test combinations of two siRNAs will be systematically tested. The advantage of this strategy is that it will yield not only the most potent combinations of two siRNAs but likely also potent components in combinations of three siRNAs. Although combinations of two siRNAs specific for different genes or different steps of the virus life cycle may be more desirable because of potential synergistic effects, it is worth testing combinations of siRNAs specific for different components of the transcriptase because they are non-abundant proteins and critical for virus production. Assuming that one potent siRNA for each gene (PA, PB1, PB2, NP, M, and NS) is identified, it will be necessary to test 15 combinations to cover all possible combinations of two siRNAs.

siRNAs will be introduced into MDCK cells by electroporation individually or in combinations of two. Eight hrs later, cells will be infected with PR8 or WSN virus at a pre-determined MOI and culture supernatants will be harvested 60 hrs later for assaying the virus titer by hemagglutination. The precise titers in samples that have substantially lower hemagglutinin units will be determined by plaque assay. The combinations of siRNAs will be assayed in chicken embryos to confirm the results from the cell line.

Results from this series of experiments will reveal the relative potencies of combinations of two siRNAs, and whether a combination of two siRNAs has synergistic effects. For example, if the combination of NP-1496 and PA-2087 is more than the additive effect of NP-1496 plus PA-2087 individually, the combination would have a synergistic effect. These results will provide an indication as to which combinations of three siRNAs are likely to be optimally effective. For example, assuming that the combination of NP-1496 and PA-2087 is more effective than NP-1496 or PA-2087 alone, and the combination MIT 9926 of PA-2087 and PB1-2257 is more effective than PA-2087 or PB1-2257 alone, the three siRNAs in a cocktail containing NP-1496, PA-2087, and PB1-2257 will be likely especially effective. The potencies of at least three siRNA cocktails that are most likely to be effective in MDCK cells and chicken embryos will be measured. If the results from the combination of two siRNAs are not helpful, the potencies of three siRNA cocktails will be systematically tested as described for testing two siRNA cocktails. To cover all possibilities, 10 different combinations will need to be tested.

In summary, results obtained from the proposed experiments will likely identify the most potent siRNAs from the conserved regions of a number of the eight influenza virus genes and their most effective combinations in inhibiting influenza virus production.

### Example 9

### Evaluation of Non-Viral Delivery Agents That Facilitate Cellular Uptake of siRNA

This example describes testing a variety of non-viral delivery agents for their ability to enhance cellular uptake of siRNA. Subsequent examples provide data showing positive results with a number of the polymers that were tested as described below and in the examples themselves. Other delivery agents may be similarly tested.

Cationic polymers. The ability of cationic polymers to promote intracellular uptake of DNA is believed to result partly from their ability to bind to DNA and condense large plasmid DNA molecules into smaller DNA/polymer complexes for more efficient endocytosis. siRNA duplexes are short (e.g., only 21 nucleotides in length), suggesting that they probably cannot be condensed much further. siRNA precursors such as shRNAs are also relatively short. However, the ability of cationic polymers to bind negatively charged siRNA and interact with the negatively charged cell surface may facilitate intracellular uptake of siRNAs and shRNAs. Thus, known cationic polymers including, but not limited to, PLL, modified PLL (e.g., modified with acyl, succinyl, acetyl, or imidazole groups (32)), polyethyleneimine (PEI) (37), polyvinylpyrrolidone (PVP) (38), and chitosan (39, 40) are promising candidates as delivery agents for siRNA and shRNA.

In addition, novel cationic polymers and oligomers developed in Robert Langer's laboratory are promising candidates as delivery agents. Efficient strategies to synthesize and test large libraries of novel cationic polymers and oligomers from diacrylate and amine monomers for their use in DNA transfection have been developed. These polymers are referred to herein as poly(β-amino ester) (PAE) polymers. In a first study, a library of 140 polymers from 7 diacrylate monomers and 20 amine monomers was synthesized and tested (34). Of the 140 members, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, they found two of the 56 polymers mediated DNA transfection into COS-7 cells. Transfection efficiencies of the novel polymers were 4-8 times higher than PEI and equal or better than Lipofectamine 2000.

Since the initial study, a library of 2,400 cationic polymers has been constructed and screened, and another approximately 40 polymers that promote efficient DNA transfection have been obtained (118). Because structural variations could have a significant impact on DNA binding and transfection efficacies (33), it is preferable to test many polymers for their ability to promote intracellular uptake of siRNA. Furthermore, it is possible that in the transition to an in vivo system, i.e., in mammalian subjects, certain polymers will likely be excluded as a result of studies of their in vivo performance, absorption, distribution, metabolism, and excretion (ADME). Thus testing in intact organisms is important.

Together, at least approximately 50 cationic polymers will be tested in siRNA transfection experiments. Most of them will be PAE and imidazole group-modified PLL as described above. PEI, PVP, and chitosan will be purchased from commercial sources. To screen these polymers rapidly and efficiently, the library of PAE polymers that successfully transfects cells has already been moved into solution into a 96-well plate. Storage of the polymers in this standard 96 well format allows for the straightforward development of a semi-automated screen, using a sterile Labcyte EDR 384S/96S micropipettor robot. The amount of polymer will be titrated (using a predetermined amount of siRNA) to define proper polymer siRNA ratios and the most efficient delivery conditions. Depending on the specific assay, the semi-automated screen will be slightly different as described below.

Characterization of siRNA/polymer complexes. For various cationic polymers to facilitate intracellular uptake of siRNA, they should be able to form complexes with siRNA. This issue will be examined this by electrophoretic mobility shift assay (EMSA) following a similar protocol to that described in (34). Briefly, NP-1496 siRNA will be mixed with each of the 50 or so polymers at the ratios of 1:0.1, 1:0.3, 1:0.9, 1:2.7, 1:8.1, and 1:24.3 (siRNA/polymer, w/w) in 96-well plates using micropipettor robot. The mixtures will be loaded into 4% agarose gel slab capable of assaying up to 500 samples using a multichannel pipettor. Migration patterns of siRNA will be visualized by ethidium bromide staining. If the mobility of an siRNA is reduced in the presence of a polymer, the siRNA forms complexes with that polymer. Based on the ratios of siRNA to polymer, it may be possible to identify the neutralizing ratio. Those polymers that do not bind siRNA will be less preferred and further examination will focus on those polymers that do bind siRNA.

Cytotoxicity of imidazole group-modified PLL, PEI, PVP, chitosan, and some PAE polymers has been measured alone or in complexes with DNA in cell lines. Because cytotoxicity changes depending on bound molecules, the cytotoxicity of various polymers and modified polymers in complexes with siRNA will be measured in MDCK cells. Briefly, NP-1496 will be mixed with different amounts of polymers as above, using the sterile Labcyte micropipettor robot. The complexes will be applied to MDCK cells in 96-well plates for 4 hrs. Then, the polymer-containing medium will be replaced with normal growth medium. 24 hrs later, the metabolic activity of the cells will be measured in the 96-well format using the MTT assay (41). Those polymers that kill 90% or more cells at the lowest amount used will be less preferred, and the focus of further investigation will be polymers that do not kill more than 90% of the cells at the lowest amount used.

While in some cases similar studies have been performed using DNA/polymer compositions, it will be important to determine whether similar results (e.g., cytotoxicity, promotion of cellular uptake) are obtained with RNA/polymer compositions.

siRNA uptake by cultured cells. Once siRNA/polymer complexes have been characterized, their ability to promote cellular uptake of siRNA will be tested, starting with cultured cells using two different assay systems. In the first approach, a GFP-specific siRNA (GFP-949) will be tested on GFP-expressing MDCK cells, because a decrease in GFP expression is easily quantified by measuring fluorescent intensity. Briefly, GFP-949/polymer at the same ratios as above will be applied to MDCK cells in 96-well plates. As negative controls, NP-1496 or no siRNA will be used. As a positive control, GFP-949 will be introduced into cells by electroporation. 36 hrs later, cells will be lysed in 96-well plates and fluorescent intensity of the lysates measured by a fluorescent plate reader. The capacities of various polymers to promote cellular uptake of siRNA will be indicated by the overall decrease of GFP intensity. Alternatively, cells will be analyzed for GFP expression using a flow cytometer that is equipped to handle samples in the 96-well format. The capacities of various polymers to promote cellular uptake of siRNA will be indicated by percentage of cells with reduced GFP intensity and the extent of decrease in GFP intensity. Results from these assays will also shed light on the optimal siRNA:polymer ratio for most efficient transfection.

In the second approach, inhibition of influenza virus production in MDCK cells will be measured directly. As described above, NP-1496 siRNA/polymer at various ratios will be applied to MDCK cells in 96-well plates. As a positive control, siRNA will be introduced into MDCK cells by electroporation. As negative controls, GFP-949 or no siRNA will be used. Eight hrs later, cells will be infected with PR8 or WSN virus at a predetermined MOI. Culture supernatants will be harvested 60 hrs later and assayed for virus without dilution by hemagglutination in 96-well plates. Supernatants from wells that have low virus titers in the initial assay will be diluted (thus indicating that the siRNA/polymer composition inhibited virus production) and assayed by hemagglutination. Alternatively, infected cultures at 60 hrs will be assayed for metabolic activity by the MTT assay. Because infected cells eventually lyse, the relative level of metabolic activity should also give an indication of inhibition of virus infection.

If the virus titer or metabolic activity is substantially lower in cultures that are treated with siRNA/polymer than those that are not treated, it will be concluded that the polymer promotes siRNA transfection. By comparing the virus titers in cultures in which siRNA is introduced by electroporation, the relative transfection efficiency of siRNAs and siRNA/polymer compositions will be estimated.

A number of the most effective cationic polymers from the initial two screens will be verified in the virus infection assay in 96-well plates by titrating both siRNA and polymers. Based on the results obtained, the capacity of the six polymers at the most effective siRNA:polymer ratios will be further analyzed in MDCK cells in 24-well plates and 6-well plates. A number of the most effective polymers will be selected for further studies in mice as described in Example 10.

Alternative approaches. As an alternative to cationic polymers for efficient promotion of intracellular uptake of siRNA in cultured cells, arginine-rich peptides will be investigated in siRNA transfection experiments. Because ARPs are thought to directly penetrate the plasma membrane by interacting with the negatively charged phospholipids (48), whereas most currently used cationic polymers are thought to promote cellular uptake of DNA by endocytosis, the efficacy of ARPs in promoting intracellular uptake of siRNA will be investigated. Like cationic polymers, ARPs and polyarginine (PLA) are also positively charged and likely capable of binding siRNA, suggesting that it is probably not necessary to covalently link siRNA to ARPs or PLAs. Therefore, ARPs or PLAs will be treated similarly to other cationic polymers. The ability of the ARP from Tat and different length of PLAs (available from Sigma) to promote cellular uptake of siRNA will be determined as described above.

### Example 10

### Testing of siRNAs and siRNA/Delivery Agent Compositions in Mice

Rationale: The ability of identified polymers to promote siRNA uptake by cells in the respiratory tract in mice will be evaluated, and the efficacies of siRNAs in preventing and treating influenza virus infection in mice will be examined. Demonstration of siRNA inhibition of influenza virus infection in mice will provide evidence for their potential use in humans to prevent or treat influenza virus infection, e.g., by intranasal or pulmonary administration of siRNAs. Methodology for identifying siRNA-containing compositions that effectively deliver siRNA to cells and effectively treat or prevent influenza virus infection are described in this Example. For simplicity the Example describes testing of siRNA/polymer compositions. Analogous methods may be used for testing of other siRNA/delivery agent compositions such as siRNA/cationic polymer compositions, siRNA/arginine-rich peptide compositions, etc.

Routes of administration. Because influenza virus infects epithelial cells in the upper airways and the lung, a focus will be on methods that deliver siRNAs into epithelial cells in the respiratory tract. Many different methods have been used to deliver small molecule drugs, proteins, and DNA/polymer complexes into the upper airways and/or lungs of mice, including instillation, aerosol (both liquid and dry-powder) inhalation, intratracheal administration, and intravenous injection. By instillation, mice are usually lightly anesthetized and held vertically upright. Therapeutics (i.e. siRNA/polymer complexes) in a small volume (usually 30-50 μl) are applied slowly to one nostril where the fluid is inhaled (52). The animals are maintained in the upright position for a short period of time to allow instilled fluid to reach the lungs (53). Instillation is effective to deliver therapeutics to both the upper airways and the lungs and can be repeated multiple times on the same mouse.

By aerosol, liquid and dry-powder are usually applied differently. Liquid aerosols are produced by a nebulizer into a sealed plastic cage, where the mice are placed (52). Because aerosols are inhaled as animals breathe, the method may be inefficient and imprecise. Dry-powder aerosols are usually administered by forced ventilation on anesthetized mice. This method can be very effective as long as the aerosol particles are large and porous (see below) (31). For intratracheal administration, a solution containing therapeutics is injected via a tube into the lungs of anesthetized mice (54). Although it is quite efficient for delivery into the lungs, it misses the upper airways. Intravenous injection of a small amount of DNA (˜1 μg) in complexes with protein and polyethyleneimine has been shown to transfect endothelial cells and cells in interstitial tissues of the lung (55). Based on this consideration, siRNA/polymer complexes will first be administered to mice by instillation. Intravenous delivery and aerosol delivery using large porous particles will also be explored. In addition, other delivery methods including intravenous and intraperitoneal injection will also be tested.

siRNA uptake by cells in the respiratory tract. A number of the most effective polymers identified as described in Example 9 will be tested for their ability to promote intracellular uptake of siRNA in the respiratory tract in mice. To facilitate investigations, inhibition of GFP expression by GFP-specific siRNA (GFP-949) in GFP-expressing transgenic mice will be used. The advantage of using GFP-specific siRNA initially is that the simplicity and accuracy of the assays may speed up the identification of effective polymers in mice. In addition, the results obtained may shed light on the areas or types of cells that take up siRNA in vivo. The latter information will be useful for modifying delivery agents and methods of administration for optimal delivery of siRNA into the epithelial cells in the respiratory tract.

Briefly, graded doses of GFP-949/polymer complexes (at the most effective ratio as determined in Example 9) will be administered to GFP transgenic mice by instillation. As controls, mice will be given siRNA alone, or polymers alone, or nothing, or non-specific siRNA/polymer complexes. Tissues from the upper airways and the lung will be harvested 36 to 48 hrs after siRNA administration, embedded in OCT, and frozen. Sections will be visualized under a fluorescence microscope for the GFP intensity, and adjacent sections will be stained with hematoxylin/eosin (H/E). Alternatively, tissues will be fixed in paraformaldehyde and embedded in OCT. Some sections will be stained with H&E and adjacent sections will be stained with HRP-conjugated anti-GFP antibodies. Overlay of histology and GFP images (or anti-GFP staining) may be able to identify the areas or cell types in which GFP expression is inhibited. For increased sensitivity, the tissues may be examined by confocal microscopy to identify areas where GFP intensity is decreased.

Based on findings from DNA transfection by instillation (52, 56), it is expected that siRNA will be most likely taken up by epithelial cells on the luminal surface of the respiratory tract. If a significant decrease in GFP intensity is observed in GFP-949/polymer treated mice compared to control mice, this would indicate that the specific polymer promotes cellular uptake of siRNA in vivo.

siRNA inhibition of influenza virus infection in mice. In addition to the above GFP-949 study in GFP transgenic mice, a number of the most effective polymers in promoting siRNA uptake in mice will be examined using siRNA specific for influenza virus, such as NP-1496 or more likely two or three siRNA “cocktails”. For the initial study, siRNA/polymer complexes and influenza virus will be introduced into mice at the same time by mixing siRNA/polymer complexes and virus before instillation. Graded doses of siRNA/polymer complexes and PR8 virus (at a predetermined dose) will be used. As controls, mice will be given siRNA alone, or polymers alone, or nothing, or GFP-949/polymer. At various times following infection (e.g., 2-3 days, or longer, e.g., several days or a week or more) after infection, nasal lavage will be prepared and lungs will be homogenized to elute virus by freeze and thaw. The virus titer in the lavage and the lungs will be measured by hemagglutination. If the titer turns out to be too low to detect by hemagglutinin assay, virus will be amplified in MDCK cells before hemagglutinin assay. For more accurate determination of virus titer, plaque assays will be performed on selected samples.

If a single dose of siRNA/polymer is not effective in inhibiting influenza infection, multiple administrations of siRNA (at a relatively high dosage) will be investigated to determine whether multiple administrations are more effective. For example, following the initial siRNA/polymer and virus administration, mice will be given siRNA/polymer every 12 hrs for 2 days (4 doses). The titer of virus in the lung and nasal lavage will be measured at various times after the initial infection.

Results from these experiments should show whether siRNAs are effective in inhibiting influenza virus infection in the upper airways and the lungs, and point to the most effective single dose. It is expected that multiple administrations of siRNA/polymer are likely to be more effective than a single administration in treating influenza virus infection. Other polymers or delivery agents may also be explored as well as different approaches for siRNA/polymer delivery, e.g., those described below.

siRNA/polymer delivery using large porous particles. Another efficient delivery method to the upper airway and the lungs is using large porous particles originally developed by Robert Langer's group. In contrast to instillation, which is liquid-based, the latter method depends on inhalation of large porous particles (dry-powder) carrying therapeutics. In their initial studies, they showed that double-emulsion solvent evaporation of therapeutics and poly(lactic acid-co-glycolic acid) (PLGA) or poly(lactic acid-co-lysine-graft-lysine) (PLAL-Lys) leads to the generation of large porous particles (31). These particles have mass densities less than 0.4 gram/cm³ and mean diameters exceeding 5 μm. They can be efficiently inhaled deep into the lungs because of their low densities. They are also less efficiently cleared by macrophages in the lungs (57). Inhalation of large porous insulin-containing particles by rats results in elevated systemic levels of insulin and suppression of systemic glucose levels for 96 hrs, as compared to 4 hrs by small nonporous particles.

A procedure for producing large porous particles using excipients that are either FDA-approved for inhalation or endogenous to the lungs (or both) has been developed (58). In this procedure, water-soluble excipients (i.e. lactose, albumin, etc.) and therapeutics were dissolved in distilled water. The solution was fed to a Niro Atomizer Portable Spray Dryer (Niro, Inc., Colombus, Md.) to produce the dry powders, which have a mean geometric diameters ranged between 3 and 15 μm and tap density between 0.04 and 0.6 g/cm³.

The spray-dry method will be used to produce large porous low-density particles carrying siRNA/polymer described by Langer except that the therapeutics are replaced with siRNA/polymer. The resulting particles will be characterized for porosity, density, and size as described in (31, 58). Those that reach the aforementioned criteria will be administered to anesthetized mice by forced ventilation using a Harvard ventilator. Depending on whether siRNA specific for either GFP or influenza virus is used, different assays will be performed as described above. If GFP expression or the virus titer in mice that are given specific siRNA/polymer in large porous particles is significantly lower than in control mice, aerosol inhalation via large porous particles would appear to be an effective method for siRNA delivery.

Prophylactic and therapeutic application of siRNAs/polymer complexes. The efficacy of siRNA/polymer complexes as prophylaxis or therapy for influenza virus infection in mice will be examined. Assuming a single dose of siRNA/polymer complexes is effective, the length of time after their administration over which the siRNAs remain effective in interfering with influenza infection will be assessed. siRNA/polymer complexes will be administered to mice by instillation or large porous aerosols (depending on which one is more effective as determined above). Mice will be infected with influenza virus immediately, or 1, 2, or 3 days later, and virus titer in the nasal lavage and the lung will be measured on 24 or 48 hrs after virus infection. If siRNA is found to be still effective after 3 days, mice will be infected 4, 5, 6, and 7 days after siRNA/polymer administration, and tissues will be harvested for assaying virus titer 24 hrs after the infection. Results from these experiments will likely reveal how long after administration, siRNAs remain effective in interfering with virus production in mice and will guide use in humans.

To evaluate therapeutic efficacy of siRNAs, mice will be infected with influenza virus and then given siRNA/polymer complexes at different times after infection. Specifically, mice will be infected intranasally, and then given an effective dose (as determined above) of siRNA/polymer immediately, or 1, 2, or 3 days later. As controls, mice will be given GFP-949 or no siRNA at all immediately after infection. The virus titer in the nasal lavage and the lung will be measured 24 or 48 hrs after siRNA administration.

In addition, mice will be infected with a lethal dose of influenza virus and into five groups (5-8 mice per group). Group 1 will be given an effective dose of siRNA/polymer complexes immediately. Groups 2 to 4 will be given an effective dose of siRNA/polymer complexes on day 1 to 3 after infection, respectively. Groups 5 will be given GFP-specific siRNA immediately after infection and used as controls. Survival of the infected mice will be followed. Results from these experiments will likely reveal how long after infection administration of siRNAs still exerts a therapeutic effect in mice.

### Example 11

### Inhibition of Influenza Virus Infection by siRNAs Transcribed from Templates Provided by DNA Vectors or Lentiviruses

Rationale: Effective siRNA therapy of influenza virus infection depends on the ability to deliver a sufficient amount of siRNA into appropriate cells in vivo. To prevent the emergence of resistant virus, it may be preferable to use two or three siRNAs together. Simultaneous delivery of two or three siRNAs into the same cells will require an efficient delivery system. As an alternative to the approaches described above, the use of DNA vectors from which siRNA precursors can be transcribed and processed into effective siRNAs will be explored.

We have previously shown that siRNA transcribed from a DNA vector can inhibit CD8α expression to the same extent as synthetic siRNA introduced into the same cells. Specifically, we found that one of the five siRNAs designed to target the CD8α gene, referred to as CD8-61, inhibited CD8 but not CD4 expression in a mouse CD8⁺ CD4⁺ T cell line (27). By testing various hairpin derivatives of CD8-61 siRNA, we found that CD8-61F had a similar inhibitory activity as CD8-61 (FIGS. 20A and 20B) (59). Because of its hairpin structure, CD8-61F was constructed into pSLOOP III, a DNA vector (FIG. 20C) in which CD8-61F was driven by the H1 RNA promoter. The H1 RNA promoter is compact (60) and transcribed by polymerase III (pol III). The Pol III promoter was used because it normally transcribes short RNAs and has been used to generate siRNA-type silencing previously (61). To test the DNA vector, we used HeLa cells that had been transfected with a CD8α expressing vector. As shown in FIG. 20D, transient transfection of the pSLOOP III-CD8-61F plasmid into CD8α-expressing HeLa cells resulted in reduction of CD8α expression to the same extent as HeLa cells that were transfected with synthetic CD8-61 siRNA. In contrast, transfection of a promoter-less vector did not significantly reduce CD8α expression. These results show that a RNA hairpin can be transcribed from a DNA vector and then processed into siRNA for RNA silencing. A similar approach will be used to design DNA vectors that express siRNA precursors specific for the influenza virus.

Investigation of siRNA transcribed from DNA templates in cultured cells. To express siRNA precursors from a DNA vector, hairpin derivatives of siRNA (specific for influenza virus) that can be processed into siRNA duplexes will be designed. In addition, vectors from which two or more siRNA precursors can be transcribed will be produced. To speed up these investigations, GFP-949 and NP-1496 siRNAs will be used in MDCK cells that express GFP. Following the CD8-61F design, hairpin derivatives of GFP-949 and NP-1496, referred to as GFP-949H and NP-1496H, respectively will be synthesized (FIG. 21A).

Both GFP-949 and GFP-949H will be electroporated into GFP-expressing MDCK cells. NP-1496 or mock electroporation will be used as negative controls. 24 and 48 hrs later, cells will be assayed for GFP expression by flow cytometry. If the percentage of GFP-positive cells and the intensity of GFP level are significantly reduced in cultures that are given GFP-949H, the hairpin derivative's effectiveness will have been demonstrated. Its efficacy will be indicated by comparing GFP intensity in cells given standard GFP-949.

Similarly, NP-1496 and NP-1496H will be electroporated into MDCK cells. GFP-949 or mock electroporation will be used as negative controls. 8 hrs later after transfection, cells will be infected with PR8 or WSN virus. The virus titers in the culture supernatants will be measured by hemagglutination 60 hrs after the infection. If the virus titer is significantly reduced in cultures given NP-1496H, the hairpin derivative inhibits virus production. It is expected that the hairpin derivatives will be functional based on studies with CD8-61F. If not, different designs of hairpin derivatives similar to those described in (59, 61, 62) will be synthesized and tested.

Designing DNA vectors and testing them in cultured cells. Once GFP-949H and NP-1496H are shown to be functional, the corresponding expression vectors will be constructed. GFP-949H and NP-1496H will be cloned individually behind the H1 promoter in the pSLOOP III vector (FIG. 21C, top). The resulting vectors will be transiently transfected into GFP-expressing MDCK cells by electroporation. Transfected cells will be analyzed for GFP intensity or infected with virus and assayed for virus production. The U6 Pol III promoter, which has also been shown to drive high levels of siRNA precursor expression will be tested this in addition to other promoters to identify a potent one for siRNA precursor transcription.

Once vectors that transcribe a single siRNA precursor are shown to be effective, vectors that can transcribe two siRNA precursors will be constructed. For this purpose, both GFP-949H and NP-1496H will be cloned into pSLOOP III vector in tandem, either GFP-949H at the 5′ and NP-1496H at the 3′, or the other way around (FIG. 21C, middle). In the resulting vectors, the two siRNA precursors will be linked by extra nucleotides present in the hairpin structure (FIG. 21B). Because it is not known whether two siRNAs can be processed from a single transcript, vectors in which both GFP-949H and NP-1496H are transcribed by independent promoters will also be constructed (FIG. 21C, bottom).

Because transfection efficiency in MDCK cells is about 50%, transient transfection may not be ideal for evaluating vectors that encode two siRNA precursors. Therefore, stable transfectants will be established by electroporating GFP-expressing MDCK cells with linearized vectors plus a neo-resistant vector. DNA will be isolated from multiple transfectants to confirm the presence of siRNA expressing vectors by Southern blotting. Positive transfectants will be assayed for GFP expression to determine if GFP-specific siRNA transcribed from the stably integrated vector can inhibit GFP expression. Those transfectants in which GFP expression is inhibited will be infected with PR8 or WSN virus and the virus titer will be measured by hemagglutination. The finding that both GFP expression and virus production are inhibited in a significant fraction of transfectants would establish that two siRNA precursors can be transcribed and processed from a single DNA vector.

Constructing vectors from which a single siRNA precursor will be transcribed should be straightforward because a similar approach has been successfully used in previous studies (59). Since many studies have shown that two genes can be transcribed independently from the same vector using identical promoter and termination sequences, it is likely that two siRNA precursors can be transcribed from the same vector. In the latter approach, siRNA precursors are independently transcribed. The length of the resulting dsRNA precursors is likely less than 50 nucleotides. In contrast, when two siRNA precursors are transcribed in tandem (FIGS. 21B and C), the resulting dsRNA precursor would be longer than 50 nucleotides. The presence of dsRNA longer than 50 nucleotides activates an interferon response in mammalian cells (22, 23). Thus, another advantage of independent transcription of two siRNA precursors from the same vector is that it would avoid an interferon response. Interferon inhibits virus infection and therefore could be useful, but the response also shuts down many metabolic pathways and therefore interferes with cellular function (63).

To determine if an interferon response is induced in MDCK cells transfected with various DNA vectors, the level of total and phosphorylated dsRNA-dependent protein kinase (PKR) will be assayed since phosphorylation of PKR is required for the interferon response (23). Cell lysates prepared from vector- and mock-transfected cells will be fractionated on SDS-PAGE. Proteins will be transferred onto a membrane and the membrane probed with antibodies specific to phosphorylated PKR or total PKR. If the assay is not sufficiently sensitive, immunoprecipitation followed by Western blotting will be performed. If no difference in the level of activated PKR is detected, dsRNA precursors transcribed from the DNA vectors do not activate the interferon response. Preferred DNA vectors for intracellular synthesis of siRNAs do not activate the interferon response, and the invention thus provides such vectors.

Investigation of DNA vectors in mice. Once it is shown that siRNA transcribed from DNA vectors can inhibit influenza virus production in MDCK cells, their efficacies in mice will be investigated. To minimize the integration of introduced plasmid DNA into the cellular genome, supercoiled DNA will be used for transient expression. The other advantage of transient expression is that the level of expression tends to be high, probably because the plasmid copy numbers per cell is high prior to integration. To facilitate DNA transfection in mice, cationic polymers that have been developed for gene therapy, including imidozole group-modified PLL, PEI, PVP, and PAE as described in Example 8, will be used.

Specifically, DNA vectors expressing GFP-949H or NP-1496H alone or both NP-1496H and GFP-949H will be mixed with specific polymers at a predetermined ratio. Graded amounts of the complexes plus PR8 or WSN virus will be introduced into anesthetized GFP transgenic mice by instillation. As controls, mice will be given DNA alone, or polymers alone, or nothing. Two and three days after infection, nasal lavage and lungs will be harvested for assaying for virus titer as described in Example 10. In addition, the upper airways and the lung sections will be examined for reduction in GFP expression.

DNA/polymer complexes will also be administered multiple times, e.g. together with the virus initially and once a day for the following two days. The effect of multiple administrations will be examined on day 3 after the infection. In addition, DNA vectors that encode two or three influenza-specific siRNA precursors will be constructed and their efficacies in inhibiting influenza infection in mice will be tested.

Lentiviruses. The constructs described above will be inserted into lentiviral transfer plasmids and used for production of infectious lentivirus. The lentivirus thus provides a template for synthesis of shRNA within cells infected with the virus. The ability of lentiviral vectors to inhibit production of influenza virus will be tested in tissue culture and in mice as described above for DNA vectors. The lentiviruses may be administered to mice using any of the delivery agents of the invention or delivery agents previously used for administration of lentivirus or other viral gene therapy vectors.

### Example 12

### Inhibition of Influenza Virus Production in Mice by siRNAs

This example describes experiments showing that administration of siRNAs targeted to influenza virus NP or PA transcripts inhibit production of influenza virus in mice when administered either prior to or following infection with influenza virus. The inhibition is dose-dependent and shows additive effects when two siRNAs targeted to transcripts expressed from two different influenza virus genes were administered together.

Materials and Methods

SiRNA preparation. This was performed as described above.

SiRNA delivery. siRNAs (30 or 60 μg of GFP-949, NP-1496, or PA-2087) were incubated with jetPEI™ for oligonucleotides cationic polymer transfection reagent, N/P ratio=5 (Qbiogene, Inc., Carlsbad, Calif.; Cat. No. GDSP20130; NIP refers to the number of nitrogen residues per nucleotide phosphate in the jetPEI reagent) or with poly-L-lysine (MW (vis) 52,000; MW (LALLS) 41,800, Sigma Cat. No. P2636) for 20 min at room temperature in 5% glucose. The mixture was injected into mice intravenously, into the retro-orbital vein, 200 μl per mouse, 4 mice per group. 200 μl 5% glucose was injected into control (no treatment) mice. The mice were anesthetized with 2.5% Avertin before siRNA injection or intranasal infection.

Viral infection. B6 mice (maintained under standard laboratory conditions) were intranasally infected with PR8 virus by dropping virus-containing buffer into the mouse's nose with a pipette, 30 ul (12,000 pfu) per mouse.

Determination of viral titer. Mice were sacrificed at various times following infection, and lungs were harvested. Lungs were homogenized, and the homogenate was frozen and thawed twice to release virus. PR8 virus present in infected lungs was titered by infection of MDCK cells. Flat-bottom 96-well plates were seeded with 3×10⁴ MDCK cells per well, and 24 hrs later the serum-containing medium was removed. 25 μl of lung homogenate, either undiluted or diluted from 1×10⁻¹ to 1×10⁻⁷, was inoculated into triplicate wells. After 1 h incubation, 175 μl of infection medium with 4 μg/ml of trypsin was added to each well. Following a 48 h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 h. Wells containing an adherent, homogeneous layer of erythrocytes were scored as positive. The virus titers were determined by interpolation of the dilution end point that infected 50% of wells by the method of Reed and Muench (TCID₅₀). The data from any two groups were compared by Student t test, which was used throughout the experiments described herein to evaluate significance.

Results

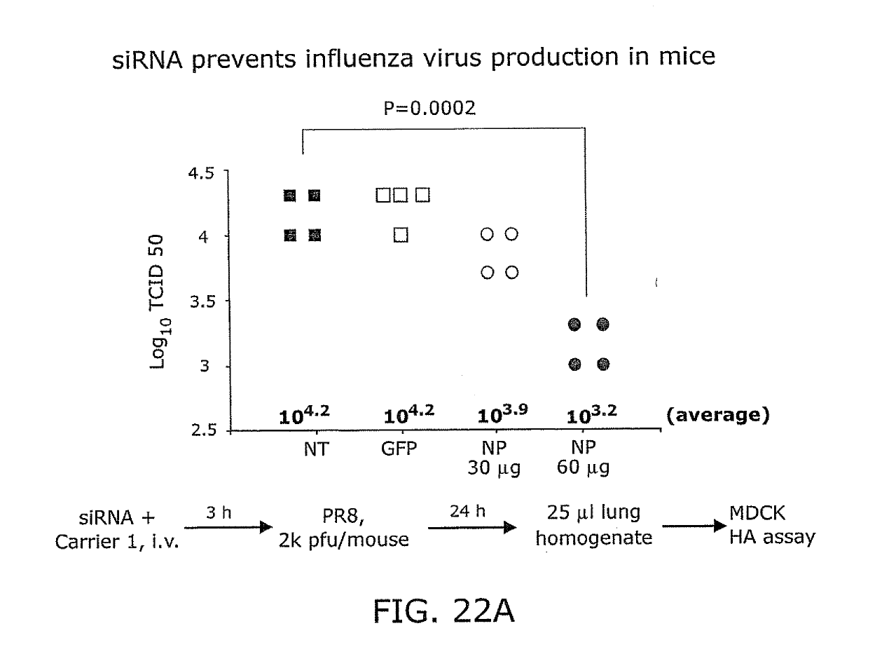

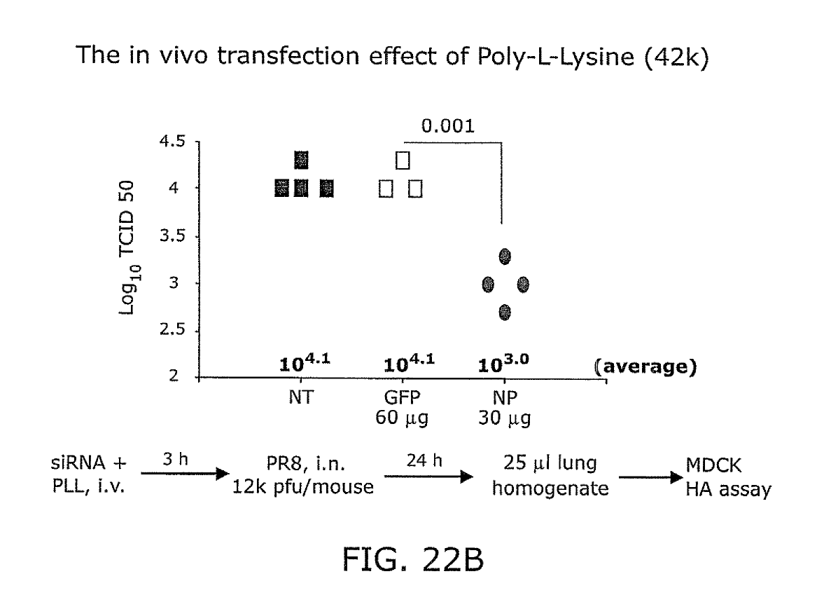

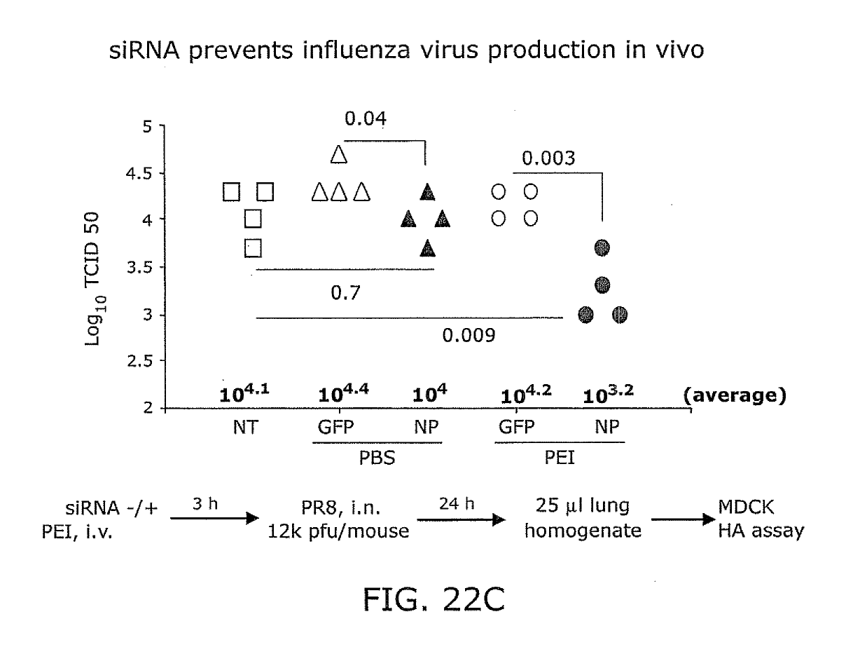

FIG. 22A shows results of an experiment demonstrating that siRNA targeted to viral NP transcripts inhibits influenza virus production in mice when administered prior to infection. 30 or 60 μg of GFP-949 or NP-1496 siRNAs were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 22A, the average log₁₀TCID₅₀ of the lung homogenate for mice that received no siRNA treatment (NT; filled squares) or received an siRNA targeted to GFP (GFP 60 μg; open squares) was 4.2. In mice that were pretreated with 30 μg siRNA targeted to NP(NP 30 μg; open circles) and jetPEI, the average log₁₀TCID₅₀ of the lung homogenate was 3.9. In mice that were pretreated with 60 μg siRNA targeted to NP(NP 60 μg; filled circles) and jetPEI, the average log₁₀TCID₅₀ of the lung homogenate was 3.2. The difference in virus titer in the lung homogenate between the group that received no treatment and the group that received 60 μg NP siRNA was significant with P=0.0002. Data for individual mice are presented in Table 6A (NT=no treatment).

FIG. 22B shows results of another experiment demonstrating that siRNA targeted to viral NP transcripts inhibits influenza virus production in mice when TO administered intravenously prior to infection in a composition containing the cationinc polymer PLL. 30 or 60 μg of GFP-949 or NP-1496 siRNAs were incubated with PLL and injected intravenously into mice as described above in Materials and Methods. Three hours later mice were intranasally infected with PR8 virus, 12000 pfu per mouse. Lungs were harvested 24 hours after infection. As shown in FIG. 22B, the average log₁₀TCID₅₀ of the lung homogenate for mice that received no siRNA treatment (NT; filled squares) or received an siRNA targeted to GFP (GFP 60 μg; open squares) was 4.1. In mice that were pretreated with 60 μg siRNA targeted to NP(NP 60 μg; filled circles) and PLL, the average log₁₀TCID₅₀ of the lung homogenate was 3.0. The difference in virus titer in the lung homogenate between the group that received 60 μg GFP and the group that received 60 μg NP siRNA was significant with P=0.001. Data for individual mice are presented in Table 6A (NT=no treatment). These data indicate that siRNA targeted to the influenza NP transcript reduced the virus titer in the lung when administered prior to virus infection. They also indicate that mixtures of siRNAs with cationic polymers are effective agents for the inhibition of influenza virus in the lung when administered by intravenous injection, not requiring techniques such as hydrodynamic transfection.

TABLE 6A
Inhibition of influenza virus production in mice by siRNA with
cationic polymers

| | |
|---|---|
| Treatment | log₁₀TCID50 |

| | | | | |
|---|---|---|---|---|
| NT (jetPEI experiment) | 4.3 | 4.3 | 4.0 | 4.0 |
| GFP (60 μg) + jetPEI | 4.3 | 4.3 | 4.3 | 4.0 |
| NP (30 μg) + jetPEI | 4.0 | 4.0 | 3.7 | 3.7 |
| NP (60 μg) + jetPEI | 3.3 | 3.3 | 3.0 | 3.0 |
| NT (PLL experiment) | 4.0 | 4.3 | 4.0 | 4.0 |
| GFP (60 μg) + PLL | 4.3 | 4.0 | 4.0 | (not done) |
| NP (60 μg) + PLL | 3.3 | 3.0 | 3.0 | 2.7 |

FIG. 22C shows results of a third experiment demonstrating that siRNA targeted to viral NP transcripts inhibits influenza virus production in mice when administered prior to infection and demonstrates that the presence of a cationic polymer significantly increases the inhibitory efficacy of siRNA. 60 μg of GFP-949 or NP-1496 siRNAs were incubated with phosphate buffered saline (PBS) or jetPEI and injected intravenously into mice as described above in Materials and Methods. Three hours later mice were intranasally infected with PR8 virus, 12000 pfu per mouse. Lungs were harvested 24 hours after infection. As shown in FIG. 22C, the average log₁₀TCID₅₀ of the lung homogenate for mice that received no siRNA treatment (NT; open squares) was 4.1, while the average log₁₀TCID₅₀ of the lung homogenate for mice that received an siRNA targeted to GFP in PBS (GFP PBS; open triangles) was 4.4. In mice that were pretreated with 60 μg siRNA targeted to NP in PBS (NP PBS; open circles) the average log₁₀TCID₅₀ of the lung homogenate was 4.2, showing only a modest increase in efficacy relative to no treatment or treatment with an siRNA targeted to GFP. In mice that were pretreated with 60 μg siRNA targeted to GFP injetPEI (GFP PEI; open circles), the average log₁₀TCID₅₀ of the lung homogenate was 4.2. However, in mice that received 60 μg siRNA targeted to NP in jetPEI (NP PEI; closed circles), and jetPEI, the average log₁₀TCID₅₀ of the lung homogenate was 3.9. In mice that were pretreated with 60 μg siRNA targeted to NP and jetPEI (NP PEI; filled circles), the average log₁₀TCID₅₀ of the lung homogenate was 3.2. The difference in virus titer in the lung homogenate between the group that received GFP siRNA in PBS and the group that received NP siRNA in PBS was significant with P=0.04, while the difference in virus titer in the lung homogenate between the group that received GFP siRNA with jetPEI and the group that received NP siRNA with jetPEI was highly significant with P=0.003. Data for individual mice are presented in Table 6B (NT=no treatment).

TABLE 6B
Inhibition of influenza virus production in mice by siRNA showing
increased efficacy with cationic polymer

| | |
|---|---|
| Treatment | log₁₀TCID50 |

| | | | | |
|---|---|---|---|---|
| NT | 4.3 | 4.3 | 4.0 | 3.7 |
| GFP (60 μg) + PBS | 4.3 | 4.3 | 4.7 | 4.3 |
| NP (60 μg) + PBS | 3.7 | 4.3 | 4.0 | 4.0 |
| GPP (60 μg) + jetPEI | 4.3 | 4.3 | 4.0 | 3.0 |
| NT (60 μg) + jetPEI | 3.3 | 3.0 | 3.7 | 3.0 |

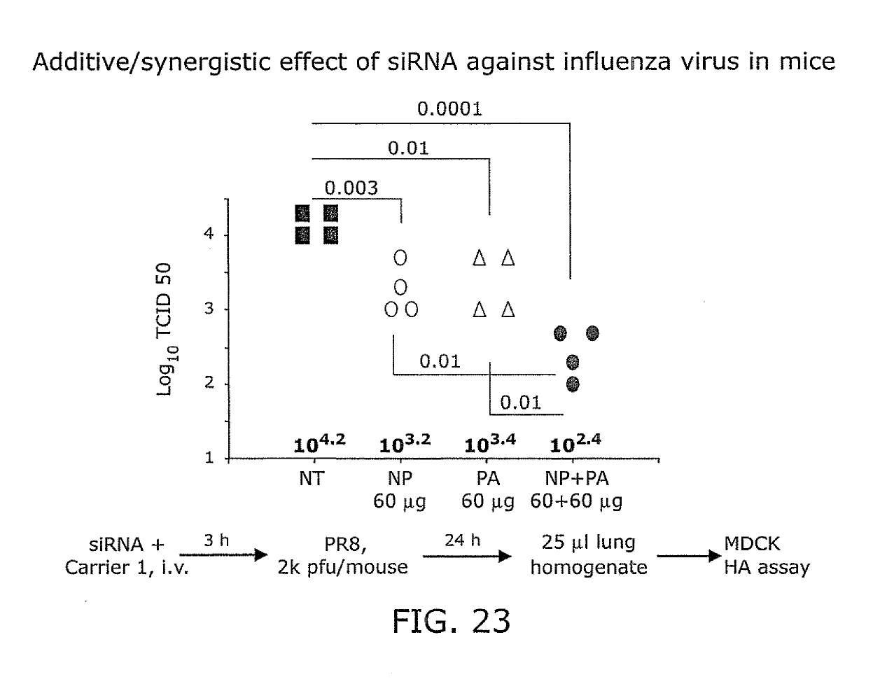

FIG. 23 shows results of an experiment demonstrating that siRNAs targeted to different influenza virus transcripts exhibit an additive effect. Sixty μg of NP-1496 siRNA, 60 μg PA-2087 siRNA, or 60 μg NP-1496 siRNA+60 μg PA-2087 siRNA were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 23, the average log₁₀TCID₅₀ of the lung homogenate for mice that received no siRNA treatment (NT; filled squares) was 4.2. In mice that received 60 μg siRNA targeted to NP(NP 60 μg; open circles), the average log₁₀TCID₅₀ of the lung homogenate was 3.2. In mice that received 60 μg siRNA targeted to PA (PA 60 μg; open triangles), the average log₁₀TCID₅₀ of the lung homogenate was 3.4. In mice that received 60 μg siRNA targeted to NP+60 μg siRNA targeted to PA (NP+PA; filled circles), the average log₁₀TCID₅₀ of the lung homogenate was 2.4. The differences in virus titer in the lung homogenate between the group that received no treatment and the groups that received 60 μg NP siRNA, 60 μg PA siRNA, or 60 μg NP siRNA+60 μg PA siRNA were significant with P=0.003, 0.01, and 0.0001, respectively. The differences in lung homogenate between the groups that received 60 μg NP siRNA or 60 μg NP siRNA and the group that received 60 μg NP siRNA+60 μg PA siRNA were significant with P=0.01. Data for individual mice are presented in Table 7 (NT=no treatment). These data indicate that pretreatment with siRNA targeted to the influenza NP or PA transcript reduced the virus titer in the lungs of mice subsequently infected with influenza virus. The data further indicate that a combination of siRNA targeted to different viral transcripts exhibit an additive effect, suggesting that therapy with a combination of siRNAs targeted to different transcripts may allow a reduction in dose of each siRNA, relative to the amount of a single siRNA that would be needed to achieve equal efficacy. It is possible that certain siRNAs targeted to different transcripts may exhibit synergistic effects (i.e., effects that are greater than additive). The systematic approach to identification of potent siRNAs and siRNA combinations may be used to identify siRNA compositions in which siRNAs exhibit synergistic effects.

TABLE 7
Additive effect of siRNA against influenza virus in mice

| | |
|---|---|
| Treatment | log₁₀TCID50 |

| | | | | |
|---|---|---|---|---|
| NT | 4.3 | 4.3 | 4.0 | 4.0 |
| NP (60 μg) | 3.7 | 3.3 | 3.0 | 3.0 |
| PA (60 μg) | 3.7 | 3.7 | 3.0 | 3.0 |
| NP + PA (60 μg | 2.7 | 2.7 | 2.3 | 2.0 |
| each) | | | | |

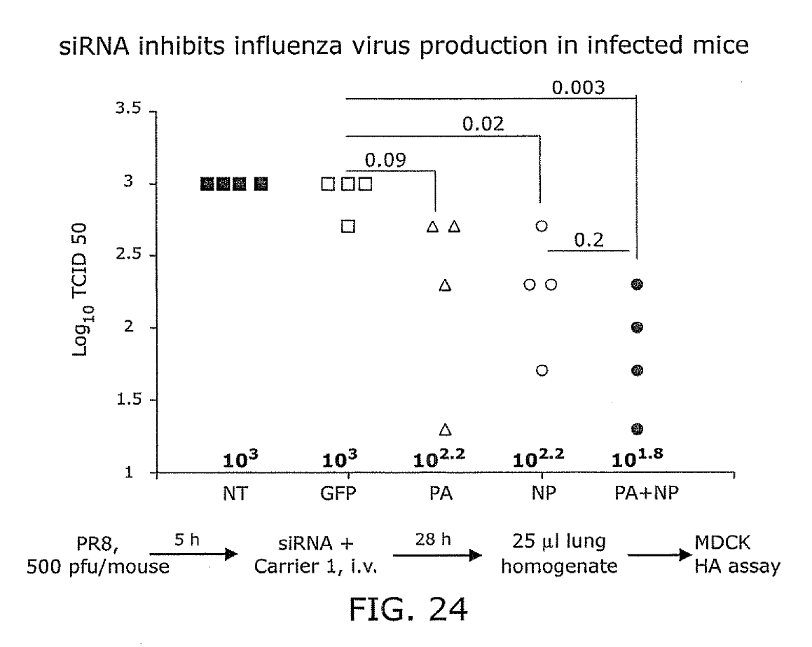

FIG. 24 shows results of an experiment demonstrating that siRNA targeted to viral NP transcripts inhibits influenza virus production in mice when administered following infection. Mice were intranasally infected with PR8 virus, 500 pfu. Sixty μg of GFP-949 siRNA, 60 μg PA-2087 siRNA, 60 μg NP-1496 siRNA, or 60 μg NP siRNA+60 μg PA siRNA were incubated with jetPEI and injected intravenously into mice 5 hours later as described above in Materials and Methods. Lungs were harvested 28 hours after administration of siRNA. As shown in FIG. 24, the average log₁₀TCID₅₀ of the lung homogenate for mice that received no siRNA treatment (NT; filled squares) or received the GFP-specific siRNA GFP-949 (GFP; open squares) was 3.0. In mice that received 60 μg siRNA targeted to PA (PA 60 μg; open triangles), the average log₁₀TCID₅₀ of the lung homogenate was 2.2. In mice that received 60 μg siRNA targeted to NP(NP 60 μg; open circles), the average log₁₀TCID₅₀ of the lung homogenate was 2.2. In mice that received 60 μg NP siRNA+60 μg PA siRNA (PA+NP; filled circles), the average log₁₀TCID₅₀ of the lung homogenate was 1.8. The differences in virus titer in the lung homogenate between the group that received no treatment and the groups that received 60 μg PA, NP siRNA, or 60 μg NP siRNA+60 μg PA siRNA were significant with P=0.09, 0.02, and 0.003, respectively. The difference in virus titer in the lung homogenate between the group that received NP siRNA and PA+NP siRNAs had a P value of 0.2. Data for individual mice are presented in Table 8 (NT=no treatment). These data indicate that siRNA targeted to the influenza NP and/or PA transcripts reduced the virus titer in the lung when administered following virus infection.

TABLE 8
Inhibition of influenza virus production in infected mice by siRNA

| | |
|---|---|
| Treatment | log₁₀TCID50 |

| | | | | |
|---|---|---|---|---|
| NT | 3.0 | 3.0 | 3.0 | 3.0 |
| GFP (60 μg) | 3.0 | 3.0 | 3.0 | 2.7 |
| PA (60 μg) | 2.7 | 2.7 | 2.3 | 1.3 |
| NP (60 μg) | 2.7 | 2.3 | 2.3 | 1.7 |
| NP + PA (60 μg | 2.3 | 2.0 | 1.7 | 1.3 |
| each) | | | | |

### Example 13

### Inhibition of Influenza Virus Production in Cells by Administration of a Lentivirus that Provides a Template for Production of shRNA

Materials and Methods

Cell culture. Vero cells were seeded in 24-well plates at 4×10⁵ cells per well in 1 ml of DMEM-10% FCS and were incubated at 37° C. under 5% CO₂.

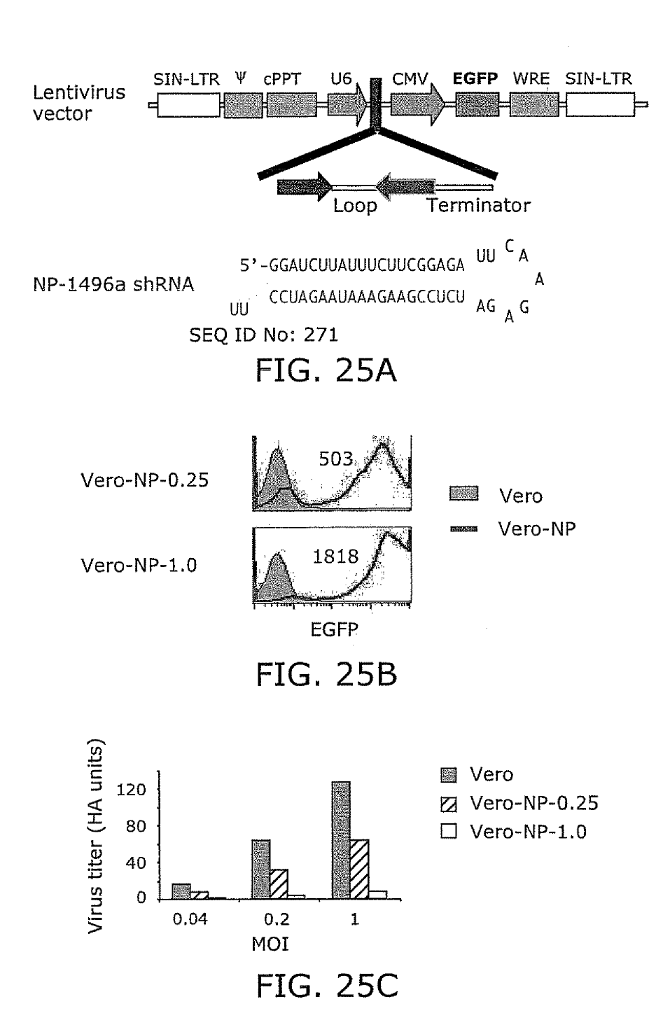

Production of lentivirus that provides a template for shRNA production. An oligonucleotide that serves as a template for synthesis of an NP-1496a shRNA (see FIG. 25A) was cloned between the U6 promoter and termination sequence of lentiviral vector pLL3.7 (Rubinson, D., et al, Nature Genetics, Vol. 33, pp. 401-406, 2003), as depicted schematically in FIG. 25A. The oligonucleotide was inserted between the HpaI and XhoI restriction sites within the multiple cloning site of pLL3.7. This lentiviral vector also expresses EGFP for easy monitoring of transfected/infected cells. Lentivirus was produced by co-transfecting the DNA vector comprising a template for production of NP-1496a shRNA and packaging vectors into 293T cells. Forty-eight h later, culture supernatant containing lentivirus was collected, spun at 2000 rpm for 7 min at 4° C. and then filtered through a 0.45 um filter. Vero cells were seeded at 1×10⁵ per well in 24-well plates. After overnight culture, culture supernatants containing that contained the insert (either 0.25 ml or 1.0 ml) were added to wells in the presence of 8 ug/ml polybrene. The plates were then centrifuged at 2500 rpm, room temperature for 1 h and returned to culture. Twenty-four h after infection, the resulting Vero cell lines (Vero-NP-0.25, and Vero-NP-1.0) were analyzed for GFP expression by flow cytometry along with parental (non-infected) Vero cells. It is noted that NP-1496a differs from NP-1496 due to the inadvertent inclusion of an additional nucleotide (A) at the 3′ end of the sense portion and a complementary nucleotide (U) at the 5′ end of the antisense portion, resulting in a duplex portion that is 20 nt in length rather than 19 as in NP-1496. (See Table 2). According to other embodiments of the invention NP-1496 sequences rather than NP-1496a sequences are used. In addition, the loop portion of NP-1496a shRNA differs from that of NP-1496 shRNA shown in FIG. 21.

Influenza virus infection and determination of viral titer. Control Vero cells and Vero cells infected with lentivirus containing the insert (Vero-NP-0.25 and Vero-NP-1.0) were infected with PR8 virus at MOI of 0.04, 0.2 and 1. Influenza virus titers in the supernatants were determined by hemagglutination (HA) assay 48 hrs after infection as described in Example 12.

Results

Lentivirus containing templates for production of NP-1496a shRNA were tested for ability to inhibit influenza virus production in Vero cells. The NP-1496a shRNA includes two complementary regions capable of forming a stem-loop structure containing a double-stranded portion that has the same sequence as the NP-1496a siRNA described above. As shown in FIG. 25B, incubation of lentivirus-containing supernatants with Vero cells overnight resulted in expression of EGFP, indicating infection of Vero cells by lentivirus. The shaded curve represents mean fluorescence intensity in control cells (uninfected). When 1 ml of supernatant was used, almost all cells became EGFP positive and the mean fluorescence intensity was high (1818) (Vero-NP-1.0). When 0.25 ml of supernatant was used, most cells (˜95%) were EGFP positive and the mean fluorescence intensity was lower (503) (Vero-NP-0.25).

Parental Vero cells and lentivirus-infected Vero cells were then infected with influenza virus at MOI of 0.04, 0.2, and 0.1, and virus titers were assayed 48 hrs after influenza virus infection. With increasing MOI, the virus titers increased in the supernatants of parental Vero cell cultures (FIG. 25C). In contrast, the virus titers remained very low in supernatants of Vero-NP-1.0 cell cultures, indicating influenza virus production was inhibited in these cells. Similarly, influenza virus production in Vero-NP-0.25 cell cultures was also partially inhibited. The viral titers are presented in Table 9. These results suggest that NP-1496 shRNA expressed from lentivirus vectors can be processed into siRNA to inhibit influenza virus production in Vero cells. The extent of inhibition appears to be proportional to the extent of virus infection per cell (indicated by EGFP level).

TABLE 9
Inhibition of influenza virus production by siRNA expressed
in cells in tissue culture

| Cell Line | Viral Titer | | |
|---|---|---|---|
| Vero | 16 | 64 | 128 |
| Vero-NP-0.25 | 8 | 32 | 64 |
| Vero-NP-1.0 | 1 | 4 | 8 |

### Example 14

### Inhibition of Influenza Production in Mice by Intranasal Administration of a DNA Vector from which siRNA Precursors can be Transcribed

Materials and Methods

Construction of plasmids that serves as template for shRNA. Construction of a plasmid from which NP-1496a shRNA is expressed is described in Example 13. Oligonucleotides that serve as templates for synthesis of PB1-2257 shRNA or RSV-specific shRNA were cloned between the U6 promoter and termination sequence of lentiviral vector pLL3.7 as described in Example 13 and depicted schematically in FIG. 25A for NP-1496a shRNA. The sequences of the oligonucleotides were as follows:

NP-1496a sense:

(SEQ ID NO: 179)

5′-TGGATCTTATTTCTTCGGAGATTCAAGAGATCTCCGAAGAAATAAGA
TCCTTTTTTC-3′
NP-1496a antisense:

(SEQ ID NO: 180)

5′-TCGAGAAAAAAGGATCTTATTTCTTCGGAGATCTCTTGAATCTCCGA
AGAAATAAGATCCA-3′
PB1-2257 sense:

(SEQ ID NO: 181)

5′-TGATCTGTTCCACCATTGAATTCAAGAGATTCAATGGTGGAACAGAT
CTTTTTTC-3′
PB1-2257 antisense

(SEQ ID NO: 182)

5′-TCGAGAAAAAAGATCTGTTCCACCATTGAATCTCTTGAATTCAATGG
TGGAACAGATCA-3′
RSV sense:

(SEQ ID NO: 183)

5′-TGCGATAATATAACTGCAAGATTCAAGAGATCTTGCAGTTATATTAT
CGTTTTTTC-3′
RSV antisense:

(SEQ ID NO: 184)

5′-TCGAGAAAAAACGATAATATAACTGCAAGATCTCTTGAATCTTGCAG
TTATATTATCGCA-3′

The RSV shRNA expressed from the vector comprising the above oligonucleotide is processed in vivo to generate an siRNA having sense and antisense strands with the following sequences:

| | |
|---|---|
| Sense: | |
| 5′-CGATAATATAACTGCAAGA-3′ | (SEQ ID NO: 185) |
| Antisense: | |
| 5′-TCTTGCAGTTATATTATCG-3′ | (SEQ ID NO: 186) |

A PA-specific hairpin may be similarly constructed using the following oligonucleotides:

PA-2087 sense:

(SEQ ID NO: 187)

5′-TGCAATTGAGGAGTGCCTGATTCAAGAGATCAGGCACTCCTCAATTG
CTTTTTTC-3′
PA-2087 antisense:

(SEQ ID NO: 270)

5′-TCGAGAAAAAAGCAATTGAGGAGTGCCTGATCTCTTGAATCAGGCAC
TCCTCAATTGCA-3′

Viral infection and determination of viral titer. These were performed as described in Example 12.

DNA Delivery. Plasmid DNAs capable of serving as templates for expression of NP-1496a shRNA, PB1-2257 shRNA, or RSV-specific shRNA (60 μg each) were individually mixed with 40 μl Infasurf® (ONY, Inc., Amherst N.Y.) and 20 μl of 5% glucose and were administered intranasally to groups of mice, 4 mice each group, as described above. A mixture of 40 μl Infasurf and 20 μl of 5% glucose was administered to mice in the no treatment (NT) group. The mice were intranasally infected with PR8 virus, 12000 pfu per mouse, 13 hours later, as described above. Lungs were harvested and viral titer determined 24 hours after infection.

Results

The ability of shRNAs expressed from DNA vectors to inhibit influenza virus infection in mice was tested. For these experiments, plasmid DNA was mixed with Infasurf, a natural surfactant extract from calf lung similar to vehicles previously shown to promote gene transfer in the lung (74). The DNA/Infasurf mixtures were instilled into mice by dropping the mixture into the nose using a pipette. Mice were infected with PR8 virus, 12000 pfu per mouse, 13 hours later. Twenty-four hrs after influenza virus infection, lungs were harvested and virus titers were measured by MDCK/hemagglutinin assay.

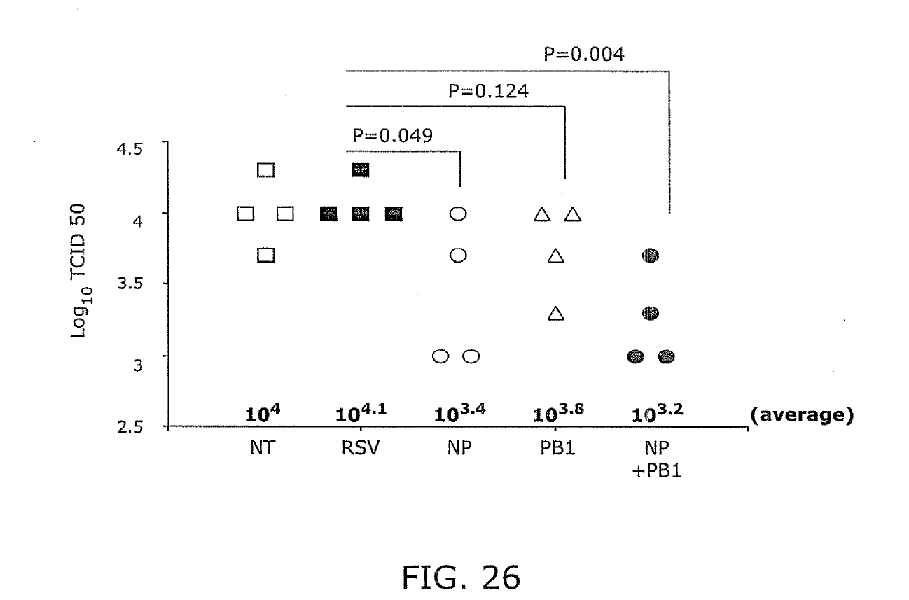

As shown in FIG. 26, virus titers were high in mice that were not given any plasmid DNA or were given a DNA vector expressing a respiratory syncytial virus (RSV)-specific shRNA. Lower virus titers were observed when mice were given plasmid DNA that expresses either NP-1496a shRNA or PB1-2257 shRNA. The virus titers were more significantly decreased when mice were given both influenza-specific plasmid DNAs together, one expressing NP-1496a shRNA and the other expressing PB1-2257 shRNA. The average log₁₀TCID₅₀ of the lung homogenate for mice that received no treatment (NT; open squares) or received a plasmid encoding an RSV-specific shRNA (RSV; filled squares) was 4.0 or 4.1, respectively. In mice that received plasmid capable of serving as a template for NP-1496a shRNA (NP; open circles), the average log₁₀TCID₅₀ of the lung homogenate was 3.4. In mice that received plasmid capable of serving as a template for PB1-2257 shRNA (PB; open triangles), the average log₁₀TCID₅₀ of the lung homogenate was 3.8. In mice that received plasmids capable of serving as templates for NP and PB shRNAs (NP+PB1; filled circles), the average log₁₀TCID₅₀ of the lung homogenate was 3.2. The differences in virus titer in the lung homogenate between the group that received no treatment or RSV-specific shRNA plasmid and the groups that received NP shRNA plasmid, PB1 shRNA plasmid, or NP and PB1 shRNA plasmids had P values of 0.049, 0.124, and 0.004 respectively. Data for individual mice are presented in Table 10 (NT=no treatment). Preliminary experiments involving intranasal administration of NP-1496 siRNA rather than NP shRNA in the presence of PBS or jetPEI but in the absence of Infasurf did not result in effective inhibition of influenza virus. These results show that shRNA expressed from DNA vectors can be processed into siRNA to inhibit influenza virus production in mice and demonstrate that Infasurf is a suitable vehicle for the delivery of plasmids from which shRNA can be expressed. In particular, these data indicate that shRNA targeted to the influenza NP and/or PB1 transcripts reduced the virus titer in the lung when administered following virus infection.

>>production.

TABLE 10
Inhibition of influenza virus production by shRNA expressed in mice

| | |
|---|---|
| Treatment | log₁₀TCID50 |

| | | | | |
|---|---|---|---|---|
| NT | 4.3 | 4.0 | 4.0 | 4.3 |
| RSV (60 μg) | 4.3 | 4.0 | 4.0 | 4.0 |
| NP (60 μg) | 4.0 | 3.7 | 3.0 | 3.0 |
| PB1 (60 μg) | 4.0 | 4.0 | 3.7 | 3.3 |
| NP + PB1 (60 μg | 3.7 | 3.3 | 3.0 | 3.0 |
| each) | | | | |

### Example 15

### Cationic Polymers Promote Cellular Uptake of siRNA

Materials and Methods

Reagents. Poly-L-lysines of two different average molecular weights [poly-L-lysine (MW (vis) 52,000; MW (LALLS) 41,800, Cat. No. P2636) and poly-L-lysine (MW (vis) 9,400; MW (LALLS) 8,400, Cat. No. P2636], poly-L-arginine (MW 15,000-70,000 Cat. No. P7762) and 3-(4,5-dimethylthiazol-2-yl)-2,5-diphenyl tetrazolium bromide (MTT) were purchased from Sigma. For purposes of description molecular weights obtained using the LALLS method will be assumed, but it is to be understood that molecular weights are approximate since the polymers display some heterogeneity in size.

Gel retardation assay. siRNA-polymer complexes were formed by mixing 10 μl of siRNA (10 pmol in 10 mM Hepes buffer, pH 7.2) with 10 μl of polymer solution containing varying amounts of polymer. Complexes were allowed to form for 30 min at room temperature, after which 20 μl was run on a 4% agarose gel. Bands were visualized with ethidium-bromide staining.

Cytotoxicity assay. siRNA-polymer complexes were formed by mixing equal amounts (50 μmol) of siRNA in 10 mM Hepes buffer, pH 7.2 with polymer solution containing varying amounts of polymer for 30 min at room temperature. Cytotoxicity was evaluated by MTT assay. Cells were seeded in 96-well plates at 30,000 cells per well in 0.2 ml of DMEM containing 10% fatal calf serum (FCS). After overnight incubation at 37° C., the medium was removed and replaced with 0.18 ml OPTI-MEM (GIBCO/BRL). siRNA-polymer complexes in 20 μl of Hepes buffer were added to the cells. After a 6-h incubation at 37° C., the polymer-containing medium was removed and replaced with DMEM-10% FCS. The metabolic activity of the cells was measured 24 h later using the MTT assay according to the manufacturer's instructions. Experiments were performed in triplicate, and the data was averaged.

Cell culture, transfection, siRNA-polymer complex formation, and viral titer determination. Vero cells were grown in DMEM containing 10% heat-inactivated FCS, 2 mM L-glutamine, 100 units/ml penicillin, and 100 μg/ml streptomycin at 37° C. under a 5% CO2/95% air atmosphere. For transfection experiments, logarithmic-phase Vero cells were seeded in 24-well plates at 4×10⁵ cells per well in 1 ml of DMEM-10% FCS. After overnight incubation at 37° C., siRNA-polymer complexes were formed by adding 501 of siRNA (400 pmol (about 700 ng) in 10 mM Hepes buffer, pH 7.2) to 50 μl of polymer vortexing. Different concentrations of polymer were used in order to achieve complete complex formation between the siRNA and polymer. The mixture was incubated at room temperature for 30 min to complete complex formation. The cell-growth medium was removed and replaced with OPTI-MEM I (Life Technologies) just before the complexes were added.

After incubating the cells with the complexes for 6 h at 37° C. under 5% CO₂, the complex-containing medium was removed and 200 μl of PR8 virus in infection medium, MOI=0.04, consisting of DMEM, 0.3% BSA (Sigma), 10 mM Hepes, 100 units/ml penicillin, and 100 μg/ml streptomycin, was added to each well. After incubation for 1 h at room temperature with constant rocking, 0.8 ml of infection medium containing 4 μg/ml trypsin was added to each well and the cells were cultured at 37° C. under 5% CO₂. At different times after infection, supernatants were harvested from infected cultures and the virus titer was determined by hemagglutination (HA) assay as described above.

Transfection of siRNA by Lipofectamine 2000 (Life Technology) was carried out according to the manufacturer's instruction for adherent cell lines. Briefly, logarithmic-phase Vero cells were seeded in 24-well plate at 4×10⁵ cells per well in 1 ml of DMEM-10% FCS and were incubated at 37° C. under 5% CO₂. On the next day, 50 μl of diluted Lipofectamine 2000 in OPTI-MEM I were added to 50 μl of siRNA (400 μmol in OPTI-MEM I) to form complexes. The cell were washed and incubated with serum-free medium. The complexes were applied to the cells and the cells were incubated at 37° C. for 6 h before being washed and infected with influenza virus as described above. At different times after infection, supernatants were harvested from infected cultures and the virus titer was determined by hemagglutination (HA) assay as described above.

Results

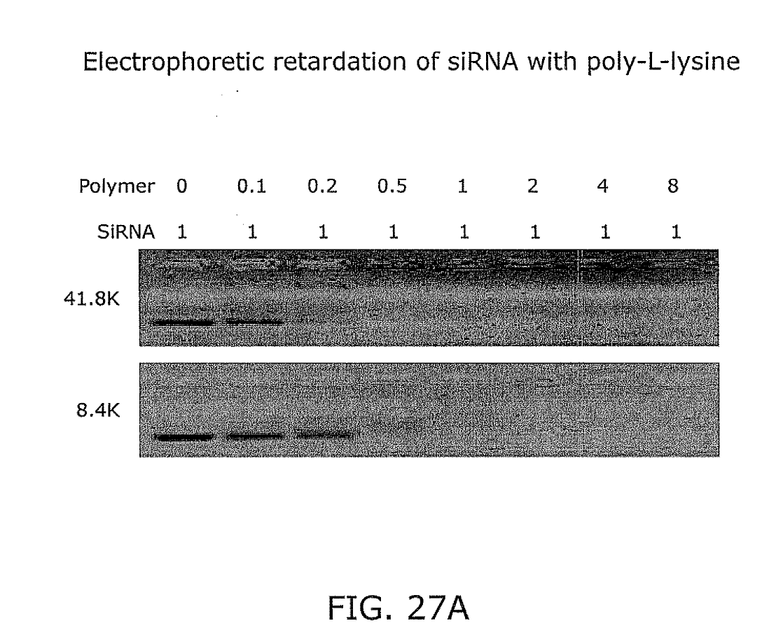

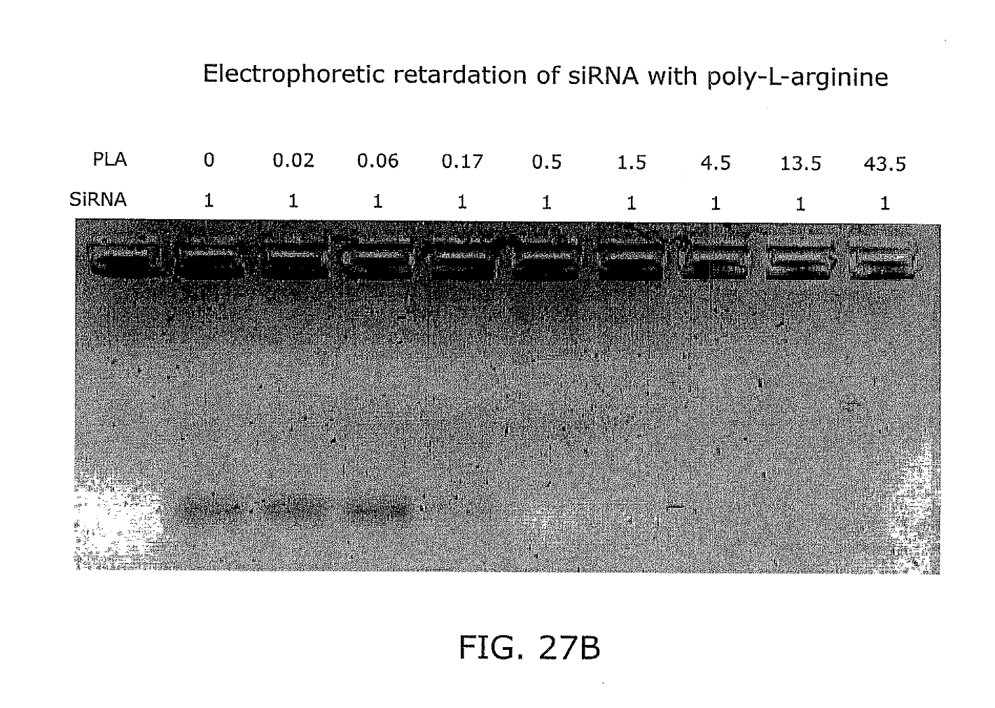

The ability of poly-L-lysine (PLL) and poly-L-arginine (PLA) to form complexes with siRNA and promote uptake of siRNA by cultured cells was tested. To determine whether PLL and/or PLA form complexes with siRNA, a fixed amount of NP-1496 siRNA was mixed with increasing amounts of polymer. Formation of polymer/siRNA complexes was then visualized by electrophoresis in a 4% agarose gel. With increasing amounts of polymer, electrophoretic mobility of siRNA was retarded (FIGS. 27A and 27B), indicating complex formation. FIGS. 27A and 27B represent complex formation between siRNAs and PLL (41.8K) or PLA, respectively. The amount of polymer used in each panel increases from left to right. In FIGS. 27A and 27B in each panel, a band can be seen in the lanes on the left, indicating lack of complex formation and hence entry of the siRNA into the gel and subsequent migration. As one moves to the right, the band disappears, indicating complex formation and failure of the complex to enter the gel and migrate.

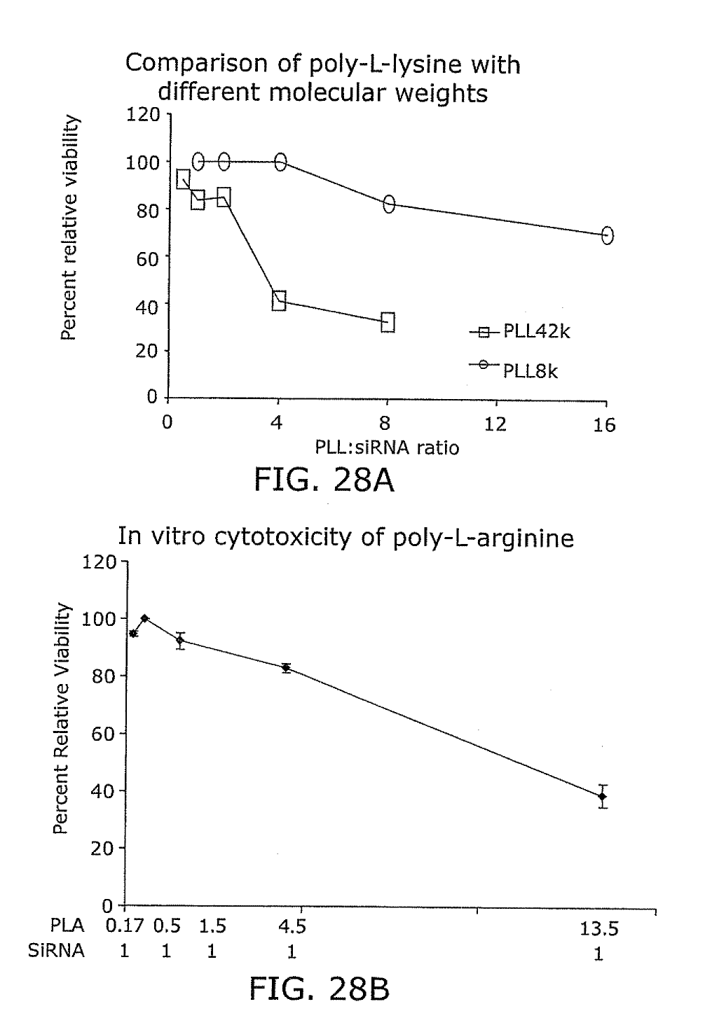

To investigate cytotoxicity of siRNA/polymer complexes, mixtures of siRNA and PLL or PLA at different ratios were added to Vero cell cultures in 96-well plates. The metabolic activity of the cells were measured by MTT assay (74). Experiments were performed in triplicate, and data was averaged. Cell viability was significantly reduced with increasing amounts of PLL (MW ˜42K) whereas PLL (˜8K) showed significantly lower toxicity, exhibiting minimal or no toxicity at PLL/siRNA ratios as high as 4:1 (FIG. 28A; circles=PLL (MW˜8K); squares=PLL (MW ˜42K)). Cell viability was reduced with increasing PLA/siRNA ratios as shown in FIG. 28B, but viability remained above 80% at PLA/siRNA ratios as high as 4.5:1. The polymer/siRNA ratio is indicated on the x-axis in FIGS. 28A and 28B. The data plotted in FIGS. 28A and 28B are presented in Tables 11 and 12. In Table 11 the numbers indicate % viability of cells following treatment with polymer/siRNA complexes, relative to % viability of untreated cells. ND=Not done. In Table 12 the numbers indicate PLA/siRNA ratio, % survival, and standard deviation as shown.

TABLE 11
Cytotoxicity of PLL/siRNA complexes (% survival)

polymer/siRNA ratio

| Treatment | 0.5 | 1.0 | 2.0 | 4.0 | 8.0 | 16.0 |
|---|---|---|---|---|---|---|
| PLL ~8.4K | 92.26 | 83.57 | 84.39 | 41.42 | 32.51 | ND |
| PLL ~41.8K | ND | 100 | 100 | 100 | 82.55 | 69.63 |

TABLE 12
Cytotoxicity of PLA/siRNA complexes (% survival)

| | polymer/siRNA ratio | | | | |
|---|---|---|---|---|---|
| | 0.17 | 0.5 | 1.5 | 4.5 | 13.5 |
| % survival | 94.61 | 100 | 92.33 | 83 | 39.19 |
| Standard deviation | .74 | 1.91 | 2.92 | 1.51 | 4.12 |

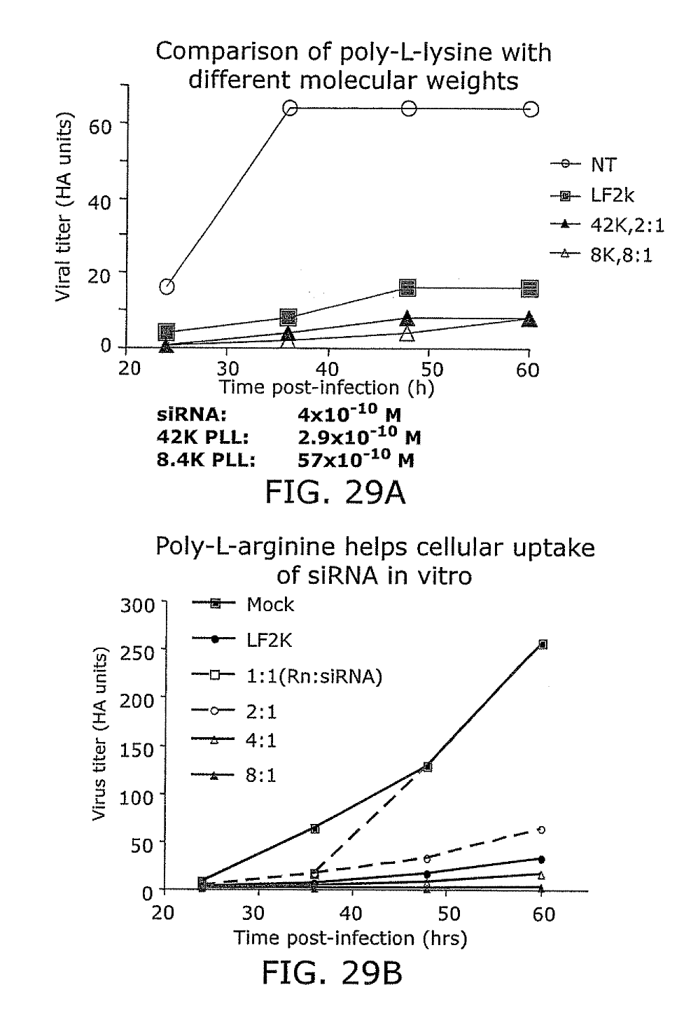

To determine whether PLL or PLA promotes cellular uptake of siRNA, various amounts of polymer and NP-1496 were mixed at ratios at which all siRNA was complexed with polymer. Equal amounts of siRNA were used in each case. A lower polymer/siRNA ratio was used for ˜42K PLL than for ˜8K PLL since the former proved more toxic to cells. The complexes were added to Vero cells, and 6 hrs later the cultures were infected with PR8 virus. At different times after infection, culture supernatants were harvested and assayed for virus by HA assay. FIG. 29A is a plot of virus titers over time in cells receiving various transfection treatments (circles=no treatment; squares=Lipofectamine; filled triangles=PLL (˜42K at PLL/siRNA ratio=2); open triangles=PLL (˜8K at PLL/siRNA ratio=8). As shown in FIG. 29A, virus titers increased with time in the non-transfected cultures. Virus titers were significantly lower in cultures that were transfected with NP-1496/Lipofectamine and were even lower in cultures treated with PLL/NP-1496 complexes. The data plotted in FIG. 29A are presented in Table 13 (NT=no treatment; LF2K=Lipofectamine. The PLL:siRNA ratio is indicated in parentheses.

PLA was similarly tested over a range of polymer/siRNA ratios. FIG. 29B is a plot of virus titers over time in cells receiving various transfection treatments (filled squares=mock transfection; filled circles=Lipofectamine; open squares=PLA at PLA/siRNA ratio=1; open circles=PLA at PLA/siRNA ratio=2; open triangles=PLA at PLA/siRNA ratio=4; filled triangles=PLA at PLA/siRNA ratio=8). As shown in FIG. 29B, virus titers increased with time in the control (mock-transfected) culture and in the culture treated with PLA/siRNA at a 1:1 ratio. Virus titers were significantly lower in cultures that were transfected with NP-1496/Lipofectamine and were even lower in cultures treated with PLA/siRNA complexes containing complexes at PLA/siRNA ratios of 4:1 or higher. Increasing amounts of polymer resulted in greater reduction in viral titer. The data plotted in FIG. 29B are presented in Table 14.

TABLE 13
Inhibition of influenza virus production by polymer/siRNA complexes

Time (hours)

| Treatment | 24 | 36 | 48 | 60 |
|---|---|---|---|---|
| mock transfection | 16 | 64 | 64 | 64 |
| LF2K | 4 | 8 | 16 | 16 |
| PLL ~42K (2:1) | 1 | 4 | 8 | 8 |
| PLL ~8K (8:1) | 1 | 2 | 4 | 8 |

TABLE 14
Inhibition of influenza virus production by polymer/siRNA complexes

Time (hours)

| Treatment | 24 | 36 | 48 | 60 |
|---|---|---|---|---|
| mock transfection | 8 | 64 | 128 | 256 |
| LF2K | 2 | 6 | 16 | 32 |
| PLA (1:1) | 4 | 16 | 128 | 256 |
| PLA (2:1) | 4 | 16 | 32 | 64 |
| PLA (4:1) | 1 | 4 | 8 | 16 |
| PLA (8:1) | 1 | 1 | 1 | 2 |

Thus, cationic polymers promote cellular uptake of siRNA and inhibit influenza virus production in a cell line and are more effective than the widely used transfection reagent Lipofectamine. These results also suggest that additional cationic polymers may readily be identified to stimulate cellular uptake of siRNA and describe a method for their identification. PLL and PLA can serve as positive controls for such efforts.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

### REFERENCES

1. Lamb, R. A., and R. M. Krug. 2001. Orthomyxoviridae: the viruese and their replication. Fundamental Virology Ed. D. M. Knipe & P. M. Hpwley:725-770.
2. Simonsen, L., K. Fukuda, L. B. Schomberger, and N. J. Cox. 2000. The impact of influenza epidemics on hospitalizations. J. Infect. Dis. 181:831-837.
3. Webster, R. G., W. J. Bean, O. T. Gorman, T. M. Chambers, and Y. Kawaoka. 1992. Evolution and ecology of influenza A viruses. Microbiol. Rev. 56:1.52-179.
4. Parvin, J. D., A. Moscona, W. T. Pan, J. M. Leider, and P. Palese. 1986. Measurement of the mutation rates of animal viruses: influenza A virus and poliovirus type 1. J. Virol. 59:377-383.
5. Smith, F. L., and P. Palese. 1989. Variation in influenza virus genes:epidemiology, pathogenic, and evolutionary consequences. The influenza viruses Krug, R. M. ed.:New York:Plenum.
6. Webster, R. G., W. G. Layer, G. M. air, and S. G. C. 1982. Molecular mechanisms of variation in influenza viruses. Nature 296:115-121.
7. Webby, R. J., and R. G. Webster. 2001. Emergence of influenza A viruses. Phil. Trans. R. Soc. Lond. 356:1817-1828.
8. Patterson, K. D., and G. F. Pyle. 1991. The geography and mortality of the 1918 influenza pandemic. Bull. Hist. Med. 65:4-21.
9. Taubenberger, J. K., A. H. Reid, T. A. Janczewski, and T. G. GFanning. 2001. Integrating historical, clinical and molecular genetic data in order to explain the origin and virulence of the 1918 Spanish influenza virus. Phil. Trans. R. Soc. Lond. 356:1829-1839.
10. Claas, E. C., A. D. Osterhaus, R. van Beek, J. C. De Jong, G. F. Rimmelzwaan, D. A. Senne, S. Krauss, K. F. Shortridge, and R. G. Webster. 1998. Human influenza A H₅N₁ virus related to a highly pathogenic avian influenza virus. Lancet 351:472-477.
11. Yuen, K. Y., P. K. Chan, M. Peiris, D. N. Tsang, T. L. Que, K. F. Shortridge, P. T. Cheung, W. K. To, E. T. Ho, R. Sung, and A. F. Cheng. 1998. Clinical features and rapid viral diagnosis of human disease associated with avian influenza A H5N1 virus. Lancet 351:467-471.
12. Fukuda, F., C. B. Bridges, and T. L. e.a. Brammer. 1999. Prevention and control of influenza: recommendations of the advisory committee on immunization practices (ACIP). MMWR Morb. Mortal. Wkly. Rep. 48:1-28.
13. Castle, S. C. 2000. Clinical relevane of age-related immune dysfunction. Clin. Infect. Dis. 31:578-585.
14. Luscher-Mattli, M. 2000. Influenza chemotherapy: a review of the present state of art and of new drugs in development. Arch. Virol. 145:2233-2248.
15. Cox, N. J., and K. Subbarao. 1999. Influenza. Lancet 354:1277-1282.
16. Vaucheret, H., C. Beclin, and M. Fagard. 2001. Post-transcriptional gene silencing in plants. J. Cell Sci. 114:3083-3091.
17. Sharp, P. A. 2001. RNA interference-2001. Genes Dev. 15:485-490.
18. Brantl, S. 2002. Antisense-RNA regulation and RNA interference. Biochem. Biophy. Acta 1575:15-25.
19. Baulcombe, D. 2002. RNA silencing. Curr. Biol. 12:R82-R84.
20. Fire, A., S. Xu, M. K. Montgomery, S. A. Kostas, S. E. Driver, and M. C. C. 1998. Potent and specific genetic interference by double-stranded RNA in Caenorhabditis elegans. Nature 391:806-811.
21. Elbashir, S., J. Harborth, W. Lendeckel, A. Yalcin, K. Weber, and T. Tuschl. 2001. Duplexes of 21-nucleotide RNAs mediate RNA interference in cultured mammalian cells. Nature 411:494-498.
22. McManus, M. T., and P. A. Sharp. 2002. Gene silencing in mammals by short interfering RNAs. Nature Rev. Gene. 3:737-747.
23. Kumar, M., and G. G. Carmichael. 1998. Antisense RNA: function and fate of duplex RNA in cells of higher eukaryotes. Microbiol. Mol. Biol. Rev. 62:1415-1434.
24. Gitlin, L., S. Karelsky, and R. Andino. 2002. Short interfering RNA confers intracellular antiviral immunity in human cells. Nature 418:430-434.
25. Pderoso de Lima, M. C., S. Simoes, P. Pires, H. Faneca, and N. Duzgunes. 2001. Cationic lipid-DNA complexes in gene delivery: from biophysics to biological applications. Adv. Drug Deliv. Rev. 47:277-294.
26. Holen, T., M. Amarzguioui, M. T. Wiiger, E. Babaie, and H. Prydz. 2002. Positional effects of short interfering RNAs targeting the human coagulation trigger tissue factor. Nucleic Acids Res. 30:1757-1766.
27. McManus, M. T., Haines, B. B., Dillon, C. P., Whitehurst, C. E., van Parijs, L., Chen, J., and Sharp, P. A. (2002). Small interfering RNA-mediated gene silencing in T lymphocytes. J Immunol 169, 5754-5760.
28. Elbashir, S. M., J. Martinez, A. Patkaniowska, W. Lendeckel, and T. Tuschl. 2001. Functional anatomy of siRNAs for mediating efficient RNAi in Drosophila melanogaster embryo lysate. EMBO J. 20:6877-6888.
29. Yang, D., H. Lu, and J. W. Erickson. 2000. Evidence that processed small dsRNAs may mediate sequence-specific mRNA degradation during RNAi in Drosophila embryos. Curr. Biol. 10:1191-1200.
30. Caplen, N. J., J. Fleenor, A. Fire, and R. A. Morgan. 2000. dsRNA-mediated gene silencing in cultured Drosophila cells: a tissue culture model for the analysis of RAN interference. Gene 252:95-105.
31. Edwards, D. A., J. Hanes, G. Caponetti, J. Hrkach, A. Ben-Jebria, M. L. Eskew, J. Mintzes, D. Deaver, N. Lotan, and R. Langer. 1997. Large porous particles for pulmonary drug delivery. Science 276:1868-1871.
32. Putnam, D., C. A. Gentry, D. W. Pack, and R. Langer. 2001. Polymer-based gene delivery with low cytotoxicity by a unique balance of side-chain termini. Proc. Natl. Acad. Sci. USA 98:1200-1205.
33. Lynn, D. M., and R. Langer. 2000. Degradable Poly(-amino esters): Synthesis, Characterization, and Self-Assembly with Plasmid DNA. J. Am. Chem. Soc. 122:10761-10768.
34. Lynn, D. M., D. G. Anderson, D. Putnam, and R. Langer. 2001. Accelerated discovery of synthetic transfection vectors: parallel synthesis and screening of a degrable polymer library. J. Am. Chem. Soc. 123:8155-8156.
35. Han, S.-O., R. I. Mahato, Y. K. Sung, and S. W. Kim. 2000. Development of Biomaterials for gene therapy. Mol. Therapy. 2:302-317.
36. Soane, R. J., M. Frier, A. C. Perkins, N. S. Jones, S. S. Davis, and L. Illum. 1999. Evaluation of the clearance characteristics of bioadhesive systems in humans. Int. J. Pharm. 178:55-65.
37. Boussif, O., F. Lezoualc'h, M. A. Zanta, M. D. Mergny, D. Scherman, B. Demeneix, and J. P. Behr. 1995. A versatile vector for gene and oligonucleotide transfer into cells in culture and in vivo: polyethylenimine. Proc. Natl. Acad. Sci. USA 92:7297-7301.
38. Astafieva, I., I. Maksimova, E. Lukanidin, V. Alakhov, and A. Kabanov. 1996. Enhancement of the polycation-mediated DNA uptake and cell transfection with Pluronic P85 block copolymer. FESB Lett. 389:278-280.
39. Davis, S. S. 1999. Delivery of peptide and non-peptide drugs through the respiratory tract. Pharm. Sci. Technol. Today 2:450-457.
40. Roy, K., H.-Q. Mao, S.-K. Huang, and K. W. Leong. 1999. Oral delivery with chitosan/DNA nanoparticles generates immunologic protection in murine model of peanut allergy. Nat. Med. 5:387-391.
41. Hansen, M. B., S. E. Nielsen, and K. Berg. 1989. Re-examination and further development of a precise and rapid dye method for measuring cell growth/cell kill. J. Immunol. Methods 119:203-210.
42. Green, M., and P. M. Loewenstein. 1988. Autonomous functional domains of chemically synthesized human immunodeficiency virus tat trans-activator protein. Cell 55:1179-1188.
43. Frankel, A. D., and C. O. Pabo. 1988. Cellular uptake of the tat protein from human immunodeficiency virus. Cell 55:1189-1193.
44. Elliott, G., and P. O'Hare. 1997. Intercellular trafficking and protein delivery by a herpesvirus structural protein. Cell 88:223-233.
45. Joliot, A., C. Pernelle, H. Deagostini-Bazin, and A. Prochiantz. 1991. Antennapedia homeobox peptide regulates neural morphogenesis. Proc. Natl. Acad. Sci. USA 88:1864-1868.
46. Fawell, S., J. Seery, Y. Daikh, C. Moore, L. L. Chen, B. Pepinsky, and J. Barsoum. 1994. Tat-mediated delivery of heterologous proteins into cells. Proc. Natl. Acad. Sci. USA 91:664-668.
47. Schwarze, S. R., A. Ho, A. Vocero-Akbani, and S. F. Dowdy. 1999. In vivo protein transduction:delivery of a biologically active protein. Science 285:1569-1572.
48. Derossi, D., G. Chassaing, and A. Prochiantz. 1998. Trojan peptides: the penetratin system for intracellular delivery. Trends Cell Biol. 8:84-87.
49. Troy, C. M., D. Derossi, A. Prochiantz, L. A. Greene, and M. L. Shelanski. 1996. Downregulation of Cu/Zn superoxide dismutase leads to cell death via the nitric oxide-peroxynitrite pathway. J. Neurosci. 16:253-261.
50. Allinquant, B., P. Hantrayc, P. Mailleux, K. Moya, C. Bouillot, and A. Prochiantz. 1995. Downregulation of amyloid precursor protein inhibits neurite outgrowth in vitro. J. Cell Biol. 128:919-927.
51. Futaki, S., T. Suzuki, W. Ohashi, T. Yagami, S. Tanaka, K. Ueda, and Y. Sugiura. 2001. Arginine-rich peptides. An abundant source of membrane-permeable peptides having potential as carriers for intracellular protein delivery. J. Biol. Chem. 276:5836-5840.
52. Densmore, C. L., F. M. Orson, B. Xu, B. M. Kinsey, J. C. Waldrep, P. Hua, B. Bhogal, and V. Knight. 1999. Aerosol delivery of robust polyethyleneimine-DNA complexes for gene therapy and genetic immunization. Mol. Therapy. 1:180-188.
53. Arppe, J., M. Widgred, and J. C. Waldrep. 1998. Pulmonary pharmacokinetics of cyclosporin A liposomes. Intl. J. Pharm. 161:205-214.
54. Griesenbach, U., A. chonn, R. Cassady, V. Hannam, C. Ackerley, M. Post, A. K. Transwell, K. Olek, H. O'Brodovich, and L.-C. Tsui. 1998. Comparison between intratracheal and intravenous administration of liposome-DNA complexes for cystic fibrosis lung gene therapy. Gene Ther. 5:181-188.
55. Orson, F. M., L. Song, A. Gautam, C. L. DEnsmore, B. Bhogal, and B. M. Kinsey. 2002. Gene delivery to the lung using protein/polyethyleneimine/plasmid complexes. Gene Therapy 9:463-471.
56. Gautam, A., C. L. Densmore, E. Golunski, B. Xu, and J. C. Waldrep. 2001. Transgene expression in mouse airway epithelium by aerosol gene therapy with PEI-DNA complexes. Mol. Therapy. 3:551-556.
57. Tabata, Y., and Y. Ikada. 1988. Effect of size and surface charge of polymer microspheres on their phagocytosis by macrophage. J. Biomed. Mater. Res. 22:837-842.
58. Vanbever, R., J. D. Mintzes, J. Wang, J. Nice, D. chen, R. Batycky, L. R., and D. A. Edwards. 1999. Formulation and physical characterization of large porous particles for inhalation. Pharmaceutical Res. 16:1735-1742.
59. McManus, M. T., C. P. Peterson, B. B. Haines, J. Chen, and P. A. Sharp. 2002. Gene silencing using micro-RNA designed hairpins. RNA 8:842-850.
60. Myslinski, E., J. C. Ame, A. Krol, and P. Carbon. 2001. An unusually compact external promoter for RNA polymerase III transcription of the human H1 RNA gene. Nucleic Acids Res. 29:2502-2509.
61. Brummelkamp, T. R., R. Bernards, and R. Agami. 2002. A system for stable expression of short interfering RNAs in mammalian cells. Science 296:550-553.
62. Paddison, P. J., A. A. Caudy, E. Bernstein, G. J. Hannon, and D. S. Conklin. 2002. Short hairpin RNAs (shRNAs) induce sequence-sepcific silencing in mammalian cells. Genes Dev. 16:948-958.
63. Gil, J., and M. Esteban. 2000. Induction of apoptosis by the dsRNA-dependent protein kinase (PKR): mechanism of action. Apoptosis 5:107-114.
64. Bitko, V., and S. Barik. 2001. Phenotypic silencing of cytoplasmic genes using sequence-specific double-stranded short interfering RNA and its application in the reverse genetic of wild type negative-strand RNA viruses. BMC Microbiol. 1:34-43.
65. Garcia-Sastre, A. (2002) Microbes & Inf 4, 647-655.
66. Katze, M. G., He, Y. & Gale Jr., M. (2002) Nature Rev. Immunol. 2, 675-687.
67. Diaz, M. O., Ziemin, S., Le Beau, M. M., Pitha, P., Smith, S. D., Chilcote, R. R. & Rowley, J. D. (1988) Proc. Natl. Acad. Sci. USA 85, 5259-5263.
68. Diaz, M. O., Pomykala, H. M., Bohlander, S. K., Maltepe, E., Malik, K., Brownstein, B. & Olopade, O. 1. (1994) Genomics 22, 540-552.
69. Kim, M.-J., Latham, A. G. & Krug, R. M. (2002) Proc. Natl. Acad. Sci. USA 99, 10096-10101.
70. Medcalf, L., Poole, E., Elton, D. & Digard, P. (1999) J. Virol. 73, 7349-7356.
71. Shapiro, G. I. & Krug, R. M. (1988) J. Virol. 62, 2285-2290.
72. Beaton, A. R. & Krug, R. M. (1986) Proc. Natl. Acad. Sci. USA 83, 6282-6286
73. Lois, C., E. J. Hong, S. Pease, E. J. Brown, and D. Baltimore. (2002) Science 295:868-872.
74. Weiss, D. J., G. M. Mutlu, L. Bonneau, M. Mendez, Y. Wang, V. Dumasius, and P. Factor. (2002) Mol. Ther. 6:43-49.
75. Hansen, M. B., S. E. Nielsen, and K. Berg. (1989) J. Immunol. Methods 119:203-210.
76. Kunath, K, von Harpe A, Fischer D, Petersen H, Bickel U, Voigt K, Kissel T. (2003) J Control Release 89(1):113-25.
77. Jobe A. Surfactant treatment for respiratory distress syndrome. Respir Care 1986; 31(6):467-476.
78. Berry D. Neonatology in the 1990's: surfactant replacement therapy becomes a reality. Clin Pediatr 1991; 30(3):167-170.
79. Avery M E, Mead J. Surface properties in relation to atelectasis and hyaline membrane disease. Am J Dis Child 1959; 97:517-523.
80. von Neergard K. Neue Auffassungen über einen Grundbegriff der Atemmechanik: die Retraktionskraft der Lunge, äbhangig von der Oberflächenspannung in den Alveolen. Z Ges Exp Med 1929; 66:373.
81. Hallman M, Teramo K, Ylikorkala O, Merritt T A. Natural surfactant substitution in respiratory distress syndrome. J Perinat Med 1987; 15:463-468.
82. Bloom B T, Kattwinkel J, Hall R T, et al. Comparison of Infasurf (calf lung surfactant extract) to Survanta (beractant) in the treatment and prevention of respiratory distress syndrome. Pediatrics. 1997; 100:31-38.
83. Mizuno K, Ikegami M, Chen C-M, et al. Surfactant protein-B supplementation improves in vivo function of a modified natural surfactant. Pediatr Res. 1995; 37:271-276.
84. Hall S B, Venkitaraman A R, Whitsett J A, et al. Importance of hydrophobic apoproteins as constituents of clinical exogenous surfactants. Am Rev Respir Dis. 1992; 145:24-30.
85. C. H. Ahn, S. Y. Chae, Y. H. Bae and S. W. Kim, Biodegradable poly(ethylenimine) for plasmid DNA delivery. J Control Release 80, 273-82, 2002.
86. Kichler, A., Leborgne C, Coeytaux E, Danos O. Polyethylenimine-mediated gene delivery: a mechanistic study. J Gene Med. 2001 March-April; 3(2):135-44.
87. Brissault B, Kichler A, Guis C, Leborgne C, Danos O, Cheradame H. Synthesis of linear polyethylenimine derivatives for DNA transfection Bioconjug Chem. 2003 May-June; 14(3):581-7
88. Kichler A, Leborgne C, Marz J, Danos O, Bechinger B. Histidine-rich amphipathic peptide antibiotics promote efficient delivery of DNA into mammalian cells Proc Natl Acad Sci USA. 2003 Feb. 18; 100(4):1564-8
89. Brantl, S. (2002). Antisense-RNA regulation and RNA interference. Biochim Biophys Acta 1575, 15-25.
90. Semizarov, D., Frost, L., Sarthy, A., Kroeger, P., Halbert, D. N., and Fesik, S. W. (2003). Specificity of short interfering RNA determined through gene expression signatures. Proc Natl Acad Sci USA 100, 6347-6352.
91. Chi, J. T., Chang, H. Y., Wang, N. N., Chang, D. S., Dunphy, N., and Brown, P. O. (2003). Genomewide view of gene silencing by small interfering. RNAs. Proc Natl Acad Sci USA 100, 6343-6346.
92. Bitko, V., and Barik, S. (2001). Phenotypic silencing of cytoplasmic genes using sequence-specific double-stranded short interfering RNA and its application in the reverse genetics of wild type negative-strand RNA viruses. BMC Microbiol 1, 34.
93. Anderson, D. G., Lynn, D. M., and Langer, R. (2003). Semi-Automated Synthesis and Screening of a Large Library of Degradable Cationic Polymers for Gene Delivery. Angew Chem Int Ed Engl 42, 3153-3158.
94. Ge, Q., McManus, M., Nguyen, T., Shen, C.-H., Sharp, P. A., Eisen, H. N., and Chen, J. (2003). RNA interference of influenza virus production by directly targeting mRNA for degradation and indirectly inhibiting all viral RNA transcription. Proc Natl Acad Sci USA 100, 2718-2723.
95. Kumar, M., and Carmichael, G. G. (1998). Antisense RNA: function and fate of duplex RNA in cells of higher eukaryotes. Microbiol. Mol Biol Rev 62, 1415-1434.
96. Lèuscher-Mattli, M. (2000). Influenza chemotherapy: a review of the present state of art and of new drugs in development. Arch Virol 145, 2233-2248.
97. McCaffrey, A. P., Meuse, L., Pham, T. T., Conklin, D. S., Hannon, G. J., and Kay, M. A. (2002). RNA interference in adult mice. Nature 418, 38-39.
98. McCaffrey, A. P., Nakai, H., Pandey, K., Huang, Z., Salazar, F. H., Xu, H., Wieland, S. F., Marion, P. L., and Kay, M. A. (2003). Inhibition of hepatitis B virus in mice by RNA interference. Nat Biotechnol 21, 639-644.
99. Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirus-based system to functionally silence genes in primary mammalian cells, stem cells and transgenic mice by RNA interference. Nat Genet. 33, 401-406.
100. Shapiro, G. I., and Krug, R. M. (1988). Influenza virus RNA replication in vitro: synthesis of viral template RNAs and virion RNAs in the absence of an added primer. J Virol 62, 2285-2290.
101. Shen, C., Buck, A. K., Liu, X., Winkler, M., and Reske, S. N. (2003). Gene silencing by adenovirus-delivered siRNA. FEBS lett 539, 111-114.
102. Simeoni, F., Morris, M. C., Heitz, F., and Divita, G. (2003). Insight into the mechanism of the peptide-based gene delivery system MPG: implications for delivery of siRNA into mammalian cells. Nucleic Acids Res 31, 2717-2724.
103. Soane, R. J., Frier, M., Perkins, A. C., Jones, N. S., Davis, S. S., and Illum, L. (1999). Evaluation of the clearance characteristics of bioadhesive systems in humans. Int J Pharm 178, 55-65.
104. Stiver, G. (2003). The treatment of influenza with antiviral drugs. CMAJ 168, 49-56.
105. Tachibana, R., Harashima, H., Ide, N., Ukitsu, S., Ohta, Y., Suzuki, N., Kikuchi, H., Shinohara, Y., and Kiwada, H. (2002). Quantitative analysis of correlation between number of nuclear plasmids and gene expression activity after transfection with cationic liposomes. Pharm Res 19, 377-381.
106. Thomas, C. E., Ehrhardt, A., and Kay, M. A. (2003). Progress and problems with the use of viral vectors for gene therapy. Nat Rev Genet. 4, 346-358.
107. Xia, H., Mao, Q., Paulson, H. L., and Davidson, B. L. (2002). siRNA-mediated gene silencing in vitro and in vivo. Nat Biotechnol 20, 1006-1010.
108. Zhang, G., Song, Y. K., and Liu, D. (2000). Long-term expression of human alpha1-antitrypsin gene in mouse liver achieved by intravenous administration of plasmid DNA using a hydrodynamics-based procedure. Gene Ther 7, 1344-1349
109. Cheung, M., and Lieberman, J. M. (2002). Influenza: update on strategies for managment. Contemporay Pediatrics 19, 82-114.
110. Yang, P., Althage, A., Chung, J., and Chisari, F. (2000) Hydrodynamic injection of viral DNA: A mouse model of acute hepatitis B virus infection, Proc. Natl. Acad. Sci., 99(21): 13825-13830.
111. Zhang, G., Budker, V., Wolff, JA (1999) High levels of foreign gene expression in hepatocytes after tail vein injections of naked plasmid DNA, Hum Gene Ther, 10:1735-1737.
112. Liu, F., Song, Y. K., Liu, D. (1999) Hydrodynamics-based transfection in animals by systemic administration of plasmid DNA. Gene Therapy; 6:1258-1266.
113. Naldini, L. Lentiviruses as gene transfer agents for delivery to non-dividing cells. Curr Opin Biotechnol 9, 457-63 (1998).
114. Naldini, L. et al. In vivo gene delivery and stable transduction of nondividing cells by a lentiviral vector. Science 272, 263-7 (1996).
115. Miyoshi, H., Blomer, U., Takahashi, M., Gage, F. H. & Verma, I. M. Development of a self-inactivating lentivirus vector. J Virol 72, 8150-7 (1998).
116. Pfeifer, A., Ikawa, M., Dayn, Y. & Verma, I. M. Transgenesis by lentiviral vectors: lack of gene silencing in mammalian embryonic stem cells and preimplantation embryos. Proc Natl Acad Sci USA 99, 2140-5 (2002).
117. Lois, C., Hong, E. J., Pease, S., Brown, E. J. & Baltimore, D. Germline transmission and tissue-specific expression of transgenes delivered by lentiviral vectors. Science 295, 868-72 (2002).
118. Anderson D G, Lynn D M, Langer R., Semi-Automated Synthesis and Screening of a Large Library of Degradable Cationic Polymers for Gene Delivery. Angew Chem Int Ed Engl. 2003 Jul. 14; 42(27):3153-3158.
119. McCown, M., Diamond, M. S., and Pekosz, A., Virology, 313: 514-524 (2003).
120. Gratton, J. P., Yu, J., Griffith, J. W., Babbitt, R. W., Scotland, R. S., Hickey, R., Giordano, F. J., and Sessa, W. C., Cell-permeable peptides improve cellular uptake and therapeutic gene delivery of replication-deficient viruses in cells and in vivo. Nat. Med., 9(3): 357-362 (2003).
121. McKenzie, D. L., Kwok, K. Y., and Rice, K. G., A Potent New Class of Reductively Activated Peptide Gene Delivery Agents. J. Biol. Chem., 275(14): 9970-9977 (2000).
122. Park, Y., Kwok, K. Y., Boukarim, C., and Rice, K. G., Synthesis of Sulfhydryl Cross-Linking Poly(Ethylene Glycol Peptides and Glycopeptides as Carriers for Gene Delivery. Bioconjugate Chem., 13: 232-239 (2002).
123. Zhang, X., Sawyer, G., J., Dong, X., Qiu, Y., Collins, L., and Fabre, J. W. The in vivo use of chloroquine to promote non-viral gene delivery to the liver via the portal vein and bile duct. J. Gene Med., 5:209-218, 2003.
124. Thomas, N., and Klibanov, A. M. Non-viral gene therapy: polycation-mediated DNA delivery. Appl. Microbiol. Biotechnol. 62:27-34 (2003).
125. S.-O. Han, R. I. Mahato, Y. K. Sung, S. W. Kim, “Development of biomaterials for gene therapy”, Molecular Therapy 2:302317, 2000.
126. Weiss, D., Delivery of Gene Transfer Vectors to the Lung: Obstacles and the Role of Adjunct Techniques for Airway Administration. Mol. Therapy. 6(2) (2002).
127. Ferrari S, Geddes D M, Alton E W. Barriers to and new approaches for gene therapy and gene delivery in cystic fibrosis. Adv Drug Deliv Rev, 54(11): 1373-93 (2002).
128. Orson F M, Song L, Gautam A, Densmore C L, Bhogal B S, Kinsey B M. Gene delivery to the lung using protein/polyethylenimine/plasmid complexes. Gene Ther. 2002 April; 9(7):463-71.
129. Tiyaboonchai, W., Woiszwillo, J., and Middauth, C. R. Formulation and characterization of DNA-polyethylenemimine-dextran sulfate nanoparticles. Eur. J. Pharm. Sci. 19:191-202 (2003).
130. Benns, J., Mahato, R., and Kim, S. W., Optimization of factors influencing the transfection efficiency of folate-PEG-folate-graft polyethyleneimine, J. Cont. Release 79:255-269 (2002).
131. Putnam, D., Zelikin, A. N., Izumrudov, V. A., and Langer, R., Polyhistidine-PEG:DNA nanocomposites for gene delivery, Biomaterials 24: 4425-4433 (2003).
132. Benns, J., Choi, J-S., Mahato, R. I., Park, J-H., and Kim, S. W., pH-Sensitive Cationic Polymer Gene Delivery Vehicle: N-Ac-poly(L-histidine)-graft-poly(L-lysine) Comb Shaped Polymer, Bioconj. Chem. 11:637-645 (2000).
133. Panyam, J., Zhou, W-Z., Prabha, S., Sahoo, S. K., and Labhasetwar, V., Rapid endolysosomal escape of poly(DL-lactide-co-glycolide) nanoparticles: implications for drug and gene delivery, FASEB J., 16: 1217-1226 (2002).
134. Lindgren, M., Hallbrink, M., Prochiantz, A., and Langel, U. Cell-penetrating peptides. Trends Pharmacol. Sci. 21:99-103 (2000).
135. Morris, M. C., Depollier, J., Mery, J., Heitz, F., and Divita, G., A peptide carrier for the biologically active proteins into mammalian cells. Nat. Biotechnol. 19:1174-1176 (2001).
136. Schwarze, S. R. and Dowdy, S. F. In vivo protein transduction: intracellular delivery of biologically active proteins, compounds, and DNA. Trends Pharmacol. Sci. 21:99-103 (2000).
137. Amarzguioui, A., Holen, T., Babaie, E., and Prydz, H. Tolerance for mutations and chemical modifications in a siRNA. Nuc. Acids. Res. 31(2): 589-595 (2003).
138. Braasch, D. A., Jensen, S., Liu, Y., Kaur, K., Arar, K., White, M. A., and Corey, D. R>RNA Interference in Mammalian Cells by Chemically Modified RNA. Biochemistry 42: 7967-7975 (2003).
139. Chiu, Y-L. and Rana, T. M. siRNA function in RNAi: A chemical modification analysis. RNA 9(9): 1034-1048 (2003).
140. Satishchandran C. Characterization of a new class of DNA delivery complexe formed by the local anesthetic bupivacaine. Biochimica et Biophysica Acta 1468: 20-30 (2000).
141. Jeong J H, Park T G., Poly(L-lysine)-g-poly(D,L-lactic-co-glycolic acid) micelles for low cytotoxic biodegradable gene delivery carriers. J Control Release, 82(1):159-66 (2002)

1-200. (canceled)

201. A siRNA sequence against the constant region of the influenza virus nucleoprotein gene comprising:

| | |
|---|---|
| Sense strand: | 5′ UGAAGGAUCUUAUUUCUUCdTdT 3 |
| Anti sense strand: | 3′ dTdTACUUCCUAGAAUAAAGAAG 5′ |

said sequence being inhibitory against influenza virus in animals including humans.

202. The siRNA sequence of claim 201 in the form of an aqueous suspension suitable for nasal inhalation.

203. The siRNA sequence of claim 201 in the form of a plasmid expressing intracellularly in animals including humans.

204. The siRNA sequence of claim 201 in the form of an AAV vector adapted to express intercellularly and establish a permanent inhibitory effect against influenza virus by integrating to the cellular chromosome of animals including humans.

205. A method comprising the administration to an animal including humans of a therapeutically effective amount of the siRNA sequence of claim 1.

206. The method of claim 205 wherein the administration is by nasal inhalation in the form of an aqueous mist.

207. The method of claim 205 wherein the administration is in the form of a plasmid.

208. The method of claim 205 wherein the administration is in the form of a AAV vector.

209. The method of claim 205 wherein the administration is effective against influenza virus A, B or C.

210. The method of claim 205 wherein the administration is effective against avian influenza (H5N1).

211. A siRNA sequence against the constant region of the influenza virus nucleoprotein gene comprising:
Sense strand:
5′ UGAAGGAUCUUAUUUCUUCGGdTdT 3′
Anti sense strand:
3′ dTdTACUUCCUAGAAUAAAGAAGCC 5′
said sequence being inhibitory against influenza virus in animals including humans.

212. The siRNA sequence of claim 211 in the form of an aqueous suspension suitable for nasal inhalation.

213. The siRNA sequence of claim 211 in the form of a plasmid expressing intracellularly in animals including humans.

214. The siRNA sequence of claim 211 in the form of an AAV vector adapted to express intercellularly and establish a permanent inhibitory effect against influenza virus by integrating to the cellular chromosome of animals including humans.

215. A method comprising the administration to an animal including humans of a therapeutically effective amount of the siRNA sequence of claim 1.

216. The method of claim 215 wherein the administration is by nasal inhalation in the form of an aqueous mist.

217. The method of claim 215 wherein the administration is in the form of a plasmid.

218. The method of claim 215 wherein the administration is in the form of a AAV vector.

219. The method of claim 215 wherein the administration is effective against influenza virus A, B or C.

220. The method of claim 215 wherein the administration is effective against avian influenza (H5N1).

221. A siRNA sequence against the constant region of the influenza virus nucleoprotein gene comprising:
Sense strand:
5′ GGAUCUUAUUUCUUCGGAGACdTdT 3′
Anti sense strand:
3′ dTdTCCUAGAAUAAAGAAGCCUCUG 5′
said sequence being inhibitory against influenza virus in animals including humans.

222. The siRNA sequence of claim 221 in the form of an aqueous suspension suitable for nasal inhalation.

223. The siRNA sequence of claim 221 in the form of a plasmid expressing intracellularly in animals including humans.

224. The siRNA sequence of claim 221 in the form of an AAV vector adapted to express intercellularly and establish a permanent inhibitory effect against influenza virus by integrating to the cellular chromosome of animals including humans.

225. A method comprising the administration to an animal including humans of a therapeutically effective amount of the siRNA sequence of claim 1.

226. The method of claim 225 wherein the administration is by nasal inhalation in the form of an aqueous mist.

227. The method of claim 225 wherein the administration is in the form of a plasmid.

228. The method of claim 225 wherein the administration is in the form of a AAV vector.

229. The method of claim 225 wherein the administration is effective against influenza virus A, B or C.

230. The method of claim 225 wherein the administration is effective against avian influenza (H5N1).

231. An RNA molecule comprising:
a core duplex region comprising a sense strand and an antisense strand, wherein the sequence of the sense strand or portion of the core duplex region comprises at least 15 consecutive nucleotides of any of the sequences presented in any one of SEQ ID NOs: 42,43,93, and 188.

232. An RNA molecule comprising:
a core duplex region comprising a sense strand and an antisense strand, wherein the sequence of the sense strand or portion of the core duplex region comprises 15 consecutive nucleotides of the sequence presented in SEQ ID NO: 42.

233. The KNA molecule of claim 232, wherein the 15 consecutive nucleotides of the sequence presented in SEQ ID NO: 42 is 5′ GGAUCUUAUUUCUUC 3′.

234. An RNA molecule comprising:
a core duplex region comprising a sense strand and an antisense strand, wherein the sequence of the sense strand or portion of the core duplex region comprises 19 consecutive nucleotides of the sequence presented in SEQ ID NO: 42.

235. The RNA molecule of claim 234, wherein the 19 consecutive nucleotides of the sequence presented in SEQ ID NO: 42 is 5′ UGAAGGAUCUUAUUUCUUC 3′.

236. An RNA molecule comprising:
a core duplex region comprising a sense strand and an antisense strand, wherein the sequence of the sense strand or portion of the core duplex region comprises 17 consecutive nucleotides of the sequence presented in SEQ ID NO: 43.

237. The RNA molecule of claim 236, wherein the 17 consecutive nucleotides of the sequence presented in SEQ ID NO: 43 is 5′ AAGGAUCUUAUUUCUUC 3′.

238. An RNA molecule comprising:
a core duplex region comprising a sense strand and an antisense strand, wherein the sequence of the sense strand or portion of the core duplex region comprises 18 consecutive nucleotides of the sequence presented in SEQ ID NO: 43.

239. The RNA molecule of claim 238, wherein the 15 consecutive nucleotides of the Sequence presented in SEQ ID NO: 43 is 5′ GGAUCUUAUUUCUUCGGA 3′.

240. An RNA molecule comprising:
a core duplex region comprising a sense strand and an antisense strand, wherein the sequence of the sense strand or portion of the core duplex region comprises 20 consecutive nucleotides of the sequence presented in SEQ ID NO: 43.

241. The RNA molecule of claim 240, wherein the 20 consecutive nucleotides of the sequence presented in SEQ ID NO: 43 is 5′ AAGGAUCUUAUUUCUUCGGA 3′.

242. An RNA molecule comprising:
a core duplex region comprising a sense strand and an antisense strand, wherein the sequence of the sense strand or portion of the core duplex region comprises 15 consecutive nucleotides of the sequence presented in SEQ ID NO: 93.

243. The RNA molecule of claim 242, wherein the 15 consecutive nucleotides of the sequence presented in SEQ ID NO: 93 is 5′ GGAUCUUAUUUCUUC 3′.

244. An RNA molecule comprising:
a core duplex region comprising a sense strand and an antisense strand, wherein the sequence of the sense strand or portion of the core duplex region comprises 19 consecutive nucleotides of the sequence presented in SEQ ID NO: 93.

245. The RNA molecule of claim 244, wherein the 19 consecutive nucleotides of the sequence presented in SEQ ID NO: 93 is 5′ GGAUCUUAUUUCUUCGGAG 3′.

246. An RNA molecule comprising:
a core duplex region comprising a sense strand and an antisense strand, wherein the sequence of the sense strand or portion of the core duplex region comprises 15 consecutive nucleotides of the sequence presented in SEQ ID NO: 188.

247. The RNA molecule of claim 246, wherein the 15 consecutive nucleotides of the sequence presented in SEQ ID NO: 188 is 5′ GGAUCUUAUUUCUUC 3′.

248. An RNA molecule comprising:
a core duplex region comprising a sense strand and an antisense strand, wherein the sequence of the sense strand or portion of the core duplex region comprises 19 consecutive nucleotides of the sequence presented in SEQ ID NO: 188.

249. The RNA molecule of claim 248, wherein the 19 consecutive nucleotides of the sequence presented in SEQ ID NO: 188 is 5′ GGAUCUUAUUUCUUCGGAG 3′.

250. An RNA molecule comprising:
a core duplex region comprising a sense strand and an antisense strand, wherein the sequence of the sense strand or portion of the core duplex region comprises 20 consecutive nucleotides of the sequence presented in SEQ ID NO: 188.

251. The RNA molecule of claim 250, wherein the 20 consecutive nucleotides of the sequence presented in SEQ ID NO: 188 is 5′ GGAUCUUAUUUCUUCGGAGA 3′.

252. An RNA molecule comprising a sense strand and an antisense strand, wherein the sense strand or portion of the RNA molecule comprises a sequence of the first 19 nucleotides of SEQ ID NO: 93, reading in a 5′ to 3, direction.

253. An RNA molecule comprising a sense strand and an antisense strand, wherein the sense strand or portion of the RNA molecule comprises a sequence of the first 19 nucleotides of SEQ ID NO: 188, reading in a 5′ to 3′ direction.

254. The RNA molecule of claim 231, further comprising 0 to 6 nucleotides at the 5′ end of the RNA molecule.

255. The RNA molecule of claim 231, further comprising 0 to 6 nucleotides at the 3′ end of the RNA molecule.

256. The RNA molecule of claim 231, further comprising 0 to 6 nucleotides at the 5′ end and at the 3′ end of the RNA molecule.

257. The RNA molecule of claim 231, wherein the RNA molecule is an siRNA or shRNA.

258. The RNA molecule of claim 231, wherein the RNA molecule is useful for treating or preventing influenza infection.

259. A cell comprising the RNA molecule of claim 231.

260. A transgenic animal comprising the RNA molecule of claim 231.

261. The transgenic animal of claim 260, wherein the transgenic animal is a human.

262. A vector comprising a nucleic acid operably linked to expression signals active in a host cell so that, when the construct is introduced into the host cell, the RNA molecule of claim 231 is produced inside the host cell that is targeted to a transcript specific to influenza virus, which transcript is involved in infection by or replication of influenza virus.

263. The vector of claim 262, wherein the vector is an adeno-associated virus (AAV) vector.

264. The vector of claim 263, wherein, when transformed into a host cell, the AAV vector expresses intracellularly and integrates into the host cell genome, thereby establishing a permanent inhibitory effect against influenza virus.

265. A method of treating or preventing infection by an influenza virus, the method comprising steps of: administering to a subject prior to, simultaneously with, or after exposure of the subject to the influenza virus, the vector of claim 262.

266. A method of treating or preventing infection by an influenza virus, the method comprising steps of: administering to a subject prior to, simultaneously with, or after exposure of the subject to the influenza virus, a composition comprising the RNA molecule of claim 231.

267. The method of claim 266, wherein the influenza virus is an influenza A virus, an influenza B virus, or an influenza C virus.

268. The method of claim 266, wherein the influenza virus is an influenza A virus or an influenza B virus.

269. The method of claim 266, wherein the influenza virus is H5N₁ influenza.

270. The method of claim 266, wherein the composition is administered intranasally.

271. The method of claim 266, wherein the composition is administered by inhalation.

272. A pharmaceutical composition comprising:
the RNA molecule of claim 231; and
a pharmaceutically acceptable carrier.

273. The pharmaceutical composition of claim 272, wherein the composition is formulated as an aerosol.

274. The pharmaceutical composition of claim 272, wherein the composition is formulated as a nasal spray.

275. A method of treating or preventing influenza virus replication, pathogenicity, or infectivity comprising administering the RNA molecule of claim 231 to a subject at risk of or suffering from influenza virus infection.

276. The method of claim 275, wherein the composition is administered by inhalation.

277. The method of claim 275, wherein the composition is administered as an aerosol.

278. The method of claim 275, wherein the composition is administered as an aerosol.


SEQUENCE LISTING (271 sequences)

SEQ ID NO: 1 - DNA, 21 bp, Artificial
  Influenza virus type A PB2 segment
aatcaagaagtacacatcagg

SEQ ID NO: 2 - DNA, 21 bp, Artificial
  Influenza virus type A PB2 segment
aagtacacatcaggaagacag

SEQ ID NO: 3 - DNA, 21 bp, Artificial
  Influenza virus type A PB2 segment
aatggatgatggcaatgaaat

SEQ ID NO: 4 - DNA, 21 bp, Artificial
  Influenza virus type A PB2 segment
aattacagcagacaagaggat

SEQ ID NO: 5 - DNA, 21 bp, Artificial
  Influenza virus type A PB2 segment
aacttactcatcgtcaatgat

SEQ ID NO: 6 - DNA, 21 bp, Artificial
  Influenza virus type A PB2 segment
aatgtgaggggatcaggaatg

SEQ ID NO: 7 - DNA, 21 bp, Artificial
  Influenza virus type A PB2 segment
aagcatcaatgaactgagcaa

SEQ ID NO: 8 - DNA, 21 bp, Artificial
  Influenza virus type A PB2 segment
aaggagacgtggtgttggtaa

SEQ ID NO: 9 - DNA, 21 bp, Artificial
  Influenza virus type A PB2 segment
aacgggactctagcatactta

SEQ ID NO: 10 - DNA, 21 bp, Artificial
  Influenza virus type A PB2 segment
aagaattcggatggccatcaa

SEQ ID NO: 11 - DNA, 21 bp, Artificial
  Influenza virus type A PB1 segment
aagcaggcaaaccatttgaat

SEQ ID NO: 12 - DNA, 21 bp, Artificial
  Influenza virus type A PB1 segment
aaccatttgaatggatgtcaa

SEQ ID NO: 13 - DNA, 21 bp, Artificial
  Influenza virus type A PB1 segment
aatccgaccttacttttctta

SEQ ID NO: 14 - DNA, 21 bp, Artificial
  Influenza virus type A PB1 segment
aagtgccagcacaaaatgcta

SEQ ID NO: 15 - DNA, 21 bp, Artificial
  Influenza virus type A PB1 segment
aacaggatacaccatggatac

SEQ ID NO: 16 - DNA, 21 bp, Artificial
  Influenza virus type A PB1 segment
aatgttctcaaacaaaatggc

SEQ ID NO: 17 - DNA, 21 bp, Artificial
  Influenza virus type A PB1 segment
aatgatgatgggcatgttcaa

SEQ ID NO: 18 - DNA, 21 bp, Artificial
  Influenza virus type A PB1 segment
aagatctgttccaccattgaa

SEQ ID NO: 19 - DNA, 21 bp, Artificial
  Influenza virus type A PA segment
aagcaggtactgatccaaaat

SEQ ID NO: 20 - DNA, 21 bp, Artificial
  Influenza virus type A PA segment
aatggaagattttgtgcgaca

SEQ ID NO: 21 - DNA, 21 bp, Artificial
  Influenza virus type A PA segment
ttgtgcgacaatgcttcaatc

SEQ ID NO: 22 - DNA, 21 bp, Artificial
  Influenza virus type A PA segment
aatgcttcaatccgatgattg

SEQ ID NO: 23 - DNA, 21 bp, Artificial
  Influenza virus type a PA segment
aatccgatgattgtcgagctt

SEQ ID NO: 24 - DNA, 21 bp, Artificial
  Influenza virus type A PA segment
aacaaatttgcagcaatatgc

SEQ ID NO: 25 - DNA, 21 bp, Artificial
  Influenza virus type A PA segment
aagagacaattgaagaaaggt

SEQ ID NO: 26 - DNA, 21 bp, Artificial
  Influenza virus type A PA segment
tagagcctatgtggatggatt

SEQ ID NO: 27 - DNA, 21 bp, Artificial
  Influenza virus type A PA segment
aacggctacattgagggcaag

SEQ ID NO: 28 - DNA, 21 bp, Artificial
  Influenza virus type A PA segment
aaccacacgaaaagggaataa

SEQ ID NO: 29 - DNA, 21 bp, Artificial
  Influenza virus type A PA segment
aacctgggacctttgatcttg

SEQ ID NO: 30 - DNA, 21 bp, Artificial
  Influenza virus type A PA segment A/WSN/33HIN1) strain
aagcaattgaggagtgcctga

SEQ ID NO: 31 - DNA, 21 bp, Artificial
  Influenza virus type A PA segment
aatgatccctgggttttgctt

SEQ ID NO: 32 - DNA, 21 bp, Artificial
  Influenza virus type A PA segment
aatgcttcttggttcaactcc

SEQ ID NO: 33 - DNA, 21 bp, Artificial
  Influenza virus type A HA segment
ttggagccattgccggtttta

SEQ ID NO: 34 - DNA, 21 bp, Artificial
  Influenza virus type A HA segment
ggagccattgccggttttatt

SEQ ID NO: 35 - DNA, 21 bp, Artificial
  Influenza virus type A HA segment
aatgggacttatgattatccc

SEQ ID NO: 36 - DNA, 21 bp, Artificial
  Influenza virus type A NP segment
aatcactcactgagtgacatc

SEQ ID NO: 37 - DNA, 21 bp, Artificial
  Influenza virus type A NP segment
aatcatggcgtcccaaggcac

SEQ ID NO: 38 - DNA, 21 bp, Influenza virus type A NP segment
aatagagagaatggtgctctc

SEQ ID NO: 39 - DNA, 21 bp, Influenza virus type A NP segment
aataaggcgaatctggcgcca

SEQ ID NO: 40 - DNA, 21 bp, Artificial
  Influenza virus type A NP segment
aaggcgaatctggcgccaagc

SEQ ID NO: 41 - DNA, 21 bp, Artificial
  Influenza virus type A NP segment
aatgtgcaacattctcaaagg

SEQ ID NO: 42 - DNA, 21 bp, Artificial
  Influenza virus type A NP segment
aatgaaggatcttatttcttc

SEQ ID NO: 43 - DNA, 21 bp, Artificial
  Influenza virus type A NP segment
aaggatcttatttcttcggag

SEQ ID NO: 44 - DNA, 21 bp, Artificial
  Influenza virus type A NP segment
aatgcagaggagtacgacaat

SEQ ID NO: 45 - DNA, 21 bp, Artificial
  Influenza virus type A NA segment
aatgaatccaaatcagaaaat

SEQ ID NO: 46 - DNA, 21 bp, Artificial
  Influenza virus NA segment
gaggacacaagagtctgaatg

SEQ ID NO: 47 - DNA, 21 bp, Artificial
  Influenza virus NA segment
gaggaatgttcctgttaccct

SEQ ID NO: 48 - DNA, 21 bp, Artificial
  Influenza virus type A NA segment
gtgtgtgcagagacaattggc

SEQ ID NO: 49 - DNA, 21 bp, Artificial
  Influenza M segment
aatggctaaagacaagaccaa

SEQ ID NO: 50 - DNA, 21 bp, Artificial
  Influenza virus type A M segment
aatcctgtcacctctgactaa

SEQ ID NO: 51 - DNA, 21 bp, Artificial
  Influenza virus type A M segment
acgctcaccgtgcccagtgag

SEQ ID NO: 52 - DNA, 21 bp, Artificial
  Influenza virus type A NA segment
actgcagcgtagacgctttgt

SEQ ID NO: 53 - DNA, 21 bp, Artificial
  Influenza virus type A M segment
actcagttattctgctggtgc

SEQ ID NO: 54 - DNA, 21 bp, Artificial
  Influenza virus type A M segment
agttattctgctggtgcactt

SEQ ID NO: 55 - DNA, 21 bp, Artificial
  Influenza virus type A M segment
aacagattgctgactcccagc

SEQ ID NO: 56 - DNA, 21 bp, Artificial
  Influenza virus type A M segment
aaggctatggagcaaatggct

SEQ ID NO: 57 - DNA, 21 bp, Artificial
  Influenza virus type A M segment
aatggctggatcgagtgagca

SEQ ID NO: 58 - DNA, 21 bp, Influenza virus type A M segment
actcatcctagctccagtgct

SEQ ID NO: 59 - DNA, 21 bp, Artificial
  Influenza virus type A M segment
aatttgcaggcctatcagaaa

SEQ ID NO: 60 - DNA, 21 bp, Artificial
  Influenza virus type A M segment
attgtggattcttgatcgtct

SEQ ID NO: 61 - DNA, 21 bp, Artificial
  Influenza virus type A M segment
aagaatatcgaaaggaacagc

SEQ ID NO: 62 - DNA, 21 bp, Artificial
  Influenza virus type A M segment
attttgtcagcatagagctgg

SEQ ID NO: 63 - DNA, 21 bp, Artificial
  Influenza NS segment
aagaactaggtgatgccccat

SEQ ID NO: 64 - DNA, 21 bp, Artificial
  Influenza virus type A NS segment
aactaggtgatgccccattcc

SEQ ID NO: 65 - DNA, 21 bp, Artificial
  Influenza virus type A NS segment
atcggcttcgccgagatcaga

SEQ ID NO: 66 - DNA, 21 bp, Artificial
  Influenza virus type A NS segment
gccgagatcagaaatccctaa

SEQ ID NO: 67 - DNA, 21 bp, Artificial
  Influenza virus type A NS segment
ggagtcctcatcggaggactt

SEQ ID NO: 68 - DNA, 21 bp, Artificial
  Influenza virus type A NS segment
aatgataacacagttcgagtc

SEQ ID NO: 69 - DNA, 21 bp, Artificial
  targeted to influenza virus type A PB2 segment
ggagacgugguguugguaatt

SEQ ID NO: 70 - DNA, 21 bp, Artificial
  targeted to influenza virus type A PB2 segment
uuaccaacaccacgucucctt

SEQ ID NO: 71 - DNA, 21 bp, Artificial
  targeted to influenza virus type A PB2 segment
cgggacucuagcauacuuatt

SEQ ID NO: 72 - DNA, 21 bp, Artificial
  targeted to influenza virus type A PB2 segment
uaaguaugcuagagucccgtt

SEQ ID NO: 73 - DNA, 23 bp, Artificial
  targeted to imfluenza virus type A PB1 segment
gcaggcaaaccauuugaaudtdt

SEQ ID NO: 74 - DNA, 21 bp, Artificial
  targeted to influenza virus type A PB1 segment
auucaaaugguuugccugctt

SEQ ID NO: 75 - DNA, 23 bp, Artificial
  targeted to influenza virus type A PB1 segment
caggauacaccauggauacdtdt

SEQ ID NO: 76 - DNA, 23 bp, Artificial
  targeted to influenza virus type A PB1 segment
guauccaugguguauccugdtdt

SEQ ID NO: 77 - DNA, 21 bp, Artificial
  targeted to influenza virus type A PB1 segment
gaucuguuccaccauugaatt

SEQ ID NO: 78 - DNA, 21 bp, Artificial
  targeted to influenza virus type A PB1 segment
uucaaugguggaacagauctt

SEQ ID NO: 79 - DNA, 23 bp, Artificial
  targeted to influenza virus type A PA segment
ugcuucaauccgaugauugdtdt

SEQ ID NO: 80 - DNA, 23 bp, Artificial
  targeted to influenza virus type A PA segment
caaucaucggauugaagcadtdt

SEQ ID NO: 81 - DNA, 21 bp, Artificial
  targeted to influenza virus type A PA segment
cggcuacauugagggcaagtt

SEQ ID NO: 82 - DNA, 21 bp, Artificial
  targeted to influenza virus type A PA segment
cuugcccucaauguagccgtt

SEQ ID NO: 83 - DNA, 21 bp, Artificial
  targeted to influenza virus type A PA segment
gcaauugaggagugccugatt

SEQ ID NO: 84 - DNA, 21 bp, Artificial
  targeted to influenza virus type A PA segment
ucaggcacuccucaauugctt

SEQ ID NO: 85 - DNA, 21 bp, Artificial
  targeted to influenza virus type A PA segment
ugaucccuggguuuugcuutt

SEQ ID NO: 86 - DNA, 23 bp, Artificial
  targeted to influenza virus type A PA segment
aagcaaaacccagggaucadtdt

SEQ ID NO: 87 - DNA, 21 bp, Artificial
  targeted to influenza virus type A PA segment
ugcuucuugguucaacucctt

SEQ ID NO: 88 - DNA, 21 bp, Artificial
  targeted to influenza virus type A PA segment
ggaguugaaccaagaagcatt

SEQ ID NO: 89 - DNA, 21 bp, Artificial
  targeted to influenza virus type A NP segment
uagagagaauggugcucuctt

SEQ ID NO: 90 - DNA, 23 bp, Artificial
  targeted to influenza virus type A NP segment
gagagcaccauucucucuadtdt

SEQ ID NO: 91 - DNA, 21 bp, Artificial
  targeted to influenza virus type A NP segment
uaaggcgaaucuggcgccatt

SEQ ID NO: 92 - DNA, 21 bp, Artificial
  targeted to influenza virus type A NP segment
uggcgccagauucgccuuatt

SEQ ID NO: 93 - DNA, 21 bp, Artificial
  targeted to infuenza virus type A NP segment
ggaucuuauuucuucggagtt

SEQ ID NO: 94 - DNA, 21 bp, Artificial
  targeted to influenza virus type A NP segment
cuccgaagaaauaagaucctt

SEQ ID NO: 95 - DNA, 23 bp, Artificial
  targeted to influenza type A M segment
ccgaggucgaaacguacgudtdt

SEQ ID NO: 96 - DNA, 21 bp, Artificial
  targeted to influenza virus type A M segment
acguacguuucgaccucggtt

SEQ ID NO: 97 - DNA, 21 bp, Artificial
  targeted to influenza virus type A M segment
cagauugcugacucccagctt

SEQ ID NO: 98 - DNA, 21 bp, Artificial
  targeted to influenza virus type A M segment
gcugggagucagcaaucugtt

SEQ ID NO: 99 - DNA, 21 bp, Artificial
  targeted to influenza virus type A M segment
uggcuggaucgagugagcatt

SEQ ID NO: 100 - DNA, 23 bp, Artificial
  targeted to influenza virus type A M segment
ugcucacucgauccagccadtdt

SEQ ID NO: 101 - DNA, 23 bp, Artificial
  targeted to influenza virus type A M segment
gaauaucgaaaggaacagcdtdt

SEQ ID NO: 102 - DNA, 21 bp, Artificial
  targeted to influenza virus type A M segment
gcuguuccuuucgauauuctt

SEQ ID NO: 103 - DNA, 21 bp, Artificial
  targeted to influenza virus type A NS segment
cggcuucgccgagaucagaat

SEQ ID NO: 104 - DNA, 21 bp, Artificial
  targeted to influenza virus type A NS segment
ucugaucucggcgaagccgat

SEQ ID NO: 105 - DNA, 21 bp, Artificial
  targeted to influenza virus type A NS segment
guccuccgaugaggacucctt

SEQ ID NO: 106 - DNA, 21 bp, Artificial
  targeted to influenza virus type A NS segment
ggaguccucaucggaggactt

SEQ ID NO: 107 - DNA, 21 bp, Artificial
  targeted to influenza virus type A NS segment
ugauaacacaguucgaguctt

SEQ ID NO: 108 - DNA, 21 bp, Artificial
  targeted to influenza virus type A NS segment
gacucgaacuguguuaucatt

SEQ ID NO: 109 - DNA, 19 bp, Artificial
  influenza virus type A PA segment
tgcttcaatccgatgattg

SEQ ID NO: 110 - DNA, 21 bp, Artificial
  Aequoria victoria green fluorescent protein
ggcuacguccaggagcgcauu

SEQ ID NO: 111 - DNA, 21 bp, Artificial
  Aequoria victoria green fluorescent protein
ugcgcuccuggacguagccuu

SEQ ID NO: 112 - DNA, 18 bp, Artificial
  mRNA
tttttttttttttttttt

SEQ ID NO: 113 - DNA, 22 bp, Artificial
  influenza virus type A NP v RNA
ctcgtcgcttatgacaaagaag

SEQ ID NO: 114 - DNA, 36 bp, Artificial
  influenza virus type A NP cRNA
atatcgtctcgtattagtagaaacaagggtattttt

SEQ ID NO: 115 - DNA, 21 bp, Artificial
  influenza virus type A NS vRNA
caggacatactgatgaggatg

SEQ ID NO: 116 - DNA, 35 bp, Artificial
  influenza virus type A NS cRNA
atatcgtctcgtattagtagaaacaagggtgtttt

SEQ ID NO: 117 - DNA, 22 bp, Artificial
  influenza virus type A NP RNA
ctcgtcgcttatgacaaagaag

SEQ ID NO: 118 - DNA, 21 bp, Artificial
  influenza virus type A NP RNA
agatcatcatgtgagtcagac

SEQ ID NO: 119 - DNA, 21 bp, Artificial
  influenza virus type A NS RNA
caggacatactgatgaggatg

SEQ ID NO: 120 - DNA, 21 bp, Artificial
  influenza virus type A NS RNA
gtttcagagactcgaactgtg

SEQ ID NO: 121 - DNA, 2233 bp, Artificial
  influenza virus type A PA segment
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 122 - DNA, 2183 bp, Artificial
  influenza virus type A PA segment
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 123 - DNA, 2233 bp, Artificial
  influenza virus type A PA segment
agcaaaagcaggtactgatccgaaatggaagaatttgtgcgacaatgcttcaatccgatgattgtcgagcttgctgaaaaagcaatgaaagagtatggagaggatcggaaaatcgaaacaaacaaatttgcagcaatatgcactcacttggaagtatgcttcatgtattcagattttcatttcatcaatgagcaaggcgagtcaataatagtagagcttgatgatccaaatgcacttttgaagcacagatttgaaataatagagggaagagatcgcacaatggcctggacagtagtaaacagtatttgcaacactacaggagctgagaaaccgaagtttctgccagatttgtatgattacaaggagaatagattcatcgagattggagtgacaaggagggaagtccacatatactatcttgaaaaggccaataaaattaaatctgagaagacacacatccacattttctcattcactggggaagaaatggccacaaaggccgactacactctcgatgaggaaagcagggctaggatcaagaccagactattcaccataagacaagaaatggctagcagaggcctctgggattcctttcgtcagtccgaaagaggcgaagaaacaattgaagaaagatttgaaatcacagggacaatgcgcaggctcgccgaccaaagtctcccgccgaacttctcctgccttgagaattttagagcctatgtggatggattcgaacccaacggctacattgagggcaagctttctcaaatgtccaaagaagtaaatgctaaaattgagccttttctgaaaacaacaccaagaccaattaaacttccggatgggcctccttgctctcagcggtccaaattcctgctgatggatgctttaaaattaagcattgaggacccaagtcacgaaggagagggaataccactatatgatgcgatcaagtgtatgagaacattctttggatggaaagaaccctatgttgttaaaccacacgataagggaataaatccaaattatctgctgtcatggaagcaattactggcagaactgcaggacattgagaatgaggagaagattccaagaaccaaaaacatgaagaaaacgagtcagctaaagtgggcacttggtgagaacatggcaccagagaaggtagactttgacgactgtagagatataagcgatttgaagcaatatgatagtgatgaacctgaattaaggtcactttcaagctggatccagaatgagttcaacaaggcatgcgagctgaccgattcaatctggatagagctcgatgagattggagaagatgtggctccaattgaacacattgcaagcatgagaaggaattacttcacagcagaggtgtctcagtgcagagccacagaatatataatgaagggggtatacattaatactgccttgcttaatgcatcctgtgcagcaatggacgatttccaactaattcccatgataagcaaatgtagaactaaagagggaaggcgaaagaccaatttatatggtttcatcataaaaggaagatctcacttaaggaatgacaccgacgtggtaaactttgtgagcatggagttttctctcactgacccaagacttgagccacacaaatgggagaagtactgtgttcttgagataggagatatgctactaagaagtgccataggccaggtgtcaaggcccatgttcttgtatgtgaggacaaatggaacatcaaagattaaaatgaaatggggaatggagatgaggcgttgcctccttcagtcactccaacaaatcgagagtatgattgaagccgagtcctctgtcaaggagaaagacatgaccaaagagtttttcgagaataaatcagaaacatggcccattggagagtcccccaaaggagtggaagaaggttccattgggaaggtctgcaggactttattagccaagtcggtattcaatagcctgtatgcatctccacaattagaaggattttcagctgaatcaagaaaactgcttcttgtcgttcaggctcttagggacaatcttgaacctgggacctttgatcttggggggctatatgaagcaattgaggagtgcctgattaatgatccctgggttttgcttaatgcgtcttggttcaactccttcctaacacatgcattaagatagttgtggcaatgctactatttgctatccatactgtccaaaaaagtaccttgtttctact

SEQ ID NO: 124 - DNA, 2209 bp, Artificial
  influenza virus type A PA segment
atggaagattttgtacgacaatgctttaatccgatgattgtcgaacttgcggaaaaggcaatgaaagagtatggagaggatcttaaaatcgaaacaaacaaatttgcagcaatatgcactcacttggaagtatgcttcatgtattcagattttcatttcatcaatgagcaaggcgagtcaatagtggtagaacttgatgatccaaatgcacttttgaagcacagatttgaaataatagagggaagagaccgcacaatggcctggacagtagtaaacagtatttgcaacaccacaggagctgagaaaccgaagtttctgccagatttgtatgattacaaggagaatagattcatcgagattggagtgacaaggagagaagtccacatatactaccttgaaaaggccaataaaattaaatctgagaatacacacatccacattttctcattcactggggaagaaatggccacaaaggccgactacactctcgatgaggaaagcagggctaggatcaaaaccagactattcaccataagacaagagatggccaacagaggcctctgggattcctttcgtcagtccgaaagaggcgaagaaacaattgaagaaagatttgaaatcacagggacaatgcgcaggcttgccgaccaaagtctcccgccgaacttctcctgccttgagaattttagagcctatgtggatggattcgaaccgaacggctacattgagggcaagctttctcaaatgtccaaagaagtgaatgcaaaaattgaaccttttctgaaaacaacaccaagaccaattagacttccggatgggcctccttgttttcagcggtccaaattccttctgatggatgctttaaagttaagcattgaggatccaagtcacgagggggagggaataccactatatgatgcgatcaaatgcatgagaacattttttggatggaaagaaccctatattgttaaaccacacgaaaaggggataaatccaaattatctgctgtcatggaagcaagtactggcagaactgcaggacattgaaaatgaggagaaaattccaagaactaaaaacatgaagaaaacgagtcagctaaagtgggcacttggtgagaacatggcaccagagaaggtagactttgacaactgtagagacgtaagcgatttgaagcaatatgatagtgacgaacctgaattaaggtcactttcaagctggatccagaatgagttcaacaaggcatgcgagctgaccgattcaacttggatagagctcgatgagattggagaagacgtggctccaattgaatacattgcaagcatgagaaggaattacttcacagcagaggtgtcccattgcagagccacagaatatataatgaagggggtatacattaatactgccttgcttaatgcatcctgtgcagcaatggacgatttccaactaattcccatgataagcaagtgtagaactaaagaaggaaggcgaaagaccaatttatatggcttcatcataaaaggaagatctcacttaaggaatgacaccgacgtggtaaactttgtgagcatggagttttctctcactgacccgagacttgagccacacaaatgggagaaatactgtgtccttgagataggagatatgctactaagaagtgctataggccagatgtcaaggcctatgttcttgtatgtgagaacaaatggaacatcaaagattaaaatgaaatggggaatggagatgaggcgttgcctccttcagtcactccaacaaatcgagagtatgattgaagccgagtcctctgtcaaggagaaagacatgaccaaagagttttttgagaataaatcagaaacatggcccattggggagtcccccaagggagtggaagatggttccattgggaaggtctgcaggactttattggccaagtcggtattcaatagcctgtatgcatccccgcaattggaagggttttcagctgagtcaagaaaactgcttcttgtcgttcaggctcttaaggacaatcttgaacctggaacctttgatcttgaggggctatatgaagcaattgaggagtgcctgattaatgatccctgggttttgcttaatgcgtcgtggttcaactccttcctaacacatgcattaagatagttgtggcaatgctactatttgctatccatactgtccaaaaaagtaccttgtttctact

SEQ ID NO: 125 - DNA, 2233 bp, Artificial
  influenza virus type A PA segment
agcaaaagcaggtactgatccaaaatggaagactttgtgcgacaatgcttcaatccaatgattgtcgagcttgcggaaaagacaatgaaggagtatggggaagatccgaagattgaaacaaacaagttcgctgcaatatgcacacacttagaagtctgcttcatgtattcagacttccatttcattgacgaacgaggcgaatcaataattgtggaatctggtgatccgaatgcattgttgaaacaccggtttgaaataattgaaggaagagaccgagcaatggcctggacagtggtgaatagcatctgcaacaccacaggagtcgataaacccaaatttcttccggatctatacgactacaaggaaaaccgattcactgaaattggtgtgacacggagggaagttcatatatattacttagagaaagctaacaagataaaatccgagaaaacacatatccacatcttctcattcactggagaagaaatggccactaaagctgactacacccttgatgaagagagcagggcaagaatcaaaaccagactattcaccataagacaggaaatggcaagcaggggtctatgggactcctttcgtcagtccgagagaggcgaagagacaattgaagaaagatttgaaatcacagggaccatgcgtaggcttgccgaccaaagtctcccacctaacttctccagccttgaaaactttagagcctatgtggatggatttaaaccgaacggctgcattgagggcaagctttctcaaatgtcgaaagaagtgaacgccagaattgagccatttctgaagacaacaccacgtcctctcagattgcctgatggacctccctgctcccagcggtcgaaattcttgctgatggatgctctgaaattaagcattgaggacccgagccatgagggggaggggataccgctatatgatgcgatcaaatgcatgaaaacattctttggctggagagagcccaacatcatcaaaccacacgaaaaaggcataaatccaaattatctcctggcttggaagcaggtgctggcagaactccaggatattgaaaatgaggataaaatcccaaaaacaaagaacatgaagaaaacaagccaattaatgtgggcactcggagagaatatggcaccggaaaaagtggactttgaggattgcaaagacattgatgatctgaaacagtaccacagtgatgagccagagcttagatcgctagcaagctggatccagaatgagttcaacaaggcatgtgaattgaccgattcgagctggatagaacttgatgagataggggaagatgttgccccaattgagcacattgcaagtatgagaaggaactacttcacagcggaggtgtcccattgcagggctactgagtacataatgaagggggtttacataaatacagctttgctcaatgcatcttgtgcagccatggatgacttccaactgattccaatgataagcaaatgcagaacaaaagaaggaaggaggaggacaaacctgtatgggttcattgtaaaaggaaggtcccatttgagaaatgatactgacgtggtgaactttgtgagtatggaattctcccttactgacccaaggctggagccacacaaatgggaaaagtactgtgttcttgaaataggggaaatgctcttgcggactgcaataggtcaagtgtcaaggcccatgttcctgtatgtgagaaccaacggaacctcaaaaattaagatgaaatgggggatggaaatgaggcgctgccttcttcaatctcttcaacagattgagagcatgatcgaggctgagtcttctatcaaagagaaagacatgaccaaagaattctttgaaaacagatcggagacatggccaattggagagtcacctaagggagtggaggaaggctccatcgggaaggtgtgcagaaccttactagcaaaatctgtgttcaacagcctatattcatctccacaactcgaaggattttcagctgaatcgagaaaactactactcattgttcaagcacttagggacaacctggaacctggaaccttcgatcttgaagggctatatggagcaattgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 126 - DNA, 2233 bp, Artificial
  influenza virus type A PA segment
agcaaaagcaggtactgatccgaaatggaagactttgtgcgacaatgcttcaatccaatgattgtcgagcttgcggaaaagacaatgaaggaatatggggaagacccgaaaattgaaacaaataagttcgctgcaatatgcacacacttagaagtctgcttcatgtattcagacttccatttcattgacgaacgaggcgaatcaataattgtggaatctggtgatccaaatgcattgttgaagcacaggtttgaaataattgaaggaagagaccgagcaatggcctggacagtggtgaatagcatctgcaacacaacaggagtcgataaacccaaatttcttccggatctatacgactacaaggaaaaccgattcactgaaattggtgtgacacggagggaagttcacatatattacttagaaaaagctaacaagataaaatccgagaaaacacatatccacatcttttcattcactggagaagaaatggccactaaagctgactacacccttgatgaagagagcagggcaagaataaaaaccagactattcaccataagacaggaaatggcaagcaggggtctatgggattcctttcgtcagtccgagagaggcgaagagacaattgaagaaagatttgaaatcacagggaccatgcgtaggcttgccgaccaaagtctcccacctaacttctccagccttgaaaactttagagcctatgtggatggattcaaaccgaacggctgcattgagggcaagctttctcaaatgtcgaaagaagtgaacgccagaattgagccatttctgaagacaacaccacgtcccctcagattgcctgatggacctccctgctcccagcggtcgaaattcttgctgatggatgctctgaaattaagcattgaggacccgagccatgagggggaggggataccgctatatgatgcgataaaatgcatgaaaacattcttcggctggagagagcccaacatcatcaagccacacgagaagggcataaatcccaattatcttctggcttggaagcaggtgctggcagaactccaggatattgaaaatgaggataaaatcccaaaaacaaagaacatgaagaaaacaagccaattaatgtgggcactcggggagaatatggcaccggaaaaattggactttgaggactgcaaagatattggcgatctgaaacagtatcaaagtgatgagccagagctcagatcgatagcaagctggatccagagtgagttcaacaaggcatgtgaattgaccgattcgagctggatagaactcgatgagataggggaagatgttgccccaattgagcacattgcaagcatgagaaggaactacttcacagcggaagtgtctcattgcagggccactgagtacataatgaagggggtttacataaatacagctttgctcaatgcatcttgtgcagccatggatgacttccaactgattccaatgataagcaaatgcagaacaaaagaaggaagaaggaagacaaacctgtatgggttcattataaaaggaaggtcccatttgagaaatgatactgacgtggtgaactttgtgagtatggaattctcccttactgacccaaggctggagccacacaaatgggaaaagtactgtgttcttgaagtaggggaaatgctcttgcggactgcaataggccaggtgtcaaggcccatgttcctgtatgtgagaactaacggaacctccaaaattaagatgaaatgggggatggaaatgagacgctgccttcttcaatctcttcaacagattgagagcatgatcgaggctgagtcttctatcaaagagaaagacatgaccaaagaattctttgaaaacagatcggagacatggccaattggagagtcacctaagggagtggaggaaggctcaatcgggaaggtgtgcagaaccttactagcaaaatctgtgttcaacagcctatattcatctccacaactcgaaggattttcagctgaatcgagaaaactactactcattgttcaagcacttagggacaacctggaacctggaacctttgatcttgaagggctatatggagcaattgaggagtgcctgattaatgatccctgggttttgcttaatgcatcttggttcaactccttcctcacacatgcactaaaatagttgtggcaatgctactatttgctatccatactgtccaaaaaagtaccttgtttctact

SEQ ID NO: 127 - DNA, 2182 bp, Artificial
  influenza virus type A PA segment
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcactatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactcttccttacacatgcattgagttagttgtggcagtgctactatttgctatccatactgtccaaaaaagtaccttgtttctact

SEQ ID NO: 128 - DNA, 2233 bp, Artificial
  influenza virus type A PA segment
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 129 - DNA, 2183 bp, Artificial
  influenza virus type A PA segment
agcgaaagcaggtactgatccaaaatggaagaatttgtgcgacaatgcttcaatccaatgatcgtcgagcttgcggaaaagacaatgaaagaatatggagaggacccgaagattgaaacaaacaaattcgcagcaatatgcacacatttggaagtgtgtttcatgtattcagatttccactttattgatgaacggggagagtcaaagattgtagaatctggtgacccaaatgcactcttgaagcaccgatttgagataattgaaggaagagatcgcacgatggcctggacggtggtgaatgtatgattataaggagaaccgattcattgaaattggagtgacaagaagggaggtccacatatactacttagaaaaagccaataagataaaatctgagaagacacacatccacatcttctcattcactggggaagaaatggccactaaggcggactacactcttgatgaagagagcagagcaaggatcaaaaccaggctattcaccataagacaagaaatggccagtagaggcctctgggattcctttcgtcagtccgagagaggcgaagagacaattgaagaaagatttgaaatcacaggaaccatgcgcaggcttgccgaacaaagtctcccaccgaacttctccagccttgaaaactttagagcctatgtggatggattcaaaccgaacggctgcattgagggcaagctttctcaaatgtccaaagaagtaaatgcaagaatcgagccattcttgaagacaacaccacgccctctcagattacctgatgggcctccctgttctcagcggtcaaaattcctactgatggatgctctgaaattaagcattgaagacccaagtcatgagggggaggggataccactacatgatgcaatcaaatgcatgaagacattctttggctggaaagagcccaatattgtcaaaccgcatgagaagggcataaatcccaactatctcctggcttggaaccaggtgctagcagaactgaaggatattgagaatgaggagaaaattccaaaaacaaaaaatatgaagaaaacaagccagttaaagtgggcacttggtgagaacatggcaccagagaaagtagactttgaggattgcaaggacattagcgatctgaagcagtatgacagtgatgagccagaacagagatcactagcgagttggatccagagtgaattcaacaaagcatgtgagctgaccgattcaggttggatagaacttgatgaaataggagaagatgtagccccaatcgagcacattgcaagtatgaggagaaactatttcacagcggaagtgtctcactgcagggcaacggagtacataatgaaaggggtatacataaacacggccttgctcaatgcatcttgtgcagctatggatgacttccagctgatcccaatgataagcaaatgcaggaccaaagaaggaagacggaagacgaatctgtatggattcattataaaaggaagatctcacttgaggaatgatactgatgtggtgaattttgtgagcatggagttctctctcactgacccgaggcttgagccacacaagtgggagaagtattgtgttcttgaaataggagacatgctcctgcggactgcaataggccaagtatcaaggcccatgttcctgtatgtgagaaccaatggaacctccaaaatcaagatgaaatggggtatggagatgagacgttgccttcttcagtcccttcaacaggttgaaagcatggttgaggctgagtcctctgtcaaggagaaagacatgactaaggaattctttgaaaacaagtcaaagacgtggcccattggagaatcacctaaaggagtggaagaaggttccatcgggaaagtgtgcaggaccttactggcgaagtctgtatttaacagcttatatgcatccccacaacttgagggattttcagcggaatctagaaaactgctcctcattgttcaggctcttagagacaacctggaacctggaaccttcgatcttggagggctatatggagcaattgaggagtgcctgattaatgatccctgggttttgcttaatgcatcttggttcaactccttcctcacacatgcactgaaatagttgtggcaatgctactatttgctatccatactgtccaaaaaagtaccttgtttctact

SEQ ID NO: 130 - DNA, 2090 bp, Artificial
  influenza virus type A PA segment
atggaagacttcgtgcgacaatgcttcaatccaatgattgtcgagcttgcggaaaaggcaatgaaagaatatggagagggcccgaaaatcgaaacaaacaaatttgcagcaatatgcactcatttggaagtgtgtttcatgtattcagactttcacttcatcgatgagcgaggcgaatcaataattgtagaatccggagatccgaatgccctcttgaagcacagatttgaaataattgagggaagagatcgcacaatggcctggacagtggtgaacagcatctgtaacactacaggggttgagaagccaaggtttctcccagatctatatgactacaaggagaacaggttcattgagattggagtgacaaggagagaagtccacatatactacctggaaaaggccaataaaataaagtctgagaagacacatatccacatcttctcgttcacaggagaagagatggccacaaaggctgactacactcttgatgaagaaagtagggccagaatcaaaactagactgttcaccataaggcaggaaatggccagtagaggtctctgggattcctttcgtcagtccgagagaggcgaagagacaattgaagaaagatttgaaatcacaggaacaatgcgcaggcttgccgaccaaagtctcccaccgaacttctccagccttgaaaactttagagcctatgtggatggattcgaaccgaacggctgcattgagggcaagctttctcaaatgtccaaagaagtgaatgcaagaattgaaccctttttgaagacaacaccacgcccactcaagctaccagatgggcctccctgctcccagcggtccaaattcctgctaatggacgctttgaaattaagcattgaggacccaagccatgaaggagaagggataccgctatatgatgcaatcaaatgcatgaaaacattctttgggtggaaagaacccaatattgttaaaccacatgaaaaaggaataaatccgaattacctcttggcatggaaacaagtactagcggaactacaggatcttgaaaatgaagagaaaattccaaagactaaaaacatgaagaaaacaagccaattaaagtgggcacttggtgagaatatggcaccagaaaaagtggattttgaggactgcaaggatgtcagcgatctgaagcaatatgacagtgatgaaccggaaccgagatcgcttgcaagttggattcagagtgagttcaataaggcgtgtgaactgactgattcaagctggatagagcttgacgagattggggaagatgttgccccaattgagcacattgcaagcatgaggaggaattatttcacagcggaggtgtctcattgtagagccacagaatacataatgaaaggggtatacatcaatactgccttgctcaatgcatcctgtgcggctatggatgactttcaactgattccaatgatcagcaagtgtagaactaaagagggaagaagaaagacaaatttgtatgggttcattataaaagggagatcccacctgaggaacgacaccgatgtggtaaactttgtgagcatggagttttccctcactgacccgaggcttgaaccgcacaaatgggagaagtactgtgttcttgaaataggggacatgcttctaagaactgccataggccaagtttcgaggcccatgttcctgtatgtgagaacgaatgggacctccaaaatcaaaatgaaatgggggatggaaatgagacgctgtcttctccagtcccttcaacaaattgagagtatgattgaagccgagtcctctgtcaaagagaaggacatgaccaaagggggtggaggaaggatccattggaaaggtctgcaggactctgttggcaaagtctgtattcaacagcttgtacgcatctccacagctggaaggtttctcagctgaatcaaggaaactgcttcttatcgttcaggctcttagggacaacctggaacctggaacctttgatcttggaggattgcatgaagcaattgaggagtgcctgattaatgacccctgggttttgcttaatgcatcttggtttaactccttcctcacacatg

SEQ ID NO: 131 - DNA, 2133 bp, Artificial
  influenza virus type A PA segment
agcaaaagcaggtactgatccaaaatggaagactttgtgcgacaatgcttcaatccaatgatcgtcgagcttgcggaaaaggcgatgaaagaatatggagaggacccgaaaattgaaacaaacaaatttgcagcaatatgcactcacttggaagtctgcttcatgtactcggatttccactttattaatgaactgggcgagtcagtgatcatagagtctggtgatccaaatgctcttttgaagcacagatttgaaatcattgaagggagagatcgaacaatggcatggacagtagtgaacagtatctgcaacaccacaagagctgaaaaacccaagttcctcccagatttgtacgactataaagagaacaggtttgttgaaattggtgtgacaaggagagaagttcacatatactacttggagaaagccaacaaaataaagtctgagaaaacacatattcacattttctcatttacaggagaggaaatggctacaaaagcggattatacccttgatgaagaaagtagagccaggatcaaaaccagactattcaccataagacaagaaatggccagcagaggcctttgggactcctttcgtcagtccgagagaggcgaagagacaattgaagaaagatttgaaatcacagggacaatgcgcaggcttgccgattacagtctcccaccgaacttctccagccattttcaaagacaacaccccggccactcaggacaccgggtggtccaccctgttatcagcgatccaaattcttgctgatggatgctctgaaatttagcattgaggatccaagtcacgagggagagggaataccgctgtatgatgccatcaaatgcatgaaaaccttctttggatggaaagagcccaatattgttaaaccacatgaaaagggtataaacccaaactatctccaggcttggaagcaagtgttagcagaactacaggacctcgaaaacgaagaaaaaatccctaagaccaagaatatgaaaaaaacaagtcaattgaaatgggcacttggtgagaatatggcgccagagaaagtggattttgaggattgtaaagacatcagtgatttgaaacagtatgacagtgatgagccagaaacaaggtcccttgcaagttggattcaaagtgagttcaacaaagcttgtgagctgacagattcaagctggatagagctcgatgaaattggggaggatgttgccccaatagaacacattgcgagcatgaggaggaattattttactgctgaggtttcccattgtagagcaactgaatatataatgaagggagtatacatcaacactgctctactcaatgcatcctgcgctgcgatggatgacttccaattaatcccgatgataagcaaatgcaggaccaaggaagggagaaggaagacaaatttgtatggattcatcataaagggaaggtcccatttaagaaatgacactgacgtggtaaactttgtaagcatggagttttctctcaccgatccaagacttgagccacacaattgggagaagtactgtgttctagaaatcggagacatgctcctaagaactgctgtaggccaagtgtcaagacccatgtttttgtatgtaaggaccaatgggacctctaaaattaaaatgaaatggggaatggaaatgaggcgctgcctccttcagtctctacagcagattgaaagcatgactgaagctgagtcctcagtcaaagaaaaggacatgaccaaagaattctttgagaacaaatcggagacatggcctataggagagtcccccaaaggagtggaagaaggctcaatcgggaaagtttgcaggaccttgttagcaaaatctgtgtttaacagtttatatgcatctccacaactcgaagggttttcagctgaatctaggaaaatacttctcattgttcaggcccttagggacaacctggaacctggaacctttgatattggggggctatatgaatcaattgaggagtgtctgattaatgatccctgggttttgctcaatgcatcttggttcaactccttccttacacatgcactaaagtagttgtggcaatgctactatttgctatccatactgtccaaaaaagtaccttgtttctact

SEQ ID NO: 132 - DNA, 2233 bp, Artificial
  influenza virus type A PA segment
agcaaaagcaggtactgattcaaaatggaagactttgtgcgacaatgcttcaatccaatgatcgtcgagcttgcggaaaaggcaatgaaagaatatggagaggatccaaaaatcgagacaaacaaattcgctgcaatatgcacacacctggaagtgtgtttcatgtattcagacttccatttcattgatgaacggggtgagtcgataattgttgagtctggtgatccaaatgcactcttaaaacatcgatttgaaataatcgaaggaagagaccgtactatggcctggacagtggtgaatagcatttgcaacaccacaggagttgagaagccaaagtttcttccggacttatatgattataaagaaaatcgtttcattgaaattggagtgacaaggagggaggtccatatatactatctagaaaaggccaataagataaagtctgagaagacacacatccatatcttttcattcactggagaagaaatggccacaaaagcagactacactcttgatgaagagagtagagcaaggatcaaaaccagactattcactataagacaagaaatggccagtagaggtctctgggattcctttcgtcagtccgagagaggcgaagagacaattgaagaaagatttgaaattacaggaaccatgcgcaggctcgccgaccaaagtctcccaccgaacttctccagccttgaaaactttagagcctatgtggatggattcgaaccgaacggctgcattgagggcaagctttctcaaatgtccaaagaagtaaatgcaagaattgaaccatttttgaagacaacaccacgccctctcagattaccagaagggcctccctgctctcagcggtcaaaatttctgttgatggatgctctgaagcttagcattgaagacccgagtcatgagggtgagggaataccactgtatgatgctatcaaatgtatgaagaccttttttggctggaaagagcccaacattgtcaagccacatgagaggggcataaaccctaattatctcctggcttggaagcaagtgcaagcagaactgcaggatattgaaaatgaagacaagattccaaagacaaaaaacatgaagaaaacaagccaattaaagtgggcacttggtgagaacatggcaccagagaaagtggactttgaagattgcaaggatgtcagcgatttgaaacagtatgacagcgatgagccagaacaaaggtcgctagcaagttgggtccaaagtgaattcaacaaagcttgtgaattgactgattcaagctggatagaactcgatgaaataggggaaaatgtcgccccaatcgagcatattgcaagcatgaggaggaattattttacagctgaagtgtctcactgcagggcaacagagtacataatgaagggagtgtacataaactcagctttactcaacgcctcttgtgcagccatggatgattttcagttgatcccaatgataagcaaatgcagaaccaaagaaggacgacggaaaacaaatttgtatggattcatcataaagggaaggtctcatttgaggaatgatactgatgtggtgaattttgtgagcatggaattttctcttactgaccctagattagaaccacacaagtgggagaagtattgtgtccttgaaataggggatatgctcctacgaactgcaataggccaagtttcaagacccatgtttctgtatgtgacaaccaatggaacttccaagatcaaaatgaaatggggtatggagatgaggcgttgtcttcttcaatccctccagcaaatcgaaagcatgattgaggccgagtcctctgtcaaggaaaaagacatgactaaagaattctttgaaaacaagtcggagacatggcccattggagaatcacccaaaggagtagaagaaggttccattgggaaagtatgcaggactctgctagcaaagtctgtattcaacagcttgtatgcatctccacaacttgaaggtttttcagctgagtcaagaaagctgcttctcattgttcaggcacttagggacaacctggaacctggcaccttcgatcttggggggctatatgaagcaattgaggagtgcctgattaatgatccctgggttttgcttaatgcatcttggttcaactccttcctcacacatgcactgaaatagttgtggcaatgctactatttgctatacatcctgtccaaaaaagtaccttgtttctact

SEQ ID NO: 133 - DNA, 1635 bp, Artificial
  influenza virus type A PA segment
agcaaaagcagggcaaggatcaaaactaggctgttcaccataagacaggaactggctagcaggggtctatgggattcctttcgtcagtccgagagaggcgaagagacaattgaagaaagatttgaaatcacaggaacaatgcgcaggcttgccgaccaaagtctcccaccgaatttctccagccttgaaaattttagagcctatgtggatggattcgaaccgaacggctgcattgagggcaagctttctcagatgtcaaaagaagtaacggccagaattgagccctttcttaaaacaacaccacgtcctctaagactgccgggtggacctccctgttcccaaaggtcaaaattcttactgatggatgctctgaaattaagcattgaggacccgagtcatgagggagaggggataccgctgtatgatgcgatcaaatgcatgaaaacatttttcggctggaaagagcccaaaattatcaagtcacatgagaagggtataaacccaaattatctcctagcttggaagcaggtgctggcagagctccaggacattgaaaatgatgaaaagatcccaaaaacaaagaacatgaagaaaacaagccaattaaagtgggcattaggtgagaacatggcaccagagaaagtggactttgaggattgcaaagacgttagtgacctgaaacaatatgatagtgatgaaccagagcccaaatcgctagcaagttggatccagagtgaatttaacaaggcatgtgagttgaccgattcaagctgggtagaacttgatgaaataggagaagatgttgctccaatcgagcacattgcgagtatgagaaggaattacttcacagcagaagtgtcacactgccgggctactgagtatataatgaagggagtgtatattaacacagcgttgctcaatgcatcttgtgcagccatggatgacttccaattgattccaatgataagcaaatgcagaacaaaagaagggagacggaaaacaaacctgtatgggttcattatcaagggaaggtcccatttgaggaatgatactgatgtggtaaactttgtgagcatggaattttctcttacagacccgaaactggaaccacacaagtgggagaagtactgtgttcttgaagtaggggacatgctcctgagaacttcaataggccaggtgtcaaggcccatgttcctatacgtgagaaccaatggaacctccaaaattaaaatgaaatggggaatggagatgaggcgttgcctccttcaatcccttcaacaaattgagagcatgattgaggcagagtcttctatcaaagagaaggacatgaccaaagaattttttgaaaacaagtcggagacgtggccgattggagagtcacctaagggagtggaggaaggctccatcgggaaggtgtgcaggaccttactagcaaagtctgtgttcaacagcttgtatgcatctccacaactcgaggggttttcagctgaatcaagaaaactgttactcattgttcaggcacttagggacaacctggaacctggaaccttcgacattgaaggactgtatgaagcaattgaggagtgcctgattaatgatccctgggttttgcttaatgcatcttggttcaactcc

SEQ ID NO: 134 - DNA, 99 bp, Artificial
  influenza virus type A NP segment
ttcggacgaaaaggcagcgagcccgatcgtgccttcctttgacatgagtaatgaaggatcttatttcttcggagacaatgcagaggagtacgacaatta

SEQ ID NO: 135 - DNA, 100 bp, Artificial
  influenza virus type A NP segment
tctcggacgaaaaggcagcgagcccgatcgtgccctcctttgacatgagtaatgaaggatcttatttcttcggagacaatgcagaggagtacgacaatta

SEQ ID NO: 136 - DNA, 100 bp, Artificial
  influenza virus type A NP segment
tctcggactaaaaggcaacgaaccccatcgtgccctcttttgacatgagtaatgaaggatcttatttcttcggagacaatgcagaggagtacgacaatta

SEQ ID NO: 137 - DNA, 100 bp, Artificial
  influenza virus type A NP segment
tctcggacgaaaaggcagcgaacccgatcgtgccctcttttgacatgagtaatgaaggatcttatttcttcggagacaatgcagaggagtacgacaatta

SEQ ID NO: 138 - DNA, 100 bp, Artificial
  influenza virus type A NP segment
tctcagacgaaaaggcaacgaacccgatcgtgccctcttttgacatgagtaatgaaggatcttatttcttcggagacaatgcagaggagtacgacaatta

SEQ ID NO: 139 - DNA, 100 bp, Artificial
  influenza virus type A NP segment
tctcagacgaaaaggcaacgaacccgatcgtgccttcctttgacatgagtaatgaaggatcttatttcttcggagacaatgcagaggaatatgacaattg

SEQ ID NO: 140 - DNA, 100 bp, Artificial
  influenza virus type A NP segment
tctcggacgaaaaggcgacgaacccgatcgtgccttcctttgacatgagtaacgagggatcttatttcttcggagacaatgcagaggaatatgacaatta

SEQ ID NO: 141 - DNA, 100 bp, Artificial
  influenza virus type A NP segment
tctcggacgaaaaggcaacgaacccgatcgtgccttcctttgacatgagcaatgaagggtcttatttcttcggagacaatgctgaggagtttgacagtta

SEQ ID NO: 142 - DNA, 100 bp, Artificial
  influenza virus type A NP segment
tctcagacgaaaaggcaacgaacccgatcgtgccttcctttgacatgagtaatgagcgatcttatttcttcggagacaatgctgaggagtatgacaattg

SEQ ID NO: 143 - DNA, 100 bp, Artificial
  influenza virus type A NP segment
tctcggacgaaaaggcaacgaacccgatcgtgccttcctttgacatgagtaatgaaggatcttatttcttcggagacaatgcataggagtatgacaatta

SEQ ID NO: 144 - DNA, 100 bp, Influenza virus type A NP segment
tctcggacgaaaaggcaacgaacccgatcgtgccttcctttgacatgagtaacgaagggtcttatttcttcggagacaatgcagaggaatatgacaatta

SEQ ID NO: 145 - DNA, 13 bp, Artificial
  Influenza virus type A NP segment
aguagaaacaagg

SEQ ID NO: 146 - DNA, 12 bp, Artificial
  influenza virus type A vRNA
ccugcuuucgcu

SEQ ID NO: 147 - DNA, 12 bp, Artificial
  influenza virus type a mRNA
agcgaaagcagg

SEQ ID NO: 148 - DNA, 12 bp, Artificial
  influenza virus type A RNA
agcgaaagcagg

SEQ ID NO: 149 - DNA, 13 bp, Artificial
  influenza virus type A RNA
ccuuguuucuacu

SEQ ID NO: 150 - DNA, 21 bp, Artificial
  targeted to CD8 gene human
gcuacaacuacuacaugactt

SEQ ID NO: 151 - DNA, 21 bp, Artificial
  targeted to CD8 gene human
gucauguaguaguuguagctt

SEQ ID NO: 152 - DNA, 62 bp, Artificial
  targeted to CE8 gene human
cgggggugcuacaacuacuacaugacgcagguccagucauguaguaguuguagcuuccccug

SEQ ID NO: 153 - DNA, 21 bp, Artificial
  targeted to influenza virus type A NP segment
ggaucuuauuucuucggagtt

SEQ ID NO: 154 - DNA, 21 bp, Artificial
  targeted to influenza virus type A NP segment
cuccgaagaaauaagaucctt

SEQ ID NO: 155 - DNA, 60 bp, Artificial
  targeted to influenza virus type A NP segment
cgggguggaucuuauuucuucggaggcagguccacuccgaagaaauaagauccuucccug

SEQ ID NO: 156 - DNA, 21 bp, Artificial
  targeted to Aequonia victoria green fluorescent protein
ugcgcuccuggacguagcctt

SEQ ID NO: 157 - DNA, 21 bp, Artificial
  targeted to Aequonia victoria green fluorescent protein
ggcuacguccaggagcgcatt

SEQ ID NO: 158 - DNA, 60 bp, Artificial
  targeted to Aequonia victoria green fluorescent protein
cgggguugcgcuccuggacguagccgcagguccaggcuacguccaggagcgcauucccug

SEQ ID NO: 159 - DNA, 119 bp, Artificial
  construct targeted to influenza virus type A NP segment and green fluorescent protein
cgggguggaucuuauuucuucggaggcagcgggguugcgcuccuggacguagccgcagguccaggcuacguccaggagcgcauucccuguccacuccgaagaaauaagauccuucccug

SEQ ID NO: 160 - DNA, 119 bp, Artificial
  construct targeted to influenza virus type A NP segment and green fluorescent protein
cgggguugcgcuccuggacguagccgcagcgggguggaucuuauuucuucggaggcagguccacuccgaagaaauaagauccuucccuguccaggcuacguccaggagcgcauucccug

SEQ ID NO: 161 - DNA, 24 bp, Artificial
  influenza virus type A M vRNA
cgctcagacatgagaacagaatgg

SEQ ID NO: 162 - DNA, 36 bp, Artificial
  influenza virus type A M cRNA
atatcgtctcgtattagtagaaacaaggtagttttt

SEQ ID NO: 163 - DNA, 24 bp, Artificial
  influenza virus type A M RNA
cgctcagacatgagaacagaatgg

SEQ ID NO: 164 - DNA, 23 bp, Artificial
  influenza virus type A M RNA
taactagcctgactagcaacctc

SEQ ID NO: 165 - DNA, 25 bp, Artificial
  influenza virus type A PB1 vRNA
gtgcagaaatcagcccgaatggttc

SEQ ID NO: 166 - DNA, 33 bp, Artificial
  influenza virus type A PB1 cRNA
atatcgtctcgtattagtagaaacaaggcattt

SEQ ID NO: 167 - DNA, 24 bp, Artificial
  influenza virus type A PB2 vRNA
gcgaaaggagagaaggctaatgtg

SEQ ID NO: 168 - DNA, 34 bp, Artificial
  influenza virus type A PB2 cRNA
atatggtctcgtattagtagaaacaaggtcgttt

SEQ ID NO: 169 - DNA, 25 bp, Artificial
  influenza virus type A PA vRNA
gcttcttatcgttcaggctcttagg

SEQ ID NO: 170 - DNA, 33 bp, Artificial
  influenza virus type A PA cRNA
atatcgtctcgtattagtagaaacaaggtactt

SEQ ID NO: 171 - DNA, 24 bp, Artificial
  influenza virus type A PB 1 RNA
cggattgatgcacggattgatttc

SEQ ID NO: 172 - DNA, 27 bp, Artificial
  influenza virus type A PB 1 RNA
gacgtctgagctcttcaatggtggaac

SEQ ID NO: 173 - DNA, 24 bp, Artificial
  influenza virus type A PB2 RNA
gcgaaaggagagaaggctaatgtg

SEQ ID NO: 174 - DNA, 24 bp, Artificial
  influenza virus type A PB2 RNA
aatcgctgtctggctgtcagtaag

SEQ ID NO: 175 - DNA, 25 bp, Artificial
  influenza virus type A PA RNA
gcttcttatcgttcaggctcttagg

SEQ ID NO: 176 - DNA, 25 bp, Artificial
  influenza virus type A PA RNA
ccgagaagcattaagcaaaacccag

SEQ ID NO: 177 - DNA, 20 bp, Artificial
  gamma action
tctgtcagggttggaaagtc

SEQ ID NO: 178 - DNA, 20 bp, Artificial
  gamma action
aaatgcaaaccgcttccaac

SEQ ID NO: 179 - DNA, 57 bp, Artificial
  oligenucleotide for plasmid construction
tggatcttatttcttcggagattcaagagatctccgaagaaataagatccttttttc

SEQ ID NO: 180 - DNA, 61 bp, Artificial
  oligenucleotide for plasmid construction
tcgagaaaaaaggatcttatttcttcggagatctcttgaatctccgaagaaataagatcca

SEQ ID NO: 181 - DNA, 55 bp, Artificial
  Oligenucleotide for plasmid construction
tgatctgttccaccattgaattcaagagattcaatggtggaacagatcttttttc

SEQ ID NO: 182 - DNA, 59 bp, Artificial
  Oligenucleotide for plasmid construction
tcgagaaaaaagatctgttccaccattgaatctcttgaattcaatggtggaacagatca

SEQ ID NO: 183 - DNA, 56 bp, Artificial
  Oligenucleotide for plasmid construction
tgcgataatataactgcaagattcaagagatcttgcagttatattatcgttttttc

SEQ ID NO: 184 - DNA, 60 bp, Artificial
  Oligenucleotide for plasmid construction
tcgagaaaaaacgataatataactgcaagatctcttgaatcttgcagttatattatcgca

SEQ ID NO: 185 - DNA, 19 bp, Artificial
  respiratory syncitial virus siRNA
cgataatataactgcaaga

SEQ ID NO: 186 - DNA, 19 bp, Artificial
  respiratory syncitial virus siRNA
tcttgcagttatattatcg

SEQ ID NO: 187 - DNA, 55 bp, Artificial
  Oligenucleotide for plasmid construction
tgcaattgaggagtgcctgattcaagagatcaggcactcctcaattgcttttttc

SEQ ID NO: 188 - DNA, 22 bp, Artificial
  targeted to infuenza virus type A NP segment
ggaucuuauuucuucggagatt

SEQ ID NO: 189 - DNA, 22 bp, Artificial
  targeted to influenza virus type A NP segment
ucuccgaagaaauaagaucctt

SEQ ID NO: 190 - DNA, 21 bp, Artificial
  targeted to influenza virus type A PB2 segment
gaaagcaggucaauuauautt

SEQ ID NO: 191 - DNA, 21 bp, Artificial
  targeted to influenza virus type A PB2 segment
auauaauugaccugcuuuctt

SEQ ID NO: 192 - DNA, 21 bp, Artificial
  targeted to influenza virus type A PB2 segment
gucaauuauauucaauaugtt

SEQ ID NO: 193 - DNA, 21 bp, Artificial
  targeted to influenza virus type A PB2 segment
cauauugaauauaauugactt

SEQ ID NO: 194 - DNA, 21 bp, Artificial
  targeted to influenza virus type A PB2 segment
cucgcacccgcgagauacutt

SEQ ID NO: 195 - DNA, 21 bp, Artificial
  targeted to influenza virus type A PB2 segment
aguaucucgcgggugcgagtt

SEQ ID NO: 196 - DNA, 21 bp, Artificial
  targeted to influenza virus type A PB2 segment
auaaucaagaaguacacautt

SEQ ID NO: 197 - DNA, 23 bp, Artificial
  targeted to influenza virus type A PB2 segment
auguguacuucuugauuaudtdt

SEQ ID NO: 198 - DNA, 21 bp, Artificial
  targeted to influenza virus type A PB 2 segment
ugaaauggaugauggcaautt

SEQ ID NO: 199 - DNA, 21 bp, Artificial
  targeted to influenza virus type A PB2 segment
auugccaucauccauuucatt

SEQ ID NO: 200 - DNA, 21 bp, Artificial
  targeted to influenza virus type A PB2 segment
cuggucaugcagaucucagtt

SEQ ID NO: 201 - DNA, 21 bp, Artificial
  targeted to influenza virus type A PB2 segment
cugagaucugcaugaccagtt

SEQ ID NO: 202 - DNA, 21 bp, Artificial
  targeted to influenza virus type A PB2 segment
uaugcaaggcugcaaugggtt

SEQ ID NO: 203 - DNA, 23 bp, Artificial
  targeted to influenza virus type A PB2 segment
cccauugcagccuugcauadtdt

SEQ ID NO: 204 - DNA, 21 bp, Artificial
  targeted to influenza virus type A PB2 segment
caucgucaaugaugugggatt

SEQ ID NO: 205 - DNA, 21 bp, Artificial
  targeted to influenza virus type A PB2 segment
ucccacaucauugacgaugtt

SEQ ID NO: 206 - DNA, 21 bp, Artificial
  influenza virus type A PB1 segment
aaauaccugcagaaaugcutt

SEQ ID NO: 207 - DNA, 23 bp, Artificial
  influenza virus type A PB1 segment
agcauuucugcagguauuudtdt

SEQ ID NO: 208 - DNA, 21 bp, Artificial
  influenza virus type A PB1 segment
aacaauaugauaaacaaugtt

SEQ ID NO: 209 - DNA, 23 bp, Artificial
  influenza virus type A PB1 segment
cauuguuuaucauauuguudtdt

SEQ ID NO: 210 - DNA, 23 bp, Artificial
  influenza virus type A PA segment
cgaaagcagguacugauccdtdt

SEQ ID NO: 211 - DNA, 21 bp, Artificial
  influenza virus typ A PA segment
ggaucaguaccugcuuucgtt

SEQ ID NO: 212 - DNA, 23 bp, Artificial
  influenza virus type A PA segment
aggcuauucaccauaagacdtdt

SEQ ID NO: 213 - DNA, 21 bp, Artificial
  influenza virus type A PA segment
gucuuauggugaauagccutt

SEQ ID NO: 214 - DNA, 23 bp, Artificial
  influenza virus type A PA segment
gggauuccuuucgucagucdtdt

SEQ ID NO: 215 - DNA, 21 bp, Artificial
  influenza virus type A PA segment
gacugacgaaaggaauccctt

SEQ ID NO: 216 - DNA, 21 bp, Artificial
  influenza virus type A PA segment
gcaucuugugcagcaauggtt

SEQ ID NO: 217 - DNA, 21 bp, Artificial
  influenza virus type A PA segment
ccauugcugcacaagaugctt

SEQ ID NO: 218 - DNA, 21 bp, Artificial
  influenza virus type A PA segment
guuguggcagugcuacuautt

SEQ ID NO: 219 - DNA, 21 bp, Artificial
  influenza virus type A PA segment
auaguagcacugccacaactt

SEQ ID NO: 220 - DNA, 21 bp, Artificial
  influenza virus type A PA segment
uacuauuugcuauccauactt

SEQ ID NO: 221 - DNA, 21 bp, Artificial
  influenza virus type A PA segment
guauggauagcaaauaguatt

SEQ ID NO: 222 - DNA, 21 bp, Artificial
  influenza virus type A NP segment
uagauaaucacucacugagtt

SEQ ID NO: 223 - DNA, 23 bp, Artificial
  influenza virus type A NP segment
cucagugagugauuaucuadtdt

SEQ ID NO: 224 - DNA, 21 bp, Artificial
  influenza virus type A NP segment
cgucccaaggcaccaaacgtt

SEQ ID NO: 225 - DNA, 21 bp, Artificial
  influenza virus type A NP segment
cguuuggugccuugggacgtt

SEQ ID NO: 226 - DNA, 23 bp, Artificial
  influenza virus type A NP segment
auuucuucggagacaaugcdtdt

SEQ ID NO: 227 - DNA, 21 bp, Artificial
  influenza virus type A NP segment
gcauugucuccgaagaaautt

SEQ ID NO: 228 - DNA, 23 bp, Artificial
  influenza virus type A NP segment
ugcagaggaguacgacaaudtdt

SEQ ID NO: 229 - DNA, 23 bp, Artificial
  influenza virus type A NP segment
auugucguacuccucugcadtdt

SEQ ID NO: 230 - DNA, 21 bp, Artificial
  influenza virus type A NP segment
gagtaatgaaggatcttattt

SEQ ID NO: 231 - DNA, 23 bp, Artificial
  influenza virus type A NP segment
ataagatccttcattactcdtdt

SEQ ID NO: 232 - DNA, 21 bp, Artificial
  influenza virus type A M segment
cgaaagcagguagauauugtt

SEQ ID NO: 233 - DNA, 21 bp, Artificial
  influenza virus type A M segment
caauaucuaccugcuuucgtt

SEQ ID NO: 234 - DNA, 23 bp, Artificial
  influenza virus type A M segment
uagauauugaaagaugagudtdt

SEQ ID NO: 235 - DNA, 23 bp, Artificial
  influenza virus type A M segment
acucaucuuucaauaucuadtdt

SEQ ID NO: 236 - DNA, 21 bp, Artificial
  influenza virus type A M segment
ucauggaauggcuaaagactt

SEQ ID NO: 237 - DNA, 21 bp, Artificial
  influenza virus type A M segment
gucuuuagccauuccaugatt

SEQ ID NO: 238 - DNA, 21 bp, Artificial
  influenza virus type A M segment
accaauccugucaccucugtt

SEQ ID NO: 239 - DNA, 23 bp, Artificial
  influenza virus type A M segment
cagaggugacaggauuggudtdt

SEQ ID NO: 240 - DNA, 23 bp, Artificial
  influenza virus type A M segment
uguguucacgcucaccgugdtdt

SEQ ID NO: 241 - DNA, 21 bp, Artificial
  influenza virus type A M segment
cacggugagcgugaacacatt

SEQ ID NO: 242 - DNA, 21 bp, Artificial
  influenza virus type A M segment
cagugagcgaggacugcagtt

SEQ ID NO: 243 - DNA, 21 bp, Artificial
  influenza virus type A M segment
cugcaguccucgcucacugtt

SEQ ID NO: 244 - DNA, 23 bp, Artificial
  influenza virus type A M segment
gacgcuuuguccaaaaugcdtdt

SEQ ID NO: 245 - DNA, 21 bp, Artificial
  influenza virus type A M segment
gcauuuuggacaaagcguctt

SEQ ID NO: 246 - DNA, 21 bp, Artificial
  influenza virus type A M segment
gucaggcuaggcaaauggutt

SEQ ID NO: 247 - DNA, 23 bp, Artificial
  influenza virus type A M segment
accauuugccuagccugacdtdt

SEQ ID NO: 248 - DNA, 23 bp, Artificial
  influenza virus type A M segment
uucuugaaaauuugcaggcdtdt

SEQ ID NO: 249 - DNA, 23 bp, Artificial
  influenza virus type A M segment
gccugcaaauuuucaagaadtdt

SEQ ID NO: 250 - DNA, 23 bp, Artificial
  influenza virus type A M segment
ucauugggaucuugcacuudtdt

SEQ ID NO: 251 - DNA, 21 bp, Artificial
  influenza virus type A M segment
aagugcaagaucccaaugatt

SEQ ID NO: 252 - DNA, 23 bp, Artificial
  targeted to influenza virus type A M segment
uguggauucuugaucgucudtdt

SEQ ID NO: 253 - DNA, 21 bp, Artificial
  targeted to influenza virus type A M segment
agacgaucaagaauccacatt

SEQ ID NO: 254 - DNA, 21 bp, Artificial
  targeted to influenza virus type A M segment
ugucagcauagagcuggagtt

SEQ ID NO: 255 - DNA, 21 bp, Artificial
  targeted to influenza virus type A M segment
cuccagcucuaugcugacatt

SEQ ID NO: 256 - DNA, 23 bp, Artificial
  targeted to influenza virus type A M segment
gtcgaaacgcctatcagaadtdt

SEQ ID NO: 257 - DNA, 21 bp, Artificial
  targeted to influenza virus type A M segment
uucugauaggcguuucgactt

SEQ ID NO: 258 - DNA, 21 bp, Artificial
  targeted to influenza virus type A NS segment
aaaagcagggugacaaagatt

SEQ ID NO: 259 - DNA, 21 bp, Artificial
  targeted to influenza virus type A NS segment
ucuuugucacccugcuuuutt

SEQ ID NO: 260 - DNA, 23 bp, Artificial
  targeted to influenza virus type A NS segment
gcagggugacaaagacauadtdt

SEQ ID NO: 261 - DNA, 23 bp, Artificial
  targeted to influenza virus type NS segment
uaugucuuugucacccugcdtdt

SEQ ID NO: 262 - DNA, 21 bp, Artificial
  targeted to influenza virus type A NS segment
ggaugucaaaaaugcaguutt

SEQ ID NO: 263 - DNA, 21 bp, Artificial
  targeted to influenza virus type A NS segment
aacugcauuuuugacaucctt

SEQ ID NO: 264 - DNA, 21 bp, Artificial
  targeted to influenza virus type A NS segment
agagauucgcuuggagaagtt

SEQ ID NO: 265 - DNA, 21 bp, Artificial
  targeted to influenza virus type A NS segment
cuucuccaagcgaaucucutt

SEQ ID NO: 266 - DNA, 21 bp, Artificial
  targeted to influenza virus type A NS segment
caguaaugagaaugggagatt

SEQ ID NO: 267 - DNA, 21 bp, Artificial
  targeted to influenza virus type A NS segment
ucucccauucucauuacugtt

SEQ ID NO: 268 - DNA, 21 bp, Artificial
  targeted to influenza NS segment
uuguggauucuugaucguctt

SEQ ID NO: 269 - DNA, 21 bp, Artificial
  targeted to influenza virus type A NS segment
gacgaucaagaauccacaatt

SEQ ID NO: 270 - DNA, 59 bp, Artificial
  Oligenucleotide for plasmid construction
tcgagaaaaaagcaattgaggagtgcctgatctcttgaatcaggcactcctcaattgca

SEQ ID NO: 271 - DNA, 51 bp, Artificial
  targeted to influenza virus type A NP segment
ggaucuuauuucuucggagauucaagagaucuccgaagaaauaagauccuu